SEQUENCE LISTING (247 sequences)

SEQ ID NO: 1 - protein, 12 aa, Homo sapiens
ESKYGPPCPPCP

SEQ ID NO: 2 - DNA, 36 bp, Homo sapiens
  source 1..36: mol_type = unassigned DNA
gaatctaagtacggaccgccctgccccccttgccct

SEQ ID NO: 3 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
LEGGGEGRGSLLTCGDVEENPGPR

SEQ ID NO: 7 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
FWVLVVVGGVLACYSLLVTVAFIIFWV

SEQ ID NO: 9 - protein, 66 aa, Homo sapiens
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
RSKRSRLLHSDYMNMTPRRPGPTRKHYQPYAPPRDFAAYRS

SEQ ID NO: 11 - protein, 41 aa, Homo sapiens
RSKRSRGGHSDYMNMTPRRPGPTRKHYQPYAPPRDFAAYRS

SEQ ID NO: 12 - protein, 42 aa, Homo sapiens
KRGRKKLLYIFKQPFMRPVQTTQEEDGCSCRFPEEEEGGCEL

SEQ ID NO: 13 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 30 aa, synthetic construct
PGGGSGGGGSGGGGSGGGGSGGGGSGGGGP

SEQ ID NO: 17 - protein, 17 aa, synthetic construct
GSADDAKKDAAKKDGKS

SEQ ID NO: 18 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 19 - protein, 111 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 20 - protein, 122 aa, synthetic construct
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 21 - protein, 107 aa, synthetic construct
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 22 - protein, 116 aa, synthetic construct
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 23 - protein, 111 aa, synthetic construct
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 24 - protein, 118 aa, synthetic construct
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 25 - protein, 105 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 26 - protein, 5 aa, synthetic construct
GGGGS

SEQ ID NO: 27 - protein, 4 aa, synthetic construct
GGGS

SEQ ID NO: 28 - protein, 15 aa, synthetic construct
GGGGSGGGGSGGGGS

SEQ ID NO: 29 - protein, 18 aa, synthetic construct
GSTSGSGKPGSGEGSTKG

SEQ ID NO: 30 - protein, 21 aa, synthetic construct
SRGGGGSGGGGSGGGGSLEMA

SEQ ID NO: 31 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 122 aa, synthetic construct
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 33 - protein, 111 aa, synthetic construct
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 34 - protein, 120 aa, synthetic construct
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 35 - protein, 112 aa, synthetic construct
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 36 - protein, 118 aa, synthetic construct
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 37 - protein, 112 aa, synthetic construct
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 38 - protein, 17 aa, synthetic construct
ASGGGGSGGRASGGGGS

SEQ ID NO: 39 - protein, 21 aa, synthetic construct
MALPVTALLLPLALLLHAARP

SEQ ID NO: 40 - protein, 20 aa, synthetic construct
METDTLLLWVLLLWVPGSTG

SEQ ID NO: 41 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 42 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 43 - protein, 122 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 44 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 45 - protein, 116 aa, synthetic construct
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 46 - protein, 106 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 47 - protein, 122 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 48 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 49 - protein, 119 aa, synthetic construct
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 50 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 51 - protein, 122 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 52 - protein, 107 aa, synthetic construct
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 53 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 54 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 55 - protein, 123 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 56 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 57 - protein, 121 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 58 - protein, 112 aa, synthetic construct
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 59 - protein, 117 aa, synthetic construct
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 60 - protein, 111 aa, synthetic construct
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 61 - protein, 115 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 62 - protein, 107 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 63 - protein, 120 aa, synthetic construct
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 64 - protein, 109 aa, synthetic construct
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 65 - protein, 115 aa, synthetic construct
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 66 - protein, 107 aa, synthetic construct
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 67 - protein, 115 aa, synthetic construct; identical to SEQ ID NO: 61

SEQ ID NO: 68 - protein, 109 aa, synthetic construct
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 69 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 70 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 71 - protein, 117 aa, synthetic construct
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 72 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 73 - protein, 119 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 74 - protein, 112 aa, synthetic construct
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 75 - protein, 119 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 76 - protein, 112 aa, synthetic construct
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 77 - protein, 122 aa, synthetic construct
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 78 - protein, 39 aa, synthetic construct
IEVMYPPPYLDNEKSNGTIIHVKGKHLCPSPLFPGPSKP

SEQ ID NO: 79 - protein, 25 aa, synthetic construct
IYIWAPLAGTCGVLLLSLVITLYCN

SEQ ID NO: 80 - protein, 30 aa, synthetic construct
LDNEKSNGTIIHVKGKHLCPSPLFPGPSKP

SEQ ID NO: 81 - protein, 46 aa, synthetic construct
PTTTPAPRPPTPAPTIASQPLSLRPEACRPAAGGAVHTRGLDFACD

SEQ ID NO: 82 - protein, 45 aa, synthetic construct
TTTPAPRPPTPAPTIASQPLSLRPEACRPAAGGAVHTRGLDFACD

SEQ ID NO: 83 - protein, 55 aa, synthetic construct
FVPVFLPAKPTTTPAPRPPTPAPTIASQPLSLRPEACRPAAGGAVHTRGLDFACD

SEQ ID NO: 84 - protein, 39 aa, synthetic construct
DTGLYICKVELMYPPPYYLGIGNGTQIYVIDPEPCPDSD

SEQ ID NO: 85 - protein, 24 aa, synthetic construct
FLLWILAAVSSGLFFYSFLLTAVS

SEQ ID NO: 86 - protein, 38 aa, synthetic construct
QIKESLRAELRVTERRAEVPTAHPSPSPRPAGQFQTLV

SEQ ID NO: 87 - protein, 21 aa, synthetic construct
VGVVGGLLGSLVLLVWVLAVI

SEQ ID NO: 88 - protein, 16 aa, synthetic construct
GLAVSTISSFFPPGYQ

SEQ ID NO: 89 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 457 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 457 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 466 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 466 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 454 aa, synthetic construct
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 454 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 461 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 461 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 458 aa, synthetic construct
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 458 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 461 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 461 aa, synthetic construct
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 471 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 471 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 462 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 462 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 466 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 473 aa, synthetic construct
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 470 aa, synthetic construct
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 476 aa, synthetic construct
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 475 aa, synthetic construct
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 482 aa, synthetic construct
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 476 aa, synthetic construct
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 481 aa, synthetic construct
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 479 aa, synthetic construct
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 472 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 472 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 472 aa, synthetic construct
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 466 aa, synthetic construct
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 464 aa, synthetic construct
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 653 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 650 aa, synthetic construct
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 651 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 658 aa, synthetic construct
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 654 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 653 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 462 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 469 aa, synthetic construct
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 462 aa, synthetic construct
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 464 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 467 aa, synthetic construct
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 472 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 466 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 466 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 466 aa, synthetic construct
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 482 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 440 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 469 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 655 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 440 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 469 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 655 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 28 aa, synthetic construct
IYIWAPLAGTCGVLLLSLVITLYCNHRN

SEQ ID NO: 143 - protein, 21 aa, synthetic construct
IYIWAPLAGTCGVLLLSLVIT

SEQ ID NO: 144 - protein, 4 aa, synthetic construct
RAAA

SEQ ID NO: 145 - protein, 118 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 146 - protein, 110 aa, synthetic construct
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 147 - protein, 118 aa, synthetic construct
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 148 - protein, 107 aa, synthetic construct
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 149 - protein, 118 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 150 - protein, 108 aa, synthetic construct
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 151 - protein, 122 aa, synthetic construct; identical to SEQ ID NO: 32

SEQ ID NO: 152 - protein, 111 aa, synthetic construct; identical to SEQ ID NO: 33

SEQ ID NO: 153 - protein, 16 aa, synthetic construct
MPLLLLLPLLWAGALA

SEQ ID NO: 154 - protein, 18 aa, synthetic construct
MALPVTALLLPLALLLHA

SEQ ID NO: 155 - DNA, 66 bp, synthetic construct
  source 1..66: mol_type = other DNA
atgcttctcctggtgacaagccttctgctctgtgagttaccacacccagcattcctcctgatccca

SEQ ID NO: 156 - protein, 22 aa, synthetic construct
MLLLVTSLLLCELPHPAFLLIP

SEQ ID NO: 157 - protein, 14 aa, synthetic construct
EVVVKYGPPCPPCP

SEQ ID NO: 158 - skipped

SEQ ID NO: 159 - protein, 15 aa, synthetic construct
EPKSCDKTHTCPPCP

SEQ ID NO: 160 - protein, 12 aa, synthetic construct
ERKCCVECPPCP

SEQ ID NO: 161 - protein, 61 aa, synthetic construct
ELKTPLGDTHTCPRCPEPKSCDTPPPCPRCPEPKSCDTPPPCPRCPEPKSCDTPPPCPRCP

SEQ ID NO: 162 - protein, 12 aa, synthetic construct
ESKYGPPCPSCP

SEQ ID NO: 163 - protein, 12 aa, synthetic construct
ESKYGPPCPPCP

SEQ ID NO: 164 - protein, 9 aa, synthetic construct
YGPPCPPCP

SEQ ID NO: 165 - protein, 10 aa, synthetic construct
KYGPPCPPCP

SEQ ID NO: 166 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 167 - protein, 18 aa, synthetic construct
EGRGSLLTCGDVEENPGP

SEQ ID NO: 168 - protein, 22 aa, synthetic construct
GSGATNFSLLKQAGDVEENPGP

SEQ ID NO: 169 - protein, 19 aa, synthetic construct
ATNFSLLKQAGDVEENPGP

SEQ ID NO: 170 - protein, 20 aa, synthetic construct
QCTNYALLKLAGDVESNPGP

SEQ ID NO: 171 - protein, 22 aa, synthetic construct
VKQTLNFDLLKLAGDVESNPGP

SEQ ID NO: 172 - protein, 290 aa, synthetic construct
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 5 aa, synthetic construct
DYYVY

SEQ ID NO: 174 - protein, 17 aa, synthetic construct
WINPNSGGTNYAQKFQG

SEQ ID NO: 175 - protein, 9 aa, synthetic construct
SQRDGYMDY

SEQ ID NO: 176 - protein, 7 aa, synthetic construct
GYTFIDY

SEQ ID NO: 177 - protein, 6 aa, synthetic construct
NPNSGG

SEQ ID NO: 178 - protein, 10 aa, synthetic construct
GYTFIDYYVY

SEQ ID NO: 179 - protein, 10 aa, synthetic construct
WINPNSGGTN

SEQ ID NO: 180 - protein, 8 aa, synthetic construct
GYTFIDYY

SEQ ID NO: 181 - protein, 8 aa, synthetic construct
INPNSGGT

SEQ ID NO: 182 - protein, 11 aa, synthetic construct
ARSQRDGYMDY

SEQ ID NO: 183 - protein, 8 aa, synthetic construct
TGTSSDVG

SEQ ID NO: 184 - protein, 7 aa, synthetic construct
EDSKRPS

SEQ ID NO: 185 - protein, 10 aa, synthetic construct
SSNTRSSTLV

SEQ ID NO: 186 - protein, 9 aa, synthetic construct
ISCTGTSSD

SEQ ID NO: 187 - skipped

SEQ ID NO: 188 - protein, 244 aa, synthetic construct
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 189 - protein, 5 aa, synthetic construct
DYSIN

SEQ ID NO: 190 - protein, 17 aa, synthetic construct
WINTETREPAYAYDFRG

SEQ ID NO: 191 - protein, 8 aa, synthetic construct
DYSYAMDY

SEQ ID NO: 192 - protein, 15 aa, synthetic construct
RASESVTILGSHLIH

SEQ ID NO: 193 - protein, 7 aa, synthetic construct
LASNVQT

SEQ ID NO: 194 - protein, 9 aa, synthetic construct
LQSRTIPRT

SEQ ID NO: 195 - protein, 7 aa, synthetic construct
GYTFTDY

SEQ ID NO: 196 - protein, 6 aa, synthetic construct
NTETRE

SEQ ID NO: 197 - protein, 8 aa, synthetic construct
DYSYAMDY

SEQ ID NO: 198 - protein, 15 aa, synthetic construct
RASESVTILGSHLIH

SEQ ID NO: 199 - protein, 7 aa, synthetic construct
LASNVQT

SEQ ID NO: 200 - protein, 9 aa, synthetic construct
LQSRTIPRT

SEQ ID NO: 201 - protein, 10 aa, synthetic construct
GYTFTDYSIN

SEQ ID NO: 202 - protein, 10 aa, synthetic construct
WINTETREPA

SEQ ID NO: 203 - protein, 8 aa, synthetic construct
DYSYAMDY

SEQ ID NO: 204 - protein, 15 aa, synthetic construct
RASESVTILGSHLIH

SEQ ID NO: 205 - protein, 7 aa, synthetic construct
LASNVQT

SEQ ID NO: 206 - protein, 9 aa, synthetic construct
LQSRTIPRT

SEQ ID NO: 207 - protein, 8 aa, synthetic construct
GYTFTDYS

SEQ ID NO: 208 - protein, 8 aa, synthetic construct
INTETREP

SEQ ID NO: 209 - protein, 10 aa, synthetic construct
ALDYSYAMDY

SEQ ID NO: 210 - protein, 10 aa, synthetic construct
ESVTILGSHL

SEQ ID NO: 211 - skipped

SEQ ID NO: 212 - protein, 9 aa, synthetic construct
LQSRTIPRT

SEQ ID NO: 213 - protein, 246 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 214 - DNA, 1485 bp, synthetic construct
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 215 - protein, 184 aa, synthetic construct
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 216 - protein, 243 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 217 - protein, 244 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 218 - protein, 252 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 219 - protein, 253 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 220 - protein, 240 aa, synthetic construct
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 221 - protein, 241 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 222 - protein, 247 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 223 - protein, 248 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 224 - protein, 244 aa, synthetic construct
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 225 - protein, 245 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 226 - protein, 247 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 227 - protein, 248 aa, synthetic construct
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 228 - protein, 257 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 229 - protein, 258 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 230 - protein, 248 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 231 - protein, 249 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 232 - protein, 246 aa, synthetic construct
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 233 - protein, 246 aa, synthetic construct; identical to SEQ ID NO: 232

SEQ ID NO: 234 - protein, 246 aa, synthetic construct; identical to SEQ ID NO: 232

SEQ ID NO: 235 - protein, 239 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 236 - protein, 246 aa, synthetic construct
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 237 - protein, 239 aa, synthetic construct
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 238 - protein, 241 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 239 - protein, 243 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 240 - protein, 243 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 241 - protein, 243 aa, synthetic construct
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 242 - protein, 246 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 243 - protein, 246 aa, synthetic construct; identical to SEQ ID NO: 242

SEQ ID NO: 244 - protein, 246 aa, synthetic construct; identical to SEQ ID NO: 242

SEQ ID NO: 245 - protein, 246 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 246 - protein, 246 aa, synthetic construct; identical to SEQ ID NO: 245

SEQ ID NO: 247 - protein, 246 aa, synthetic construct; identical to SEQ ID NO: 245

(19) United States
(12) Patent Application Publication
(10) Pub. No.: US 2026/0166082 A1
(43) Pub. Date: Jun. 18, 2026

(54) METHODS OF TREATMENT WITH T CELL THERAPY AND IMMUNOMODULATORY AGENT MAINTENANCE THERAPY

(71) Applicant: Celgene Corporation, Summit, NJ (US)

(72) Inventors: Mihaela Alina POPA MCKIVER, Lawrenceville, NJ (US); Julie Ann RYTLEWSKI, Seattle, WA (US); Nathan Thomas MARTIN, Seattle, WA (US); Kristen Mae HEGE, Burlingame, CA (US); Michael David AMATANGELO, Summit, NJ (US); Debashree BASUDHAR, Seattle, WA (US); Merav BAR, Madison, NJ (US); Lars STERNAS, Verona, NJ (US)

(73) Assignee: Celgene Corporation, Summit, NJ (US)

(21) Appl. No.: 19/126,580

(22) PCT Filed: Nov. 2, 2023
(86) PCT No.: PCT/US2023/078557

§ 371 Date: May 1, 2025

Related U.S. Application Data
(60) Provisional application No. 63/421,997, filed on Nov. 2, 2022.
(60) Provisional application No. 63/431,613, filed on Dec. 9, 2022.
(60) Provisional application No. 63/471,681, filed on Jun. 7, 2023.
(60) Provisional application No. 63/538,057, filed on Sep. 12, 2023.

(51) Int. Cl. A61K 35/17; A61K 31/454; A61K 31/573; A61K 31/69; A61K 35/28; A61K 38/07; A61K 40/11; A61K 40/31; A61K 40/42; A61K 45/06; A61P 35/00; C07K 16/28

(52) U.S. Cl. CPC A61K 35/17; A61K 31/454; A61K 31/573; A61K 31/69; A61K 35/28; A61K 38/07; A61K 40/11; A61K 40/31; A61K 40/4215; A61K 45/06; A61P 35/00; C07K 16/2878; A61K 2239/13; A61K 2239/38; A61K 2239/48

(57) ABSTRACT

Provided herein are adoptive cell therapy methods involving the administration of genetically engineered cells followed by an immunomodulatory agent maintenance therapy for treating disease and conditions, including certain plasma cell malignancy. The cells generally express recombinant receptors such as chimeric antigen receptors (CARs) specific to B-cell maturation antigen (BCMA). In some embodiments, the methods are for treating subjects with multiple myeloma (MM), such as high risk multiple myeloma or newly diagnosed multiple myeloma (NDMM). In some embodiments, the methods are for treating subjects who experienced an early relapse, an inadequate response or a suboptimal response after frontline autologous stem cell transplant therapy (ASCT).

### CROSS-REFERENCE TO RELATED APPLICATIONS

This application claims priority from U.S. provisional application No. 63/421,997, filed Nov. 2, 2022; 63/431,613, filed Dec. 9, 2022; 63/471,681, filed Jun. 7, 2023; 63/538,057, filed Sep. 12, 2023, all entitled “METHODS OF TREATMENT WITH T CELL THERAPY AND IMMUNOMODULATORY AGENT MAINTENANCE THERAPY,” the contents of which are incorporated by reference in their entirety.

### INCORPORATION BY REFERENCE OF SEQUENCE LISTING

The present application is being filed with a Sequence Listing in electronic format. The Sequence Listing is provided as a file entitled 683772002840seqlist.xml, created on Nov. 1, 2023, which is 257,543 bytes in size. The information in electronic format of the Sequence Listing is incorporated by reference in its entirety.

### FIELD

The present disclosure relates in some aspects to methods, uses, and compositions of T cell therapies for treating subjects with a cancer, including patients who had an early relapse, inadequate response, or a suboptimal response to a frontline therapy. In some embodiments, the cancer is a B cell malignancy, such as multiple myeloma. In some embodiments, the subject may have newly diagnosed multiple myeloma (NDMM) or high risk multiple myeloma. The frontline therapy can be a stem cell transplant therapy. The T cell therapy includes cells that express recombinant receptors such as chimeric antigen receptors (CARs).

### BACKGROUND

Various strategies are available for treating cancers, including stem cell transplant therapy. In some cases, a subject has an early relapse, an inadequate response, or a suboptimal response after a stem cell transplant therapy. Improved strategies are needed to mitigate early relapses, inadequate responses, or suboptimal responses to the disease. Provided are methods, uses, and compositions that meet such needs.

### SUMMARY

Provided herein is a method of treating multiple myeloma in a subject who had an early relapse, an inadequate response, or a suboptimal response to one or more anti-myeloma treatment, including administering to the subject a BCMA targeted CAR T cell therapy followed by an immunomodulatory agent maintenance therapy.

Provided herein is a method of treating multiple myeloma in a subject, the method including: (a) selecting a subject who had an early relapse, an inadequate response, or a suboptimal response to one or more anti-myeloma treatment; (b) administering to the selected subject a BCMA targeted CAR T cell therapy; and (c) after administering the BCMA targeted CAR T cell therapy, administering to the subject an immunomodulatory agent maintenance therapy.

Provided herein is a method of treating newly diagnosed multiple myeloma (NDMM) in a subject who had an early relapse, an inadequate response, or a suboptimal response to one or more anti-myeloma treatment, including administering to the subject a BCMA targeted CAR T cell therapy followed by an immunomodulatory agent maintenance therapy.

Provided herein is a method of treating newly diagnosed multiple myeloma (NDMM) in a subject, the method including: (a) selecting a subject who had an early relapse, an inadequate response, or a suboptimal response to one or more anti-myeloma treatment; (b) administering to the selected subject a BCMA targeted CAR T cell therapy; and (c) after administering the BCMA targeted CAR T cell therapy, administering to the subject an immunomodulatory agent maintenance therapy.

Provided herein is a method of maintenance therapy for treating multiple myeloma, the method including administering an immunomodulatory agent maintenance therapy to a subject after having received a BCMA targeted CAR T cell therapy for treating multiple myeloma, wherein prior to the BCMA target CAR T cell therapy the subject had an early relapse, an inadequate response, or a suboptimal response to one or more other anti-myeloma treatment.

In some of any embodiments, the immunomodulatory agent is a maintenance therapy with lenalidomide or a pharmaceutically acceptable salt. In some of any embodiments, the immunomodulatory agent is a maintenance therapy with lenalidomide or hydrate. In particular embodiments, the hydrate is a hemi-hydrate. In some of any embodiments, the immunomodulatory agent is a maintenance therapy with lenalidomide or a polymorph. In particular embodiments, the polymorph is a Form B polymorph. In some of any embodiments, the immunomodulatory agent is a maintenance therapy with lenalidomide or a generic thereof. In particular embodiments, the immunomodulatory agent is generic of lenalidomide. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide. In some of any embodiments, the immunomodulatory agent maintenance therapy is a maintenance therapy with lenalidomide or a pharmaceutically acceptable salt thereof. In some of any embodiments, the immunomodulatory agent maintenance therapy is a maintenance therapy with lenalidomide.

In some of any embodiments, the one or more anti-myeloma treatment includes stem cell transplant therapy. In some embodiments, the stem cell transplant therapy includes autologous stem cell therapy (ASCT).

In some of any embodiments, the stem cell transplant therapy includes an induction therapy followed by a stem cell transplant. In some of any embodiments, the stem cell transplant therapy consists of an induction therapy followed by a stem cell transplant. In some of any embodiments, stem cell transplant therapy includes an induction therapy followed by high-dose chemotherapy (HDT) and a stem cell transplant.

In some of any embodiments, the multiple myeloma is a high-risk multiple myeloma.

In some of any embodiments, the subject is a subject that had an inadequate response to one or more anti-myeloma treatment.

In some of any embodiments, the inadequate response to the one or more anti-myeloma treatment is characterized by less than very good partial response (VGPR), e.g., at 70-110 days, after last treatment of the one or more anti-myeloma treatment without use of consolidation or maintenance therapy. In some of any embodiments, the inadequate response to the one or more anti-myeloma treatment is characterized by less than very good partial response (VGPR), e.g., at 80-120 days, after last treatment of the one or more anti-myeloma treatment without use of consolidation or maintenance therapy. In some of any embodiments, the one or more anti-myeloma treatment is an ASCT with prior induction therapy and the inadequate response is characterized by less than very good partial response (VGPR), e.g., at 70-110 days, after last ASCT without use of consolidation or maintenance therapy.

In some of any embodiments, the one or more anti-myeloma treatment is an ASCT with prior induction therapy and the inadequate response is characterized by less than very good partial response (VGPR) at, e.g., 70-110 days, after last ASCT without use of consolidation or maintenance therapy. In some of any embodiments, the one or more anti-myeloma treatment is an ASCT with prior induction therapy, and the inadequate response is characterized by less than very good partial response (VGPR), e.g., at 80-120 days, after last ASCT without use of consolidation or maintenance therapy.

Provided herein is a method of treating high-risk multiple myeloma in a subject, including administering to the subject a BCMA targeted CAR T cell therapy followed by an immunomodulatory agent maintenance therapy, wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma.

Provided herein is a method of treating high-risk multiple myeloma in a subject, the method including: (a) selecting a subject with a multiple myeloma characterized by an inadequate response to an autologous stem cell transplant (ASCT) after an induction therapy for treating the multiple myeloma; (b) administering to the selected subject a BCMA targeted CAR T cell therapy; and (c) after administering the BCMA targeted CAR T cell therapy, administering to the subject an immunomodulatory agent maintenance therapy.

Provided herein is a method of maintenance therapy for treating multiple myeloma in a subject, the method including administering an immunomodulatory agent maintenance therapy to a subject after having received a BCMA targeted CAR T cell therapy for treating multiple myeloma, wherein prior to the BCMA target CAR T cell therapy the subject had an inadequate response to an autologous stem cell transplant (ASCT) after an induction therapy for treating the multiple myeloma.

Provided herein is a method of treating high-risk multiple myeloma in a subject, including administering to the subject a BCMA targeted CAR T cell therapy, wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma.

In some of any embodiments, the inadequate response to the autologous stem cell transplant (ASCT) after an induction therapy is characterized by less than very good partial response (VGPR), e.g., at 70-110 days, after last ASCT. In some of any embodiments, wherein the inadequate response to the autologous stem cell transplant (ASCT) after an induction therapy is characterized by less than very good partial response (VGPR), e.g., at 80-120 days, after last ASCT.

In some of any embodiments, the ASCT after the induction therapy is a frontline therapy for treating a newly diagnosed multiple myeloma (NDMM).

In some of any embodiments, the subject is a subject that had an early relapse to the one or more anti-myeloma treatment. In some of any embodiments, the early relapse is characterized by development of progressive disease (PD) less than 18 months from starting the one or more anti-myeloma treatment.

Provided herein is a method of treating high-risk multiple myeloma in a subject, including administering to the subject a BCMA targeted CAR T cell therapy followed by an immunomodulatory agent maintenance therapy, wherein the subject, at the time of administering the BCMA targeted CAR T cell therapy, has a high-risk multiple myeloma characterized by early relapse to autologous stem cell transplant (ASCT) after an induction therapy for treating the multiple myeloma.

Provided herein is a method of treating high-risk multiple myeloma in a subject, the method containing: (a) selecting a subject with a multiple myeloma characterized by an early relapse to an autologous stem cell transplant (ASCT) after an induction therapy for treating the multiple myeloma; (b) administering to the selected subject a BCMA targeted CAR T cell therapy; and (c) after administering the BCMA targeted CAR T cell therapy, administering to the subject an immunomodulatory agent maintenance therapy.

Provided herein is a method of maintenance therapy for treating multiple myeloma in a subject, the method containing administering an immunomodulatory agent maintenance therapy to a subject after having received a BCMA targeted CAR T cell therapy for treating multiple myeloma, wherein prior to the BCMA target CAR T cell therapy the subject had an early relapse to an autologous stem cell transplant (ASCT) after an induction therapy for treating the multiple myeloma.

Provided herein is a method of treating multiple myeloma in a subject, the method containing administering to the subject a BCMA targeted CAR T cell therapy, wherein the subject, at the time of administering the BCMA targeted CAR T cell therapy, has a multiple myeloma characterized by an early relapse to an autologous stem cell transplant (ASCT) after an induction therapy for treating the multiple myeloma.

In some of any embodiments, the subject is a subject that had a suboptimal response to one or more anti-myeloma treatment. In some of any embodiments, the suboptimal response to the one or more anti-myeloma treatment is characterized by partial response (PR) or very good partial response (VGPR), e.g., at 80-120 days, after last treatment of the one or more anti-myeloma treatment without use of consolidation or maintenance therapy. In some of any embodiments, the suboptimal response to the one or more anti-myeloma treatment is characterized by partial response (PR) or very good partial response (VGPR) at about 100 days after last treatment of the one or more anti-myeloma treatment without use of consolidation or maintenance therapy.

In some of any embodiments, the one or more anti-myeloma treatment is an ASCT with prior induction therapy and the suboptimal response is characterized by partial response or very good partial response (VGPR), e.g., at 80-120 days, after last ASCT without use of consolidation or maintenance therapy. In some of any embodiments, the one or more anti-myeloma treatment is an ASCT with prior induction therapy and the suboptimal response is characterized by partial response or very good partial response (VGPR) at about or at 100 days after last ASCT without use of consolidation or maintenance therapy. In some of any embodiments, the ASCT with prior induction therapy further contains high-dose chemotherapy (HDT).

Provided herein is a method of treating multiple myeloma in a subject, the method containing administering to the subject a BCMA targeted CAR T cell therapy followed by an immunomodulatory agent maintenance therapy, wherein the subject, at the time of administering the BCMA targeted CAR T cell therapy, has a multiple myeloma characterized by a suboptimal response to autologous stem cell transplant (ASCT) after an induction therapy for treating the multiple myeloma.

Provided herein is a method of treating multiple myeloma in a subject, the method containing: (a) selecting a subject with a multiple myeloma characterized by a suboptimal response to an autologous stem cell transplant (ASCT) after an induction therapy for treating the multiple myeloma; (b) administering to the selected subject a BCMA targeted CAR T cell therapy; and (c) after administering the BCMA targeted CAR T cell therapy, administering to the subject an immunomodulatory agent maintenance therapy.

Provided herein is a method of treating multiple myeloma in a subject, the method containing: (a) selecting a subject with a multiple myeloma characterized by a suboptimal response to an autologous stem cell transplant (ASCT) after an induction therapy for treating the multiple myeloma; (b) administering to the selected subject an immunomodulatory agent maintenance therapy prior to a leukapheresis to obtain T cells from the subject for engineering with a BCMA targeted CAR to produce a BCMA targeted CAR T cell therapy; (c) administering to the selected subject the BCMA targeted CAR T cell therapy; and (d) after administering the BCMA targeted CAR T cell therapy, administering to the subject an immunomodulatory agent maintenance therapy. In some of any embodiments, preleukapheresis administration of an immunomodulatory agent therapy involves administering a subject lenalidomide prior to a leukapheresis to obtain T cells from the subject for engineering with a BCMA targeted CAR to produce a BCMA targeted CAR T cell therapy. For example, subjects receiving CAR T cell administration with lenalidomide maintenance, a lenalidomide preleukapheresis (1 cycle) is administered orally at a dose of 10 mg once daily on Days 1 to 28 of a 28-day cycle.

Provided herein is a method of treating multiple myeloma in a subject, the method containing administering an immunomodulatory agent maintenance therapy to a subject after having received a BCMA targeted CAR T cell therapy for treating multiple myeloma, wherein prior to the BCMA target CAR T cell therapy the subject had a suboptimal response to an autologous stem cell transplant (ASCT) after an induction therapy for treating the multiple myeloma.

In some of any embodiments, the subject has been administered an immunomodulatory agent maintenance therapy prior to a leukapheresis to obtain T cells from the subject for engineering with a BCMA targeted CAR to produce a BCMA targeted CAR T cell therapy. In some of any embodiments, preleukapheresis administration of an immunomodulatory agent therapy involves administering a subject lenalidomide prior to a leukapheresis to obtain T cells from the subject for engineering with a BCMA targeted CAR to produce a BCMA targeted CAR T cell therapy. For example, subjects receiving CAR T cell administration with lenalidomide maintenance, a lenalidomide preleukapheresis (1 cycle) is administered orally at a dose of 10 mg once daily on Days 1 to 28 of a 28-day cycle.

Provided herein is a method of treating multiple myeloma in a subject, the method containing administering to the subject a BCMA targeted CAR T cell therapy, wherein the subject, at the time of administering the BCMA targeted CAR T cell therapy, has a multiple myeloma characterized by a suboptimal response an autologous stem cell transplant (ASCT) after an induction therapy for treating the multiple myeloma.

In some of any embodiments, the suboptimal response to the autologous stem cell transplant (ASCT) after an induction therapy is characterized by partial response (PR) or very good partial response (VGPR), e.g., at 80-120 days, after last ASCT. In some of any embodiments, the suboptimal response to the autologous stem cell transplant (ASCT) after an induction therapy is characterized by partial response (PR) or very good partial response (VGPR) at about or at 100 days after last ASCT. In some of any embodiments, the ASCT after induction therapy further contains high-dose chemotherapy (HDT). In some of any embodiments, the induction therapy contains one or more of a proteasome inhibitor and an immunomodulatory agent.

In some of any embodiments, the induction therapy is one or more of a proteasome inhibitor, an immunomodulatory agent, and dexamethasone.

In some of any embodiments, the induction therapy is: (i) bortezomib, lenalidomide or a pharmaceutically acceptable salt thereof, and dexamethasone (VRD); (ii) thalidomide and dexamethasone (TD); (iii) lenalidomide or a pharmaceutically acceptable salt thereof and low-dose dexamethasone (RD); (iv) bortezomib and dexamethasone (VD); (v) bortezomib, thalidomide, and dexamethasone (VTD); (vi) carfilzomib, lenalidomide or a pharmaceutically acceptable salt thereof, and dexamethasone (KRd); or (vii) ixazomib, lenalidomide or a pharmaceutically acceptable salt thereof, and dexamethasone (Ixa-Rd).

In some of any embodiments, the induction therapy is administered in ≥3 cycles. In some of any embodiments, the induction therapy is administered in 3-12 cycles. In some of any embodiments, the induction therapy is administered in 3-6 cycles. In some of any embodiments, the induction therapy is administered in 4-6 cycles. In some of any embodiments, each cycle is a 28-day cycle.

In some of any embodiments, in each cycle of the induction therapy: bortezomib is administered at or about 1.3 mg/m² intravenously or subcutaneously on days 1, 8 and 15 of the cycle; lenalidomide or a pharmaceutically acceptable salt thereof is administered at or about 25 mg orally on days 1 to 21 of the cycle or days 1 to 14 of the cycle; and/or dexamethasone is administered at or about 40 mg on days 1, 8 and 15 of the cycle.

In some of any embodiments, prior to receiving the BCMA targeted CAR T cell therapy the subject had not received a prior immunomodulatory agent maintenance therapy.

In some of any embodiments, prior to receiving the BCMA targeted CAR T cell therapy the subject had not received a consolidation therapy.

In some of any embodiments, prior to receiving the BCMA targeted CAR T cell therapy the subject received a prior immunomodulatory agent maintenance therapy after the ASCT with the induction therapy.

In some of any embodiments, the subject was diagnosed with multiple myeloma about three years or less before administering the BCMA targeted CAR T cell therapy. In some of any embodiments, the subject was diagnosed with multiple myeloma about two years or less before administering the BCMA targeted CAR T cell therapy. In some of any embodiments, the subject was diagnosed with multiple myeloma about 1.6 years or less before administering the BCMA targeted CAR T cell therapy.

In some of any embodiments, at the time of administering the BCMA targeted CAR T cell therapy the subject has R-ISS stage I disease. In some of any embodiments, at the time of administering the BCMA targeted CAR T cell therapy the subject has R-ISS stage II disease. In some of any embodiments, at the time of administering the BCMA targeted CAR T cell therapy the subject has R-ISS stage III disease.

In some of any embodiments, at the time of administering the BCMA targeted CAR T cell therapy the subject has high-risk cytogenetics. In some of any embodiments, at the time of administering the BCMA targeted CAR T cell therapy the subject has ultra high-risk cytogenetics.

In some of any embodiments, at the time of administering the BCMA targeted CAR T cell therapy the subject has bone marrow biopsy-determined high tumor burden, optionally wherein the high tumor burden is ≥50% bone marrow CD138+ plasma cells.

In some of any embodiments, at the time of administering the BCMA targeted CAR T cell therapy the subject has extramedullary disease.

In some of any embodiments, at the time of administering the BCMA targeted CAR T cell therapy the multiple myeloma was refractory to treating with one or both of an immunomodulatory agent and a proteasome inhibitor (PI).

In some of any embodiments, at the time of administering the BCMA targeted CAR T cell therapy the subject had an ECOG performance status (PS)≤1. In some of any embodiments, at the time of administering the BCMA targeted CAR T cell therapy the subject had an ECOG PS of 0.

In some of any embodiments, the immunomodulatory agent maintenance therapy prior to leukapheresis is initiated between or from 10 to 12 weeks prior to administering the BCMA targeted CAR-T cell therapy. In some of any embodiments, the immunomodulatory agent maintenance therapy prior to leukapheresis is initiated at about 10 weeks prior to administering the BCMA targeted CAR-T cell therapy. In some of any embodiments, the immunomodulatory agent maintenance therapy prior to leukapheresis is initiated at about 11 weeks prior to administering the BCMA targeted CAR-T cell therapy. In some of any embodiments, the immunomodulatory agent maintenance therapy prior to leukapheresis is initiated at about 12 weeks prior to administering the BCMA targeted CAR-T cell therapy. In some of any embodiments, the immunomodulatory agent maintenance therapy after administering the BCMA targeted CAR T cell therapy is initiated at no later than 6 months after administering the BCMA targeted CAR T cell therapy. In some of any embodiments, the immunomodulatory agent maintenance therapy after administering the BCMA targeted CAR T cell therapy is initiated at no later than 5 months after administering the BCMA targeted CAR T cell therapy. In some of any embodiments, the immunomodulatory agent maintenance therapy after administering the BCMA targeted CAR T cell therapy is initiated at no later than 4 months after administering the BCMA targeted CAR T cell therapy. In some of any embodiments, the immunomodulatory agent maintenance therapy after administering the BCMA targeted CAR T cell therapy is initiated at no later than 3 months after administering the BCMA targeted CAR T cell therapy. In some of any embodiments, the immunomodulatory agent maintenance therapy after administering the BCMA targeted CAR T cell therapy is initiated at no later than 2 months after administering the BCMA targeted CAR T cell therapy. In some of any embodiments, the immunomodulatory agent maintenance therapy after administering the BCMA targeted CAR T cell therapy is initiated at no later than 1 month after administering the BCMA targeted CAR T cell therapy. In some of any embodiments, the immunomodulatory agent maintenance therapy after administering the BCMA targeted CAR T cell therapy is initiated at about 1 month after administering the BCMA targeted CAR T cell therapy.

In some of any embodiments, the immunomodulatory agent maintenance therapy is initiated at Day 15 or later after administering the BCMA targeted CAR T cell therapy. In some of any embodiments, the immunomodulatory agent maintenance therapy is initiated at 1 month or later after administering the BCMA targeted CAR T cell therapy.

In some of any embodiments, the immunomodulatory agent maintenance therapy is initiated at at least 1 month after administering the BCMA targeted CAR T cell therapy. In some of any embodiments, the immunomodulatory agent maintenance therapy is initiated at at least 2 months after administering the BCMA targeted CAR T cell therapy. In some of any embodiments, the immunomodulatory agent maintenance therapy is initiated at at least 3 months after administering the BCMA targeted CAR T cell therapy. In some of any embodiments, the immunomodulatory agent maintenance therapy is initiated at at least 4 months after administering the BCMA targeted CAR T cell therapy.

In some of any embodiments, the immunomodulatory agent maintenance therapy is initiated at no later than 24 months after administering the BCMA targeted CAR T cell therapy.

In some of any embodiments, the immunomodulatory agent maintenance therapy is initiated at or between Day 15 and Day 730 after administering the BCMA targeted CAR T cell therapy. In some of any embodiments, the immunomodulatory agent maintenance therapy is initiated at or between 1 month and 24 months, 1 month and 12 months, 1 month and 10 months, 1 month and 6 months, or 1 month and 4 months after administering the BCMA targeted CAR T cell therapy. In some of any embodiments, the immunomodulatory agent maintenance therapy is initiated at or between 1 month and 6 months after administering the BCMA targeted CAR T cell therapy.

In some of any embodiments, the immunomodulatory agent maintenance therapy is initiated at or between 4 months and 24 months, 4 months and 12 months, or 4 months and 10 months after administering the BCMA targeted CAR T cell therapy. In some of any embodiments, the immunomodulatory agent maintenance therapy is initiated at or between 4 months and 10 months, after administering the BCMA targeted CAR T cell therapy.

In some of any embodiments, the immunomodulatory agent maintenance therapy is initiated at or between Day 30 and Day 180 after administering the BCMA targeted CAR T cell therapy.

In some of any embodiments, the immunomodulatory agent maintenance therapy is initiated at 1 month after administering the BCMA targeted CAR T cell therapy.

In some of any embodiments, the immunomodulatory agent maintenance therapy continues until disease progression. In some of any embodiments, the immunomodulatory agent maintenance therapy continues until the subject achieves complete response (CR).

In some of any embodiments, the immunomodulatory agent of the immunomodulatory agent maintenance therapy is a compound that interacts with and/or binds to cereblon (CRBN) or one or more members of the CRBN E3 ubiquitin-ligase complex; an inhibitor of Ikaros (IKZF1); an inhibitor of Aiolos (IKZF3); or a compound that enhances or promote ubiquitination and/or degradation of Ikaros (IKZF1) and Aiolos (IKZF3).

In some of any embodiments, the immunomodulatory agent of the immunomodulatory agent maintenance therapy is thalidomide, a thalidomide analog, or a thalidomide derivative.

In some of any embodiments, the immunomodulatory agent of the immunomodulatory agent maintenance therapy is lenalidomide or a pharmaceutically acceptable salt thereof, pomalidomide, avadomide, iberdomide, CC-92480 or CC-885. In some of any embodiments, the immunomodulatory agent of the immunomodulatory agent maintenance therapy is lenalidomide or a generic thereof. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide. In some of any embodiments, the immunomodulatory agent of the immunomodulatory agent maintenance therapy is lenalidomide or a pharmaceutically acceptable salt thereof.

In some of any embodiments, the immunomodulatory agent of the immunomodulatory agent maintenance therapy is administered in an amount of 0.5 mg per day to 50 mg per day. In some of any embodiments, the immunomodulatory agent of the immunomodulatory agent maintenance therapy is administered in an amount of 0.5 mg per day to 25 mg per day, 0.5 mg per day to 10 mg per day, 0.5 mg per day to 5 mg per day, 0.5 mg per day to 2.5 mg per day, 0.5 mg per day to 1 mg per day, 1 mg per day to 50 mg per day, 1 mg per day to 25 mg per day, 1 mg per day to 10 mg per day, 1 mg per day to 5 mg per day, 1 mg per day to 2.5 mg per day, 2.5 mg per day to 50 mg per day, 2.5 mg per day to 25 mg per day, 2.5 mg per day to 10 mg per day, 2.5 mg per day to 5 mg per day, 5 mg per day to 50 mg per day, 5 mg per day to 25 mg per day, 5 mg per day to 10 mg per day, 10 mg per day to 50 mg per day, 10 mg per day to 25 mg per day or 25 mg per day to 50 mg per day.

In some of any embodiments, the immunomodulatory agent of the immunomodulatory agent maintenance therapy is administered in an amount of 2.5 mg per day to 25 mg per day. In some of any embodiments, the immunomodulatory agent of the immunomodulatory agent maintenance therapy is administered in an amount of 2.5 mg per day to 10 mg per day. In some of any embodiments, the immunomodulatory agent of the immunomodulatory agent maintenance therapy is administered in an amount of at or about 1 mg per day. In some of any embodiments, the immunomodulatory agent of the immunomodulatory agent maintenance therapy is administered in an amount of at or about 2.5 mg per day. In some of any embodiments, the immunomodulatory agent of the immunomodulatory agent maintenance therapy is administered in an amount of at or about 5 mg per day. In some of any embodiments, the immunomodulatory agent of the immunomodulatory agent maintenance therapy is administered in an amount at or of about 10 mg per day. In some of any embodiments, the immunomodulatory agent of the immunomodulatory agent maintenance therapy is administered in an amount of at or about 15 mg per day. In some of any embodiments, the amount of the immunomodulatory agent of the immunomodulatory agent maintenance therapy is administered daily for 14 days in a 28-day cycle. In some of any embodiments, the amount of the immunomodulatory agent of the immunomodulatory agent maintenance therapy is administered daily for 21 days in a 28-day cycle. In some of any embodiments, the amount of the immunomodulatory agent of the immunomodulatory agent maintenance therapy is administered daily for 28 days in a 28-day cycle.

In some of any embodiments, prior to the administration of the BCMA targeted CAR T cell therapy to the subject, the subject has received a lymphodepleting therapy including the administration of fludarabine at or about 20-40 mg/m² body surface area of the subject (optionally at or about 30 mg/m²) daily for 2-4 days, and/or cyclophosphamide at or about 200-400 mg/m² body surface area of the subject (optionally at or about 300 mg/m²) daily for 2-4 days. In some of any embodiments, prior to the administration of the BCMA targeted CAR T cell therapy to the subject, the subject has received a lymphodepleting therapy including the administration of fludarabine at or about 30 mg/m² body surface area of the subject, daily, and cyclophosphamide at or about 300 mg/m² body surface area of the subject, daily, for 3 days.

In some of any embodiments, the BCMA targeted CAR T cell therapy includes a chimeric antigen receptor (CAR) containing an extracellular antigen-binding domain that binds to BCMA, a transmembrane domain, and an intracellular signaling region.

In some of any embodiments, the extracellular antigen-binding domain includes a variable heavy chain (VH) region and a variable light chain (VL) region. In some embodiments: the VH region includes a CDR-H1, a CDR-H2, and a CDR-H3 containing the amino acid sequences set forth in SEQ ID NOS: 189, 190, and 191, respectively; and the VL region includes a CDR-L1, a CDR-L2, and a CDR-L3 containing the amino acid sequences set forth in SEQ ID NOS: 192, 193, and 194, respectively; or the VH region includes a CDR-H1, a CDR-H2, and a CDR-H3 containing the amino acid sequences set forth in SEQ ID NOS: 173, 174 and 175, respectively; and the VL region includes a CDR-L1, a CDR-L2, and a CDR-L3 containing the amino acid sequences set forth in SEQ ID NOS: 183, 184 and 185, respectively. In some of any embodiments, wherein: the VH region contains an amino acid sequence set forth in SEQ ID NO: 18 and the VL region contains the amino acid sequence set forth in SEQ ID NO: 19; or the VH region contains an amino acid sequence set forth in SEQ ID NO: 24, and the VL region contains the amino acid sequence set forth in SEQ ID NO: 25.

In some of any embodiments, the extracellular antigen-binding domain is a single chain variable fragment (scFv). In some of any embodiments, the scFv contains the amino acid sequence set forth in SEQ ID NO: 213 or SEQ ID NO: 188.

In some of any embodiments, the intracellular signaling region further contains a costimulatory signaling domain. In some embodiments, the costimulatory signaling domain contains an intracellular signaling domain of CD28, 4-1BB, or ICOS, or a signaling portion thereof. In some of any embodiments, the costimulatory signaling domain is between the transmembrane domain and the cytoplasmic signaling domain of a CD3-zeta (CD35) chain.

In some of any embodiments, the transmembrane domain is or includes a transmembrane domain from CD28 or CD8, optionally human CD28 or CD8.

In some of any embodiments, the CAR further includes an extracellular spacer between the antigen binding domain and the transmembrane domain. In some embodiments, the spacer is from CD8, optionally wherein the spacer is a CD8a hinge. In some of any embodiments, the transmembrane domain and the spacer are from CD8.

In some of any embodiments, the CAR contains the amino acid sequence set forth in SEQ ID NO: 116 or SEQ ID NO: 124. In some of any embodiments, the CAR is encoded by the polynucleotide sequence set forth in SEQ ID NO: 214.

In some of any embodiments, the BCMA targeted CAR T cell therapy includes CD3+ CAR-expressing T cells. In some of any embodiments, the BCMA targeted CAR T cell therapy includes a combination of CD4+ T cells and CD8+ T cells and/or a combination of CD4+ CAR-expressing T cells and CD8+ CAR-expressing T cells.

In some embodiments, the ratio of CD4+ CAR-expressing T cells to CD8+ CAR-expressing T cells and/or of CD4+ T cells to CD8+ T cells, is or is approximately 1:1 or is between at or approximately 1:3 and at or approximately 3:1.

In some of any embodiments: the percentage of naïve-like T cells and/or central memory T cells is greater than or greater than about 60% of the total genetically engineered T cells in the dose, optionally greater than or greater than about 65%, 70%, 80%, 90% or 95%; the percentage of naïve-like T cells and/or central memory T cells is greater than or greater than about 40% of the total CD4+ genetically engineered T cells in the dose, optionally greater than or greater than about 50%, 60%, 70%, 80%, 90% or 95%; or the percentage of naïve-like T cells and/or central memory T cells is greater than or greater than about 40% of the total CD8+ genetically engineered T cells in the dose, optionally greater than or greater than about 50%, 60%, 70%, 80%, 90% or 95%.

In some embodiments, the naïve-like T cells are CCR7+CD45RA+, CD27+CCR7+, or CD62L-CCR7+.

In some of any embodiments, the BCMA targeted CAR T cell therapy is: idecabtagene vicleucel; bb21217 cells; orvacabtagene autoleucel; CT103A; ciltacabtagene autoleucel; KITE585; CT053; BCMA-CS1 cCAR (BC1cCAR); P-BCMA-101; P-BCMA-ALLO1; C-CAR088; Descartes-08; PBCAR269A; ALLO-715; PHE885; AUTO8; CTX120; CB-011; ALLO-605 (TuboCAR/MM); pCDCAR1 (TriCAR-Z136), or GC012F. In some of any embodiments, the BCMA targeted CAR T cell therapy is idecabtagene vicleucel.

In some of any embodiments, the dose of the BCMA targeted CAR T cell therapy is between about 100×10⁶ CAR-positive T cells and about 600×10⁶ CAR-positive T cells. In some of any embodiments, the dose of the BCMA targeted CAR T cell therapy is between about 150×10⁶ CAR-positive T cells and about 540×10⁶ CAR-positive T cells. In some of any embodiments, the dose of the BCMA targeted CAR T cell therapy is between about 150×10⁶ and about 450×10⁶ CAR-positive T cells.

In some of any embodiments, the dose of the BCMA targeted CAR T cell therapy is between about 300×10⁶ CAR-positive T cells and about 540×10⁶ CAR-positive T cells. In some of any embodiments, the dose of the BCMA targeted CAR T cell therapy is between about 300×10⁶ and about 460×10⁶ CAR-positive T cells. In some of any embodiments, the dose of the BCMA targeted CAR T cell therapy is between about 300×10⁶ and about 450×10⁶ CAR-positive T cells.

In some of any embodiments, the dose of the BCMA targeted CAR T cell therapy is about 150×10⁶, about 300×10⁶, about 450×10⁶, or about 540×10⁶ CAR-positive T cells. In some of any embodiments, the dose of the BCMA targeted CAR T cell therapy is about 300×10⁶, about 450×10⁶, or about 540×10⁶ CAR-positive T cells.

In some of any embodiments, the T cells of the BCMA targeted CAR T cell therapy were obtained from the subject, optionally by apheresis or leukapheresis, and the T cells were engineered ex vivo with the BCMA targeted CAR. In some of any embodiments, the dose of the BCMA targeted CAR T cell therapy is autologous to the subject. In some of any embodiments, the dose of the BCMA targeted CAR T cell therapy is allogeneic to the subject.

In some of any embodiments, the subject received a bridging therapy prior to receiving the BCMA targeted CAR T cell therapy, optionally wherein the bridging therapy was administered to the subject in the period between obtaining the T cells from the subject and administering the BCMA targeted CAR T cell therapy to the subject. The method of some of any embodiments, further containing administering to the subject a bridging therapy prior to administering the BCMA targeted CAR T cell therapy to the subject, optionally wherein the bridging therapy is administered to the subject in the period between obtaining the T cells from the subject and administering the BCMA targeted CAR T cell therapy to the subject. In some of any embodiments, the bridging therapy includes one or more of corticosteroids, alkylating agents, immunomodulatory agents, proteasome inhibitors (PI), or anti-CD38 antibodies.

In some of any embodiments, the subject is human. In some of any embodiments, the subject is 18 year of age or older.

### BRIEF DESCRIPTION OF THE DRAWINGS

FIG. 1A is a swimmer plot of individual anti-BCMA CAR-T cell-treated subjects at an earlier data cut-off date. The bars begin at M2D1. M2D1 represents Month 2 Day 1, which is equivalent to 1 month post anti-BCMA CAR-T cell infusion. The bars end at the last response assessment date or the data cut-off date, whichever occurred later. Response was defined as PR or better based on IMWG Criteria by investigator assessment. Eight subjects received lenalidomide maintenance therapy and the therapy was initiated between approximately 4 to 10 months from anti-BCMA CAR-T cell infusion. FIG. 1B is a swimmer plot of the same individuals at a subsequent data cut-off date. The x-axis is up to 48 months.

FIG. 2A depicts the best overall response, stratified by partial response (PR), very good partial response (VGPR), complete response (CR), or stringent complete response (sCR) at an earlier data cut-off date. FIG. 2B depicts also the best overall response, stratified by partial response (PR), very good partial response (VGPR), complete response (CR), or stringent complete response (sCR) at a subsequent data cut-off date. The left panel depicts all subjects whereas the right panel depicts the eight subjects who received the lenalidomide maintenance therapy.

FIG. 3A depicts the duration of the response at an earlier data cut-off date. All responders are depicted in FIG. 3A while subjects with ≥CR are depicted in FIG. 3B. FIG. 3A depicts the duration of the response at a subsequent data cut-off date. All responders are depicted in FIG. 3C while subjects with ≥CR are depicted in FIG. 3D.

FIG. 4A depicts the percentage of subjects with progression-free survival at different time-points at an earlier data cut-off date. FIG. 4B depicts the percentage of subjects with progression-free survival at different time-points at a subsequent data cut-off date.

FIG. 5 depicts soluble BCMA (sBCMA) by overall response at various time-points. Serum level of sBCMA was analyzed at Day 1 as a measure of tumor burden. The limit of detection of the assay is 4.4 mg/mL (shown by the horizontal dashed line). Data are geometric mean±standard error of the mean of sBCMA concentration. “NR” is no response. “Month 2, Month 3, Month 4, Month 5, Month 6 and Month 7” labels correspond to around 30 days, 60 days, 90 days, 120 days, 150 days and 180 days respectively after anti-BCMA CAR-T cell infusion.

FIG. 6 shows the median fold change from baseline to peak serum concentration (Cₘₐₓ) for inflammatory cytokines IFN-γ, IL-6, IL-8, and IL-10 for NDMM subjects and 4L+RRMM (relapsed and refractory multiple myeloma, R/R MM) subjects. Error bars represent 95% CI.

FIGS. 7A-7B shows the MRD negativity, defined as minimum of 1 in 10⁵ nucleated cells at an earlier data cut-off date, in subjects with ≥CR (FIG. 7A) and in all evaluable subjects (FIG. 7B). FIGS. 7C-D shows the MRD negativity, defined as minimum of 1 in 10⁵ nucleated cells at an subsequent data cut-off date, in subjects with ≥CR (FIG. 7C) and in all evaluable subjects (FIG. 7D).

FIG. 8 depicts the best overall response stratified by partial response (PR), very good partial response (VGPR), complete response (CR), or stringent complete response (sCR).

FIG. 9 shows the MRD negativity, defined as minimum of 1 in 10⁵ nucleated cells, in subjects with ≥CR (FIG. 9A) and in all evaluable subjects (FIG. 9B).

FIG. 10 depicts the duration of best response. All responders are depicted in FIG. 10A while subjects with ≥CR, who achieved VGPR, or who achieved PR are depicted in FIG. 10B.

FIG. 11A depicts the percentage of patients with progression-free survival at different time-points while FIG. 11B depicts the percentage of patients with overall survival at different time-points.

FIG. 12 depicts soluble BCMA (sBCMA) by overall response at various time-points. Serum level of sBCMA was analyzed at Day 1 as a measure of tumor burden. The limit of detection of the assay is 4.4 mg/mL (shown by the horizontal dashed line). Presented data are at baseline, and Days 1, 4, 8, 12, 15, 22, 29, and 57 within the first two months. Data are geometric mean±standard error of the mean of sBCMA concentration. “NR” is no response. “Month 2, Month 3, Month 4, Month 5, Month 6 and Month 7” labels correspond to around 30 days, 60 days, 90 days, 120 days, 150 days and 180 days respectively after anti-BCMA CAR-T cell infusion.

FIGS. 13A-13B depict the mean change from baseline score of patient-reported outcomes on health-related quality of life (HRQoL). FIG. 13A depicts European Organization for Research and Treatment of Cancer (EORTC) Quality of Life Core 30 Questionnaire (QLQ-C30) (EORTC QLQ-C30) global health status/quality of life (QoL) domain. FIG. 13B depicts EORTC Quality of Life Multiple Myeloma 20 Questionnaire (QLQ-MY20) disease symptoms domain.

### DETAILED DESCRIPTION

Provided herein are therapies involving administration of a T cell therapy followed by an immunomodulatory agent maintenance therapy to a subject having a cancer. In some aspects, the subject has exhibited an early relapse, an inadequate response, or a suboptimal response to a frontline autologous stem cell transplant therapy (ASCT; also referred to as autologous stem cell transplantation or autologous stem cell transplant). In some aspects, the subject has exhibited an early relapse to a frontline autologous stem cell transplant therapy (ASCT). In some aspects, the subject has exhibited an inadequate response to a frontline autologous stem cell transplant therapy (ASCT). In some aspects, the subject has exhibited a suboptimal response to a frontline autologous stem cell transplant therapy (ASCT). In some aspects, the subject has high risk multiple myeloma. In some aspects, the subject has newly diagnosed multiple myeloma (NDMM).

In some aspects, following administration of the T cell therapy (e.g. CAR T cells), the subject is administered an immunomodulatory agent for maintenance therapy. In some embodiments, the therapy involves administration of the T cell therapy, such as a composition including cells for adoptive cell therapy, such as a T cell therapy (e.g., CAR-expressing T cells), followed by administration of an immunomodulatory agent for maintenance therapy, such as lenalidomide or a structural or functional analog of lenalidomide.

In some aspects, the standard of care in transplant-eligible patients with NDMM is induction therapy followed by ASCT. In some aspects, the standard of care for transplant-eligible patients with NDMM is induction therapy followed by ASCT, which may be followed by maintenance therapy. See Al Hamed R, et al. 2019, Blood Cancer J, 9 (4): 44; Voorhees, et al. 2020, Blood, 136 (8): 936-945. In some aspects, the induction therapy is followed by high-dose chemotherapy and ASCT. In some aspects, the maintenance therapy includes lenalidomide or a structural or functional analog of lenalidomide.

Studies have shown that maintenance therapy following ASCT can achieve a deeper response and improve survival outcomes. See Al Hamed R, et al. 2019, Blood Cancer J, 9 (4): 44; Voorhees, et al. 2020, Blood, 136 (8): 936-945. Maintenance with lenalidomide following ASCT can, for example, lead to a complete response rate of 60.9% and 20.4 of patients achieving minimal residual disease negativity at 22.1 months follow up. See Voorhees, et al. 2020, Blood, 136 (8): 936-945.

Patients with multiple myeloma (MM) who do not achieve a deep response post-autologous stem cell transplantation (ASCT) have an increased risk of progressive disease (PD) and death. See Van de Helde et al. 2017, Eur J Haematol, 98 (3): 269-279. Survival in these patients is poor. Patients who achieve a response ≥very good partial response (VGPR) post-induction therapy and ASCT have better outcomes than those with <very good partial response (VGPR). In some embodiments, a subject is considered to have a suboptimal response, if the subject, at first post-ASCT assessment between 80-120 days after ASCT, achieves partial response (PR) or very good partial response (VGPR). In some embodiments, a subject is considered to have a suboptimal response, if the subject, at first post-ASCT assessment approximately 100 days after ASCT, achieves partial response (PR) or very good partial response (VGPR). In some embodiments, a subject is considered to have an inadequate response if the subject, at first post-ASCT assessment between 70 to 110 days after ASCT, has less than very good partial response (excluding partial response). Provided herein are improvements to therapies that can improve patient outcomes.

Similarly, patients with multiple myeloma (MM) who relapse early after frontline therapy with autologous stem cell therapy (ASCT; also referred to as autologous stem cell transplantation or autologous stem cell transplant) have high-risk features and markedly inferior survival compared to the features and survival of later relapsing patients. See Paiva B, et al. 2012, Blood, 119 (3): 687-691; Bygrave et al. 2021, Br J Haematol, 193 (3): 551-555; Davies et al. 2022, Blood Cancer Discov, 3 (4): 273-284. 4; and Majithia et al. 2016, Leukemia, 30 (11): 2208-2213. It has been shown that patients who experience early relapse have shorter lives than those who experience later relapse. Patients who relapse earlier have a median overall survival after ASCT of 26 months whereas patients who relapse later have a median overall survival after ASCT of 91 months. See Bygrave et al. 2021, Br J Haematol, 193 (3): 551-555.

In some embodiments, early relapse can be disease progression after a period of response to treatment. In some embodiments, a patient is considered to relapse early if the relapse occurs <12 months after starting the frontline treatment. In some embodiments, a patient is considered to relapse early if the relapse occurs within 18 months after starting the frontline treatment. In particular embodiments, frontline treatment includes induction therapy, ASCT, and lenalidomide maintenance therapy.

In some aspects, available approaches for treatment of cancer, such as multiple myeloma, are complex and may not always be entirely satisfactory. In some aspects, choosing a treatment regimen can depend on numerous factors including drug availability, response to prior therapy, aggressiveness of the relapse, eligibility for autologous stem cell transplantation (ASCT), and whether the relapse occurred on or off therapy.

In some embodiments, the methods can be used for treating a cancer, e.g., a B cell malignancy or hematological malignancy, and in particular, such diseases, conditions, or malignancies in which responses, e.g., complete response, to treatment with the T cell therapy (e.g., CAR-expressing T cells) alone is relatively low compared to treatment also including an immunomodulatory agent maintenance therapy. In some embodiments, the cancer is a myeloma, leukemia, or lymphoma. In some embodiments, the cancer is multiple myeloma. In some embodiments, the cancer is newly diagnosed multiple myeloma (NDMM).

In some embodiments, the methods provided herein are for use in a subject having a cancer in which prior to initiation of administration of the T cell therapy, the subject had an early relapse, an inadequate response, or a suboptimal response to a frontline therapy (such as frontline ASCT), and following administration of the T cell therapy, the subject is treated with an immunomodulatory agent maintenance therapy. In some embodiments, the methods provided herein are for use in a subject having a cancer in which prior to initiation of administration of the T cell therapy, the subject had an early relapse to a frontline therapy (such as frontline ASCT), and following administration of the T cell therapy, the subject is treated with an immunomodulatory agent maintenance therapy. In some embodiments, the methods provided herein are for use in a subject having a cancer in which prior to initiation of administration of the T cell therapy, the subject had an inadequate relapse to a frontline therapy (such as frontline ASCT), and following administration of the T cell therapy, the subject is treated with an immunomodulatory agent maintenance therapy. In some embodiments, the methods provided herein are for use in a subject having a cancer in which prior to initiation of administration of the T cell therapy, the subject had a suboptimal response to a frontline therapy (such as frontline ASCT), and following administration of the T cell therapy, the subject is treated with an immunomodulatory agent maintenance therapy. In some embodiments, the methods provided herein are for use in a subject having a cancer in which prior to initiation of administration of the T cell therapy, the subject had a suboptimal response to a frontline therapy (such as frontline ASCT), and prior to administration of the T cell therapy, the subject is treated with a first immunomodulatory agent maintenance therapy and, following administration of the T cell therapy, the subject is treated with a second immunomodulatory agent maintenance therapy.

In some embodiments, the methods provided herein are for use in a subject having a cancer, in which the T cell therapy administered without an immunomodulatory agent maintenance therapy is insufficient to ameliorate, reduce, or prevent the disease or condition in the subject or a symptom or outcome thereof.

Also provided are methods for engineering, preparing, and producing the T cell therapy, compositions containing the T cell therapy and/or the maintenance therapy (e.g., immunomodulatory agent) for using, producing and administering the T cell therapy and/or the maintenance therapy, such as in accord with the provided methods. In some embodiments, the provided methods lower the incidence rates of cytokine release syndrome (CRS), thrombocytopenia, and neurotoxicity (NT) in subjects who were treated with anti-BCMA CAR T-cell treatment in earlier lines of therapy compared to the rates in subjects who were treated with CAR-T cells in later lines of therapy. See Munshi et al. 2021, N Engl J Med, 384:705-716.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods of Treatment and Uses

Among the provided methods and uses herein are methods of treating multiple myeloma in a subject who had an early relapse, an inadequate response, or a suboptimal response to one or more anti-myeloma treatment, which may include administering to the subject a BCMA targeted CAR T cell therapy followed by an immunomodulatory agent maintenance therapy. Also among the provided methods and uses are methods of treating multiple myeloma in a subject, which may include (a) selecting a subject who had an early relapse, an inadequate response, or a suboptimal response to one or more anti-myeloma treatment; (b) administering to the selected subject a BCMA targeted CAR T cell therapy; and (c) after administering the BCMA targeted CAR T cell therapy, administering to the subject an immunomodulatory agent maintenance therapy.

Among the provided methods and uses are methods of treating newly diagnosed multiple myeloma (NDMM) in a subject who had an early relapse, an inadequate response, or a suboptimal response to a stem cell transplant therapy, which may include administering to the subject a BCMA targeted CAR T cell therapy followed by an immunomodulatory agent maintenance therapy. Also among the provided methods and uses are methods of treating newly diagnosed multiple myeloma (NDMM) in a subject, which may include (a) selecting a subject who had an early relapse, an inadequate response, or a suboptimal response to one or more anti-myeloma treatment; (b) administering to the selected subject a BCMA targeted CAR T cell therapy; and (c) after administering the BCMA targeted CAR T cell therapy, administering to the subject an immunomodulatory agent maintenance therapy.

Among the provided methods and uses are methods of maintenance therapy for treating multiple myeloma, which may include administering an immunomodulatory agent maintenance therapy to a subject after having received a BCMA targeted CAR T cell therapy for treating multiple myeloma, wherein prior to the BCMA target CAR T cell therapy the subject had an early relapse, an inadequate response, or a suboptimal response to one or more other anti-myeloma treatment.

Among the provided methods and uses are methods of treating high-risk multiple myeloma in a subject, which may include administering to the subject a BCMA targeted CAR T cell therapy followed by an immunomodulatory agent maintenance therapy, wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma. Also among the provided methods and uses are methods of treating high-risk multiple myeloma in a subject, which may include (a) selecting a subject with a multiple myeloma characterized by an inadequate response to an autologous stem cell transplant (ASCT) after an induction therapy for treating the multiple myeloma; (b) administering to the selected subject a BCMA targeted CAR T cell therapy; and (c) after administering the BCMA targeted CAR T cell therapy, administering to the subject an immunomodulatory agent maintenance therapy.

Among the provided methods and uses are methods of maintenance therapy for treating multiple myeloma in a subject, which may include administering an immunomodulatory agent maintenance therapy to a subject after having received a BCMA targeted CAR T cell therapy for treating multiple myeloma, wherein prior to the BCMA target CAR T cell therapy the subject had an inadequate response to an autologous stem cell transplant (ASCT) after an induction therapy for treating the multiple myeloma.

Among the provided the methods and uses are methods of treating high-risk multiple myeloma in a subject, which may include administering to the subject a BCMA targeted CAR T cell therapy followed by an immunomodulatory agent maintenance therapy, wherein the subject, at the time of administering the BCMA targeted CAR T cell therapy, has a high-risk multiple myeloma characterized by early relapse to autologous stem cell transplant (ASCT) after an induction therapy for treating the multiple myeloma. Also among the provided methods and uses are methods of treating high-risk multiple myeloma in a subject, which may include (a) selecting a subject with a multiple myeloma characterized by an early relapse to an autologous stem cell transplant (ASCT) after an induction therapy for treating the multiple myeloma; (b) administering to the selected subject a BCMA targeted CAR T cell therapy; and (c) after administering the BCMA targeted CAR T cell therapy, administering to the subject an immunomodulatory agent maintenance therapy.

Among the provided methods and uses are methods of maintenance therapy for treating multiple myeloma in a subject, which may include administering an immunomodulatory agent maintenance therapy to a subject after having received a BCMA targeted CAR T cell therapy for treating multiple myeloma, wherein prior to the BCMA target CAR T cell therapy the subject had an early relapse to an autologous stem cell transplant (ASCT) after an induction therapy for treating the multiple myeloma.

Among the provided the methods and uses are methods of treating multiple myeloma in a subject, which may include administering to the subject a BCMA targeted CAR T cell therapy followed by an immunomodulatory agent maintenance therapy, wherein the subject, at the time of administering the BCMA targeted CAR T cell therapy, has a multiple myeloma characterized by suboptimal response to autologous stem cell transplant (ASCT) after an induction therapy for treating the multiple myeloma. Also among the provided methods and uses are methods of treating multiple myeloma in a subject, which may include (a) selecting a subject with a multiple myeloma characterized by an suboptimal response to an autologous stem cell transplant (ASCT) after an induction therapy for treating the multiple myeloma; (b) administering to the selected subject an immunomodulatory agent maintenance therapy prior to a leukapheresis to obtain T cells from the subject for engineering with a BCMA targeted CAR to produce a BCMA targeted CAR T cell therapy; (c) administering to the selected subject the BCMA targeted CAR T cell therapy; and (d) after administering the BCMA targeted CAR T cell therapy, administering to the subject an immunomodulatory agent maintenance therapy. In some of any embodiments, preleukapheresis administration of an immunomodulatory agent therapy involves administering a subject lenalidomide prior to a leukapheresis to obtain T cells from the subject for engineering with a BCMA targeted CAR to produce a BCMA targeted CAR T cell therapy. For example, subjects receiving CAR T cell administration with lenalidomide maintenance, a lenalidomide preleukapheresis (1 cycle) is administered orally at a dose of 10 mg once daily on Days 1 to 28 of a 28-day cycle.

Among the provided methods and uses are methods of maintenance therapy for treating multiple myeloma in a subject, which may include administering an immunomodulatory agent maintenance therapy to a subject after having received a BCMA targeted CAR T cell therapy for treating multiple myeloma, wherein prior to the BCMA target CAR T cell therapy the subject had a suboptimal response to an autologous stem cell transplant (ASCT) after an induction therapy for treating the multiple myeloma.

Among the provided methods and uses are methods of treating high-risk multiple myeloma in a subject, which may include administering to the subject a BCMA targeted CAR T cell therapy, wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma.

Also among the provided methods and uses are methods of treating high-risk multiple myeloma in a subject, which may include administering to the subject a BCMA targeted CAR T cell therapy, wherein the subject, at the time of administering the BCMA targeted CAR T cell therapy, has a high-risk multiple myeloma characterized by early relapse to autologous stem cell transplant (ASCT) after an induction therapy for treating the multiple myeloma.

Also among the provided methods and uses are methods of treating multiple myeloma in a subject, which may include administering to the subject a BCMA targeted CAR T cell therapy, wherein the subject, at the time of administering the BCMA targeted CAR T cell therapy, has a multiple myeloma characterized by suboptimal response to autologous stem cell transplant (ASCT) after an induction therapy for treating the multiple myeloma.

### A. Subjects

In some embodiments, the subject is one who has multiple myeloma. In some embodiments, the subject who has multiple myeloma had an early relapse to one or more anti-myeloma treatment. In some embodiments, the subject who has multiple myeloma had an early relapse to a frontline anti-myeloma treatment. In some embodiments, the subject who has multiple myeloma had an inadequate response to one or more anti-myeloma treatment. In some embodiments, the subject who has multiple myeloma had an inadequate response to a frontline anti-myeloma treatment. In some embodiments, the subject who has multiple myeloma had a suboptimal response to one or more anti-myeloma treatment. In some embodiments, the subject who has multiple myeloma had a suboptimal response to a frontline anti-myeloma treatment. In some embodiments, the one or more anti-myeloma treatment can be stem cell transplant therapy. In some embodiments, the stem cell transplant therapy can be autologous stem cell therapy (ASCT) with induction therapy. In some embodiments, the stem cell transplant therapy can be ASCT with induction therapy and with a maintenance therapy. In some embodiments, the stem cell transplant therapy can be autologous stem cell therapy (ASCT) with induction therapy and high-dose chemotherapy (HDT). In some embodiments, the stem cell transplant therapy can be ASCT with induction therapy and high-dose chemotherapy (HDT) as well as with a maintenance therapy.

In some embodiments, the subject is one who has high risk multiple myeloma (MM). In some embodiments, the subject who has high risk MM had an early relapse to a stem cell transplant therapy. In some embodiments, the subject who has high risk MM had an inadequate response to a stem cell transplant therapy. In some embodiments, the subject who has high risk MM had a suboptimal response to a stem cell transplant therapy.

In some embodiments, the subject who has high risk multiple myeloma had an early relapse to one or more anti-myeloma treatment. In some embodiments, the subject who has high risk multiple myeloma had an early relapse to a frontline anti-myeloma treatment. In some embodiments, the subject who has high risk multiple myeloma had an inadequate response to one or more anti-myeloma treatment. In some embodiments, the subject who has high risk multiple myeloma had an inadequate response to a frontline anti-myeloma treatment. In some embodiments, the subject who multiple myeloma had a suboptimal response to one or more anti-myeloma treatment. In some embodiments, the subject who has multiple myeloma had a suboptimal response to a frontline anti-myeloma treatment. In some embodiments, the one or more anti-myeloma treatment can be stem cell transplant therapy. In some embodiments, the stem cell transplant therapy can be autologous stem cell therapy (ASCT). In some embodiments, the stem cell transplant therapy can be autologous stem cell therapy (ASCT) with induction therapy. In some embodiments, the stem cell transplant therapy can be ASCT with induction therapy and with a maintenance therapy. In some embodiments, the stem cell transplant therapy can be autologous stem cell therapy (ASCT) with induction therapy and high-dose chemotherapy (HDT). In some embodiments, the stem cell transplant therapy can be ASCT with induction therapy and high-dose chemotherapy (HDT) as well as with a maintenance therapy.

In some embodiments, the subject is one who has newly diagnosed multiple myeloma (NDMM). In some embodiments, the subject who has NDMM had an early relapse to a stem cell transplant therapy. In some embodiments, the subject who has NDMM had an inadequate response to a stem cell transplant therapy. In some embodiments, the subject who has NDMM had a suboptimal response to a stem cell transplant therapy.

In some embodiments, the subject who has NDMM had an early relapse to one or more anti-myeloma treatment. In some embodiments, the subject who has NDMM had an early relapse to a frontline anti-myeloma treatment. In some embodiments, the subject who has NDMM had an inadequate response to one or more anti-myeloma treatment. In some embodiments, the subject who has NDMM had an inadequate response to a frontline anti-myeloma treatment. In some embodiments, the subject who has NDMM had a suboptimal response to one or more anti-myeloma treatment. In some embodiments, the subject who has NDMM had a suboptimal response to a frontline anti-myeloma treatment. In some embodiments, the one or more anti-myeloma treatment can be stem cell transplant therapy. In some embodiments, the stem cell transplant therapy can be autologous stem cell therapy (ASCT). In some embodiments, the stem cell transplant therapy can be autologous stem cell therapy (ASCT) with induction therapy. In some embodiments, the stem cell transplant therapy can be ASCT with induction therapy and with a maintenance therapy. In some embodiments, the stem cell transplant therapy can be autologous stem cell therapy (ASCT) with induction therapy and high-dose chemotherapy (HDT). In some embodiments, the stem cell transplant therapy can be ASCT with induction therapy and high-dose chemotherapy (HDT) as well as with a maintenance therapy.

In some embodiments, the stem cell transplant therapy includes induction therapy, ASCT (single or tandem), and maintenance therapy. In some embodiments, the stem cell transplant therapy includes induction therapy, high-dose chemotherapy (HDT), ASCT and maintenance therapy. In specific embodiments, the maintenance therapy is a lenalidomide-containing therapy. In some embodiments, induction with or without ASCT and with or without maintenance therapy is considered a single regimen.

In some embodiments, the subject is one with a newly diagnosed multiple myeloma (NDMM). In some embodiments, the ASCT with the induction therapy is a frontline therapy for treating the NDMM subject. In some embodiments, the ASCT after the induction therapy is a frontline therapy for treating a NDMM. In some embodiments, the ASCT after the induction therapy and high-dose chemotherapy (HDT) is a frontline therapy for treating a NDMM. In some embodiments, the ASCT after the induction therapy with a maintenance therapy is a frontline therapy for treating a NDMM. In some embodiments, the ASCT after the induction therapy and high-dose chemotherapy (HDT) with a maintenance therapy is a frontline therapy for treating a NDMM.

In some embodiments, the subject is one who had an inadequate response to one or more anti-myeloma treatment. In some embodiments, the inadequate response is numerically lower than a very good partial response or <VGPR after ASCT. In some embodiments, the inadequate response can be characterized by less than very good partial response (VGPR) at 70-110 days after last treatment of the one or more anti-myeloma treatment without use of consolidation or maintenance therapy. In some embodiments, the inadequate response can be characterized by less than very good partial response (VGPR) at 80-120 days after last treatment of the one or more anti-myeloma treatment without use of consolidation or maintenance therapy. In some embodiments, the one or more anti-myeloma treatment can be autologous stem cell therapy (ASCT). In some embodiments, the stem cell transplant therapy can be autologous stem cell therapy (ASCT) with induction therapy.

In some embodiments, the subject is one who had an inadequate response to an ASCT with prior induction therapy. In some embodiments, the inadequate response is numerically lower than a very good partial response or <VGPR after ASCT. In some embodiments, the inadequate response can be characterized by less than very good partial response (VGPR) at 70-110 days after last treatment of the ASCT without use of consolidation or maintenance therapy. In some embodiments, the inadequate response can be characterized by less than very good partial response (VGPR) at 80-120 days after last treatment of the ASCT without use of consolidation or maintenance therapy.

In some embodiments, the inadequate response to the ASCT with prior induction therapy is characterized by less than very good partial response (VGPR) at 70-110 days after last ASCT. In some embodiments, the inadequate response to the ASCT with prior induction therapy is characterized by less than very good partial response (VGPR) at 80-120 days after last ASCT.

In some embodiments, the subject is one who had a suboptimal response to one or more anti-myeloma treatment. In some embodiments, the suboptimal response can be characterized by partial response or very good partial response (VGPR) at 80-110 days after last treatment of the one or more anti-myeloma treatment without use of consolidation or maintenance therapy. In some embodiments, the suboptimal response can be characterized by partial response or very good partial response (VGPR) at about 100 days after last treatment of the one or more anti-myeloma treatment without use of consolidation or maintenance therapy. In some embodiments, the one or more anti-myeloma treatment can be autologous stem cell therapy (ASCT). In some embodiments, the stem cell transplant therapy can be autologous stem cell therapy (ASCT) with induction therapy and high-dose chemotherapy (HDT).

In some embodiments, the subject is one who had a suboptimal response to an ASCT with prior induction therapy. In some embodiments, the subject is one who had a suboptimal response to an ASCT with prior induction therapy and high-dose chemotherapy (HDT). In some embodiments, the suboptimal response can be characterized by partial response or very good partial response (VGPR) at 80-110 days after ASCT. In some embodiments, the suboptimal response can be characterized by partial response or very good partial response (VGPR) at about 100 days after ASCT.

In some embodiments, the suboptimal response to the ASCT with prior induction therapy and high-dose chemotherapy (HDT) is characterized by partial response or very good partial response (VGPR) at 80-110 days after last ASCT. In some embodiments, the suboptimal response to the ASCT with prior induction therapy and high-dose chemotherapy (HDT) is characterized by partial response or very good partial response (VGPR) at about 100 days after last ASCT.

In some embodiments, the multiple myeloma is a newly diagnosed multiple myeloma (NDMM). In some embodiments, the subject is one who has a NDMM characterized by an inadequate response to an autologous stem cell transplant (ASCT) with an induction therapy for treating the multiple myeloma. In some embodiments, the subject is one who has a NDMM characterized by an early relapse to an autologous stem cell transplant (ASCT) with an induction therapy for treating the multiple myeloma. In some embodiments, the subject is one who has a NDMM characterized by an early relapse to an autologous stem cell transplant (ASCT) after an induction therapy for treating the multiple myeloma. In some embodiments, the subject is one who has a NDMM characterized by a suboptimal response to an autologous stem cell transplant (ASCT) with an induction therapy and high-dose chemotherapy (HDT) for treating the multiple myeloma. In some embodiments, the subject is one who has a NDMM characterized by a suboptimal response to an autologous stem cell transplant (ASCT) after an induction therapy and high-dose chemotherapy (HDT) for treating the multiple myeloma.

In some embodiments, the subject is one who had an early relapse to the one or more anti-myeloma treatment. In some embodiments, early relapse can be disease progression after a period of response to treatment. In some embodiments, early relapse is within 18 months of starting frontline therapy. In some embodiments, early relapse is <12 months after starting frontline therapy. In some embodiments, early relapse is within 18 months of treatment after starting frontline therapy. In some embodiments, frontline therapy includes induction therapy, ASCT, and maintenance therapy, which can include lenalidomide.

In some embodiments, subjects who relapse earlier (<12 months) after treatment with ASCT have poorer outcomes than subjects who relapse later. See Paiva B, et al. 2012, Blood, 119 (3): 687-691; Bygrave et al. 2021, Br J Haematol, 193 (3): 551-555; Davies et al. 2022, Blood Cancer Discov, 3 (4): 273-284. 4; and Majithia et al. 2016, Leukemia, 30 (11): 2208-2213. In some embodiments, patients who experience early relapse have shorter lives than those who experience later relapse. In some embodiments, the median overall survival after ASCT for early relapse patients is 26 months. See Bygrave et al. 2021, Br J Haematol, 193 (3): 551-555. In some embodiments, the median overall survival after ASCT for later relapse patients is 91 months. See Bygrave et al. 2021, Br J Haematol, 193 (3): 551-555.

In some embodiments, early relapse can be characterized by development of progressive disease (PD) less than 18 months from starting the one or more anti-myeloma treatment. In some embodiments, early relapse can be characterized by development of progressive disease (PD) less than 18 months from starting a frontline anti-myeloma treatment. In some embodiments, early relapse can be characterized by the development of progressive disease (PD) less than 18 months since the date of start of initial therapy of the one or more anti-myeloma treatment or frontline anti-myeloma treatment. In particular embodiments, the initial therapy of the one or more anti-myeloma treatment may contain induction, ASCT (single or tandem) and lenalidomide containing maintenance. In particular embodiments, the frontline anti-myeloma treatment may contain induction, ASCT (single or tandem) and lenalidomide containing maintenance. In some embodiments, the subject may be a candidate for single-agent lenalidomide maintenance. In some embodiments, the subject must be a candidate for single-agent lenalidomide maintenance.

In some embodiments, early relapse can be characterized by early relapse to an autologous stem cell transplant (ASCT) with an induction therapy for treating the multiple myeloma. In some embodiments, early relapse can be characterized by early relapse to an autologous stem cell transplant (ASCT) after an induction therapy for treating the multiple myeloma.

In some embodiments, the multiple myeloma is a high-risk multiple myeloma. In some embodiments, the subject is one who has a high-risk multiple myeloma characterized by an inadequate response to an autologous stem cell transplant (ASCT) with an induction therapy for treating the multiple myeloma. In some embodiments, the subject is one who has a high-risk multiple myeloma characterized by an inadequate response to an autologous stem cell transplant (ASCT) after an induction therapy for treating the multiple myeloma. In some embodiments, the subject is one who has a high-risk multiple myeloma characterized by an early relapse to an autologous stem cell transplant (ASCT) with an induction therapy for treating the multiple myeloma. In some embodiments, the subject is one who has a high-risk multiple myeloma characterized by an early relapse to an autologous stem cell transplant (ASCT) after an induction therapy for treating the multiple myeloma. In some embodiments, the subject is one who has a high-risk multiple myeloma characterized by a suboptimal response to an autologous stem cell transplant (ASCT) with an induction therapy for treating the multiple myeloma. In some embodiments, the subject is one who has a high-risk multiple myeloma characterized by a suboptimal response to an autologous stem cell transplant (ASCT) after an induction therapy for treating the multiple myeloma.

In some embodiments the subject may have received ≥3 cycles of induction therapy. In some embodiments the subject must have received ≥3 cycles of induction therapy. In some embodiments, the subject may have received minimum 3 cycles of induction therapy, which may contain at minimum, a proteasome inhibitor, an immunomodulatory agent and dexamethasone. In some embodiments, the subject must have received minimum 3 cycles of induction therapy, which must contain at minimum, a proteasome inhibitor, an immunomodulatory agent and dexamethasone.

In some embodiments, the subject may have received 4 to 6 cycles of induction therapy, which may contain at minimum, a proteasome inhibitor, an immunomodulatory agent and dexamethasone. In some embodiments, the subject must have received 4 to 6 cycles of induction therapy, which must contain at minimum, a proteasome inhibitor, an immunomodulatory agent and dexamethasone. In some embodiments, the subject may have received 4 to 6 cycles of induction therapy, which may contain at minimum, a proteasome inhibitor and an immunomodulatory agent. In some embodiments, the subject must have received 4 to 6 cycles of induction therapy, which must contain at minimum, a proteasome inhibitor and an immunomodulatory agent.

In some embodiments, the subject may have had single ASCT within 6 months prior to consent (e.g., time of screening). In some embodiments, the subject must have had single ASCT within 6 months prior to consent (e.g., time of screening). In some embodiments, the subject may have had single ASCT within 60 to 180 days prior to consent (e.g., time of screening). In some embodiments, the subject must have had single ASCT within 60 to 180 days prior to consent (e.g., time of screening). In some embodiments, the subject may have had single ASCT within 60 to 150 days prior to consent (e.g., time of screening). In some embodiments, the subject must have had single ASCT within 60 to 150 days prior to consent (e.g., time of screening). In some embodiments, the subject may have had single ASCT within 80 to 120 days prior to consent (e.g., time of screening). In some embodiments, the subject must have had single ASCT within 80 to 120 days prior to consent (e.g., time of screening).

In some embodiments, consent can be at the time of screening. In some embodiments, consent or screening can occur about 8-12 weeks before peripheral blood stem cells (PBSCs) are collected. In some embodiments, consent or screening can occur about 9-10 weeks before peripheral blood stem cells (PBSCs) are collected. In some embodiments, consent or screening can occur at about 9 weeks before peripheral blood stem cells (PBSCs) are collected. In some embodiments, consent or screening can occur at about 10 weeks before peripheral blood stem cells (PBSCs) are collected. In some embodiments, consent or screening can occur about 8-12 weeks before peripheral blood mononuclear cells (PBMCs) are collected. In some embodiments, consent or screening can occur about 9-10 weeks before peripheral blood mononuclear cells (PBMCs) are collected. In some embodiments, consent or screening can occur at about 9 weeks before peripheral blood mononuclear cells (PBMCs) are collected. In some embodiments, consent or screening can occur at about 10 weeks before peripheral blood mononuclear cells (PBMCs) are collected. In some embodiments, consent or screening can occur about 8-12 weeks before apheresis. In some embodiments, consent or screening can occur about 9-10 weeks before apheresis. In some embodiments, consent or screening can occur at about 9 weeks before apheresis. In some embodiments, consent or screening can occur at about 10 weeks before apheresis.

In some embodiments, the subject may have recovery to Grade 1 or baseline of any non-hematologic toxicities due to prior treatments, excluding alopecia and Grade 2 neuropathy. In some embodiments, the subject must have recovery to Grade 1 or baseline of any non-hematologic toxicities due to prior treatments, excluding alopecia and Grade 2 neuropathy.

In some embodiments, the subject may have had ASCT (single or tandem) and <VGPR (excluding PD) at first assessment between 70 to 110 days after last ASCT, with initial therapy without consolidation and maintenance. In some embodiments, the subject must have had ASCT (single or tandem) and <VGPR (excluding PD) at first assessment between 70 to 110 days after last ASCT, with initial therapy without consolidation and maintenance.

In some embodiments, the subject may have had ASCT and partial response or very good partial response (VGPR) at 80-120 days after last ASCT, with initial therapy without consolidation and maintenance. In some embodiments, the subject must have had ASCT and partial response or very good partial response (VGPR) at 80-120 days after last ASCT, with initial therapy without consolidation and maintenance. In some embodiments, the subject may have had ASCT and partial response or very good partial response (VGPR) at about 100 days after last ASCT, with initial therapy without consolidation and maintenance. In some embodiments, the subject must have had ASCT and partial response or very good partial response (VGPR) at about 100 days after last ASCT, with initial therapy without consolidation and maintenance.

In some embodiments, the subject is one who had not received a prior immunomodulatory agent maintenance therapy. In some embodiments, the subject is one who had not received a consolidation therapy. In some embodiments, the subject is one who received a prior immunomodulatory agent maintenance therapy after the ASCT with induction therapy. In some aspects, the subject can have received ≤7 days of lenalidomide (LEN) maintenance therapy if the investigator documents that there is no impact to the overall benefit/risk assessment due to the temporary interruption of LEN.

In some embodiments, the subject is one who was diagnosed with multiple myeloma about three years or less before administering the BCMA targeted CAR T cell therapy. In some embodiments, the subject is one who was diagnosed with multiple myeloma about two years or less before administering the BCMA targeted CAR T cell therapy. In some embodiments, the subject is one who was diagnosed with multiple myeloma about 1.6 years or less before administering the BCMA targeted CAR T cell therapy.

In some embodiments, the subject is one who, at the time of administering the BCMA targeted CAR T cell therapy, has R-ISS stage I disease. In some embodiments, the subject is one who, at the time of administering the BCMA targeted CAR T cell therapy, has R-ISS stage II disease. In some embodiments, the subject is one who, at the time of administering the BCMA targeted CAR T cell therapy, has R-ISS stage III disease.

In some embodiments, R-ISS is derived calculated using baseline values of albumin and beta-2-microglobulin. In some embodiments, R-ISS is derived using baseline ISS stage, cytogenetic abnormality, and serum lactate dehydrogenase.

In some embodiments, the subject is one who has high risk multiple myeloma. In some embodiments, high risk multiple myeloma is characterized as having high-risk cytogenetics. In some embodiments, the subject is one who, at the time of administering the BCMA targeted CAR T cell therapy, has high-risk cytogenetics. In some embodiments, high-risk cytogenetics can be characterized as del (17p), t(4; 14), or t(14; 16). In some embodiments, high risk cytogenetics features include del (17p). In some embodiments, high risk cytogenetics features include t(4; 14). In some embodiments, high risk cytogenetics features include t(14; 16).

In some embodiments, two or more high-risk cytogenetic features can be characterized as ultra high-risk cytogenetics. In some embodiments, ultra high-risk cytogenetic features can include: del (17p), t(4; 14), t(14; 16), t(14; 20), 1q amp.

In some embodiments, high-risk cytogenetics can be characterized by deletion of chromosome 13 by metaphase analysis. In some embodiments, high-risk cytogenetics can be characterized by deletion of 17p13 (p53) by Fluorescence in situ hybridization (FISH) or metaphase analysis. In some embodiments, high-risk cytogenetics can be characterized by IgH translocations. In some embodiments, high-risk cytogenetics can be characterized by deletion of t(4; 14) by FISH. In some embodiments, high-risk cytogenetics can be characterized by deletion of t(14; 16) by FISH. In some embodiments, high-risk cytogenetics can be characterized by deletion of t(8; 14) by FISH. In some embodiments, high-risk cytogenetics can be characterized by deletion of t(14; 20) by FISH. In some embodiments, high-risk cytogenetics can be characterized by hypodiploidy detected by FISH or metaphase analysis. In some embodiments, high-risk cytogenetics can be characterized by any complex cytogenetic abnormality detected by metaphase analysis, with the exception of hyperdiploidy.

In some embodiments, the subject is one who, at the time of administering the BCMA targeted CAR T cell therapy, has bone marrow biopsy-determined high tumor burden, optionally wherein the high tumor burden is ≥50% bone marrow CD138+ plasma cells.

In some embodiments, low tumor burden is determined by <50% bone marrow CD138+ plasma cells. In some embodiments, high tumor burden is determined by ≥50% bone marrow CD138+ plasma cells.

In some embodiments, the subject is one who, at the time of administering the BCMA targeted CAR T cell therapy, has extramedullary disease.

In some embodiments, the subject is one who, at the time of administering the BCMA targeted CAR T cell therapy, had had an Eastern Cooperative Oncology Group (ECOG) performance status (PS)≤1. In some embodiments, the subject is one who, at the time of administering the BCMA targeted CAR T cell therapy, had an Eastern Cooperative Oncology Group (ECOG) PS of 0.

In some embodiments, the subject may have an Eastern Cooperative Oncology Group (ECOG) performance status≤1. In some embodiments, the subject must have an Eastern Cooperative Oncology Group (ECOG) performance statue≤1. In some embodiments, the subject may have an Eastern Cooperative Oncology Group (ECOG) performance status of 2 due to pain because of underlying myeloma-associated bone lesions if an investigator deems this subject to be eligible.

In some embodiments, the subject is one who, prior to the administration of the BCMA targeted CAR T cell therapy, has received a lymphodepleting therapy comprising the administration of fludarabine at or about 20-40 mg/m² body surface area of the subject (optionally at or about 30 mg/m²) daily for 2-4 days, and/or cyclophosphamide at or about 200-400 mg/m² body surface area of the subject (optionally at or about 300 mg/m²) daily for 2-4 days.

In some embodiments, the subject is one who, prior to the administration of the BCMA targeted CAR T cell therapy, has received a lymphodepleting therapy comprising the administration of fludarabine at or about 30 mg/m² body surface area of the subject, daily, and cyclophosphamide at or about 300 mg/m² body surface area of the subject, daily, for 3 days.

In some embodiments, the subject is one who received a bridging therapy prior to receiving the BCMA targeted CAR T cell therapy, optionally wherein the bridging therapy was administered to the subject in the period between obtaining the T cells from the subject and administering the BCMA targeted CAR T cell therapy to the subject. In some embodiments, the subject is one who received a bridging therapy following leukapheresis.

In some embodiments, the duration of the bridging therapy is between 10 and 70 days, 10 and 60 days, 15 and 70 days, 15 and 60 days, 20 and 70 days or 20 and 60 days. In some embodiments, the duration of the bridging therapy is between 22 and 54 days.

In some embodiments, the median duration of the bridging therapy is between 20 to 60 days, 30 to 60 days, 40 to 60 days, 30 to 50 days, or 30 to 40 days. In some embodiments, the median duration of the bridging therapy can be 30 days, 31 days, 32 days, 34 days, 35 days, 36 days, 37 days, 38 days, 39 days, 40 days, 41 days, 42 days, 43 days, 44 days, or 45 days. In some embodiments, the median duration of the bridging therapy is 38 days. In some embodiments, the subject is a human. In some embodiments, the subject is 18 years of age or older.

Also provided are methods of administering and uses, such as therapeutic uses, of the anti-BCMA recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a multiple myeloma (MM). In some embodiments, the molecule, cell, and/or composition is/are administered in an effective amount to effect treatment of the MM. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having the MM. In some embodiments, the methods thereby treat the MM in the subject. Also provided herein are of use of any of the compositions, such as pharmaceutical compositions provided herein, for the treatment of a multiple myeloma (MM), such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, reducing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, a “subject” or an “individual” is a human.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338.

Among the diseases to be treated is multiple myeloma (MM), which is associated with BCMA expression. See Coquery et al., Crit Rev Immunol., 2012, 32 (4): 287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19 (8): 2048-2060.

In some embodiments the multiple myeloma (MM) is a high risk MM or a relapsed and/or refractory multiple myeloma. In some embodiments the multiple myeloma (MM) is a high risk MM. In some embodiments, the subject has high risk MM characterized by early relapse, inadequate response, or a suboptimal response to prior ASCT with induction therapy. In some embodiments, the subject has high risk MM characterized by early relapse, inadequate response, or suboptimal response to prior ASCT after induction therapy.

In some embodiments, a high risk MM comprises IMWG high risk cytogenetics. In some of any embodiments, at the time of administration of the cell therapy, the subject has IMWG high risk cytogenetics. In some embodiments, high risk cytogenetics comprise del(17p), t(4:14) and t(14; 16). In some embodiments, the multiple myeloma (MM) is a newly diagnosed multiple myeloma (NDMM). In some embodiments the multiple myeloma (MM) is a relapsed and/or refractory multiple myeloma. In some embodiments the multiple myeloma (MM) is a relapsed and refractory multiple myeloma (R/R MM). In some of any embodiments, at the time of administration, the subject has a R/R MM. In some embodiments, the methods may identify a subject who has, is suspected to have, or is at risk for developing a multiple myeloma. Hence, provided are methods for identifying subjects with multiple myeloma and selecting them for treatment with and/or administering to them any of the BCMA-binding recombinant receptors (e.g., CARs) described herein, or engineered cells expressing the same.

In some embodiments, the T cell therapy, e.g., adoptive cell therapy, e.g., adoptive T cell therapy, is carried out by autologous transfer, in which the cells are isolated and/or otherwise prepared from the subject who is to receive the T cell therapy, or from a sample derived from such a subject. Thus, in some aspects, the cells are derived from a subject, e.g., patient, in need of a treatment and the cells, following isolation and processing, are administered to the same subject.

In some embodiments, the T cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The subject can be male or female and can be any suitable age, including infant, juvenile, adolescent, adult, and geriatric subjects. In some embodiments, the subject is an adult (i.e., 18 years of age or older).

In some embodiments, the dose and/or frequency of administration is determined based on efficacy and/or response. In some embodiments, efficacy is determined by evaluating disease status. Exemplary methods for assessing disease status include: measurement of M protein or soluble BCMA (sBCMA) in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17 (8): e328-346). In some embodiments, response is evaluated using minimal residual disease (MRD) assessment. In some embodiments, MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQOL) of the subject to which the recombinant receptor and/or cells is/are administered. In some embodiments, dose and/or frequency of administration can be changed, i.e., increased or decreased, based on any of the above criteria.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sørensen et al., (1993) Br J Cancer 67 (4) 773-775. In some embodiments, the subjects that are to be administered according to the methods or treatment regimen provided herein include those with an ECOG performance status of 0 or 1. In some embodiments, the subject has an Eastern Cooperative Oncology Group (ECOG) performance status of 0 or 1. In some embodiments, the subject has an Eastern Cooperative Oncology Group (ECOG) performance status of 0. In some embodiments, the subject has an Eastern Cooperative Oncology Group (ECOG) performance status of 1. In some embodiments, the subject has an Eastern Cooperative Oncology Group (ECOG) performance status of 2 due to pain because of underlying myeloma-associated bone lesions and can be eligible per investigator's discretion).

### 1. ASCT

In some embodiments, the one or more anti-myeloma treatment is an autologous stem cell transplant therapy. In some embodiments, the stem cell transplant therapy can be autologous stem cell therapy (ASCT).

As used herein, autologous relates to a transplantation in which a donor and recipient is the same individual. In some embodiments, for an ASCT, autologous transplant cells are harvested from a subject and then returned to the same subject. In some embodiments, an ASCT refers to a procedure in which a sample of a subject's own stem cells are removed and subsequently transplanted back into the same subject.

In some embodiments, the stem cells can be harvested from bone marrow (BM). In some embodiments, the stem cells can be harvested from peripheral blood (PB). In some embodiments, ASCT comprises either autologous bone marrow transplant (ABMT) or peripheral blood stem cell transplant (PBSCT).

In some embodiments, once obtained, stem cells can be frozen or cryopreserved until needed. In some embodiments, stem cells can be obtained from a subject, cryopreserved at temperatures≤−85° C., and then thawed and returned (e.g., transplanted, typically by transfusion) to the subject. In particular embodiments, the stem cell aliquots can be thawed, loaded into one or more sterile syringes or infusion bags, and injected intravenously over a period of time ranging from about 30 minutes to about 45 minutes.

In some embodiments, stem cells capable of reconstituting a patient's immune system can be obtained from the patient's peripheral circulation following mobilization of such cells from the bone marrow into the peripheral blood. In some embodiments, mobilization of stem cells can be accomplished by treatment of a patient with one or more factors that can (i) stimulate an increase in proliferation of stem cells and/or progenitor cells, and/or (ii) stimulate migration of stem cells and/or progenitor cells from the BM into the peripheral circulation.

In some embodiments, stem cell factors (e.g., colony-stimulating factors) are injected to the subject to trigger release of hematopoietic stem cells into the bloodstream. In some embodiments, such factors can be administered with adjuvants and/or other accessory substances, separately or in combination as desired. In some embodiments, factors include, without limitation, granulocyte colony-stimulating factor (G-CSF), granulocyte/macrophage colony-stimulating factor (GM-CSF), c-kit ligand (stem cell factor (SCF)), interleukin-2, -7, -8, and -12 (IL-2, IL-7, IL-8, and IL-12), and flt-3 ligand. See, e.g., Bungart et al. (1990) Br. J. Haematol. 76:174; Terella et al. (1993) Bone Marrow Transplant. 11:271; Molineux et al. (1991) Blood 85:275; Grzegorzewski et al. (1994) Blood 83:377; Laterveer et al. (1995) Blood 85:2269; Jackson et al. (1995) Blood 85:2371; and Lyman et al. (1994) Blood 83:2795. In some embodiments, factors to be administered can include, without limitation, G-CSF alone (e.g., 10 μg/kg/day G-CSF), G-CSF+flt-3 ligand (e.g., 10 μg/kg/day G-CSF+50 μg/kg/day flt-3 ligand), or GM-CSF+flt-3 ligand (e.g., 5 μg/kg/day GM-CSF+50 μg/kg/day flt-3 ligand). See, e.g., Sudo et al. (1997) Blood 89:3186.

In some embodiments, factors can be administered prior to harvest or starting on the day of harvest. In some embodiments, factors can be given on a daily basis for one to seven days (e.g., for one, two, three, four, five, six, or seven days), or until stem cell harvesting is complete. In some embodiments, factors that stimulate stem cell proliferation or mobilization can be administered using any suitable method. In some embodiments, such factors can be administered parenterally (e.g., by subcutaneous, intrathecal, intraventricular, intramuscular, or intraperitoneal injection, or by intravenous drip).

In some embodiments, mobilization of stem cells can be evaluated by determining the number of CD34+ cells present before, during, and/or after treatment with one or more factors. In some embodiments, the number of CD34+ cells can be determined by FACS analysis using CD34-specific antibodies conjugated to fluorescent or other labeling moieties.

In some embodiments, following mobilization, peripheral blood stem cells (PBSC) can be collected using an apheresis procedure. In some embodiments, during mobilization, peripheral blood stem cells (PBSC) can be collected using an apheresis procedure.

Apheresis involves removal of whole blood from a patient or donor. In some embodiments, within an instrument that is essentially designed as a centrifuge, the components of the whole blood are separated. In some embodiments, the one or more of the separated portions is then withdrawn, and the remaining components can be re-transfused into the patient or donor. In some embodiments, all or most (e.g., 80%, 85%, 90%, 95%, 99%, or 100%) of the erythrocytes in a sample of whole blood can be returned to a patient during an apheresis procedure, while lymphocytes (e.g., NK cells) and stem cells can be collected.

In some embodiments, 80% of erythrocytes in a sample of whole blood is returned to a patient during an apheresis procedure. In some embodiments, 85% of erythrocytes in a sample of whole blood is returned to a patient during an apheresis procedure. In some embodiments, 90% of erythrocytes in a sample of whole blood is returned to a patient during an apheresis procedure. In some embodiments, 95% of erythrocytes in a sample of whole blood is returned to a patient during an apheresis procedure. In some embodiments, 99% of erythrocytes in a sample of whole blood is returned to a patient during an apheresis procedure. In some embodiments, 100% of erythrocytes in a sample of whole blood is returned to a patient during an apheresis procedure.

Lymphocytes are white blood cells (WBC) that are formed in lymphatic tissue throughout the human body (e.g., lymph nodes, spleen, thymus, tonsils, Peyer's Patches, and bone marrow). In normal adults, lymphocytes comprise approximately 22% to 28% of the total number of leukocytes in the circulating blood. As used herein, the term “lymphocyte” includes NK cells, B cells, and T cells (e.g., T helper cells, cytotoxic T cells, and T suppressor cells).

In some embodiments, a commercially available blood cell collection device can be used, such as the CS3000R blood cell collection device marketed by the Fenwal Division of Baxter Healthcare Corporation (Fenwal Laboratories, Deerfield, Ill.). Methods for performing apheresis with the CS3000R machine are described in Williams et al. (1990) Bone Marrow Transplantation 5:129-33, and Hillyer et al. (1993) Transfusion 33:316-21, for example, both of which are incorporated herein by reference in their entirety.

In some embodiments, a total blood volume between 9.5 and 10 L per apheresis procedure can be processed at a flow rate of 50 to 70 mL/min. Following collection, a cell count can be performed on an aliquot of the total product to determine the number of stem cells. In some embodiments, cells can be collected until the total sample taken from the patient reaches a concentration of at least 1×10⁶ CD34⁺ stem cells/kg. In some embodiments, cells can be collected until the total sample taken from the patient reaches a concentration of at least 2×10⁶ CD34⁺ stem cells/kg. In some embodiments, cell s can be collected until the total sample taken from the patient reaches a concentration of at least 3×10⁶ CD34⁺ stem cells/kg.

In some embodiments, despite various methods of PBSC mobilization, adequate numbers of PBSC for ASCT may be not collected from some patients during a single apheresis procedure. In some embodiments, for these patients, a BM harvest or a second attempt at PBSC mobilization can be performed.

In some embodiments, apheresis products can be centrifuged (e.g., at 400 g for 10 minutes), and the plasma can be removed to yield a total volume of, for example, about 100 mL. In some embodiments, the resulting cell suspension can be mixed with a physiological freezing solution [e.g., 100 mL minimal essential medium such as MEM-S (Invitrogen Life Technologies, Carlsbad, Calif.) supplemented with 20% dimethylsulfoxide (DMSO)]. In some embodiments, cell/media suspensions can be transferred to freezing bags (such as those manufactured by Delmed, Canton, Mass.) or any other freezing receptacle known in the art, and frozen to −100° C. using, for example, a computer-controlled cryopreservation device (e.g., the Cryoson-BV-6; Cryoson Deutschland GmbH, FRG). In some embodiments, the cells then can be transferred into liquid nitrogen and stored until transplantation.

In some embodiments, a patient's stem cells can be collected by BM harvest using procedures known in the art, or by a stem cell apheresis procedure as described above. In some embodiments, the collected stem cells can be cryopreserved by procedures as described above. In some embodiments, a patient can undergo a debulking procedure such as high-dose chemotherapy and/or radiation therapy.

In some embodiments, patients typically undergo a pre-transplant workup to evaluate, for example, heart, liver, kidney, and lung function, as well as current disease status. In some embodiments, patients deemed to be eligible (e.g., healthy enough) for ASCT are subjected to a tumor debulking procedure prior to ASCT. In some embodiments, a tumor debulking procedure can include treating a patient with induction therapy, high doses of chemotherapy, radiation therapy, and/or surgery (e.g., surgery with anesthesia) before the transplant.

In some embodiments, the ASCT therapy can include a high-dose chemotherapy (HDT) with autologous stem cell rescue after the completion of induction therapy. In some embodiments, the HDT can additionally include radiation therapy.

In some embodiments, the ASCT therapy can include the induction therapy, the high-dose chemotherapy (HDT) and the ASCT. In some embodiments, there is not subsequent consolidation or maintenance after the ASCT.

In some embodiments, stem cells for transplant typically are collected prior to tumor debulking regimens, since such potentially lethal procedures can be immunosuppressive and can severely damage or destroy the BM. In some embodiments, an ASCT following a debulking procedure can reconstitute the patient's immune cells with stem cells present in the transplant.

In some embodiments, after the debulking procedure is completed, the patient's stem cells can be transplanted. In some embodiments, ASCT can be done almost immediately after a debulking procedure (e.g., 24 to 48 hours after HDT). In some embodiments, a longer period of time (e.g., a week to several months) can elapse between a debulking procedure and ASCT.

In some embodiments, due to the likelihood of immunosuppression as a result of the debulking procedure, protective isolation precautions generally are taken after ASCT at least until the reinfused stem cells begin to engraft. “Engraftment” refers to a process whereby the transplanted stem cells begin to differentiate into mature blood cells.

In some embodiments, stem cells can be treated prior to transplantation with, for example, anticancer drugs or antibodies to reduce the number of cancerous cells that may be present in the sample. In some embodiments, such procedures are referred to as “purging.”

In some embodiments, a subject may have one autologous transplant, known as single ASCT. In some embodiments, a subject may have two autologous transplants, known as tandem ASCT. In some embodiments, the two autologous transplants can be 6 to 12 months apart from each other. In particular embodiments, the two autologous transplants are 6 months apart from each other. In particular embodiments, the two autologous transplants are 7 months apart from each other. In particular embodiments, the two autologous transplants are 8 months apart from each other. In particular embodiments, the two autologous transplants are 9 months apart from each other. In particular embodiments, the two autologous transplants are 10 months apart from each other. In particular embodiments, the two autologous transplants are 11 months apart from each other. In particular embodiments, the two autologous transplants are 12 months apart from each other.

### 2. Induction Therapy

In some embodiments, the stem cell transplant therapy includes an induction therapy followed by the stem cell transplant. In some embodiments, induction therapy is also referred to as induction or induction period therapy. In some embodiments, induction therapy refers to the first treatment given for a disease with the intention of reducing the amount of malignant plasma cell burden and improving the depth of response. In some embodiments, induction therapy refers to the first treatment given for multiple myeloma with the intention of reducing the amount of malignant plasma cell burden and improving the depth of response. In some embodiments, the induction therapy decreases tumor burden and increases the likelihood of engraftment.

In some embodiments, the induction therapy comprises a proteasome inhibitor. In some embodiments, the induction therapy comprises an immunomodulatory agent. In some embodiments, the induction therapy comprises a corticosteroid. In some embodiments, the induction therapy comprises an alkylating agent. In some embodiments, the induction therapy comprises a monoclonal antibody. In some embodiments, the induction therapy is a monoclonal antibody.

In some embodiments, the induction therapy comprises a proteasome inhibitor. In some embodiments, the proteasome inhibitor inhibits the 26S proteasome. In some embodiments, inhibition of the 26S proteasome inhibits or blocks targeted proteolysis by the proteasome, thereby disrupting cell signaling pathways, which can lead to cell cycle arrest, apoptosis, and inhibition of angiogenesis. In some embodiments, the proteasome inhibitor inhibits nuclear factor kappa B (NFkB).

In some embodiments, the proteasome inhibitor is selected from among the group consisting of bortezomib, carfilzomib, and ixazomib.

In some embodiments, the proteasome inhibitor reversibly inhibits the 26S proteasome. In some embodiments, the proteasome inhibitor is [(1R)-3-methyl-1-[[(2S)-3-phenyl-2-(pyrazine-2-carbonylamino) propanoyl]amino]butyl]boronic acid, also known as bortezomib or Velcade®. In some embodiments, the proteasome inhibitor is bortezomib. In some embodiments, the proteasome inhibitor has the following structure:

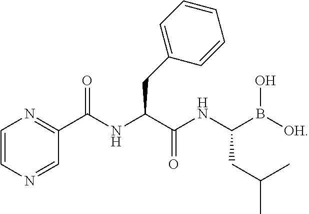 [SMILES: CC(C)C[C@H](NC(=O)[C@H](Cc1ccccc1)NC(=O)c1cnccn1)B(O)O]

or a pharmaceutically acceptable salt, solvate, hydrate, stereoisomer, tautomer or racemic mixtures thereof, including and compositions thereof. In some embodiments, the proteasome inhibitor has the following structure:

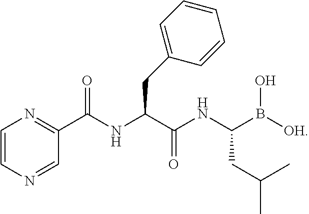 [SMILES: CC(C)C[C@H](NC(=O)[C@H](Cc1ccccc1)NC(=O)c1cnccn1)B(O)O]

Compositions of bortezomib include but are not limited to those described in U.S. Pat. Nos. 578,054, 6,083,903, 6,713,446, 6,958,319, 8,962,572, and 10,314,880; and International Publication Nos. WO 2006/052733 and WO 2016/166653 (each incorporated herein by reference in its entirety).

In some embodiments, the composition comprising bortezomib is a “ready to use” formulation that contains bortezomib in dissolved or solubilized form and is intended to be used as such or upon further dilution in intravenous diluents. In preferred embodiments, pharmaceutical compositions comprising bortezomib are formulated for parenteral administration, e.g., injection or infusion.

Suitable solvents can be selected from aqueous and non-aqueous solvents such as, but are not limited to, glycerin, ethanol, n-propanol, n-butanol, isopropanol, ethyl acetate, dimethyl carbonate, acetonitrile, dichloromethane, methyl ethyl ketone, methyl isobutyl ketone, cyclohexane, dimethylacetamide (DMA), dimethyl sulfoxide (DMSO), N-methyl-2-pyrrolidone (NMP), 1,3-dimethyl-2-imidazolidinone (DMI), acetone, tetrahydrofuran (THF), dimethylformamide (DMF), propylene carbonate (PC), dimethyl isosorbide, water and mixtures thereof. Preferred solvents are ethanol, glycerin and water.

The bortezomib formulation may comprise stabilizers such as sugars and amino acids. Suitable stabilizers include glucose, trehalose, sucrose, mannitol, sorbitol, arginine, glycine, proline, methionine, lysine and the like.

The bortezomib formulation may comprise a chelating agent. Suitable chelating agents include DOTA (1,4,7,10-tetraazacyclododecane-1,4,7,10-tetraacetic acid), DTPA (diethylene triaminepentaacetic acid), EDTA (Ethylenediaminetetraacetic acid), ODDA (1,4,10,13-tetraoxa-7,16-diazacyclooctadecane-7), TTT A (1,7,13-triaza-4,10,16-trioxacyclooctadecane-N,N′,N″-triacetate), DOTRP (tetraethyleneglycol-1,5,9-triazacyclododecane-N,N′,N″,-tris(methylene phosphonic acid), EGTA (ethylene glycol-bis(P-aminoethyl ether)-tetraacetic acid) and the like.

The bortezomib formulation may also contain one or more antioxidants. Suitable anti-oxidants include, but are not limited to, monothioglycerol, ascorbic acid, sodium bisulfite, sodium metabisulfite, L-cysteine, thioglycolic acid, citric acid, tartaric acid, phosphoric acid, gluconic acid, thiodipropionic acid, and the like. In some embodiments, the most preferred anti-oxidant is monothioglycerol.

The bortezomib formulation for use in the present invention may optionally contain other pharmaceutically acceptable adjuvants such as buffering agents, pH adjusting agents, preservatives, tonicity modifiers and the like. The lists of solvents, stabilizers, chelating agents and antioxidants listed above may also be used in pharmaceutical compositions comprising other cytotoxic agents described herein unless stated otherwise.

In some embodiments, the induction therapy comprises administering bortezomib at a dose from or from about 0.1 to 100 mg/m², from or from about 0.1 to 10 mg/m², from or from about 0.1 to 5 mg/m², from or from about 0.1 to 1 mg/m², each inclusive. In some embodiments, the dose is 1.3 mg/m². In some embodiments, the dose is 1.3 mg/m². In some embodiments, the induction therapy comprises administering bortezomib twice weekly. See Richardson et al. 2009, J Clin Oncol, 27 (21): 3518-3525; Jagannath et al. 2005, Br J Haematol, 129 (6): 776-783; and Cavo et al. 2011, Blood 117 (23): 6063-6073 (each incorporated herein by reference in its entirety). In some embodiments, the dose is 1.0 mg/m² on a twice-weekly basis. See Popat et al. 2008, Br J Haematol, 141 (4): 512-516.

In some embodiments, the dose of bortezomib can be delivered subcutaneously or intravenously. In some embodiments, the dose of bortezomib is given as a single subcutaneous injection. In some embodiments, the dose of bortezomib is given as a rapid intravenous bolus over three to five seconds.

In some embodiments, the dose of bortezomib is 1.3 mg/m² each day of a cycle. In some embodiments, the cycle is 21 days. See Richardson et al. 2010, Blood, 116:679; Kumar et al. 2012, Blood, 119:4375; Rajkumar et al. 2011, Am J Hematol, 86:57; Moreau et al. 2011, Lancet Oncol 12:431; Rajkumar et al. 2010, Lancet Oncol, 11:909; Rajkumar et al. 2010, Lancet Oncol 11:29; and Niesvizky et al. 2007, Br J Haematol, 138:640 (each incorporated by reference in its entirety). In some embodiments, the cycle is 28 days. See Reeder et al. 2010, Blood, 115:3416; Reeder et al. 2009, Leukemia, 23:1337; Kropff et al. 2007, Br J Haemtol, 138:330; Moreau et al. 2011, Lancet Oncol, 12:431 (each incorporated by reference in its entirety).

In some embodiments, the proteasome inhibitor is a selective proteasome inhibitor. In some embodiments, the proteasome inhibitor is an irreversible proteasome inhibitor. In some embodiments, the proteasome inhibitor is an irreversible and selective proteasome inhibitor. In some embodiments, the proteasome inhibitor is an analog of epoxomicin. In some embodiments, the proteasome inhibitor irreversibly and selectively binds to N-terminal threonine-containing active sites of the 20S proteasome. In some embodiments, the proteasome inhibitor is (2S)-4-methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide, also known as carfilzomib or Kyprolis®. In some embodiments, the proteasome inhibitor is carfilzomib. In some embodiments, the proteasome inhibitor has the following structure:

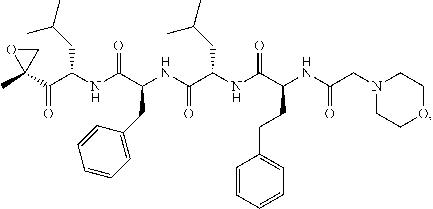 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

or a pharmaceutically acceptable salt, solvate, hydrate, stereoisomer, tautomer or racemic mixtures thereof, including and compositions thereof. In some embodiments, the proteasome inhibitor has the following structure:

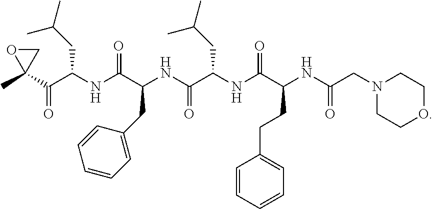 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Compositions of carfilzomib include but are not limited to those described in U.S. Pat. Nos. 7,232,818, 7,417,042, 7,491,704, 7,737,112, 8,129,346, 8,207,127, 8,207,125, 8,207,126, 8,207,297, 9,493,582, 9,511,109, and 10,098,890; and International Publication No. WO 2015/198257 (each incorporated herein by reference in its entirety).

In some embodiments, the induction therapy comprises administering carfilzomib at a dose from or from about 0.1 to 100 mg/m², from or from about 0.1 to 50 mg/m², from or from about 0.1 to 10 mg/m², from or from about 0.1 to 1 mg/m², each inclusive. In some embodiments, the dose of carfizomib is 56 mg/m². In some embodiments, the dose is 45 mg/m². In some embodiments, the dose is 36 mg/m². In some embodiments, the dose is 27 mg/m². In some embodiments, the dose is 20 mg/m². See Steward et al., 2015, N Engl J Med, 372 (2): 142-152; Dimopoulos et al. 2016, Lancet Oncol. 17 (1): 27-238; Wester et al. 2019, Haematologica, 104 (11): 2265-2273; and Sonneveld et al. 2015, Blood, 125 (3): 449-456 (all incorporated herein by reference in its entirety).

In some embodiments, the proteasome inhibitor reversibly inhibits the CT-L proteolytic (35) site of the 20S proteasome. In some embodiments, the proteasome inhibitor is [(1R)-1-[[2-[(2,5-dichlorobenzoyl)amino]acetyl]amino]-3-methylbutyl]boronic acid, also known as ixazomib or Ninlaro®. In some embodiments, the proteasome inhibitor is ixazomib. In some embodiments, the proteasome inhibitor has the following structure:

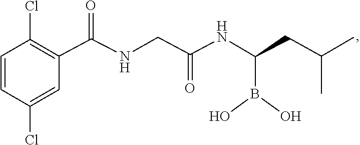 [SMILES: CC(C)C[C@H](NC(=O)CNC(=O)Cc1cc(Cl)ccc1Cl)B(O)O]

or a pharmaceutically acceptable salt, solvate, hydrate, stereoisomer, tautomer or racemic mixtures thereof, including and compositions thereof. In some embodiments, the proteasome inhibitor has the following structure:

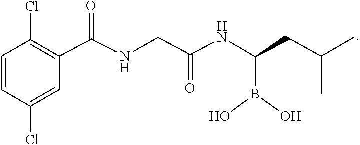 [SMILES: CC(C)C[C@H](NC(=O)CNC(=O)Cc1cc(Cl)ccc1Cl)B(O)O]

Compositions of ixazomib include but are not limited to those described in U.S. Pat. Nos. 8,871,745, 8,530,694, 7,442,830, 9,175,017, 8,003,819, 9,233,115, 8,546,608, 7,687,662, and 8,859,504; and International Publication Nos. WO 2016/165677, WO 2017/174046, WO 2017/046815 and WO2017/163190 (each incorporated herein by reference in its entirety).

In some embodiments, the induction therapy comprises administering ixazomib at a dose from or from about 0.1 to 20 mg, 0.1 mg to 10 mg, 0.1 to 5 mg, and 0.1 to 1 mg, each inclusive. In some embodiments, the dose of ixazomib is 4 mg. In certain embodiments, the dose is given on certain days in a cycle. In certain embodiments, the cycle is 28 days. See Moreau et al. 2016, N Engl J Med, 374:1621 and Rajkumar et al. 2010, Lancet Oncol, 11:29 (both incorporated herein by reference in its entirety).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

In some embodiments, the induction therapy comprises an immunomodulatory agent. In some embodiments, the immunomodulatory agent is a cereblon-modulating compound. In some embodiments, the immunomodulatory agent is a cereblon-binding compound. Cereblon functions as a substrate receptor for a CRL4 ubiquitin E3 ligase, and the binding of cereblon-modulating compounds can induce the recruitment, ubiquitination, and destruction of certain target substrates, such as Ikaros family zinc finger proteins 1 and 3 (IKZF1 and IKZF3, also known as Ikaros and Aiolos, respectively). In some embodiments, administration of the immunomodulatory agent induces ubiquitination of Aiolos and/or Ikaros. In some embodiments, administration of the immunomodulatory agent induces degradation of Aiolos and/or Ikaros. In some aspects, the degree of degradation induced by the immunomodulatory agent is associated with its antitumor effects, for instance with increased degradation associated with greater antitumor effects by the immunomodulatory agent. In some embodiments, the immunomodulatory agent is an IMiD™ or a CELMoD™.

Exemplary immunomodulatory agents include the substituted 2-(2,6-dioxopiperidin-3-yl) phthalimides and substituted 2-(2,6-dioxopiperidin-3-yl)-1-oxoisoindoles described in U.S. Pat. Nos. 6,281,230 and 6,316,471. Still other exemplary immunomodulatory agents belong to a class of isoindole-imides disclosed in U.S. Pat. Nos. 6,395,754, 6,555,554, 7,091,353, U.S. Pat. Publication No. 2004/0029832, and International Publication No. WO 98/54170.

In some embodiments, the immunomodulatory agent is selected from among the group consisting of thalidomide, lenalidomide, pomalidomide, iberdomide (CC-220), CC-92480, CC-99282, CC-91633, and CC-90009, an enantiomer or a mixture of enantiomers thereof, or a pharmaceutically acceptable salt, solvate, hydrate, co-crystal, clathrate, or polymorph thereof. In some embodiments, the immunomodulatory agent is selected from among the group consisting of thalidomide, lenalidomide, pomalidomide, iberdomide (CC-220), CC-92480, CC-99282, CC-91633, and CC-90009 or a pharmaceutically acceptable salt thereof.

In some embodiments, the immunomodulatory agent is thalidomide ((RS)-2-(2,6-dioxopiperidin-3-yl)-1H-isoindole-1,3(2H)-dione) having the structure

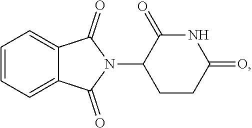 [SMILES: O=C1CCC(N2C(=O)c3ccccc3C2=O)C(=O)N1]

or an enantiomer or a mixture of enantiomers of thalidomide, or a pharmaceutically acceptable salt, solvate, hydrate, co-crystal, clathrate, or polymorph thereof. In some embodiments, the immunomodulatory agent is a pharmaceutically acceptable salt of thalidomide. In some embodiments, the immunomodulatory agent is a solvate of thalidomide. In some embodiments, the immunomodulatory agent is a hydrate of thalidomide. In some embodiments, the immunomodulatory agent is a co-crystal of thalidomide. In some embodiments, the immunomodulatory agent is a clathrate of thalidomide. In some embodiments, the immunomodulatory agent is a polymorph of thalidomide. In some embodiments, the immunomodulatory agent is thalidomide.

In some embodiments, the induction therapy comprises administering thalidomide at a dose from or from about 1 to 500 mg/day, from or from about 1 to 200 mg/day, from or from about 1 to 150 mg/day, from or from about 1 to 100 mg/day, and from or from about 1 to 50 mg/day, each inclusive. In some embodiments, the dose of thalidomide is 200 mg per day for each day of a cycle. In some embodiments, the dose of thalidomide is 50 mg per day for each day of a cycle. In some embodiments, the cycle is 28 days. See Wester et al. 2019, Haematologica, 104 (11): 2265-2273 and Cavo et al. 2011, Blood 117 (23): 6063-6073 (each incorporated herein by reference in its entirety).

In some embodiments, the induction therapy comprises administering thalidomide at about 100 mg the first 14 days and then 200 mg per day thereafter in a cycle. In some embodiments, the cycle is 8 days. See Rajkumar, 2014, Am J Hematol, 89:999 and Cavo et al. 2010, Lancet, 376:2075 (each incorporated by reference in its entirety).

In some embodiments, the dose of thalidomide is taken orally.

In some embodiments, the immunomodulatory agent is lenalidomide (3-(4-amino-1-oxo-1,3-dihydro-2H-isoindol-2-yl) piperidine-2,6-dione) having the structure:

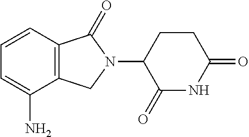 [SMILES: Nc1cccc2c1CN(C1CCC(=O)NC1=O)C2=O]

or an enantiomer or a mixture of enantiomers of lenalidomide, or a pharmaceutically acceptable salt, solvate, hydrate, co-crystal, clathrate, or polymorph thereof. In some embodiments, the immunomodulatory agent is a pharmaceutically acceptable salt of lenalidomide. In some embodiments, the immunomodulatory agent is a solvate of lenalidomide. In some embodiments, the immunomodulatory agent is a hydrate of lenalidomide. In some embodiments, the immunomodulatory agent is a co-crystal of lenalidomide. In some embodiments, the immunomodulatory agent is a clathrate of lenalidomide. In some embodiments, the immunomodulatory agent is a polymorph of lenalidomide. In some embodiments, the immunomodulatory agent is lenalidomide or a generic thereof. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide. In some embodiments, the immunomodulatory agent is lenalidomide.

In some embodiments, the induction therapy comprises administering lenalidomide at a dose from or from about 1 to 400 mg/day, from or from about 1 to 200 mg/day, from or from about 1 to 150 mg/day, from or from about 1 to 100 mg/day, from or from about 1 to 50 mg/day and from or from about 1 to 10 mg/day, each inclusive. In some embodiments, the dose of lenalidomide is 25 mg per day for each day of a cycle. In some embodiments, the cycle is 21 days. See Richardson et al. 2010, Blood, 116:679; Kumar et al. 2012, Blood, 119:4375; Rajkumar et al. 2011, Am J Hematol, 86:57; Moreau et al. 2011, Lancet Oncol 12:431; Rajkumar et al. 2010, Lancet Oncol, 11:909; Rajkumar et al. 2010, Lancet Oncol 11:29; and Niesvizky et al. 2007, Br J Haematol, 138:640 (each incorporated by reference in its entirety). In some embodiments, the cycle is 28 days. See Dimopoulos et al. 2016, N Engl J Med 375:1319; Mateos et al. 2020, Lancet Haematol, 7: e370; Rajkumar et al. 2010, Lancet Oncol, 11:29; Niesvizky et al. 2007, Br J Haematol, 138:640; Barr et al. 2018, Leukemia, 32:2495 (each reference is herein incorporated by reference in its entirety). In some embodiments, the dose of lenalidomide is administered orally.

In some embodiments, the immunomodulatory agent is pomalidomide (4-amino-2-(2,6-dioxopiperidin-3-yl) isoindole-1,3-dione) having the structure:

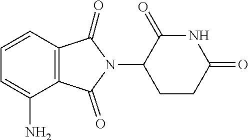 [SMILES: Nc1cccc2c1C(=O)N(C1CCC(=O)NC1=O)C2=O]

or an enantiomer or a mixture of enantiomers of pomalidomide, or a pharmaceutically acceptable salt, solvate, hydrate, co-crystal, clathrate, or polymorph thereof. In some embodiments, the immunomodulatory agent is a pharmaceutically acceptable salt of pomalidomide. In some embodiments, the immunomodulatory agent is a solvate of pomalidomide. In some embodiments, the immunomodulatory agent is a hydrate of pomalidomide. In some embodiments, the immunomodulatory agent is a co-crystal of pomalidomide. In some embodiments, the immunomodulatory agent is a clathrate of pomalidomide. In some embodiments, the immunomodulatory agent is a polymorph of pomalidomide. In some embodiments, the immunomodulatory agent is pomalidomide.

In some embodiments, the immunomodulatory agent is iberdomide ((S)-3-[4-(4-morpholin-4-ylmethyl-benzyloxy)-1-oxo-1,3-dihydro-isoindol-2-yl]-piperidine-2,6-dione; also known as CC-220) having the structure:

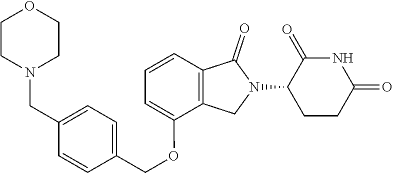 [SMILES: O=C1CC[C@H](N2Cc3c(OCc4ccc(CN5CCOCC5)cc4)cccc3C2=O)C(=O)N1]

or an enantiomer or a mixture of enantiomers of iberdomide, or a pharmaceutically acceptable salt, solvate, hydrate, co-crystal, clathrate, or polymorph thereof. Methods of preparing iberdomide are described in US Pat. Application No. 2011/0196150. In some embodiments, the immunomodulatory agent is a pharmaceutically acceptable salt of iberdomide. In some embodiments, the immunomodulatory agent is a solvate of iberdomide. In some embodiments, the immunomodulatory agent is a hydrate of iberdomide. In some embodiments, the immunomodulatory agent is a co-crystal of iberdomide. In some embodiments, the immunomodulatory agent is a clathrate of iberdomide. In some embodiments, the immunomodulatory agent is a polymorph of iberdomide. In some embodiments, the immunomodulatory agent is iberdomide.

In some embodiments, the immunomodulatory agent is CC-92480 ((S)-4-(4-(4-(((2-(2,6-dioxopiperidin-3-yl)-1-oxoisoindolin-4-yl)oxy)methyl)benzyl) piperazin-1-yl)-3-fluorobenzonitrile) having the structure:

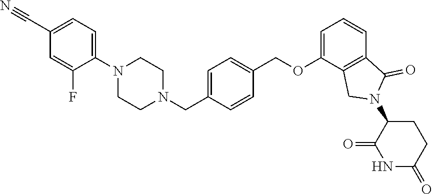 [SMILES: N#Cc1ccc(N2CCN(Cc3ccc(COc4cccc5c4CN([C@H]4CCC(=O)NC4=O)C5=O)cc3)CC2)c(F)c1]

or an enantiomer or a mixture of enantiomers of CC-92480, or a pharmaceutically acceptable salt, solvate, hydrate, co-crystal, clathrate, or polymorph thereof. In some embodiments, the immunomodulatory agent is a pharmaceutically acceptable salt of CC-92480. In some embodiments, the immunomodulatory agent is a solvate of CC-92480. In some embodiments, the immunomodulatory agent is a hydrate of CC-92480. In some embodiments, the immunomodulatory agent is a co-crystal of CC-92480. In some embodiments, the immunomodulatory agent is a clathrate of CC-92480. In some embodiments, the immunomodulatory agent is a polymorph of CC-92480. In some embodiments, the immunomodulatory agent is CC-92480.

In some embodiments, the immunomodulatory agent is CC-99282 ((S)-2-(2,6-dioxopiperidin-3-yl)-4-((2-fluoro-4-((3-morpholinoazetidin-1-yl)methyl)benzyl)amino) isoindoline-1,3-dione) having the structure:

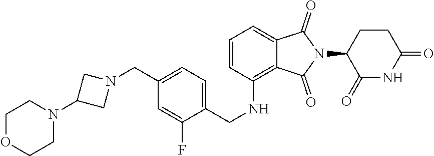 [SMILES: O=C1CC[C@H](N2C(=O)c3cccc(NCc4ccc(CN5CC(N6CCOCC6)C5)cc4F)c3C2=O)C(=O)N1]

or an enantiomer or a mixture of enantiomers of CC-99282, or a pharmaceutically acceptable salt, solvate, hydrate, co-crystal, clathrate, or polymorph thereof. Methods of preparing CC-99282 are described in US Pat. Application No. 2019/0322647. In some embodiments, the immunomodulatory agent is a pharmaceutically acceptable salt of CC-99282. In some embodiments, the immunomodulatory agent is a solvate of CC-99282. In some embodiments, the immunomodulatory agent is a hydrate of CC-99282. In some embodiments, the immunomodulatory agent is a co-crystal of CC-99282. In some embodiments, the immunomodulatory agent is a clathrate of CC-99282. In some embodiments, the immunomodulatory agent is a polymorph of CC-99282. In some embodiments, the immunomodulatory agent is CC-99282.

In some embodiments, the immunomodulatory agent is CC-91633 or an enantiomer or a mixture of enantiomers of CC-91633, or a pharmaceutically acceptable salt, solvate, hydrate, co-crystal, clathrate, or polymorph thereof. In some embodiments, the immunomodulatory agent is a pharmaceutically acceptable salt of CC-91633. In some embodiments, the immunomodulatory agent is a solvate of CC-91633. In some embodiments, the immunomodulatory agent is a hydrate of CC-91633. In some embodiments, the immunomodulatory agent is a co-crystal of CC-91633. In some embodiments, the immunomodulatory agent is a clathrate of CC-91633. In some embodiments, the immunomodulatory agent is a polymorph of CC-91633. In some embodiments, the immunomodulatory agent is CC-91633.

In some embodiments, the immunomodulatory agent is CC-90009 having the stucture:

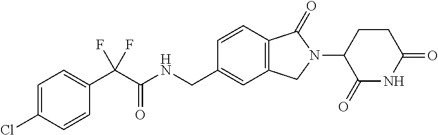 [SMILES: O=C1CCC(N2Cc3cc(CNC(=O)C(F)(F)c4ccc(Cl)cc4)ccc3C2=O)C(=O)N1]

or an enantiomer or a mixture of enantiomers of CC-90009, or a pharmaceutically acceptable salt, solvate, hydrate, co-crystal, clathrate, or polymorph thereof (see, e.g., Surka et al., Blood (2021) 137 (5): 661-677). In some embodiments, the immunomodulatory agent is a pharmaceutically acceptable salt of CC-90009. In some embodiments, the immunomodulatory agent is a solvate of CC-90009. In some embodiments, the immunomodulatory agent is a hydrate of CC-90009. In some embodiments, the immunomodulatory agent is a co-crystal of CC-90009. In some embodiments, the immunomodulatory agent is a clathrate of CC-90009. In some embodiments, the immunomodulatory agent is a polymorph of CC-90009. In some embodiments, the immunomodulatory agent is CC-90009.

In some embodiments, the immunomodulatory agent is any immunomodulatory agent as described in Section I.B.2.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

In some embodiments, the induction therapy comprises a steroid, e.g., corticosteroid. Corticosteroids typically include glucocorticoids and mineralocorticoids.

Any corticosteroid, e.g., glucocorticoid, can be used in the methods provided herein. In some embodiments, glucocorticoids include synthetic and non-synthetic glucocorticoids. Exemplary glucocorticoids include, but are not limited to: alclomethasones, algestones, beclomethasones (e.g., beclomethasone dipropionate), betamethasones (e.g., betamethasone 17-valerate, betamethasone sodium acetate, betamethasone sodium phosphate, betamethasone valerate), budesonides, clobetasols (e.g., clobetasol propionate), clobetasones, clocortolones (e.g., clocortolone pivalate), cloprednols, corticosterones, cortisones and hydrocortisones (e.g., hydrocortisone acetate), cortivazols, deflazacorts, desonides, desoximethasones, dexamethasones (e.g., dexamethasone 21-phosphate, dexamethasone acetate, dexamethasone sodium phosphate), diflorasones (e.g., diflorasone diacetate), diflucortolones, difluprednates, enoxolones, fluazacorts, flucloronides, fludrocortisones (e.g., fludrocortisone acetate), flumethasones (e.g., flumethasone pivalate), flunisolides, fluocinolones (e.g., fluocinolone acetonide), fluocinonides, fluocortins, fluocortolones, fluorometholones (e.g., fluorometholone acetate), fluperolones (e.g., fluperolone acetate), fluprednidenes, fluprednisolones, flurandrenolides, fluticasones (e.g., fluticasone propionate), formocortals, halcinonides, halobetasols, halometasones, halopredones, hydrocortamates, hydrocortisones (e.g., hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (e.g., methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g., prednisolone 25-diethylaminoacetate, prednisolone sodium phosphate, prednisolone 21-hemisuccinate, prednisolone acetate, prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g., triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone. In a particular example, the glucocorticoid is prednisone.

In some embodiments, the induction therapy comprises administering dexamethasone at a dose from or from about 100 to 1000 mg per cycle, from or from about 100 to 700 mg per cycle, from or from about 100 to 500 mg per cycle, and from or from about 100 to 300 mg per cycle, each inclusive. In some embodiments, the dose of dexamethasone is a high dose. In some embodiments, the dose of dexamethasone is 480 mg total per cycle. In some embodiments, the dose of dexamethasone is a low dose. In some embodiments, the dose of dexamethasone is 160 mg total per cycle. In some embodiments, the cycle is 28 days. See Rajkumar et al. 2010, Lancet Oncol, 11 (1): 29-37 (incorporated herein by reference in its entirety). In some embodiments, the dose of dexamethasone is 40 mg total per cycle. In some embodiments, the cycle is 21 days. See Richardson et al. 2010, Blood, 116:679; Kumar et al. 2012, Blood, 119:4375; Rajkumar et al. 2011, Am J Hematol, 86:57; Moreau et al. 2011, Lancet Oncol 12:431; Rajkumar et al. 2010, Lancet Oncol, 11:909; Rajkumar et al. 2010, Lancet Oncol 11:29; and Niesvizky et al. 2007, Br J Haematol, 138:640 (each incorporated by reference in its entirety). In some embodiments, the dose of dexamethasone is administered orally. In some embodiments, the dose of dexamethasone is 20 mg total per cycle. In some embodiments, the cycle is 28 days. See Dimopoulos et al. 2016, N Engl J Med 375:1319; Mateos et al. 2020, Lancet Haematol, 7: e370; Rajkumar et al. 2010, Lancet Oncol, 11:29; Niesvizky et al. 2007, Br J Haematol, 138:640; Barr et al. 2018, Leukemia, 32:2495 (each reference is herein incorporated by reference in its entirety).

In some embodiments, the induction therapy comprises administering dexamethasone at a dose from or from about 1 to 200 mg weekly, from or from about 1 to 150 mg weekly, from about 1 to 100 mg weekly, from or from about 1 to 50 mg weekly, or from or from about 1 to 10 mg weekly, each inclusive. In some embodiments, the dose of 40 mg weekly. Wester et al. 2019, Haematologica, 104 (11): 2265-2273 (incorporated herein by reference in its entirety).

In some embodiments, the dose of dexamethasone is administered orally or intravenously.

In some embodiments, the induction therapy comprises a monoclonal antibody. In some embodiments, the induction therapy comprises an anti-CD38 antibody. In some embodiments, the anti-CD38 antibody is selected from among the group consisting of daratumumab, ixatuximab, MOR202, and TAK-079. In some embodiments, the monoclonal antibody is daratumumab. In some embodiments, the monoclonal antibody is daratumumab.

In some embodiments, the induction therapy comprises administering daratumumab. In some embodiment, the dose of daratumumab is from or from about 100 to 3000 mg per day in a cycle, from or from about 100 to 2000 mg per day in a cycle, from or from about 100 to 1000 mg per day in a cycle, or from or from about 100 to 500 mg per day in a cycle, each inclusive. In some embodiments, the dose of daratumumab is administered with hyaluronidase. In some embodiments, the dose comprises from or from about 10,000 to 40,000 units hyaluronidase. In some embodiments, the dose comprises about 40,000 units, about 30,000 units, about 20,000 units, or about 10,000 units hyaluronidase. In some embodiments, the dose comprises 30,000 units hyaluronidase. In some embodiments, the daratumumab and hyaluronidase are administered intravenously. See Dimopoulos et al. 2016, N Engl J Med 375:1319; Mateos et al. 2020, Lancet Haematol, 7: e370; Rajkumar et al. 2010, Lancet Oncol, 11:29; Niesvizky et al. 2007, Br J Haematol, 138:640; Barr et al. 2018, Leukemia, 32:2495 (each reference is herein incorporated by reference in its entirety).

In some embodiments, the induction therapy comprises an alkylating agent. In some embodiments, the alkylating agent is selected from among the group consisting altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In some embodiments, the alkylating agent is cyclophosphamide.

In some embodiments, the induction therapy comprises administering cyclophosphamide at a dose from or from about 1 to 500 mg/m², from or from about 1 to 400 mg/m², from or from about 1 to 300 mg/m², from or from about 1 to 200 mg/m², or from or from about 1 to 100 mg/m², each inclusive. In some embodiments, this dose is provided once weekly. In some embodiments, the dose is 300 mg/m² weekly. In some embodiments, the cyclophosphamide is administered orally. In some embodiments, the cycle is 28 days. See Reeder et al. 2010, Blood, 115:3416; Reeder et al. 2009, Leukemia, 23:1337; Kropff et al. 2007, Br J Haemtol, 138:330; Moreau et al. 2011, Lancet Oncol, 12:431 (each incorporated by reference in its entirety).

In some embodiments, the induction therapy is one or more of a proteasome inhibitor, an immunomodulatory agent, and dexamethasone (Decadron® or Dexasone®). In some embodiments, the induction therapy is bortezomib (Velcade®), lenalidomide (Revlimid®) and dexamethasone (VRD regimen). In some embodiments, the induction therapy is bortezomib (Velcade®), lenalidomide (Revlimid®) and low-dose dexamethasone (VRd regimen). In some embodiments, the induction therapy is a reduced dose of bortezomib, lenalidomide, and low-dose dexamethasone (VRd Lite regimen). In some embodiments, the induction therapy is cyclophosphamide (Cytoxan® or Procytox®), bortezomib and dexamethasone (VCD or CyBorD regimen). In some embodiments, the induction therapy is thalidomide (Thalomid®) and dexamethasone (TD regimen). In some embodiments, the induction therapy is lenalidomide and low-dose dexamethasone (Rd regimen).

In some embodiments, the induction therapy is bortezomib and dexamethasone (VD regimen). See Harousseau et al. 2010, J Clin Onco, 28 (30): 4621-4629 (incorporated herein by reference in its entirety). In some embodiments, the induction therapy is bortezomib and low-dose dexamethasone (Vd regimen). In some embodiments, the induction therapy is bortezomib, thalidomide and dexamethasone (VTD regimen). In some embodiments, the induction therapy is bortezomib, cyclophosphamide, and prednisone. In some embodiments, the induction therapy is bortezomib, doxorubicin (Adriamycin®) and dexamethasone. In some embodiments, the induction therapy is dexamethasone. In some embodiments, the induction therapy is liposomal doxorubicin (Caelyx® or Doxil®), vincristine (Oncovin®) and dexamethasone. In some embodiments, the induction therapy is daratumumab and hyaluronidase (Darzalex Faspro®), lenalidomide, and dexamethasone. In some embodiments, the induction therapy is daratumumab and hyaluronidase, bortezomib, thalidomide, and dexamethasone. See US Patent Application Publication US2020/0397896. In some embodiments, the induction therapy is carfilzomib (Kyprolis®), lenalidomide and dexamethasone (KRd regimen). In some embodiments, the induction therapy is ixazomib (Ninlaro®), lenalidomide, and dexamethasone (Ixa-Rd or IRd regimen).

In some embodiments, the induction therapy is vincristine, adriamycin, and dexamethasone (VAD regimen). See Cavo et al. (2013) Blood, 106 (1): 35-39. In some embodiments, the induction therapy is thalidomide, adriamycin and dexamethasone (TAD regimen). Lockhorst et al. 2010, Blood, 115 (6): 1113-1120. In some embodiments, the induction therapy is cyclophosphamide, thalidomide and dexamethasone (CTD regimen). See Morgan et al. 2012, Hematologica, 97 (3): 442-450. In some embodiments, the induction therapy is bortezomib, adriamycin and dexamethasone (PAD regimen). See Neben et al. 2012, Blood, 119 (4): 940-948 or Oakervee et al. 2005, Br J Haematol, 129 (6): 755-762. In some embodiments, the induction therapy is bortezomib, thalidomide, and dexamethasone (VTD regimen). See Wang et al. 2007, Hematology, 12 (3): 235-239. In some embodiments, the induction therapy comprises daratumumab and hyaluronidase, bortezomib, thalidomide, and dexamethasone (DVTd regimen). See US Patent Application Publication US2020/0397896. All references are each incorporated herein by reference in its entirety.

In some embodiments, the induction therapy comprises four drugs. In some embodiments, the induction therapy comprises three drugs. In some embodiments, the induction therapy comprises two drugs. In some embodiments, the drug is administered orally. In some embodiments, the drug is administered as a tablet or capsule. In some embodiments, the drug is administered intravenously.

In some embodiments, the dose of a drug in an induction therapy is a daily dose. In some embodiments, the dose of a drug in an induction therapy is a once-daily dose. In some embodiments, the dose of a drug in an induction therapy that is administered on each of the days on which the drug in an induction therapy is administered.

In some embodiments, the dose of a drug in an induction therapy is administered daily, every other day, three times a week, twice a week, or once a week. In some embodiments, the dose of a drug in an induction therapy is administered daily. In some embodiments, the dose of a drug in an induction therapy is administered daily for a plurality of consecutive days. In some embodiments, the dose of a drug in an induction therapy is administered daily for up to about 7, 8, 9, 10, 11, 12, 13, 14, 15, 16, 17, 18, 19, 20, 21, 22, 23, 24, 25, 26, 27, 28, 29, 30, or more than 30 consecutive days.

In some embodiments, the dose of a drug in an induction therapy is administered once daily for 14 days over a 21-day treatment cycle. In some embodiments, the dose of a drug in an induction therapy is administered once daily for 21 days over a 28-day treatment cycle.

In some embodiments, the dose of a drug in an induction therapy is administered for at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, or at least 12 cycles. In some embodiments, the dose of a drug in an induction therapy is administered for 1, 2, 3, 4, 5, 6, 7, 8, 9, 10, 11, or 12 cycles.

In some embodiments, the induction therapy is administered in cycles. The total number of cycles used for an individual patient depends on whether the patient plans to proceed immediately with high dose chemotherapy (HDT), how well they tolerate the regimen, and the response to treatment. Patients not proceeding to early HDT are typically treated with 8 to 12 cycles, as tolerated, followed by maintenance until progression.

In some embodiments, the induction therapy is administered for at least 1 cycle. In some embodiments, the induction therapy is administered for at least 2 cycles. In some embodiments, the induction therapy is administered for at least 3 cycles. In some embodiments, the induction therapy is administered for at least 4 cycles. In some embodiments, the induction therapy is administered for at least 5 cycles. In some embodiments, the induction therapy is administered for at least 6 cycles.

In some embodiments, the induction therapy is given for 2, 3, 4, 5, 6, 7, 8, 9, 10 or more cycles. In some embodiments, the induction therapy is given for greater than or equal to three cycles. In some embodiments, the induction therapy is given for greater than or equal to four cycles. In some embodiments, the induction therapy is given for greater than or equal to five cycles. In some embodiments, the induction therapy is given for greater than or equal to six cycles.

In some embodiments, the induction therapy is administered in ≥3 cycles. In some embodiments, the induction therapy is administered in 3-12 cycles. In some embodiments, the induction therapy is administered in 3-11 cycles. In some embodiments, the induction therapy is administered in 3-10 cycles. In some embodiments, the induction therapy is administered in 3-9 cycles. In some embodiments, the induction therapy is administered in 3-8 cycles. In some embodiments, the induction therapy is administered in 3-7 cycles. In some embodiments, the induction therapy is administered in 3-6 cycles. In some embodiments, the induction therapy is administered in 3-5 cycles. In some embodiments, the induction therapy is administered in 3-4 cycles.

In some embodiments, the induction therapy is administered in 3 cycles. In some embodiments, the induction therapy is administered in 4 cycles. In some embodiments, the induction therapy is administered in 5 cycles. In some embodiments, the induction therapy is administered in 6 cycles. In some embodiments, the induction therapy is administered in 7 cycles. In some embodiments, the induction therapy is administered in 8 cycles. In some embodiments, the induction therapy is administered in 9 cycles. In some embodiments, the induction therapy is administered in 10 cycles. In some embodiments, the induction therapy is administered in 11 cycles. In some embodiments, the induction therapy is administered in 12 cycles.

In some embodiments, each cycle is a 28-day cycle. In some embodiments, the cycle length is 28 days. In some embodiments, each cycle is a 21-day cycle. In some embodiments, the cycle length is 21 days.

In certain embodiments, in a 21-day cycle, an induction therapy comprises of administering bortezomib at 1.3 mg/m² subcutaneously or intravenously on days 1, 8 and 15; lenalidomide at 25 mg orally daily from days 1-14; and dexamethasone at 40 mg orally on days 1, 8 and 15.

In certain embodiments, in a 28-day cycle, an induction therapy comprises of administering lenalidomide at 25 mg orally daily from days 1-21 and dexamethasone at 40 mg orally on days 1, 8, 15, and 22.

In certain embodiments, in a 28-day cycle, an induction therapy comprises of administering daratumumab-hyaluronidase at 1800 mg daratumumab plus 30,000 units hyaluronidase subcutaneously on days 1, 8, 15, and 22; 25 mg of lenalidomide orally daily from days 1 through 21; and dexamethasone at 20 mg intravenously or orally on days 1, 2, 8, 9, 16, 17, 22, and 23. In particular embodiments, the first dose of dexamethasone given in this induction therapy is given intravenously and all other doses are given orally. In certain embodiments, this 28-day cycle induction therapy is repeated anywhere from 1 to 7 cycles or beyond 7 cycles. In some embodiments, for the dose of daratumumab-hyaluronidase, for cycles 1 and 2, the dose is administered on days 1, 8, 15, and 22; for cycles 3 to 6, the dose is administered on days 1 and 15; and for cycles 7 and beyond; the dose is only administered on day 1. In some embodiments, for the dose of dexamethasone, for cycles 1 to 2, the dose is administered on days 1, 2, 8, 9, 15, 16, 22, and 23; for cycles 3 to 6, the dose is administered on days 1, 2, 15, and 16; and for cycles 7 and beyond, the dose is administered on days 1 and 2.

In certain embodiments, in a 28-day cycle, an induction therapy comprises of administering daratumumab-hyaluronidase at 1800 mg daratumumab plus 30,000 units hyaluronidase subcutaneously on days 1, 8, 15, and 22; 25 mg of lenalidomide orally daily from days 1 through 21; and dexamethasone at 40 mg orally. In some embodiments, for the dose of dexamethasone, the dose is 40 mg orally for cycles 3 to 6 on days 8 and 22. In some embodiments, for the dose of dexamethasone, the dose is 40 mg orally for cycles 7 and beyond on days 8, 15, and 22.

In certain embodiments, in a 28-day cycle, an induction therapy comprises of administering bortezomib at 1.5 mg/m² subcutaneously or intravenously on days 1, 8, 15, and 22; cyclophosphamide at 300 mg/m² orally on days 1, 8, 16, and 22; and dexamethasone at 40 mg orally on days 1, 8, 15, and 22.

In certain embodiments, in a 28-day cycle, an induction therapy comprises of administering bortezomib at 1.5 mg/m² subcutaneously or intravenously on days 1, 8, 15, and 22; thalidomide orally at 100 mg for the first 14 days and then 200 mg per day thereafter daily from days 1 through 21; and dexamethasone at 40 mg orally on days 1, 8, 15, and 22. In particular embodiments, the bortezomib is administered subcutaneously as a single injection.

In certain embodiments, in a 28-day cycle, an induction therapy comprises of administering ixazomib at 4 mg orally on days 1, 8, and 15; lenalidomide at 26 mg orally daily from days 1 through 21; and dexamethasone at 40 mg orally on days 1, 8, 15, and 22.

B. BCMA Targeted Car T Cell Therapy with Maintenance Therapy

Provided herein are methods of treating a subject having a cancer, comprising administration of a T cell therapy (e.g., BCMA CAR T cells) followed by an immunomodulatory agent maintenance therapy, e.g., wherein the subject had an early relapse or an inadequate response to one or more anti-myeloma treatment. Also provided herein are methods of treating a subject having a cancer, comprising administration of a T cell therapy (e.g., BCMA CAR T cells) followed by an immunomodulatory agent maintenance therapy, wherein the subject is a newly diagnosed multiple myeloma (NDMM) subject who had an early relapse or an inadequate response to an autologous stem cell therapy. Also provided are methods of maintenance therapy, comprising administering an immunomodulatory agent maintenance therapy to a subject after having received a BCMA targeted CAR T cell therapy for treating multiple myeloma.

### 1. BCMA Targeted Car T Cell Therapy

In some embodiments of the methods, compositions, combinations, kits and uses provided herein, the treatment includes administering to a subject a T cell therapy (e.g., CAR-expressing T cells). For example, the T cell therapy is an anti-BCMA CAR T cell therapy.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cell-based therapy is or comprises administration of cells, such as immune cells, for example T cell or NK cells, that target a molecule expressed on the surface of a lesion, such as a tumor or a cancer. In some embodiments, the cells express a recombinant receptor, e.g., a CAR, that contains an extracellular ligand-binding domain that specifically binds to an antigen. In some embodiments, the recombinant receptor is a CAR that contains an extracellular antigen-recognition domain that specifically binds to BCMA. In some embodiments, the immune cells express a recombinant receptor, such as a chimeric antigen receptor (CAR). In some embodiments, the T cell therapy includes administering T cells engineered to express a chimeric antigen receptor (CAR). In particular embodiments, the cell therapy, e.g., anti-BCMA CAR T cell therapy, is for treating a multiple myeloma, such as a relapsed and refractory multiple myeloma (R/R MM) or a newly diagnosed multiple myeloma (NDMM). In some embodiments, the cells are autologous to the subject. In some embodiments, the cells are allogeneic to the subject. Exemplary engineered cells for administering as a cell therapy in the provided methods are described in Section II. The BCMA targeted CAR T cell therapy can be any exemplary engineered cells described in that section.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4+ vs CD8+ T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and, in some cases, about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, or about 45 billion cells) or any value in between these ranges and/or per kilogram of body weight. Dosages may vary depending on attributes particular to the disease or disorder and/or patient and/or other treatments.

In some embodiments, the dose of the total recombinant receptor (e.g., CAR)-expressing cells is between about 100×10⁶ and about 900×10⁶ cells, 100×10⁶ and about 800×10⁶ cells, 100×10⁶ and about 700×10⁶ cells, 100×10⁶ and about 600×10⁶ cells, 200×10⁶ and about 900×10⁶ cells, 200×10⁶ and about 800×10⁶ cells, 200×10⁶ and about 700×10⁶ cells, 200×10⁶ and about 600×10⁶ cells, 300×10⁶ and about 00×10⁶ cells, 300×10⁶ and about 800×10⁶ cells, 300×10⁶ and about 700×10⁶ cells, or 300×10⁶ and about 600×10⁶ cells, each inclusive.

In some embodiments, the dose of the total recombinant receptor (e.g., CAR)-expressing cells is between about 100×10⁶ and about 600×10⁶ cells, inclusive. In some embodiments, the dose of the total recombinant receptor (e.g., CAR)-expressing cells is between about 150×10⁶ and about 540×10⁶ cells, inclusive. In some embodiments, the dose of the total recombinant receptor (e.g., CAR)-expressing cells is between about 150×10⁶ and about 450×10⁶ cells, inclusive. In some embodiments, the dose of the total recombinant receptor (e.g., CAR)-expressing cells is between about 300×10⁶ and about 540×10⁶ cells, inclusive. In some embodiments, the dose of the total recombinant receptor (e.g., CAR)-expressing cells is between about 300×10⁶ and about 460×10⁶ cells, inclusive. In some embodiments, the dose of the total recombinant receptor (e.g., CAR)-expressing cells is between about 300×10⁶ and about 450×10⁶ cells, inclusive.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose includes fewer than about 5×10⁸ total recombinant receptor (e.g., CAR)-expressing cells, T cells, or peripheral blood mononuclear cells (PBMCs), e.g., in the range of about 1×10⁸ to 5×10⁸ such cells, such as 1.5×10⁸, 3×10⁸, or 4.5×10⁸ or total such cells, or the range between any two of the foregoing values.

The cells can be administered by any suitable means. The cells are administered in a dosing regimen to achieve a therapeutic effect, such as a reduction in tumor burden. Dosing and administration may depend in part on the schedule of administration of the debulking, which is carried out prior to initiation of administration of the T cell therapy. Various dosing schedules of the T cell therapy include but are not limited to single or multiple administrations over various time-points, bolus administration, and pulse infusion.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is administered a preconditioning agent (lymphodepleting treatment) as described in Section I.B.3.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells is measured by assaying expression and/or secretion of one or more cytokines, such as CD107a, IFNγ, IL-2, and TNF. In some aspects, the biological activity is measured by assessing clinical outcome, such as reduction in tumor burden or load.

In some embodiments, a dose of cells is administered to subjects in accord with the provided T cell therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 350 million cells, about 450 million cells, about 650 million cells, about 800 million cells, about 900 million cells, about 3 billion cells, about 30 billion cells, or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In some embodiments, the cell therapy comprises administration of a dose comprising a number of cells from or from about 1×10⁵ to 2×10⁹ total recombinant receptor-expressing cells, total T cells, or total peripheral blood mononuclear cells (PBMCs), from or from about 5×10⁵ to 1×10⁹ total recombinant receptor-expressing cells, total T cells, or total peripheral blood mononuclear cells (PBMCs), or from or from about 1×10⁶ to 1×10⁹ total recombinant receptor-expressing cells, total T cells, or total peripheral blood mononuclear cells (PBMCs), each inclusive.

In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸, at least or about at least 1×10⁹ of such cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, at least or at least about 3.0×10⁸ CAR-expressing cells, or at least or at least about 5×10⁸ CAR-expressing cells.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 1.0×10⁷ to at or about 1.2×10⁹ such cells, such as at or about 1.0×10⁷, 1.5×10⁷, 2.0×10⁷, 2.5×10⁷, 5×10⁷, 1.5×10⁸, 3×10⁸, 4.5×10⁸, 4.6×10⁸, 5.4×10⁸, 6×10⁸, 8×10⁸ or 1.2×10⁹ total such cells, or the range between any two of the foregoing values.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1.5×10⁸, 3×10⁸, 4.5×10⁸, 4.6×10⁸, 5.4×10⁸, 6×10⁸, 8×10⁸ or 1.2×10⁹ total such cells, or the range between any two of the foregoing values.

In some embodiments, for example, where the subject is a human, the dose includes at or about 1.0×10⁷ total recombinant receptor (e.g., CAR)-expressing cells, T cells, or peripheral blood mononuclear cells (PBMCs). In some embodiments, for example, where the subject is a human, the dose includes at or about 1.5×10⁷ total recombinant receptor (e.g., CAR)-expressing cells, T cells, or peripheral blood mononuclear cells (PBMCs). In some embodiments, for example, where the subject is a human, the dose includes at or about 2.0×10⁷ total recombinant receptor (e.g., CAR)-expressing cells, T cells, or peripheral blood mononuclear cells (PBMCs). In some embodiments, for example, where the subject is a human, the dose includes at or about 2.5×10⁷ total recombinant receptor (e.g., CAR)-expressing cells, T cells, or peripheral blood mononuclear cells (PBMCs). In some embodiments, for example, where the subject is a human, the dose includes at or about 5×10⁷ total recombinant receptor (e.g., CAR)-expressing cells, T cells, or peripheral blood mononuclear cells (PBMCs). In some embodiments, for example, where the subject is a human, the dose includes at or about 1.5×10⁸ total recombinant receptor (e.g., CAR)-expressing cells, T cells, or peripheral blood mononuclear cells (PBMCs). In some embodiments, for example, where the subject is a human, the dose includes at or about 3×10⁸ total recombinant receptor (e.g., CAR)-expressing cells, T cells, or peripheral blood mononuclear cells (PBMCs). In some embodiments, for example, where the subject is a human, the dose includes at or about 4.5×10⁸ total recombinant receptor (e.g., CAR)-expressing cells, T cells, or peripheral blood mononuclear cells (PBMCs). In some embodiments, for example, where the subject is a human, the dose includes at or about 4.6×10⁸ total recombinant receptor (e.g., CAR)-expressing cells, T cells, or peripheral blood mononuclear cells (PBMCs). In some embodiments, for example, where the subject is a human, the dose includes at or about 5.4×10⁸ total recombinant receptor (e.g., CAR)-expressing cells, T cells, or peripheral blood mononuclear cells (PBMCs). In some embodiments, for example, where the subject is a human, the dose includes at or about 6×10⁸ total recombinant receptor (e.g., CAR)-expressing cells, T cells, or peripheral blood mononuclear cells (PBMCs). In some embodiments, for example, where the subject is a human, the dose includes at or about 8×10⁸ total recombinant receptor (e.g., CAR)-expressing cells, T cells, or peripheral blood mononuclear cells (PBMCs). In some embodiments, for example, where the subject is a human, the dose includes at or about 1.2×10⁹ total recombinant receptor (e.g., CAR)-expressing cells, T cells, or peripheral blood mononuclear cells (PBMCs).

In some embodiments, the dose of genetically engineered cells comprises from at or about 1×10⁵ to at or about 2×10⁹ total CAR-expressing (CAR+) T cells, from at or about 1×10⁵ to at or about 5.4×10⁸ total CAR-expressing T cells, from at or about 1×10⁵ to at or about 5×10⁸ total CAR-expressing T cells, from at or about 1×10⁵ to at or about 4.6×10⁸ total CAR-expressing T cells, from at or about 1×10⁵ to at or about 4.5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5.4×10⁸ total CAR-expressing T cells, from at or about 1×10⁶ to at or about 5×10⁸ total CAR-expressing T cells, from at or about 1×10⁶ to at or about 4.6×10⁸ total CAR-expressing T cells, from at or about 1×10⁶ to at or about 4.5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5.4×10⁸ total CAR-expressing T cells, from at or about 2.5×10⁶ to at or about 5×10⁸ total CAR-expressing T cells, from at or about 2.5×10⁶ to at or about 4.6×10⁸ total CAR-expressing T cells, from at or about 2.5×10⁶ to at or about 4.5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5.4×10⁸ total CAR-expressing T cells, from at or about 5×10⁶ to at or about 5×10⁸ total CAR-expressing T cells, from at or about 5×10⁶ to at or about 4.6×10⁸ total CAR-expressing T cells, from at or about 5×10⁶ to at or about 4.5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5.4×10⁸ total CAR-expressing T cells, from at or about 1×10⁷ to at or about 5×10⁸ total CAR-expressing T cells, from at or about 1×10⁷ to at or about 4.6×10⁸ total CAR-expressing T cells, from at or about 1×10⁷ to at or about 4.5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5.4×10⁷ total CAR-expressing T cells, from at or about 1×10⁷ to at or about 5×10⁷ total CAR-expressing T cells, from at or about 1×10⁷ to at or about 4.6×10⁷ total CAR-expressing T cells, from at or about 1×10⁷ to at or about 4.5×10⁷ total CAR-expressing T cells, from at or about 1×10⁷ to at or about 2.5×10⁷ total CAR-expressing T cells, from at or about 2.5×10⁷ to at or about 5.4×10⁸ total CAR-expressing T cells, from at or about 2.5×10⁷ to at or about 5×10⁸ total CAR-expressing T cells, from at or about 2.5×10⁷ to at or about 4.6×10⁸ total CAR-expressing T cells, from at or about 2.5×10⁷ to at or about 4.5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5.4×10⁸ total CAR-expressing T cells, from at or about 5×10⁷ to at or about 5×10⁸ total CAR-expressing T cells, from at or about 5×10⁷ to at or about 4.6×10⁸ total CAR-expressing T cells, from at or about 5×10⁷ to at or about 4.5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5.4×10⁸ total CAR-expressing T cells, from at or about about 1×10⁸ to at or about 5×10⁸ total CAR-expressing T cells, from at or about 1×10⁸ to at or about 4.6×10⁸ total CAR-expressing T cells, from at or about 1×10⁸ to at or about 4.5×10⁸ total CAR-expressing T cells, from at or about 1×10⁸ to at or about 2.5×10⁸ total CAR-expressing T cells, from at or about 2.5×10⁸ to at or about 5.4×10⁸ total CAR-expressing T cells, from at or about 2.5×10⁸ to at or about 5×10⁸ total CAR-expressing T cells, from at or about 2.5×10⁸ to at or about 4.6×10⁸ total CAR-expressing T cells, from at or about 2.5×10⁸ to at or about 4.5×10⁸ total CAR-expressing T cells, from at or about 3.0×10⁸ to at or about 5.4×10⁸ total CAR-expressing T cells, from at or about 3.0×10⁸ to at or about 5×10⁸ total CAR-expressing T cells, from at or about 3.0×10⁸ to at or about 4.6×10⁸ total CAR-expressing T cells, or from at or about 3.0×10⁸ to at or about 4.5×10⁸ total CAR-expressing T cells.

In some embodiments, the dose of genetically engineered cells comprises from at or about at or about 1.5×10⁸ to at or about 5.4×10⁸ total CAR-expressing T cells, from at or about at or about 1.5×10⁸ to at or about 4.6×10⁸ total CAR-expressing T cells or from at or about at or about 1.5×10⁸ to at or about 4.5×10⁸ total CAR-expressing T cells.

In some embodiments, the dose of genetically engineered cells comprises from at or about at or about 3.0×10⁸ to at or about 5.4×10⁸ total CAR-expressing T cells, from at or about at or about 3.0×10⁸ to at or about 4.6×10⁸ total CAR-expressing T cells or from at or about 3.0×10⁸ to at or about 4.5×10⁸ total CAR-expressing T cells.

In some embodiments, the dose of genetically engineered cells comprises from at or about 1.0×10⁷ to at or about 8×10⁸ total CAR-expressing (CAR+) T cells, from at or about 1.0×10⁷ to at or about 6.5×10⁸ total CAR+ T cells, from at or about 1.5×10⁷ to at or about 6.5×10⁸ total CAR+ T cells, from at or about 1.5×10⁷ to at or about 6.0×10⁸ total CAR+ T cells, from at or about 2.5×10⁷ to at or about 6.0×10⁸ total CAR+ T cells, from at or about 5.0×10⁷ to at or about 6.0×10⁸ total CAR+ T cells, from at or about 1.0×10⁷ to at or about 5.4×10⁸ total CAR+ T cells, from at or about 1.5×10⁷ to at or about 5.4×10⁸ total CAR+ T cells, from at or about 2.5×10⁷ to at or about 5.4×10⁸ total CAR+ T cells, or from at or about 5.0×10⁷ to at or about 5.4×10⁸ total CAR+ T cells.

In some embodiments, the dose of genetically engineered cells comprises between at or about 2.5×10⁷ CAR-expressing (CAR+) T cells, total T cells, or total peripheral blood mononuclear cells (PBMCs) and at or about 1.2×10⁹ CAR-expressing T cells, total T cells, or total PBMCs, between at or about 5.0×10⁷ CAR-expressing T cells, total T cells, or total peripheral blood mononuclear cells (PBMCs) and at or about 6.0×10⁸ CAR-expressing T cells, total T cells, or total PBMCs, between at or about 5.0×10⁷ CAR-expressing T cells, total T cells, or total peripheral blood mononuclear cells (PBMCs) and at or about 5.4×10⁸ CAR-expressing T cells, total T cells, or total PBMCs, between at or about 5.0×10⁷ CAR-expressing T cells and at or about 4.6×10⁸ CAR-expressing T cells, total T cells, or total peripheral blood mononuclear cells (PBMCs), between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+ cells, in some cases also CAR-expressing (e.g., CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 5.4×10⁸ CD3+ or CD8+ total T cells or CD3+ or CD8+ CAR-expressing cells, from or from about 5.0×10⁷ to or to about 4.6×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the dose is at or about 1.0×10⁷ CD3+ CAR-expressing cells. In some embodiments, the dose is at or about 1.5×10⁷ CD3+ CAR-expressing cells. In some embodiments, the dose is at or about 2.0×10⁷ CD3+ CAR-expressing cells. In some embodiments, the dose is at or about 2.5×10⁷ CD3+ CAR-expressing cells. In some embodiments, the dose is at or about 5×10⁷ CD3+ CAR-expressing cells. In some embodiments, the dose is at or about 1.5×10⁸ CD3+ CAR-expressing cells. In some embodiments, the dose is at or about 3×10⁸ CD3+ CAR-expressing cells. In some embodiments, the dose is at or about 4.5×10⁸ CD3+ CAR-expressing cells. In some embodiments, the dose is at or about 4.6×10⁸ CD3+ CAR-expressing cells. In some embodiments, the dose is at or about 5.4×10⁸ CD3+ CAR-expressing cells. In some embodiments, the dose is at or about 6×10⁸ CD3+ CAR-expressing cells. In some embodiments, the dose is at or about 8×10⁸ CD3+ CAR-expressing cells. In some embodiments, the dose is at or about 1.2×10⁹ CD3+ CAR-expressing cells.

In some embodiments, the dose of genetically engineered cells is with reference to the total number of CD3+ CAR-expressing (CAR+) or CD4+/CD8+ CAR-expressing (CAR+) cells. In some embodiments, the dose comprises a number of genetically engineered cells from or from about 1.0×10⁷ to or to about 1.2×10⁹ CD3+ or CD4+/CD8+ total T cells or CD3+ CAR-expressing or CD4+/CD8+ CAR-expressing cells, from or from about 1.5×10⁷ to or to about 1.2×10⁹ CD3+ or CD4+/CD8+ total T cells or CD3+ CAR-expressing or CD4+/CD8+ CAR-expressing cells, from or from about 2.0×10⁷ to or to about 1.2×10⁹ CD3+ or CD4+/CD8+ total T cells or CD3+ CAR-expressing or CD4+/CD8+ CAR-expressing cells, from or from about 2.5×10⁷ to or to about 1.2×10⁹ CD3+ or CD4+/CD8+ total T cells or CD3+ CAR-expressing or CD4+/CD8+ CAR-expressing cells, from or from about 5.0×10⁷ to or to about 6.0×10⁸ CD3+ or CD4+/CD8+ total T cells or CD3+ CAR-expressing or CD4+/CD8+ CAR-expressing cells, from or from about 5.0×10⁷ to or to about 5.4×10⁸ CD3+ or CD4+/CD8+ total T cells or CD3+ CAR-expressing or CD4+/CD8+ CAR-expressing cells, from or from about 5.0×10⁷ to or to about 4.6×10⁸ CD3+ or CD4+/CD8+ total T cells or CD3+ CAR-expressing or CD4+/CD8+ CAR-expressing cells, from or from about 5.0×10⁷ to or to about 4.5×10⁸ CD3+ or CD4+/CD8+ total T cells or CD3+ CAR-expressing or CD4+/CD8+ CAR-expressing cells, or from or from about 1.5×10⁸ to or to about 3.0×10⁸ CD3+ or CD4+/CD8+ total T cells or CD3+ CAR-expressing or CD4+/CD8+ CAR-expressing cells, each inclusive. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 4.6×10⁸, 5.4×10⁸, 6×10⁸, 8×10⁸ or 1.2×10⁹ CD3+ or CD4+/CD8+ total T cells or CD3+ CAR-expressing or CD4+/CD8+ CAR-expressing cells. In some embodiments, the dose comprises at or about 2.5×10⁷, 5×10⁷, 1.5×10⁸, 3×10⁸, 4.5×10⁸, 4.6×10⁸, 5.4×10⁸, 6×10⁸, 8×10⁸ or 1.2×10⁹ CD3+ CAR-expressing cells. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 4.6×10⁸, 5.4×10⁸, 6×10⁸, 8×10⁸ or 1.2×10⁹ CD4+/CD8+ CAR-expressing cells.

In some embodiments, the dose is at or about 1.0×10⁷ CD4+/CD8+ CAR-expressing cells. In some embodiments, the dose is at or about 1.5×10⁷ CD4+/CD8+ CAR-expressing cells. In some embodiments, the dose is at or about 2.0×10⁷ CD4+/CD8+ CAR-expressing cells. In some embodiments, the dose is at or about 2.5×10⁷ CD4+/CD8+ CAR-expressing cells. In some embodiments, the dose is at or about 5×10⁷ CD4+/CD8+ CAR-expressing cells. In some embodiments, the dose is at or about 1.5×10⁸ CD4+/CD8+ CAR-expressing cells. In some embodiments, the dose is at or about 3×10⁸ CD4+/CD8+ CAR-expressing cells. In some embodiments, the dose is at or about 4.5×10⁸ CD4+/CD8+ CAR-expressing cells. In some embodiments, the dose is at or about 4.6×10⁸ CD4+/CD8+ CAR-expressing cells. In some embodiments, the dose is at or about 5.4×10⁸ CD4+/CD8+ CAR-expressing cells. In some embodiments, the dose is at or about 6×10⁸ CD4+/CD8+ CAR-expressing cells. In some embodiments, the dose is at or about 8×10⁸ CD4+/CD8+ CAR-expressing cells. In some embodiments, the dose is at or about 1.2×10⁹ CD4+/CD8+ CAR-expressing cells. In some embodiments, the dose is at or about 2.5×10⁷ CD4+ or CD8+ CAR-expressing cells. In some embodiments, the dose is at or about 5×10⁷ CD4+ or CD8+ CAR-expressing cells. In some embodiments, the dose is at or about 1.5×10⁸ CD4+ or CD8+ CAR-expressing cells. In some embodiments, the dose is at or about 3×10⁸ CD4+ or CD8+ CAR-expressing cells. In some embodiments, the dose is at or about 4.5×10⁸ CD4+ or CD8+ CAR-expressing cells. In some embodiments, the dose is at or about 4.6×10⁸ CD4+ or CD8+ CAR-expressing cells. In some embodiments, the dose is at or about 6×10⁸ CD4+ or CD8+ CAR-expressing cells. In some embodiments, the dose is at or about 6.5×10⁸ CD4+ or CD8+ CAR-expressing cells. In some embodiments, the dose is at or about 8×10⁸ CD4+ or CD8+ CAR-expressing cells. In some embodiments, the dose is at or about 1.2×10⁹ CD4+ or CD8+ CAR-expressing cells.

In some embodiments, the T cells of the dose include CD4+ T cells, CD8+ T cells or CD4+ and CD8+ T cells.

In some embodiments, for example, where the subject is human, the total of CD4+ T cells and CD8+ T cells of the dose includes between at or about 1×10⁶ and at or about 2×10⁹ total CAR-expressing CD4+ cells and CAR-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 1.0×10⁷, at or about 2.5×10⁷, at or about 2.0×10⁷, at or about 2.5×10⁷, at or about 5×10⁷, at or about 1.5×10⁸, at or about 3×10⁸, at or about 4.5×10⁸, or such as at or about 4.6×10⁸, at or about 5.4×10⁸, at or about 6×10⁸, at or about 6.5×10⁸, at or about 8×10⁸, or at or about 1.2×10⁹ total such cells, or the range between any two of the foregoing values. In some embodiments, for example, where the subject is human, the CD8+ T cells of the dose, including in a dose including CD4+ T cells and CD8+ T cells, includes between at or about 1×10⁶ and at or about 2×10⁹ total recombinant receptor (e.g., CAR)-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 2.5×10⁷, at or about 5×10⁷, at or about 1.5×10⁸, at or about 3×10⁸, at or about 4.5×10⁸, or at or about 4.6×10⁸, at or about 5.4×10⁸, at or about 6×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values. In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as caspase 3. In some aspects, activation of caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active Caspase-3. In some embodiments, the marker of apoptosis is a reagent that detects a feature in a cell that is associated with apoptosis. In certain embodiments, the reagent is an annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. For example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4%, about 5%, about 10%, about 15%, about 20%, about 25%, about 30%, about 35%, about 40%, about 45%, or about 50% of the desired ratio, including any value in between these ranges.

In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1 or is between approximately 1:3 and approximately 3:1, such as approximately 1:1.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷ recombinant-receptor expressing cells. In some embodiments, the dose of cells contains a number of cells, that is about 1.5×10⁸ recombinant-receptor expressing cells, about 3.0×10⁸ recombinant-receptor expressing cells, about 4.5×10⁸ recombinant-receptor expressing cells, or about 4.6×10⁸ recombinant-receptor expressing cells, such as recombinant-receptor expressing cells that are CD3+. In some embodiments, the dose of cells contains a number of cells, that is between at least or at least about 5×10⁶, 6×10⁶, 7×10⁶, 8×10⁶, 9×10⁶, 10×10⁶, and about 15×10⁶ recombinant-receptor expressing cells, such as recombinant-receptor expressing cells that are CD8+. In some embodiments, the dose of cells, such as a target number of cells refers to the total recombinant-receptor expressing cells in the administered composition.

In some embodiments, for example, the lower dose contains less than about 5×10⁶ cells, recombinant receptor (e.g., CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells, PBMCs, or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, which is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

### 2. Immunomodulatory Agent Maintenance Therapy

In some embodiments of the methods, compositions, combinations, kits and uses provided herein, the treatment includes administering to a subject a T cell therapy (e.g., CAR-expressing T cells) followed by an immunomodulatory agent maintenance therapy.

In some embodiments, the immunomodulatory agent is one of a class of immunomodulatory agents that is a structural or functional analog or derivative of thalidomide and/or an inhibitor of E3 ubiquitin ligase. In particular embodiments, the immunomodulatory agent is an inhibitor of E3 ubiquitin ligase.

In some embodiments, the immunomodulatory agent binds to cereblon (CRBN). In some embodiments, the immunomodulatory agent binds to the CRBN E3 ubiquitin-ligase complex. In some embodiments, the immunomodulatory agent binds to CRBN and the CRBN E3 ubiquitin-ligase complex. In some embodiments, the immunomodulatory agent up-regulates the protein or gene expression of CRBN. In some aspects, CRBN is the substrate adaptor for the CRL4^{CRBN} E3 ubiquitin ligase, and modulates the specificity of the enzyme. In some embodiments, binding to CRB or the CRBN E3 ubiquitin ligase complex inhibits E3 ubiquitin ligase activity. In some embodiments, the immunomodulatory agent induces the ubiquitination of KZF1 (Ikaros) and IKZF3 (Aiolos) and/or induces degradation of IKZF1 (Ikaros) and IKZF3 (Aiolos). In some embodiments, the immunomodulatory agent induces the ubiquitination of casein kinase 1A1 (CK1α) by the CRL4CRBN E3 ubiquitin ligase. In some embodiments, the ubiquitination of CK1α results in CK1α degradation.

In some embodiments, the immunomodulatory agent is an inhibitor of the Ikaros (IKZF1) transcription factor. In some embodiments, the immunomodulatory agent enhances ubiquitination of Ikaros. In some embodiments, the immunomodulatory agent enhances the degradation of Ikaros. In some embodiments, the immunomodulatory agent down-regulates the protein or gene expression of Ikaros. In some embodiments, administration of the immunomodulatory agent causes a decrease in Ikaros protein levels.

In some embodiments, the immunomodulatory agent is an inhibitor of the Aiolos (IKZF3) transcription factor. In some embodiments, the immunomodulatory agent enhances ubiquitination of Aiolos. In some embodiments, the immunomodulatory agent enhances the degradation of Aiolos. In some embodiments, the immunomodulatory agent down-regulates the protein or gene expression of Aiolos. In some embodiments, administration of the immunomodulatory agent causes a decrease in Aiolos protein levels.

In some embodiments, the immunomodulatory agent is an inhibitor of both the Ikaros (IKZF1) and Aiolos (IKZF3) transcription factors. In some embodiments, the immunomodulatory agent enhances ubiquitination of both Ikaros and Aiolos. In some embodiments, the immunomodulatory agent enhances the degradation of both Ikaros and Aiolos. In some embodiments, the immunomodulatory agent enhances ubiquitination and degradation of both Ikaros and Aiolos. In some embodiments, administration of the immunomodulatory agent causes both Aiolos protein levels and Ikaros protein levels to decrease.

In some embodiments, the immunomodulatory agent is a selective cytokine inhibitory drug (SelCID). In some embodiments, the immunomodulatory agent inhibits the activity of phosphodiesterase-4 (PDE4). In some embodiments, the immunomodulatory agent suppresses the enzymatic activity of the CDC25 phosphatases. In some embodiments, the immunomodulatory agent alters the intracellular trafficking of CDC25 phosphatases.

In some embodiments, the immunomodulatory agent in the maintenance therapy is thalidomide (2-(2,6-dioxopiperidin-3-yl)-1H-isoindole-1,3(2H)-dione) or an analog or derivative of thalidomide. In certain embodiments, a thalidomide derivative includes structural variants of thalidomide that have a similar biological activity.

In some embodiments, the immunomodulatory agent is thalidomide, having the following structure:

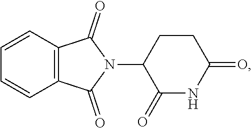 [SMILES: O=C1CCC(N2C(=O)c3ccccc3C2=O)C(=O)N1]

or is an enantiomer or a mixture of enantiomers thereof, or a pharmaceutically acceptable salt, solvate, hydrate, co-crystal, clathrate, or polymorph thereof.

Exemplary thalidomide derivatives include, but are not limited to, lenalidomide (REVLIMMUNOMODULATORY AGENT™; Celgene Corporation), pomalidomide (also known as ACTIMMUNOMODULATORY AGENT™ or POMALYST™ (Celgene Corporation)), CC-1088, CDC-501, and CDC-801, and the compounds disclosed in U.S. Pat. Nos. 5,712,291; 7,320,991; and 8,716,315; U.S. Appl. No. 2016/0313300; and PCT Pub. Nos. WO 2002/068414 and WO 2008/154353.

In some embodiments, the immunomodulatory agent is lenalidomide, having the following structure:

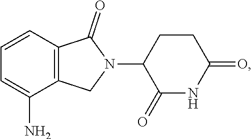 [SMILES: Nc1cccc2c1CN(C1CCC(=O)NC1=O)C2=O]

or is an enantiomer or a mixture of enantiomers thereof, or a pharmaceutically acceptable salt, solvate, hydrate, co-crystal, clathrate, or polymorph thereof. In some of any embodiments, the immunomodulatory agent is lenalidomide or a generic thereof. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide. In particular embodiments, the immunomodulatory agent is generic of lenalidomide.

In some embodiments of the methods, compositions, combinations, kits and uses provided herein, the treatment includes administering to a subject a T cell therapy (e.g., CAR-expressing T cells) followed by an immunomodulatory agent maintenance therapy. In some embodiments, the immunomodulatory agent maintenance therapy is with lenalidomide. In some embodiments of the methods, compositions, combinations, kits and uses provided herein, the treatment includes administering to a subject a T cell therapy (e.g., CAR-expressing T cells) followed by a lenalidomide maintenance therapy.

In some embodiments, the immunomodulatory agent is 1-oxo- and 1,3 dioxo-2-(2,6-dioxopiperldin-3-yl) isoindolines substituted with amino in the benzo ring as described in U.S. Pat. No. 5,635,517 which is incorporated herein by reference.

In some embodiments, the immunomodulatory agent is a compound of the following formula

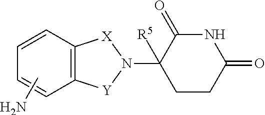

wherein one of X and Y is —C(O)— and the other of X and Y is —C(O)— or —CH₂—, and R⁵ is hydrogen or lower alkyl, or a pharmaceutically acceptable salt thereof. In some embodiments, X is —C(O)— and Y is —CH₂—. In some embodiments, both X and Y are —C(O)—. In some embodiments, R⁵ is hydrogen. In other embodiments, R⁵ is methyl.

In some embodiments, the immunomodulatory agent is a compound that belongs to a class of substituted 2-(2,6-dioxopiperidin-3-yl)-phthalimides and substituted 2-(2,6-dioxopiperldin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference.

In some embodiments, the immunomodulatory agent is a compound of the following formula

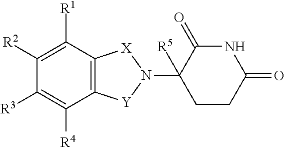

wherein one of X and Y is —C(O)— and the other of X and Y is —C(O)— or —CH₂—; (1) each of R¹, R², R³, and R⁴ are independently halo, alkyl of 1 to 4 carbon atoms, or alkoxy or 1 to 4 carbon atoms, or (2) one of R¹, R³, R⁴, and R⁵ is —NHR^{a} and the remaining of R¹, R², R³, and R⁴ is are hydrogen, wherein R^{a} is hydrogen or alkyl of 1 to 8 carbon atoms; R⁵ is hydrogen or alkyl of 1 to 8 carbon atoms, benzyl, or halo; provided that R⁵ is other than hydrogen if X and Y are —C(O)— and (i) each of R¹, R², R³, and R⁴ is fluoro; or (ii) one of R¹, R², R³, and R⁴ is amino; or a pharmaceutically acceptable salt thereof.

In some embodiments, the immunomodulatory agent is a compound that belongs to a class of isoindole-immunomodulatory agents disclosed in U.S. Pat. No. 7,091,353, U.S. Patent Publication No. 2003/0045552, and International Application No. PCT/USOI/50401 (International Publication No. WO02/059106), each of which are incorporated herein by reference. For example, in some embodiments, the immunomodulatory agent is [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo (3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo (3-piperidyl)-1,3-dioxoisoindolin-4-yl)methyl}cyclopropyl-carboxamide; 2-chloro-N-{(2-(2,6-dioxo (3-piperidyl))-1,3-dioxoisoindolin-4-yl)methyl}acetamide; N-(2-(2,6-dioxo (3-piperidyl))-1,3-dioxoisoindolin-4-yl)-3-pyridylcarboxamide; 3-{1-oxo-4-(benzylamino) isoindolin-2-yl}piperidine-2,6-dione; 2-(2,6-dioxo (3-piperidyl))-4-(benzylamino) isoindoline-1,3-dione; N-{(2-(2,6-dioxo (3-piperidyl))-1,3-dioxoisoindolin-4-yl)methyl}propanamide; N-{(2-(2,6-dioxo (3-piperidyl))-1,3-dioxoisoindolin-4-yl)methyl}-3-pyridylcarboxamide; N-{(2-(2,6-dioxo (3-piperidyl))-1,3-dioxoisoindolin-4-yl)methyl}heptanamide; N-{(2-(2,6-dioxo (3-piperidyl))-1,3-dioxoisoindolin-4-yl)methyl}-2-furylcarboxamide; {N-(2-(2,6-dioxo (3-piperidyl))-1,3-dioxoisoindolin-4-yl) carbamoyl}methyl acetate; N-(2-(2,6-dioxo (3-piperidyl))-1,3-dioxoisoindolin-4-yl) pentanamide; N-(2-(2,6-dioxo (3-piperidyl))-1,3-dioxoisoindolin-4-yl)-2-thienylcarboxamide; N-{[2-(2,6-dioxo (3-piperidyl))-1,3-dioxoisoindolin-4-yl]methyl}(butylamino) carboxamide; N-{[2-(2,6-dioxo (3-piperidyl))-1,3-dioxoisoindolin-4-yl]methyl}(octylamino) carboxamide; or N-{[2-(2,6-dioxo (3-piperidyl))-1,3-dioxoisoindolin-4-yl]methyl}(benzylamino) carboxamide.

In some embodiments, the immunomodulatory agent is a compound that belongs to a class of isoindole-immunomodulatory agents disclosed in U.S. Patent Application Publication Nos. 2002/0045643, International Publication No. WO 98/54170, and U.S. Pat. No. 6,395,754, each of which is incorporated herein by reference. In some embodiments, the immunomodulatory agent is a tetra substituted 2-(2,6-dioxopiperdin-3-yl)-1-oxoisoindolines described in U.S. Pat. No. 5,798,368, which is incorporated herein by reference. In some embodiments, the immunomodulatory agent is 1-oxo and 1,3-dioxo-2-(2,6-dioxopiperidin-3-yl) isoindolines disclosed in U.S. Pat. No. 6,403,613, which is incorporated herein by reference. In some embodiments the immunomodulatory agent is a 1-oxo or 1,3-dioxoisoindoline substituted in the 4- or 5-position of the indoline ring as described in U.S. Pat. Nos. 6,380,239 and 7,244,759, both of which are incorporated herein by reference.

In some embodiments, the immunomodulatory agent is 2-(4-amino-1-oxo-1,3-dihydro-isoindol-2-yl)-4-carbamoyl-butyric acid or 4-(4-amino-1-oxo-1,3-dihydro-isoindol-2-yl)-4-carbamoyl-butyric acid. In some embodiments, the immunomodulatory agent is 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, or 2-{4-[(furan-2-yl-methyl)-amino]-1,3-dioxo-1,3-dihydro-isoindol-2-yl}-pentanedioic acid.

In some embodiments, the immunomodulatory agent is an isoindoline-1-one or isoindoline-1,3-dione substituted in the 2-position with 2,6-dioxo-3-hydroxypiperidin-5-yl as described in U.S. Pat. No. 6,458,810, which is incorporated herein by reference. In some embodiments, the immunomodulatory agent is 3-(5-amino-2-methyl-4-oxo-4H-quinazolin-3-yl)-piperidine-2,6-dione, or an enantiomer or a mixture of enantiomers thereof; or a pharmaceutically acceptable salt, solvate, hydrate, co-crystal, clathrate, or polymorph thereof. In some embodiments, the immunomodulatory agent is 3-[4-(4-morpholin-4-ylmethyl-benzyloxy)-1-oxo-1,3-dihydro-isoindol-2-yl]-piperidine-2,6-dione.

In some embodiments, the immunomodulatory agent is a 4′arylmethoxy isoindoline compound, as described in U.S. Pat. No. 9,828,361, which is incorporated herein by reference. In some embodiments, the immunomodulatory agent is a compound of the following formula

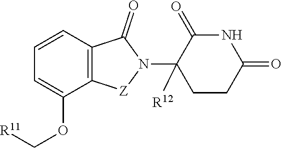

or a pharmaceutically acceptable salt, solvate or stereoisomer thereof, wherein: Z is C═O or CH₂; R¹¹ is —Z¹—R¹³; R¹² is H or (C₁-C₆)alkyl; Z¹ is 6 to 10 membered aryl, heteroaryl, or heterocycle, each of which may be optionally substituted with one or more halogen; or a bond; R¹³ is —(CH₂)ₙ-aryl, —O—(CH₂)ₙ-aryl, or —(CH₂)ₙ—O-aryl, wherein the aryl is optionally substituted with one or more: (C₁-C₆)alkyl; itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy, or halogen; —CONH₂; or —COO—(C₁-C₆)alkyl, wherein the alkyl may be optionally substituted with one or more halogen; —(CH₂)ₙ-heterocycle, —O—(CH₂)ₙ-heterocycle or —(CH₂)ₙ—O-heterocycle, wherein the heterocycle is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; or —(CH₂)ₙ-heteroaryl, —O—(CH₂)ₙ-heteroaryl or —(CH₂)ₙ—O-heteroaryl, wherein the heteroaryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; and n is 0, 1, 2 or 3.

In some embodiments, the immunomodulatory agent is a(S)-3-[4-(4-morpholin-4-ylmethyl-benzyloxy)-1-oxo-1,3-dihydro-isoindol-2-yl]-piperidine-2,6-dione, or a pharmaceutically acceptable salt, solvate, hydrate, co-crystal, clathrate, or polymorph thereof. In some embodiments, (S)-3-[4-(4-morpholin-4-ylmethyl-benzyloxy)-1-oxo-1,3-dihydro-isoindol-2-yl]-piperidine-2,6-dione is also called (3S)-3-[7-[[4-(morpholin-4-ylmethyl)phenyl]methoxy]-3-oxo-1H-isoindol-2-yl]piperidine-2,6-dione, (S)-3-(4-((4-(morpholinomethyl)benzyl)oxy)-1-oxoisoindolin-2-yl) piperidine-2,6-dione, or iberdomide. In some embodiments, the immunomodulatory agent is iberdomide or iberdomide hydrochloride.

In some embodiments, the immunomodulatory agent is iberdomide, having the following structure:

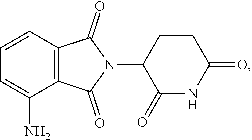 [SMILES: Nc1cccc2c1C(=O)N(C1CCC(=O)NC1=O)C2=O]

or is an enantiomer or a mixture of enantiomers thereof, or a pharmaceutically acceptable salt, solvate, hydrate, co-crystal, clathrate, or polymorph thereof.

In some embodiments, the immunomodulatory agent is an enantiomer or a mixture of enantiomers of(S)-3-[4-(4-morpholin-4-ylmethyl-benzyloxy)-1-oxo-1,3-dihydro-isoindol-2-yl]-piperidine-2,6-dione, or a pharmaceutically acceptable salt, solvate, hydrate, co-crystal, clathrate, or polymorph thereof. In some embodiments, the immunomodulatory agent is(S)-3-[4-(4-morpholin-4-ylmethyl-benzyloxy)-1-oxo-1,3-dihydro-isoindol-2-yl]-piperidine-2,6-dione, or a pharmaceutically acceptable salt, solvate, hydrate, co-crystal, clathrate, or polymorph thereof. In some embodiments, the immunomodulatory agent is (R)-3-[4-(4-morpholin-4-ylmethyl-benzyloxy)-1-oxo-1,3-dihydro-isoindol-2-yl]-piperidine-2,6-dione, or a pharmaceutically acceptable salt, solvate, hydrate, co-crystal, clathrate, or polymorph thereof. In some embodiments, the immunomodulatory agent is a solvate of(S)-3-[4-(4-morpholin-4-ylmethyl-benzyloxy)-1-oxo-1,3-dihydro-isoindol-2-yl]-piperidine-2,6-dione. In some embodiments, the immunomodulatory agent is a hydrate of(S)-3-[4-(4-morpholin-4-ylmethyl-benzyloxy)-1-oxo-1,3-dihydro-isoindol-2-yl]-piperidine-2,6-dione. In some embodiments, the immunomodulatory agent is a salt or solid form of 3-(4-((4-(morpholinomethyl)benzyl)oxy)-1-oxoisoindolin-2-yl) piperidine-2,6-dione, or a stereoisomer thereof, as described in U.S. Pat. No. 9,629,849, which is incorporated herein by reference. In some embodiments, the immunomodulatory agent is a pharmaceutically acceptable salt of 3-[4-(4-morpholin-4-ylmethyl-benzyloxy)-1-oxo-1,3-dihydro-isoindol-2-yl]-piperidine-2,6-dione. In some embodiments, the immunomodulatory agent is a pharmaceutically acceptable salt of(S)-3-[4-(4-morpholin-4-ylmethyl-benzyloxy)-1-oxo-1,3-dihydro-isoindol-2-yl]-piperidine-2,6-dione. For example, in some embodiments, the immunomodulatory agent is a hydrochloride salt of(S)-3-[4-(4-morpholin-4-ylmethyl-benzyloxy)-1-oxo-1,3-dihydro-isoindol-2-yl]-piperidine-2,6-dione. In certain embodiments, the immunomodulatory agent is the Form A crystal form of the hydrochloride salt of(S)-3-[4-(4-morpholin-4-ylmethyl-benzyloxy)-1-oxo-1,3-dihydro-isoindol-2-yl]-piperidine-2,6-dione, as disclosed in U.S. Pat. No. 9,629,849. In some embodiments, the Form A crystal form of the hydrochloride salt of(S)-3-[4-(4-morpholin-4-ylmethyl-benzyloxy)-1-oxo-1,3-dihydro-isoindol-2-yl]-piperidine-2,6-dione is characterized by XRPD peaks located at t 1, 2, 3, 4, 5, 6, 7, 8, 9, 10, 11, 12, 13, 14, 15, 16, 17, 18, 19, 20 or all of the following or approximately the following positions: 9.69, 12.82, 15.09, 15.94, 16.76, 17.65, 19.44, 19.80, 2230, 22.47, 22.95, 23.02, 24.29, 24.48, 24.70, 26.27, 26.77, 27.60, 29.43, 29.72, and 32.91 degrees 20. In some embodiments, the immunomodulatory agent is a salt, hydrate, anhydrate, or solvate of the hydrochloride salt of 3-(4-((4-(morpholinomethyl)benzyl)oxy)-1-oxoisoindolin-2-yl) piperidine-2,6-dione. In some embodiments, the immunomodulatory agent is a polymorph of (S)-3-[4-(4-morpholin-4-ylmethyl-benzyloxy)-1-oxo-1,3-dihydro-isoindol-2-yl]-piperidine-2,6-dione. In some embodiments, the immunomodulatory agent is(S)-3-[4-(4-morpholin-4-ylmethyl-benzyloxy)-1-oxo-1,3-dihydro-isoindol-2-yl]-piperidine-2,6-dione. In some embodiments, the immunomodulatory agent, is

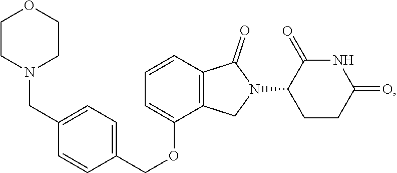 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, co-crystal, clathrate, or polymorph thereof.

In some embodiments, the immunomodulatory agent is as described in Oshima, K. et al., Nihon Rinsho., 72 (6): 1130-5 (2014); Millrine, D. et al., Trends Mol Med., 23 (4): 348-364 (2017); and Collins, et al., Biochem J., 474 (7): 1127-1147 (2017).

In some embodiments, the immunomodulatory agent is an inhibitor of E3 ubiquitin ligase. In some embodiments, the immunomodulatory agent is a derivative of thalidomide. In some embodiments, the immunomodulatory agent is a structural and/or functional analogue of thalidomide. In some embodiments, the immunomodulatory agent is lenalidomide, pomalidomide, avadomide, or a pharmaceutically acceptable salt thereof.

In some embodiments, the immunomodulatory agent is avadomide, having the following structure:

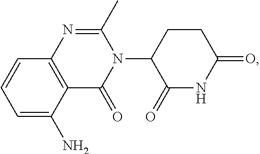 [SMILES: Cc1nc2cccc(N)c2c(=O)n1C1CCC(=O)NC1=O]

or is an enantiomer or a mixture of enantiomers thereof, or a pharmaceutically acceptable salt, solvate, hydrate, co-crystal, clathrate, or polymorph thereof.

In some embodiments, the immunomodulatory agent is lenalidomide, pomalidomide, avadomide, a stereoisomer of lenalidomide, pomalidomide, avadomide or a pharmaceutically acceptable salt, solvate, hydrate, co-crystal, clathrate, or polymorph thereof. In some embodiments, the immunomodulatory agent is lenalidomide, a stereoisomer of lenalidomide or a pharmaceutically acceptable salt, solvate, hydrate, co-crystal, clathrate, or polymorph thereof. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide.

In some embodiments, the immunomodulatory agent is pomalidomide, having the following structure:

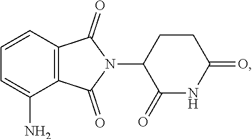 [SMILES: Nc1cccc2c1C(=O)N(C1CCC(=O)NC1=O)C2=O]

or is an enantiomer or a mixture of enantiomers thereof, or a pharmaceutically acceptable salt, solvate, hydrate, co-crystal, clathrate, or polymorph thereof.

In some embodiments, the immunomodulatory agent is avadomide, which also is known as 3-(5-amino-2-methyl-4-oxo-4H-quinazolin-3-yl)-piperidine-2,6-dione having the following structure:

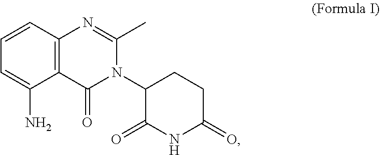 [SMILES: Cc1nc2cccc(N)c2c(=O)n1C1CCC(=O)NC1=O]

or is an enantiomer or a mixture of enantiomers thereof, or a pharmaceutically acceptable salt, solvate, hydrate, co-crystal, clathrate, or polymorph thereof.

In some embodiments, the immunomodulatory agent is an enantiomer or a mixture of enantiomers of 3-(5-amino-2-methyl-4-oxo-4H-quinazolin-3-yl)-piperidine-2,6-dione, or a pharmaceutically acceptable salt, solvate, hydrate, co-crystal, clathrate, or polymorph of 3-(5-amino-2-methyl-4-oxo-4H-quinazolin-3-yl)-piperidine-2,6-dione. In some embodiments, the immunomodulatory agent is a solvate of 3-(5-amino-2-methyl-4-oxo-4H-quinazolin-3-yl)-piperidine-2,6-dione. In some embodiments, the immunomodulatory agent is a hydrate of 3-(5-amino-2-methyl-4-oxo-4H-quinazolin-3-yl)-piperidine-2,6-dione. In some embodiments, the immunomodulatory agent is a pharmaceutically acceptable salt of 3-(5-amino-2-methyl-4-oxo-4H-quinazolin-3-yl)-piperidine-2,6-dione. In some embodiments, the immunomodulatory agent is a polymorph of 3-(5-amino-2-methyl-4-oxo-4H-quinazolin-3-yl)-piperidine-2,6-dione. In some embodiments, the immunomodulatory agent is 3-(5-amino-2-methyl-4-oxo-4H-quinazolin-3-yl)-piperidine-2,6-dione. In some embodiments, the immunomodulatory agent has the structure of Formula I.

In some embodiments, the immunomodulatory agent is lenalidomide, which also is known as 3-(4-amino-1-oxo-1,3-dihydro-2H-isoindol-2-yl) piperidine-2,6-dione, or is an enantiomer or a mixture of enantiomers thereof, or a pharmaceutically acceptable salt, solvate, hydrate, co-crystal, clathrate, or polymorph thereof. In some embodiments, lenalidomide is 2,6-Piperidinedione, 3-(4-amino-1,3-dihydro-1-oxo-2H-isoindol-2-yl)-, 3-(4-Amino-1-oxo-1,3-dihydro-2H-isoindol-2-yl)-2,6-piperidinedione, 3-(4-Amino-1-oxo-1,3-dihydro-2H-isoindol-2-yl)-2,6-pipéridinedione, 3-(4-Amino-1-oxo-1,3-dihydro-2H-isoindol-2-yl) piperidin-2,6-dion, 3-(4-amino-1-oxo-1,3-dihydro-2H-isoindol-2-yl) piperidine-2,6-dione, 3-(4-amino-1-oxo-1,3-dihydro-2H-isoindol-2-yl) pipéridine-2,6-dione, all of which can be used interchangeably, or is an enantiomer or a mixture of enantiomers thereof, or a pharmaceutically acceptable salt, solvate, hydrate, co-crystal, clathrate, or polymorph thereof.

In some embodiments, the immunomodulatory agent is (R)-3-(4-amino-1-oxo-1,3-dihydro-2H-isoindol-2-yl) piperidine-2,6-dione. In some embodiments, the immunomodulatory agent is(S)-3-(4-amino-1-oxo-1,3-dihydro-2H-isoindol-2-yl) piperidine-2,6-dione. In some embodiments, the immunomodulatory agent is a mixture of (R)-3-(4-amino-1-oxo-1,3-dihydro-2H-isoindol-2-yl) piperidine-2,6-dione and(S)-3-(4-amino-1-oxo-1,3-dihydro-2H-isoindol-2-yl) piperidine-2,6-dione.

In some embodiments, the immunomodulatory agent is

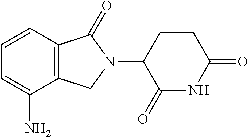 [SMILES: Nc1cccc2c1CN(C1CCC(=O)NC1=O)C2=O]

(Formula II), or an enantiomer or a mixture of enantiomers thereof; or a pharmaceutically acceptable salt, solvate, hydrate, co-crystal, clathrate, or polymorph thereof. In some embodiments, the immunomodulatory agent is

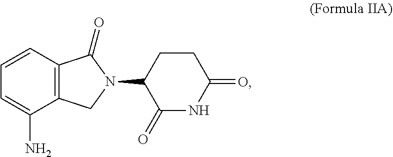

or a pharmaceutically acceptable salt, solvate, hydrate, co-crystal, clathrate, or polymorph thereof. In other embodiments, the immunomodulatory agent is

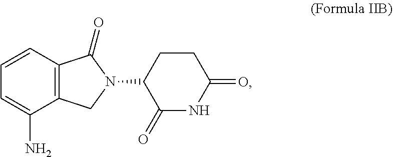

or a pharmaceutically acceptable salt, solvate, hydrate, co-crystal, clathrate, or polymorph thereof. In certain embodiments, the immunomodulatory agent comprises a mixture of

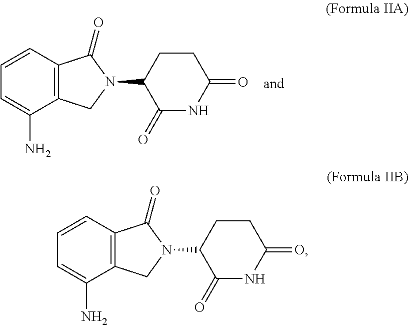

or pharmaceutically acceptable salts, solvates, hydrates, co-crystals, clathrates, or polymorphs thereof.

In some embodiments, the immunomodulatory agent is an enantiomer or a mixture of enantiomers of 3-(4-Amino-1-oxo-1,3-dihydro-2H-isoindol-2-yl) piperidine-2,6-dione, or a pharmaceutically acceptable salt, solvate, hydrate, co-crystal, clathrate, or polymorph of 3-(4-Amino-1-oxo-1,3-dihydro-2H-isoindol-2-yl) piperidine-2,6-dione. In some embodiments, the immunomodulatory agent is a solvate of (R)-3-(4-Amino-1-oxo-1,3-dihydro-2H-isoindol-2-yl) piperidine-2,6-dione and/or(S)-3-(4-Amino-1-oxo-1,3-dihydro-2H-isoindol-2-yl) piperidine-2,6-dione. In some embodiments, the immunomodulatory agent is a hydrate of (RS)-3-(4-Amino-1-oxo-1,3-dihydro-2H-isoindol-2-yl) piperidine-2,6-dione and/or(S)-3-(4-Amino-1-oxo-1,3-dihydro-2H-isoindol-2-yl) piperidine-2,6-dione. In some embodiments, the immunomodulatory agent is a pharmaceutically acceptable salt of (R)-3-(4-Amino-1-oxo-1,3-dihydro-2H-isoindol-2-yl) piperidine-2,6-dione and/or(S)-3-(4-Amino-1-oxo-1,3-dihydro-2H-isoindol-2-yl) piperidine-2,6-dione. In some embodiments, the immunomodulatory agent is lenalidomide, or 3-(4-amino-1-oxo-1,3-dihydro-2H-isoindol-2-yl) piperidine-2,6-dione. In some embodiments, the immunomodulatory agent has the structure of Formula II. In some embodiments, the immunomodulatory agent has the structure of Formula IIA or Formula IIB or a mixture thereof.

In some embodiments, the immunomodulatory agent is pomalidomide, which is also known as 4-amino-2-(2,6-dioxopiperidin-3-yl) isoindoline-1,3-dione, or is an enantiomer or a mixture of enantiomers thereof; or a pharmaceutically acceptable salt, solvate, hydrate, co-crystal, clathrate, or polymorph thereof. In some embodiments, the immunomodulatory agent is

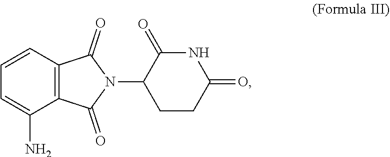 [SMILES: Nc1cccc2c1C(=O)N(C1CCC(=O)NC1=O)C2=O]

or an enantiomer or a mixture of enantiomers thereof; or a pharmaceutically acceptable salt, solvate, hydrate, co-crystal, clathrate, or polymorph thereof. In some embodiments, the immunomodulatory agent is

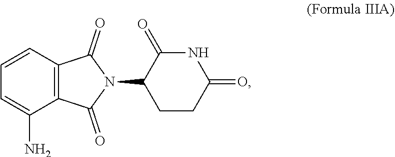

or a pharmaceutically acceptable salt, solvate, hydrate, co-crystal, clathrate, or polymorph thereof. In other embodiments, the immunomodulatory agent is

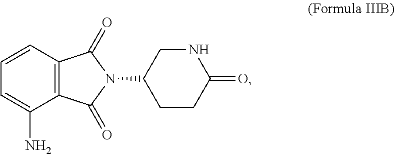

or a pharmaceutically acceptable salt, solvate, hydrate, co-crystal, clathrate, or polymorph thereof. In certain embodiments, the immunomodulatory agent comprises a mixture of

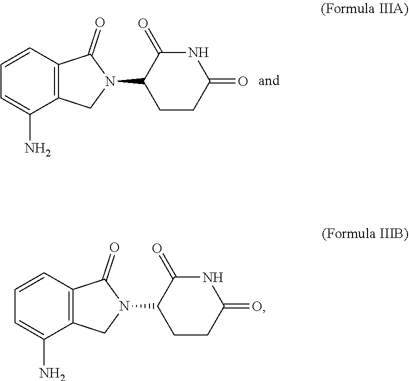

or pharmaceutically acceptable salts, solvates, hydrates, co-crystals, clathrates, or polymorphs thereof.

In some embodiments, the immunomodulatory agent is an enantiomer or a mixture of enantiomers of 4-amino-2-(2,6-dioxopiperidin-3-yl) isoindoline-1,3-dione, or a pharmaceutically acceptable salt, solvate, hydrate, co-crystal, clathrate, or polymorph of 4-amino-2-(2,6-dioxopiperidin-3-yl) isoindoline-1,3-dione. In some embodiments, the immunomodulatory agent is (R)-4-amino-2-(2,6-dioxopiperidin-3-yl) isoindoline-1,3-dione and/or(S)-4-amino-2-(2,6-dioxopiperidin-3-yl) isoindoline-1,3-dione, or a pharmaceutically acceptable salt, solvate, hydrate, co-crystal, clathrate, or polymorph of (R)-4-amino-2-(2,6-dioxopiperidin-3-yl) isoindoline-1,3-dione and/or(S)-4-amino-2-(2,6-dioxopiperidin-3-yl) isoindoline-1,3-dione. In some embodiments, the immunomodulatory agent is a solvate of (R)-4-amino-2-(2,6-dioxopiperidin-3-yl) isoindoline-1,3-dione and/or(S)-4-amino-2-(2,6-dioxopiperidin-3-yl) isoindoline-1,3-dione. In some embodiments, the immunomodulatory agent is a hydrate of (R)-4-amino-2-(2,6-dioxopiperidin-3-yl) isoindoline-1,3-dione and/or(S)-4-amino-2-(2,6-dioxopiperidin-3-yl) isoindoline-1,3-dione. In some embodiments, the immunomodulatory agent is a pharmaceutically acceptable salt of (R)-4-amino-2-(2,6-dioxopiperidin-3-yl) isoindoline-1,3-dione and/or(S)-4-amino-2-(2,6-dioxopiperidin-3-yl) isoindoline-1,3-dione. In some embodiments, the immunomodulatory agent is (R)-4-amino-2-(2,6-dioxopiperidin-3-yl) isoindoline-1,3-dione, (S)-4-amino-2-(2,6-dioxopiperidin-3-yl) isoindoline-1,3-dione, or a mixture thereof in any ratio. In some embodiments, the immunomodulatory agent has the structure of Formula III. In some embodiments, the immunomodulatory agent has the structure of Formula IIIA or Formula IIIB or a mixture thereof.

In some embodiments, the immunomodulatory agent is iberdomide, which also is known as(S)-3-[4-(4-morpholin-4-ylmethyl-benzyloxy)-1-oxo-1,3-dihydro-isoindol-2-yl]-piperidine-2,6-dione, having the following structure:

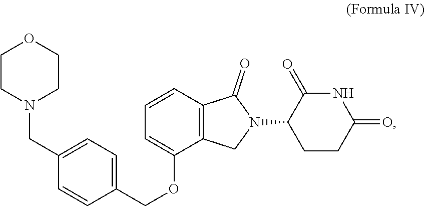 [SMILES: O=C1CC[C@H](N2Cc3c(OCc4ccc(CN5CCOCC5)cc4)cccc3C2=O)C(=O)N1]

or is an enantiomer or a mixture of enantiomers thereof, or a pharmaceutically acceptable salt, solvate, hydrate, co-crystal, clathrate, or polymorph thereof. In some embodiments, the immunomodulatory agent is iberdomide hydrochloride.

In some embodiments, the immunomodulatory agent is or comprises lenalidomide. Lenalidomide is FDA approved for the treatment of multiple myeloma, myelodysplastic syndrome associated with deletion 5q, and most recently in relapsed/refractory mantle-cell lymphoma (MCL). Lenalidomide is a synthetic derivative of thalidomide, and is currently understood to have multiple immunomodulatory effects, including enforcement of immune synapse formation between T cell and antigen presenting cells (APCs). For example, in some cases, lenalidomide modulates T cell responses and results in increased interleukin (IL)-2 production in CD4⁺ and CD8⁺ T cells, induces the shift of T helper (Th) responses from Th2 to Th1, inhibits expansion of regulatory subset of T cells (Tregs), and improves functioning of immunological synapses in follicular lymphoma (FL) and chronic lymphocytic leukemia (CLL) (Otahal et al., Oncoimmunology (2016) 5 (4): e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. In some embodiments, the immunomodulatory agent can include a generic of lenalidomide, which may include a pharmaceutically active salt, solvate, hydrate, co-crystal, clathrate, polymorph, stereoisomer or enantiomer of lenalidomide.

In some embodiments, the immunomodulatory agent is CC-92480 ((S)-4-(4-(4-(((2-(2,6-dioxopiperidin-3-yl)-1-oxoisoindolin-4-yl)oxy)methyl)benzyl) piperazin-1-yl)-3-fluorobenzonitrile) having the structure:

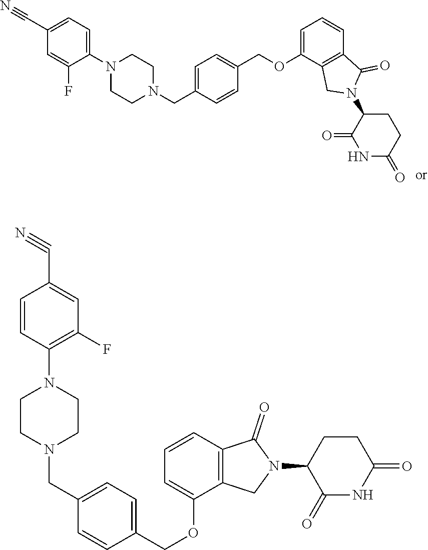 [SMILES: N#Cc1ccc(N2CCN(Cc3ccc(COc4cccc5c4CN([C@H]4CCC(=O)NC4=O)C5=O)cc3)CC2)c(F)c1 ; N#Cc1ccc(N2CCN(Cc3ccc(COc4cccc5c4CN([C@H]4CCC(=O)NC4=O)C5=O)cc3)CC2)c(F)c1]

or an enantiomer or a mixture of enantiomers of CC-92480, or a pharmaceutically acceptable salt, solvate, hydrate, co-crystal, clathrate, or polymorph thereof. In some embodiments, the immunomodulatory agent is a pharmaceutically acceptable salt of CC-92480. In some embodiments, the immunomodulatory agent is a solvate of CC-92480. In some embodiments, the immunomodulatory agent is a hydrate of CC-92480. In some embodiments, the immunomodulatory agent is a co-crystal of CC-92480. In some embodiments, the immunomodulatory agent is a clathrate of CC-92480. In some embodiments, the immunomodulatory agent is a polymorph of CC-92480. In some embodiments, the immunomodulatory agent is CC-92480.

In some embodiments, the immunomodulatory agent is CC-885 having the structure

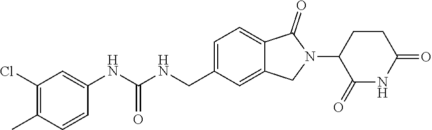 [SMILES: Cc1ccc(NC(=O)NCc2ccc3c(c2)CN(C2CCC(=O)NC2=O)C3=O)cc1Cl]

or an enantiomer or a mixture of enantiomers of CC-90009, or a pharmaceutically acceptable salt, solvate, hydrate, co-crystal, clathrate, or polymorph thereof.

In some embodiments, the immunomodulatory agent is selected from among the group consisting of thalidomide, lenalidomide, pomalidomide, iberdomide (CC-220), CC-92480, CC-99282, CC-91633, and CC-90009, an enantiomer or a mixture of enantiomers thereof, or a pharmaceutically acceptable salt, solvate, hydrate, co-crystal, clathrate, or polymorph thereof. In some embodiments, the immunomodulatory agent is selected from among the group consisting of thalidomide, lenalidomide, pomalidomide, iberdomide (CC-220), CC-92480, CC-99282, CC-91633, and CC-90009 or a pharmaceutically acceptable salt thereof. In some embodiments, the immunomodulatory agent is selected from among the group consisting of thalidomide, lenalidomide, pomalidomide, iberdomide (CC-220), CC-92480, CC-99282, and CC-90009 or a pharmaceutically acceptable salt thereof.

In some embodiments, the term “pharmaceutically acceptable salt” refers to a salt prepared from a pharmaceutically acceptable non-toxic acid or base including an inorganic acid and base and an organic acid and base. Suitable pharmaceutically acceptable base addition salts include metallic salts made from aluminum, calcium, lithium, magnesium, potassium, sodium, and zinc or organic salts made from lysine, N,N′-dibenzylethylenediamine, chloroprocaine, choline, diethanolamine, ethylenediamine, meglumine (N-methyl-glucamine), and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Others are well-known in the art, see for example Remington's Pharmaceutical Sciences, 18th eds., Mack Publishing, Easton PA (1990) or Remington: The Science and Practice of Pharmacy, 19th eds., Mack Publishing, Easton PA (1995).

In some embodiments, the term “stereoisomer” or “stereomerically pure” means one stereoisomer of a drug that is substantially free of other stereoisomers of that drug. For example, a stereomerically pure drug having one chiral center will be substantially free of the opposite enantiomer of the drug. A stereomerically pure drug having two chiral centers will be substantially free of other diastereomers of the drug. A typical stereomerically pure drug comprises greater than about 80% by weight of one stereoisomer of the drug and less than about 20% by weight of other stereoisomers of the drug, greater than about 90% by weight of one stereoisomer of the drug and less than about 10% by weight of the other stereoisomers of the drug, greater than about 95% by weight of one stereoisomer of the drug and less than about 5% by weight of the other stereoisomers of the drug, or greater than about 97% by weight of one stereoisomer of the drug and less than about 3% by weight of the other stereoisomers of the drug. The drugs can have chiral centers and can occur as racemates, individual enantiomers or diastereomers, and mixtures thereof. Methods involving administration of any such isomeric forms of the immunomodulatory agent are included within the embodiments provided herein, including administration of mixtures thereof.

In some embodiments, the immunomodulatory agent contains one chiral center, and can exist as a mixture of enantiomers, e.g., a racemic mixture. This disclosure encompasses the use of stereomerically pure forms of such a drug, as well as the use of mixtures of those forms. For example, mixtures comprising equal or unequal amounts of the enantiomers of the immunomodulatory agent may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E L. Eliel, Ed., Univ. of Notre Dame Press, Notre Dame, IN, 1972).

It is to be understood that the chiral centers of the immunomodulatory agent may undergo epimerization in vivo. As such, one of skill in the art will recognize that in the case of epimerization in vivo, administration of the immunomodulatory agent in its (R) form may be equivalent to administration of the immunomodulatory agent in its(S) form.

Optically active (+) and (−), (R)- and (S)-, or (D)- and (L)-isomers may be prepared using chiral synthons or chiral reagents, or resolved using conventional techniques, such as chromatography on a chiral stationary phase.

In some embodiments, the term “solvate” means a physical association of a drug with one or more solvent molecules, whether organic or inorganic. This physical association includes hydrogen bonding. In certain instances, the solvate will be capable of isolation, for example when one or more solvent molecules are incorporated in the crystal lattice of the crystalline solid. In some embodiments, “solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

It is understood that, independently of stereomerical or isotopic composition, the immunomodulatory agent can be administered in the form of any of the pharmaceutically acceptable salts described herein. Equally, it is understood that the isotopic composition may vary independently from the stereomerical composition of the immunomodulatory agent. Further, the isotopic composition, while being restricted to those elements present in immunomodulatory agent or salt thereof, may otherwise vary independently from the selection of the pharmaceutically acceptable salt of immunomodulatory agent.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

In some embodiments, the immunomodulatory agent of the immunomodulatory therapy is administered at a dose of from or from about 0.1 mg to 100 mg, from or from about 0.1 mg to 75 mg, from or from about 0.1 mg to 50 mg, from or from about 0.1 mg to 25 mg, from or from about 0.1 mg to 10 mg, from or from about 0.1 mg to 5 mg, from or from about 0.1 mg to 1 mg, from or from about 1 mg to 100 mg, from or from about 1 mg to 75 mg, from or from about 1 mg to 50 mg, from or from about 1 mg to 25 mg, from or from about 1 mg to 10 mg, from or from about 1 mg to 5 mg, from or from about 5 mg to 100 mg, from or from about 5 mg to 75 mg, from or from about 5 mg to 50 mg, from or from about 5 mg to 25 mg, from or from about 5 mg to 10 mg, from or from about 10 mg to 100 mg, from or from about 10 mg to 75 mg, from or from about 10 mg to 50 mg, from or from 10 mg to 25 mg, from or from about 25 mg to 100 mg, from or from about 25 mg to 75 mg, from or from about 25 mg to 50 mg, from or from about 50 mg to 100 mg, from or from about 50 mg to 75 mg, or from or from about 75 mg to 100 mg, each inclusive. In some embodiments, the immunomodulatory agent of the immunomodulatory therapy is administered at a dose of 5 mg. In some embodiments, the immunomodulatory agent of the immunomodulatory therapy is administered at a dose of 10 mg. In some embodiments, the immunomodulatory agent of the immunomodulatory therapy is administered at a dose of 15 mg. In some embodiments, the dose is a daily dose. In some embodiments, the dose is a once-daily dose. In some embodiments, the dose is the amount of the immunomodulatory agent of the immunomodulatory therapy that is administered on each of the days on which the immunomodulatory agent of the immunomodulatory therapy is administered.

In some embodiments, the immunomodulatory agent of the immunomodulatory therapy is administered at a dose of from or from about 0.1 mg to about 1.0 mg, from or from about 0.1 mg to 0.9 mg, from or from about 0.1 mg to 0.8 mg, from or from about 0.1 mg to 0.7 mg, from or from about 0.1 mg to 0.6 mg, from or from about 0.1 mg to 0.5 mg, from or from about 0.1 mg to 0.4 mg, from or from about 0.1 mg to 0.3 mg, from or from about 0.1 mg to 0.2 mg, from or from about 0.2 mg to 1.0 mg, from or from about 0.2 mg to 0.9 mg, from or from about 0.2 mg to 0.8 mg, from or from about 0.2 mg to 0.7 mg, from or from about 0.2 mg to 0.6 mg, from or from about 0.2 mg to 0.5 mg, from or from about 0.2 mg to 0.4 mg, from or from about 0.2 mg to 0.3 mg, from or from about 0.3 mg to 1.0 mg, from or from about 0.3 mg to 0.9 mg, from or from about 0.3 mg to 0.8 mg, from or from about 0.3 mg to 0.7 mg, from or from about 0.3 mg to 0.6 mg, from or from about 0.3 mg to 0.5 mg, from or from about 0.3 mg to 0.4 mg, from or from about 0.4 mg to 1.0 mg, from or from about 0.4 mg to 0.9 mg, from or from about 0.4 mg to 0.8 mg, from or from about 0.4 mg to 0.7 mg, from or from about 0.4 mg to 0.6 mg, from or from about 0.4 mg to 0.5 mg, from or from about 0.5 mg to 1.0 mg, from or from about 0.5 mg to 0.9 mg, from or from about 0.5 mg to 0.8 mg, from or from about 0.5 mg to 0.7 mg, from or from about 0.5 mg to 0.6 mg, from or from about 0.6 mg to 1.0 mg, from or from about 0.6 mg to 0.9 mg, from or from about 0.6 mg to 0.8 mg, from or from about 0.6 mg to 0.7 mg, from or from about 0.7 mg to 1.0 mg, from or from about 0.7 mg to 0.9 mg, from or from about 0.7 mg to 0.8 mg, from or from about 0.8 mg to 1.0 mg, from or from about 0.8 mg to 0.9 mg, or from or from about 0.8 mg to 1.0 mg, each inclusive. In some embodiments, the dose is a daily dose. In some embodiments, the dose is a once-daily dose. In some embodiments, the dose is the amount of the immunomodulatory agent of the immunomodulatory therapy that is administered on each of the days on which the immunomodulatory agent of the immunomodulatory therapy is administered.

In some embodiments, the immunomodulatory agent of the immunomodulatory therapy is administered several times a day, twice a day, daily, every other day, three times a week, twice a week, or once a week. In some embodiments, the immunomodulatory agent of the immunomodulatory therapy is administered daily. In some embodiments, the immunomodulatory agent of the immunomodulatory therapy is administered daily for a plurality of consecutive days. In some embodiments, the immunomodulatory agent of the immunomodulatory therapy is administered daily for up to about 7, 8, 9, 10, 11, 12, 13, 14, 15, 16, 17, 18, 19, 20, 21, 22, 23, 24, 25, 26, 27, 28, 29, 30, or more than 30 consecutive days. In some embodiments, the immunomodulatory agent of the immunomodulatory therapy is administered daily for up to about 28 consecutive days.

In some embodiments, immunomodulatory agent of the immunomodulatory agent maintenance therapy is administered at a dose of from or from about 0.5 mg per day to 25 mg per day, from or from about 0.5 mg per day to 10 mg per day, from or from about 0.5 mg per day to 5 mg per day, from or from about 0.5 mg per day to 2.5 mg per day, from or from about 0.5 mg per day to 1 mg per day, from or from about 1 mg per day to 50 mg per day, from or from about 1 mg per day to 25 mg per day, from or from about 1 mg per day to 10 mg per day, from or from about 1 mg per day to 5 mg per day, 1 from or from about mg per day to 2.5 mg per day, from or from about 2.5 mg per day to 50 mg per day, from or from about 2.5 mg per day to 25 mg per day, from or from about 2.5 mg per day to 10 mg per day, from or from about 2.5 mg per day to 5 mg per day, from or from about 5 mg per day to 50 mg per day, from or from about 5 mg per day to 25 mg per day, from or from about 5 mg per day to 10 mg per day, from or from about 10 mg per day to 50 mg per day, from or from about 10 mg per day to 25 mg per day or from or from about 25 mg per day to 50 mg per day, each inclusive. In some embodiments, the immunomodulatory agent of the immunomodulatory therapy is administered at a dose of 5 mg. In some embodiments, the immunomodulatory agent of the immunomodulatory therapy is administered at a dose of 10 mg. In some embodiments, the immunomodulatory agent of the immunomodulatory therapy is administered at a dose of 15 mg. In some embodiments, the dose is a daily dose. In some embodiments, the dose is a once-daily dose. In some embodiments, the dose is the amount of the immunomodulatory agent of the immunomodulatory agent maintenance therapy that is administered on each of the days on which the immunomodulatory agent is administered.

In particular embodiments, the immunomodulatory agent of the immunomodulatory agent maintenance therapy is administered in an amount from or from about 0.5 mg per day to 50 mg per day. In particular embodiments, the immunomodulatory agent of the immunomodulatory agent maintenance therapy is administered in an amount of 2.5 mg per day to 25 mg per day. In particular embodiments, the immunomodulatory agent of the immunomodulatory agent maintenance therapy is administered in an amount of 2.5 mg per day to 10 mg per day.

In particular embodiments, the immunomodulatory agent of the immunomodulatory agent maintenance therapy is administered in an amount of at or about 1 mg per day. In particular embodiments, the immunomodulatory agent of the immunomodulatory agent maintenance therapy is administered in an amount of at or about 2.5 mg per day. In particular embodiments, the immunomodulatory agent of the immunomodulatory agent maintenance therapy is administered in an amount of at or about 5 mg per day. In particular embodiments, the immunomodulatory agent of the immunomodulatory agent maintenance therapy is administered in an amount of at or about 10 mg per day. In particular embodiments, the immunomodulatory agent of the immunomodulatory agent maintenance therapy is administered in an amount of at or about 15 mg per day. In particular embodiments, the immunomodulatory agent of the immunomodulatory agent maintenance therapy is administered in an amount of at or about 20 mg per day. In particular embodiments, the immunomodulatory agent of the immunomodulatory agent maintenance therapy is administered in an amount of at or about 25 mg per day.

In some embodiments, an immunomodulatory agent maintenance therapy prior to a leukapheresis to obtain T cells from the subject for engineering with a BCMA targeted CAR to produce a BCMA targeted CAR T cell therapy is initiated any time at or between 7-15 weeks, 7-14 weeks, 7-13 weeks, 7-12 weeks, 8-15 weeks, 8-14 weeks, 8-13 weeks, 8-12 weeks, 9-15 weeks, 9-14 weeks, 9-13 weeks, 9-12 weeks, 10-15 weeks, 10-14 weeks, 10-13 weeks, 10-12 weeks, 11-15 weeks, 11-14 weeks, 11-13 weeks or 11-12 weeks prior to administering the BCMA targeted CAR T cell therapy, each inclusive. In some embodiments, an immunomodulatory agent maintenance therapy prior to a leukapheresis to obtain T cells from the subject for engineering with a BCMA targeted CAR to produce a BCMA targeted CAR T cell therapy is initiated any time at or between 10-12 to weeks prior to administering the BCMA targeted CAR T cell therapy, inclusive. In some embodiments, an immunomodulatory agent maintenance therapy prior to a leukapheresis to obtain T cells from the subject for engineering with a BCMA targeted CAR to produce a BCMA targeted CAR T cell therapy is initiated any time at about 10 weeks prior to administering the BCMA targeted CAR T cell therapy. In some embodiments, an immunomodulatory agent maintenance therapy prior to a leukapheresis to obtain T cells from the subject for engineering with a BCMA targeted CAR to produce a BCMA targeted CAR T cell therapy is initiated any time at about 11 weeks prior to administering the BCMA targeted CAR T cell therapy. In some embodiments, an immunomodulatory agent maintenance therapy prior to a leukapheresis to obtain T cells from the subject for engineering with a BCMA targeted CAR to produce a BCMA targeted CAR T cell therapy is initiated any time at about 12 weeks prior to administering the BCMA targeted CAR T cell therapy. Preleukapheresis administration of an immunomodulatory agent therapy is administering a subject lenalidomide prior to a leukapheresis to obtain T cells from the subject for engineering with a BCMA targeted CAR to produce a BCMA targeted CAR T cell therapy.

In some embodiments, leukapheresis is performed at least 1 week but not more than 8 weeks, at least 1 week but not more than 7 weeks, at least 2 weeks but not more than 8 weeks or at least 2 weeks but not more than 7 weeks, each inclusive, after completion of the immunomodulatory agent maintenance therapy prior to a leukapheresis to obtain T cells from the subject for engineering with a BCMA targeted CAR to produce a BCMA targeted CAR T cell therapy. In some embodiments, leukapheresis is performed at least 2 weeks but nor more than 7 weeks, inclusive, after completion of the immunomodulatory agent maintenance therapy prior to a leukapheresis to obtain T cells from the subject for engineering with a BCMA targeted CAR to produce a BCMA targeted CAR T cell therapy. In some embodiments, leukapheresis is performed at least 2 weeks after completion of the immunomodulatory agent maintenance therapy prior to a leukapheresis to obtain T cells from the subject for engineering with a BCMA targeted CAR to produce a BCMA targeted CAR T cell therapy. In some embodiments, leukapheresis is performed not more than 7 weeks after completion of the immunomodulatory agent maintenance therapy prior to a leukapheresis to obtain T cells from the subject for engineering with a BCMA targeted CAR to produce a BCMA targeted CAR T cell therapy. Preleukapheresis administration of an immunomodulatory agent therapy is administering a subject lenalidomide prior to a leukapheresis to obtain T cells from the subject for engineering with a BCMA targeted CAR to produce a BCMA targeted CAR T cell therapy.

In some embodiments, the immunomodulatory agent maintenance therapy after administering the BCMA targeted CAR T cell therapy is initiated any time at or between 1 to 365 days, 1 to 330 days, 1 to 300 days, 1 to 270 days, 1 to 240 days, 1 to 210 days, 1 to 180 days, 1 to 150 days, 1 to 120 days, 1 to 90 days, 1 to 60 days, 1 to 30 days, or 1 to 15 days after administering the BCMA targeted CAR T cell therapy, each inclusive. In some embodiments, the immunomodulatory agent maintenance therapy after administering the BCMA targeted CAR T cell therapy is initiated any time at or between 15 to 365 days, 15 to 330 days, 15 to 300 days, 15 to 270 days, 15 to 240 days, 15 to 210 days, 15 to 180 days, 15 to 150 days, 15 to 120 days, 15 to 90 days, 15 to 60 days, or 15 to 30 days after administering the BCMA targeted CAR T cell therapy, each inclusive. In some embodiments, the immunomodulatory agent maintenance therapy after administering the BCMA targeted CAR T cell therapy is initiated anywhere at or between 30 to 365 days, 30 to 330 days, 30 to 300 days, 30 to 270 days, 30 to 240 days, 30 to 210 days, 30 to 180 days, 30 to 150 days, 30 to 120 days, 30 to 90 days, or 30 to 60 days after administering the BCMA targeted CAR T cell therapy, each inclusive.

In some embodiments, the immunomodulatory agent maintenance therapy after administering the BCMA targeted CAR T cell therapy is initiated any time at or between 30 to 180 days after administering the BCMA targeted CAR T cell therapy, inclusive. In some embodiments, the immunomodulatory agent maintenance therapy after administering the BCMA targeted CAR T cell therapy is initiated at about 30 days after administering the BCMA targeted CAR T cell therapy. In some embodiments, the immunomodulatory agent maintenance therapy after administering the BCMA targeted CAR T cell therapy is initiated at about 60 days after administering the BCMA targeted CAR T cell therapy. In some embodiments, the immunomodulatory agent maintenance therapy after administering the BCMA targeted CAR T cell therapy is initiated at about 90 days after administering the BCMA targeted CAR T cell therapy. In some embodiments the immunomodulatory agent maintenance therapy after administering the BCMA targeted CAR T cell therapy is initiated at about 120 days after administering the BCMA targeted CAR T cell therapy. In some embodiments, the immunomodulatory agent maintenance therapy after administering the BCMA targeted CAR T cell therapy is initiated at about 150 days after administering the BCMA targeted CAR T cell therapy. In some embodiments, the immunomodulatory agent maintenance therapy after administering the BCMA targeted CAR T cell therapy is initiated at about 180 days after administering the BCMA targeted CAR T cell therapy. In some embodiments, the immunomodulatory agent maintenance therapy after administering the BCMA targeted CAR T cell therapy must initiated at 180 days after administering the BCMA targeted CAR T cell therapy.

In some embodiments, the immunomodulatory agent maintenance therapy after administering the BCMA targeted CAR T cell therapy is initiated any time at or between 1 month and 12 months, 1 month and 10 months, 1 month and 8 months, 1 month and 6 months, 1 month and 4 months, or 1 month and 2 months after administering the BCMA targeted CAR T cell therapy, each inclusive. In some embodiments, the immunomodulatory agent maintenance therapy after administering the BCMA targeted CAR T cell therapy is initiated at or between 1 month and 6 months after administering the BCMA targeted CAR T cell therapy, inclusive.

In some embodiments, the immunomodulatory agent maintenance therapy is initiated any time at or between 1 month and 6 months after administering the BCMA targeted CAR T cell therapy, inclusive.

In some embodiments, the immunomodulatory agent maintenance therapy after administering the BCMA targeted CAR T cell therapy is initiated at at least 1 month, at least 2 months, at least 3 months, at least 4 months, at least 5 months, at least 6 months, at least 7 months, at least 8 months, at least 9 months, at least 10 months, at least 11 months, or at least 12 months, each inclusive, after administering the BCMA targeted CAR T cell therapy. In some embodiments, the immunomodulatory agent maintenance therapy after administering the BCMA targeted CAR T cell therapy is initiated at least 1 month, inclusive, after administering the BCMA targeted CAR T cell therapy. In some embodiments, the immunomodulatory agent maintenance therapy after administering the BCMA targeted CAR T cell therapy is initiated at at most 6 months, inclusive, after administering the BCMA targeted CAR T cell therapy. In some embodiments, the immunomodulatory agent maintenance therapy after administering the BCMA targeted CAR T cell therapy must be initiated at at most 6 months, after administering the BCMA targeted CAR T cell therapy.

In some embodiments the immunomodulatory agent maintenance therapy after administering the BCMA targeted CAR T cell therapy is initiated at no later than 8 months, no later than 7 months, no later than 6 months, no later than 5 months, or no later than 4 months, each inclusive, after administering the BCMA targeted CAR T cell therapy, each inclusive. In some embodiments, the immunomodulatory agent maintenance therapy after administering the BCMA targeted CAR T cell therapy is initiated at no later than 6 months after administering the BCMA targeted CAR T cell therapy, inclusive. In some embodiments, the immunomodulatory agent maintenance therapy after administering the BCMA targeted CAR T cell therapy must be initiated at no later than 6 months after administering the BCMA targeted CAR T cell therapy.

In some embodiments, the immunomodulatory agent maintenance therapy after administering the BCMA targeted CAR T cell therapy is initiated at or at about 1 month, 2 months, 3 months, 4 months, 5 months, 6 months, 7 months, 8 months, 9 months, 10 months, 11 months, or 12 months, after administering the BCMA targeted CAR T cell therapy. In some embodiments, the immunomodulatory agent maintenance therapy after administering the BCMA targeted CAR T cell therapy is initiated at or at about 1 month after administering the BCMA targeted CAR T cell therapy. In some embodiments, the immunomodulatory agent maintenance therapy after administering the BCMA targeted CAR T cell therapy is initiated at or at about 2 months after administering the BCMA targeted CAR T cell therapy. In some embodiments, the immunomodulatory agent maintenance therapy after administering the BCMA targeted CAR T cell therapy is initiated at or at about 3 months after administering the BCMA targeted CAR T cell therapy. In some embodiments, the immunomodulatory agent maintenance therapy after administering the BCMA targeted CAR T cell therapy is initiated at or at about 4 months after administering the BCMA targeted CAR T cell therapy. In some embodiments, the immunomodulatory agent maintenance therapy after administering the BCMA targeted CAR T cell therapy is initiated at or at about 5 months after administering the BCMA targeted CAR T cell therapy. In some embodiments, the immunomodulatory agent maintenance therapy after administering the BCMA targeted CAR T cell therapy is initiated at or at about 6 months after administering the BCMA targeted CAR T cell therapy. In some embodiments, the immunomodulatory agent maintenance therapy after administering the BCMA targeted CAR T cell therapy must be initiated at or at about 6 months after administering the BCMA targeted CAR T cell therapy.

In some embodiments, an immunomodulatory agent maintenance therapy prior to a leukapheresis to obtain T cells from the subject for engineering with a BCMA targeted CAR to produce a BCMA targeted CAR T cell therapy is initiated any time at or between 2-5 weeks, 2-4 weeks, 2-3 weeks, 3-5 weeks or 3-4 weeks prior to screening, each inclusive. In some embodiments, an immunomodulatory agent maintenance therapy prior to a leukapheresis to obtain T cells from the subject for engineering with a BCMA targeted CAR to produce a BCMA targeted CAR T cell therapy is initiated at or between 2-4 weeks prior to screening, inclusive. In some embodiments, an immunomodulatory agent maintenance therapy prior to a leukapheresis to obtain T cells from the subject for engineering with a BCMA targeted CAR to produce a BCMA targeted CAR T cell therapy is initiated at about 2 weeks prior to screening. In some embodiments, an immunomodulatory agent maintenance therapy prior to a leukapheresis to obtain T cells from the subject for engineering with a BCMA targeted CAR to produce a BCMA targeted CAR T cell therapy is initiated at about 3 weeks prior to screening. In some embodiments, an immunomodulatory agent maintenance therapy prior to a leukapheresis to obtain T cells from the subject for engineering with a BCMA targeted CAR to produce a BCMA targeted CAR T cell therapy at about 4 weeks prior to screening. Preleukapheresis administration of an immunomodulatory agent therapy is administering a subject lenalidomide prior to a leukapheresis to obtain T cells from the subject for engineering with a BCMA targeted CAR to produce a BCMA targeted CAR T cell therapy.

In some embodiments, time of screening can be within 7-12 weeks, 7-11 weeks, 7-10 weeks, 7-9 weeks, 7-8 weeks, 8-12 weeks, 8-11 weeks, 8-10 weeks, 8-9 weeks, 9-12 weeks, 9-11 weeks, 9-10 weeks, 10-12 weeks, 10-11 weeks or 11-12 weeks prior to a leukapheresis to obtain T cells from the subject for engineering with a BCMA targeted CAR to produce a BCMA targeted CAR T cell therapy. In some embodiments, time of screening can be within 9-10 weeks prior to a leukapheresis to obtain T cells from the subject for engineering with a BCMA targeted CAR to produce a BCMA targeted CAR T cell therapy. In some embodiments, time of screening can be within about 9 weeks prior to a leukapheresis to obtain T cells from the subject for engineering with a BCMA targeted CAR to produce a BCMA targeted CAR T cell therapy. In some embodiments, time of screening can be within about 10 weeks prior to a leukapheresis to obtain T cells from the subject for engineering with a BCMA targeted CAR to produce a BCMA targeted CAR T cell therapy.

In some embodiments, the immunomodulatory agent maintenance therapy after administering the BCMA targeted CAR T cell therapy is initiated any time at or between 12-18 weeks, 12-17 weeks, 12-16 weeks, 12-15 weeks, 12-14 weeks, 12-13 weeks, 13-18 weeks, 13-17 weeks, 13-16 weeks, 13-15 weeks, 13-14 weeks, 14-18 weeks, 14-17 weeks, 14-16 weeks, 14-15 weeks, 15-18 weeks, 15-17 weeks, 15-16 weeks, 16-18 weeks or 16-17 weeks after screening, each inclusive. In some embodiments, the immunomodulatory agent maintenance therapy after administering the BCMA targeted CAR T cell therapy is initiated at or between 14-16 weeks after screening, inclusive. In some embodiments, the immunomodulatory agent maintenance therapy after administering the BCMA targeted CAR T cell therapy is initiated at about 14 weeks after screening. In some embodiments, the immunomodulatory agent maintenance therapy after administering the BCMA targeted CAR T cell therapy is initiated at about 15 weeks after screening. In some embodiments, the immunomodulatory agent maintenance therapy after administering the BCMA targeted CAR T cell therapy is initiated at about 16 weeks after screening.

In some embodiments, the immunomodulatory agent maintenance therapy is initiated any time at or between 1 to 730 days, 1 to 575 days, 1 to 545 days, 1 to 485 days, 1 to 455 days, 1 to 425 days, 1 to 395 days, 1 to 365 days, 1 to 330 days, 1 to 300 days, 1 to 270 days, 1 to 240 days, 1 to 210 days, 1 to 180 days, 1 to 150 days, 1 to 120 days, 1 to 90 days, 1 to 60 days, 1 to 30 days, or 1 to 15 days after administering the BCMA targeted CAR T cell therapy, each inclusive. In some embodiments, the immunomodulatory agent maintenance therapy is initiated any time at or between 15 to 730 days, 15 to 575 days, 15 to 545 days, 15 to 485 days, 15 to 455 days, 15 to 425 days, 15 to 395 days, 15 to 365 days, 15 to 330 days, 15 to 300 days, 15 to 270 days, 15 to 240 days, 15 to 210 days, 15 to 180 days, 15 to 150 days, 15 to 120 days, 15 to 90 days, 15 to 60 days, or 15 to 30 days after administering the BCMA targeted CAR T cell therapy, each inclusive. In some embodiments, the immunomodulatory agent maintenance therapy is initiated anywhere at or between 30 to 730 days, 30 to 575 days, 30 to 545 days, 30 to 485 days, 30 to 455 days, 30 to 425 days, 30 to 395 days, 30 to 365 days, 30 to 330 days, 30 to 300 days, 30 to 270 days, 30 to 240 days, 30 to 210 days, 30 to 180 days, 30 to 150 days, 30 to 120 days, 30 to 90 days, or 30 to 60 days after administering the BCMA targeted CAR T cell therapy, each inclusive.

In some embodiments, the immunomodulatory agent maintenance therapy is initiated any time at or between 1 month and 24 months, 1 month and 22 months, 1 month and 20 months, 1 month and 18 months, 1 month and 16 months, 1 month and 14 months, 1 month and 12 months, 1 month and 10 months, 1 month and 8 months, 1 month and 6 months, 1 month and 4 months, or 1 month and 2 months after administering the BCMA targeted CAR T cell therapy. In some embodiments, the immunomodulatory agent maintenance therapy is initiated any time at or between 1 months and 6 months after administering the BCMA targeted CAR T cell therapy. In some embodiments, the immunomodulatory agent maintenance therapy is initiated any time at or between 4 months and 24 months, 4 months and 22 months, 4 months and 20 months, 4 months and 18 months, 4 months and 16 months, 4 months and 14 months, 4 months and 12 months, 4 months and 10 months, 4 months and 8 months, or 4 months and 6 months after administering the BCMA targeted CAR T cell therapy. In some embodiments, the immunomodulatory agent maintenance therapy is initiated any time at or between 4 months and 10 months after administering the BCMA targeted CAR T cell therapy.

In some embodiments, the immunomodulatory agent maintenance therapy is initiated at at least 1 month, at least 2 months, at least 3 months, at least 4 months, at least 5 months, at least 6 months, at least 7 months, at least 8 months, at least 8 months, at least 9 months, at least 10 months, at least 11 months, or at least 12 months, each inclusive, after administering the BCMA targeted CAR T cell therapy. In some embodiments, the immunomodulatory agent maintenance therapy is initiated at at least 1 month, inclusive, after administering the BCMA targeted CAR T cell therapy. In some embodiments, the immunomodulatory agent maintenance therapy is initiated at at least 4 months, inclusive, after administering the BCMA targeted CAR T cell therapy.

In some embodiments, the immunomodulatory agent maintenance therapy is initiated at no later than 24 months, no later than 23 months, no later than 22 months, no later than 21 months, no later than 20 months, no later than 19 months, no later than 18 months, no later than 17 months, no later than 16 months, no later than 15 months, no later than 14 months, no later than 13 months, no later than 12 months, no later than 11 months, or no later than 10 months, each inclusive, after administering the BCMA targeted CAR T cell therapy. In some embodiments, the immunomodulatory agent maintenance therapy is initiated at no later than 24 months, inclusive, after administering the BCMA targeted CAR T cell therapy. In some embodiments, the immunomodulatory agent maintenance therapy is initiated at no later than 12 months, inclusive, after administering the BCMA targeted CAR T cell therapy. In some embodiments, the immunomodulatory agent maintenance therapy is initiated at no later than 10 months, inclusive, after administering the BCMA targeted CAR T cell therapy.

In some embodiments, the immunomodulatory agent maintenance therapy is initiated at or at about 1 month, 2 months, 3 months, 4 months, 5 months, 6 months, 7 months, 8 months, 9 months, 10 months, 11 months, or 12 months, after administering the BCMA targeted CAR T cell therapy. In some embodiments, the immunomodulatory agent maintenance therapy is initiated at or at about 1 month after administering the BCMA targeted CAR T cell therapy. In some embodiments, the immunomodulatory agent maintenance therapy is initiated at or at about 4 months after administering the BCMA targeted CAR T cell therapy. In some embodiments, the immunomodulatory agent maintenance therapy is initiated at or at about 5 months after administering the BCMA targeted CAR T cell therapy. In some embodiments, the immunomodulatory agent maintenance therapy is initiated at or at about 6 months after administering the BCMA targeted CAR T cell therapy. In some embodiments, the immunomodulatory agent maintenance therapy is initiated at or at about 7 months after administering the BCMA targeted CAR T cell therapy. In some embodiments, the immunomodulatory agent maintenance therapy is initiated at or at about 8 months after administering the BCMA targeted CAR T cell therapy. In some embodiments, the immunomodulatory agent maintenance therapy is initiated at or at about 9 months after administering the BCMA targeted CAR T cell therapy. In some embodiments, the immunomodulatory agent maintenance therapy is initiated at or at about 10 months after administering the BCMA targeted CAR T cell therapy.

In some embodiments, the immunomodulatory agent maintenance therapy is initiated at or at about Day 1, at or at about Day 2, at or at about Day 3, at or at about Day 4, at or at about Day 5, at or at about Day 6, at or at about Day 7, at or at about Day 8, at or at about Day 9, at or at about Day 10, at or at about Day 11, at or at about Day 12, at or at about Day 13, at or at about Day 14, at or at about Day 15 after administering the BCMA targeted CAR T cell therapy.

In some embodiments, the immunomodulatory agent maintenance therapy is initiated at Day 15 or more after administering the BCMA targeted CAR T cell therapy. In some embodiments, the immunomodulatory agent maintenance therapy is initiated at or at about Day 16, at or at about Day 17, at or at about Day 18, at or at about Day 19, at or at about Day 20, at or at about Day 21, at or at about Day 22, at or at about Day 23, at or at about Day 24, at or at about Day 25, at or at about Day 26, at or at about Day 27, at or at about Day 28, at or at about Day 29, or at or at about Day 30 after administering the BCMA targeted CAR T cell therapy.

In some embodiments, the immunomodulatory agent maintenance therapy is initiated at or at about Day 16 after administering the BCMA targeted CAR T cell therapy. In some embodiments, the immunomodulatory agent maintenance therapy is initiated at or at about Day 17 after administering the BCMA targeted CAR T cell therapy. In some embodiments, the immunomodulatory agent maintenance therapy is initiated at or at about Day 18 after administering the BCMA targeted CAR T cell therapy. In some embodiments, the immunomodulatory agent maintenance therapy is initiated at or at about Day 19 after administering the BCMA targeted CAR T cell therapy. In some embodiments, the immunomodulatory agent maintenance therapy is initiated at or at about Day 20 after administering the BCMA targeted CAR T cell therapy. In some embodiments, the immunomodulatory agent maintenance therapy is initiated at or at about Day 21 after administering the BCMA targeted CAR T cell therapy. In some embodiments, the immunomodulatory agent maintenance therapy is initiated at or at about Day 22 after administering the BCMA targeted CAR T cell therapy. In some embodiments, the immunomodulatory agent maintenance therapy is initiated at or at about Day 23 after administering the BCMA targeted CAR T cell therapy. In some embodiments, the immunomodulatory agent maintenance therapy is initiated at or at about Day 24 after administering the BCMA targeted CAR T cell therapy. In some embodiments, the immunomodulatory agent maintenance therapy is initiated at or at about Day 25 after administering the BCMA targeted CAR T cell therapy. In some embodiments, the immunomodulatory agent maintenance therapy is initiated at or at about Day 26 after administering the BCMA targeted CAR T cell therapy. In some embodiments, the immunomodulatory agent maintenance therapy is initiated at or at about Day 27 after administering the BCMA targeted CAR T cell therapy. In some embodiments, the immunomodulatory agent maintenance therapy is initiated at or at about Day 28 after administering the BCMA targeted CAR T cell therapy. In some embodiments, the immunomodulatory agent maintenance therapy is initiated at or at about Day 29 after administering the BCMA targeted CAR T cell therapy. In some embodiments, the immunomodulatory agent maintenance therapy is initiated at or at about Day 30 after administering the BCMA targeted CAR T cell therapy.

In some embodiments, the immunomodulatory agent maintenance therapy continues until disease progression. In some embodiments, the immunomodulatory agent maintenance therapy continues until relapse of disease. In some embodiments, the immunomodulatory agent maintenance therapy continues until inadequate response to disease. In some embodiments, the immunomodulatory agent maintenance therapy continues until the subject achieves complete response (CR).

In some embodiments, the immunomodulatory agent is lenalidomide. In some embodiments, lenalidomide is dosed at 10 mg per day for the first 3 months. In some embodiments, lenalidomide is increased to 15 mg per day if tolerated until relapse. See Attal et al. 2012, N Engl J Med, 366 (19): 1782-1791.

In some embodiments, the immunomodulatory agent of the immunomodulatory agent maintenance therapy is administered in a cycle. In some embodiments, the cycle includes an administration period in which the immunomodulatory agent of the immunomodulatory agent maintenance therapy is administered followed by a rest period during which the immunomodulatory agent of the immunomodulatory agent maintenance therapy is not administered. In some embodiments, the rest period is greater than about 1 day, greater than about 3 consecutive days, greater than about 5 consecutive days, greater than about 7 consecutive days, greater than about 8 consecutive days, greater than about 9 consecutive days, greater than about 10 consecutive days, greater than about 11 consecutive days, greater than about 12 consecutive days, greater than about 13 consecutive days, greater than about 14 consecutive days, greater than about 15 consecutive days, greater than about 16 consecutive days, greater than about 17 consecutive days, greater than about 18 consecutive days, greater than about 19 consecutive days, greater than about 20 consecutive days, greater than about 21 consecutive days, or greater than about 28 or more consecutive days.

In some embodiments, the immunomodulatory agent of the immunomodulatory agent maintenance therapy is administered once daily for 14 days over a 21-day cycle. In some embodiments, the immunomodulatory agent of the immunomodulatory agent maintenance therapy is administered once daily for 14 days over a 28-day cycle. In some embodiments, the immunomodulatory agent of the immunomodulatory agent maintenance therapy is administered once daily for 14 days over a 28-day cycle. In some embodiments, the immunomodulatory agent of the immunomodulatory agent maintenance therapy is administered once daily for 21 days over a 28-day cycle.

In some embodiments, the immunomodulatory agent of the immunomodulatory agent maintenance therapy is administered for at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, or at least 12 cycles. In some embodiments, the immunomodulatory agent of the immunomodulatory agent maintenance therapy is administered for at least 3 cycles. In some embodiments, the immunomodulatory agent of the immunomodulatory agent maintenance therapy is administered for 1, 2, 3, 4, 5, 6, 7, 8, 9, 10, 11, 12, 13, 14, 15, 16, 17, 18, 19, 20, 21, 22, 23, or 24 cycles.

In some embodiments, lenalidomide is dosed at 5 mg per day on days 1 to 28 of a first 28-day cycle and then increased to 10 mg per day on days 1 to 28 of a second 28-day cycle. In some embodiments, after 3 cycles of lenalidomide maintenance at 10 mg per day, the dose can be increased to 15 mg per day.

In some embodiments, lenalidomide is dosed at 10 mg per day on days 1 to 28 of a 28-day cycle. In some embodiments, after 3 cycles of lenalidomide maintenance at 10 mg per day, the dose can be increased to 15 mg per day.

In some embodiments, the immunomodulatory agent of the immunomodulatory agent maintenance therapy is administered orally. In some embodiments, the immunomodulatory agent of the immunomodulatory agent maintenance therapy is administered as a tablet or capsule. In some embodiments, the immunomodulatory agent of the immunomodulatory agent maintenance therapy is administered intravenously.

In some embodiments, the maintenance therapy can be in accord with the regimens used for maintenance after ASCT. See Karam et al. 2021, Oncol Ther 9:69-88 or Mohyuddin et al. 2019, Blood 134 (Supplement 1): 5700.

### 3. Lymphodepleting Therapy

In some embodiments, the subject may receive a bridging therapy after leukapheresis and before lymphodepleting chemotherapy. A treating physician can determine if bridging therapy is necessary, for example for disease control, during manufacturing of the provided composition or cells. In some embodiments, bridging therapies are discontinued prior to initiation of lymphodepletion. In some embodiments, bridging therapies are discontinued 1 day, 2 days, 3 days, 4 days, 5 days, 7 days, 10 days, 14 days, 21 days, 28 days, 45 days, or 60 days before lymphodepletion.

In some aspects, the provided methods can further include administering one or more lymphodepleting therapies, such as prior to initiation of administration of the T cell therapy. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17 (13): 4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119:2709-2720 (2012); Kalos et al., Sci Transl Med, 3 (95): 95ra73 (2011); Clinical Trial Study Record Nos.: NCT02315612; NCT01822652.

Such preconditioning can be carried out with the goal of reducing the risk of one or more of various outcomes that could dampen efficacy of the adoptive cell therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3 (12): 668-681 (2006).

Thus, in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In particular embodiments, prior to the administration of the BCMA targeted CAR T cell therapy to the subject, the subject has received a lymphodepleting therapy comprising the administration of fludarabine at or about 20-40 mg/m² body surface area of the subject (optionally at or about 30 mg/m²) daily for 2-4 days, and/or cyclophosphamide at or about 200-400 mg/m² body surface area of the subject (optionally at or about 300 mg/m²) daily for 2-4 days.

In particular embodiments, prior to the administration of the BCMA targeted CAR T cell therapy to the subject, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines, such as CD107a, IFNγ, IL-2, and TNF. In some aspects, the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject.

### C. Response and Treatment Outcomes and Methods for Assessing the Same

In some embodiments of the methods, uses, kits and articles of manufacture provided herein, the provided T cell therapy followed by an immunomodulatory agent maintenance therapy results in one or more treatment outcomes, such as a feature associated with any one or more of the parameters associated with the therapy or treatment, as described below. In some embodiments, subjects are treated with the immunomodulatory agent maintenance therapy until they have achieved a complete response. In some embodiments, subjects are treated with the immunomodulatory agent maintenance therapy until they have progression of disease. Methods of assessment are provided herein.

Complete response (CR) and stringent complete response (sCR) are defined by the International Myeloma Working Group (IMWG) Standard Response Criteria as set forth in Table 1 below (Kumar et al., Lancet Oncol (2016) 17 (8): e328-46).

TABLE 1
Standard IMWG Response Criteria for sCR and CR

| | |
|---|---|
| Stringent complete | Complete response as defined below plus normal FLC ratio** and |
| response (sCR) | absence of clonal cells in bone marrow biopsy by |
| | immunohistochemistry (κ/λ ratio ≤4:1 or ≥1:2 for κ and λ patients, |
| | respectively, after counting ≥100 plasma cells)†† |
| Complete response | Negative immunofixation on the serum and urine and |
| (CR) | disappearance of any soft tissue plasmacytomas and <5% plasma |
| | cells in bone marrow aspirates |

**All recommendations regarding clinical uses relating to serum FLC levels or FLC ratio are based on results obtained with the validated Freelite test (Binding Site, Birmingham, UK).
††Presence/absence of clonal cells on immunohistochemistry is based upon the κ/λ/L ratio. An abnormal κ/λ ratio by immunohistochemistry requires a minimum of 100 plasma cells for analysis. An abnormal ratio reflecting presence of an abnormal clone is κ/λ of >4:1 or <1:2.

In some embodiments, stringent complete response can be no more myeloma protein in blood and urine, no soft tissue plasmacytomas, and no detectable myeloma cells in the bone marrow. In some embodiments, complete response can be no more myeloma protein in blood and urine, no soft tissue plasmacytomas, and less than 5% myeloma cells in the bone marrow. In some embodiments, very good partial response can be a 90% or more decrease in myeloma proteins in blood and less than 100 mg per 24 hours in urine. In some embodiments, a 50% or larger decrease in myeloma protein in blood and a 90% or larger decrease in myeloma protein in urine in 24 hours, with a plasmacytoma size reduction of 50% or more.

Aside from sCR and CR, IMWG has also defined very good partial response (VGPR), partial response (PR), minimal response (MR), stable disease (SD), and progressive disease (PD).

Very good partial response (VGPR), as defined by the IMWG, requires any of the following: serum and urine M-protein detected by immunofixation but not electrophoresis; both of the following: ≥90% decrease in serum M-protein and urine M-protein <100 mg/24 h; or ≥90% decrease in the difference between involved and uninvolved serum free light chain (FLC) levels if serum and urine M-protein unmeasurable

Partial response (PR), as defined by the IMWG, requires all of the following: ≥50% decrease in the sum of products of two longest perpendicular diameters (SPD) of soft tissue plasmacytoma(s) if present at baseline; ≥50% decrease in serum M-protein; ≥90% decrease in urine M-protein or to <200 mg/24 h; ≥50% decrease in the difference between involved and uninvolved serum free light chain (FLC) levels if serum and urine M-protein unmeasurable; and ≥50% decrease in bone marrow plasma cells (if the percentage was ≥30% at baseline) if serum and urine M-protein unmeasurable and involved free light chain unmeasurable.

Minimal response (MR), as defined by the IMWG, requires all of the following: ≥50% decrease in the sum of products of two longest perpendicular diameters (SPD) of soft tissue plasmacytoma(s) if present at baseline; 25-49% decrease in serum M-protein and 50-89% decrease in urine M-protein.

Stable disease (SD), as defined by the IMWG, is a category of exclusion, i.e., the patient does not meet criteria for progressive disease, minimal response, partial response, very good partial response, or complete response.

Progressive disease (PD), as defined by IMWG, is defined by any of the following: any of the following imaging findings: new lesion; ≥50% increase in the longest diameter of a lesion that previously measured >1 cm in short axis; or ≥50% increase from the nadir in the sum of products of the two longest perpendicular diameters (SPD) of more than one lesion; ≥25% increase from the nadir in any of the following: serum M-protein (with ≥0.5 g/dL absolute increase); urine M-protein (with ≥200 mg/24 h absolute increase); difference between involved and uninvolved serum free light chain (FLC) levels (with >10 mg/dL absolute increase) if serum and urine M-protein unmeasurable; or bone marrow plasma cell percentage (with ≥10% absolute increase) if serum and urine M-protein unmeasurable and involved free light chain unmeasurable; or ≥1 g/dL absolute increase in serum M-protein if the nadir was ≥5 g/dL; or ≥50% increase in circulating plasma cells (with minimum of 200 cells/μL) if this is the only measure of disease.

In some embodiments, the subjects treated in accord with provided methods have a suboptimal response to the first line of therapy with or without prior induction therapy received for treating the multiple myeloma. In some embodiments, wherein the first line of therapy is ASCT, a subject is considered to have a suboptimal response if the subject, at first post-ASCT assessment, has less than complete response. In particular embodiments, less than complete response includes very good partial response, partial response and stable disease. In particular embodiments, less than very good partial response includes very good partial response, partial response, stable disease and progressive disease. In certain embodiments, a subject is considered to have a suboptimal response if the subject, at first post-ASCT assessment, has very good partial response, partial response or stable disease. In certain embodiments, a subject is considered to have a suboptimal response if the subject, at first post-ASCT assessment, has very good partial response, partial response, stable disease or progressive disease. In some embodiments, the presence of a suboptimal response is determined at a time when a response to the frontline therapy would be expected to have an effect. In some embodiments, the presence of a suboptimal response is determined within six months after being given the frontline therapy, such as 1 month to 6 months. In accord with provided methods, at the time of screening or consent for treatment with CAR T cell therapy, the subject is to have been determined to have had a suboptimal response. For example, this can be at a time within 80-120 days prior to determining eligibility for treatment (i.e., at screening or consent) with the CAR T cells. As another example, this can be at a time about 100 days prior to determining eligibility for treatment (i.e., at screening or consent) with the CAR T cells. In some embodiments, a subject must have had a single ASCT within 6 months prior to consent. In some embodiments, a subject must have had a single ASCT 80 to 120 days prior to consent. In some embodiments, a subject must have achieved partial response or very good partial response at first post-ASCT assessment approximately 80 to 120 days after ASCT. In some embodiments, a subject must have achieved partial response or very good partial response at first post-ASCT assessment approximately 100 days after ASCT. In some embodiments, the documented partial response or very good partial response must be maintained at screening (i.e., time of consent). In some embodiments, a subject must have documented suboptimal response at time of consent. In some embodiments, a subject must have documented partial response or very good partial response at time of consent.

In some embodiments, the subjects treated in accord with provided methods have an inadequate response to the first line of therapy with or without prior induction therapy received for treating the multiple myeloma. In some embodiments, wherein the first line of therapy is ASCT, a subject is considered to have an inadequate response if the subject, at first post-ASCT assessment, has less than very good partial response. In particular embodiments, less than very good partial response includes partial response and stable disease. In particular embodiments, less than very good partial response includes partial response, stable disease and progressive disease. In embodiments, a subject is considered to have an inadequate response if the subject, at first post-ASCT assessment, has partial response or stable disease. In embodiments, a subject is considered to have an inadequate response if the subject, at first post-ASCT assessment, has partial response, stable disease or progressive disease. In some embodiments, the presence of an inadequate response is determined at a time when a response to the frontline therapy would be expected to have an effect. In some embodiments, the presence of an inadequate response is determined within six months after being given the frontline therapy, such as 1 month to 6 months. In accord with provided methods, at the time of screening or consent for treatment with CAR T cell therapy, the subject is to have been determined to have had an inadequate response. For example, this can be at a time within 80-120 days prior to determining eligibility for treatment (i.e., at screening or consent) with the CAR T cells. In another example, this can be at a time within 70-110 days prior to determining eligibility for treatment (i.e., at screening or consent) with the CAR T cells. In some embodiments, a subject must have had a single or tandem ASCT within 6 months prior to consent. In some embodiments, a subject must have had a single or tandem ASCT 80 to 120 days prior to consent. In some embodiments, a subject must have had a single or tandem ASCT 70 to 110 days prior to consent. In some embodiments, a subject must have achieved partial response or stable disease at first post-ASCT assessment approximately 80 to 120 days after ASCT. In some embodiments, a subject must have achieved partial response or stable disease at first post-ASCT assessment approximately 100 days after ASCT. In some embodiments, the documented partial response or stable disease must be maintained at screening (i.e., time of consent). In some embodiments, a subject must have documented inadequate response at time of consent. In some embodiments, a subject must have documented partial response or stable disease at time of consent. In some embodiments, subject must have had ASCT (single or tandem) and <VGPR (excluding PD) at first assessment between 70 to 110 days prior to consent. In some embodiments, subject must have had ASCT (single or tandem) and <VGPR (excluding PD) at first assessment between 70 to 110 days after last ASCT.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines, such as CD 107a, IFNγ, IL-2, and TNF. In some aspects, the biological activity is measured by assessing clinical outcome, such as reduction in tumor burden or load.

In some embodiments, the method includes assessment of one or more high risk (HR) tumor features following administration of the T cell therapy and/or following administration of the immunomodulatory agent maintenance therapy. In some embodiments, the method includes assessment of the cytotoxicity of the T cells toward cancer cells, e.g., T cells administered for the T cell based therapy. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g., T cell therapy, in the methods provided herein, can be measured by assessing the characteristics of the T cells in vitro or ex vivo. In some embodiments, such assays can be used to determine or confirm the function of the T cells, e.g., T cell therapy, before, during, or after administering the T cell therapy provided herein.

In some embodiments, the T cell therapy can further include one or more screening steps to identify subjects for treatment with the T cell therapy and/or continuing the T cell therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the T cell therapy provided herein.

In some embodiments, the immunomodulatory agent maintenance therapy can further include one or more screening steps to identify subjects for treatment with the immunomodulatory agent maintenance therapy and/or continuing the immunomodulatory agent maintenance therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the T cell therapy provided herein.

In some embodiments, any of the screening steps and/or assessment of treatment of outcomes described herein can be used prior to, during, during the course of, or subsequent to administration of one or more steps of the provided T cell therapy (e.g., anti-BCMA CAR T cells). In some embodiments, any of the screening steps and/or assessment of treatment of outcomes described herein can be used prior to, during, during the course of, or subsequent to administration of one or more steps of the provided immunomodulatory agent maintenance therapy. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of one or more steps of the provided T cell therapy, for example, to screen and identify patients suitable and/or susceptible to receive the T cell therapy. In some embodiments, the assessment is performed during, during the course of, or subsequent to administration of one or more steps of the provided T cell therapy, for example, to assess the intermediate or final treatment outcome, e.g., to determine the efficacy of the treatment and/or to determine whether to continue or repeat the treatments and/or to determine whether to administer the remaining steps of the T cell therapy. In some embodiments, the assessment is performed during, during the course of, or subsequent to administration of one or more steps of the provided T cell therapy, for example, to determine whether to administer the immunomodulatory agent maintenance therapy. In some embodiments, the assessment is performed prior to administration of the immunomodulatory agent maintenance therapy, for example, to screen and identify patients suitable and/or susceptible to receive the immunomodulatory agent maintenance therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g., CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the T cell therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the cytotoxicity of recombinant receptor-expressing, e.g., CAR-expressing, cells in the subject following administration of the dose of cells in the method with debulking, is greater as compared to that achieved via a method without debulking. In some embodiments, the cytotoxicity of recombinant receptor-expressing, e.g., CAR-expressing, cells in the subject following administration of the dose of cells in the method wherein a subject is selected for treatment if the subject achieves MRD negative status following administration of the T cell therapy and following achievement of MRD negative status, the cancer progresses (e.g., disease progression), is greater as compared to that achieved via a method without selecting the subject. In some embodiments, the cytotoxicity of recombinant receptor-expressing, e.g., CAR-expressing, cells in the subject following administration of the dose of cells in the method wherein, a subject is selected for treatment if, following administration of the T cell therapy, the cancer no longer exhibits at least one of the HR tumor features exhibits by the cancer prior to administration of the T cell therapy, is greater as compared to that achieved via a method without selecting the subject. In some embodiments, cytotoxicity in the subject of the administered T cell therapy, e.g., CAR-expressing T cells is assessed as compared to a method in which the T cell therapy is administered to a subject who receives a T cell therapy in the absence of receiving the immunomodulatory agent maintenance therapy. In some embodiments, the methods result in the administered T cells exhibiting increased or prolonged cytotoxicity in the subject as compared to a method in which the T cell therapy is administered to a subject receives a T cell therapy in the absence of receiving the immunomodulatory agent maintenance therapy.

In some embodiments, the subject can be screened prior to the administration of one or more steps of the methods. For example, the subject can be screened for HR tumor features prior to administration of the immunomodulatory agent maintenance therapy, to determine suitability, responsiveness and/or susceptibility to administering the immunomodulatory agent maintenance therapy. For example, the subject can be screened for MRD negative status following administration of the T cell therapy, to determine suitability, responsiveness and/or susceptibility to administering the immunomodulatory agent maintenance therapy. In some cases, the subject can be screened for characteristics of the disease prior to administration of the T cell therapy, to determine suitability, responsiveness and/or susceptibility to administering the T cell therapy. In some embodiments, the screening step and/or assessment of treatment outcomes can be used to determine the dose, frequency, duration, timing and/or order of the T cell therapy provided herein. The subject can be screened for characteristics of the disease prior to administration of the immunomodulatory agent maintenance therapy, to determine suitability, responsiveness and/or susceptibility to administering the immunomodulatory agent maintenance therapy. In some embodiments, the screening step and/or assessment of treatment outcomes can be used to determine the dose, frequency, duration, timing and/or order of the immunomodulatory agent maintenance therapy provided herein.

In some embodiments, a primary efficacy endpoint or primary outcome measure can be progression free survival (PFS), up to approximately 49 months after the first subject is randomized. PFS is as assessed by Independent Review Committee (IRC). In some embodiments, a secondary endpoint or secondary outcome measure can include overall survival (OS), event-free survival (EFS), duration of response (DOR), percentage of subjects with complete response (CR), complete response rate (CRR) as assessed by IRC, time to progression (TTP), progression post-next line of treatment (PFS2) and time to next treatment (TTNT), all assessed up to approximately 60 months after the last subject is randomized. In some embodiments, a secondary endpoint or secondary outcome measure can include percentage of subjects with minimal residual disease negative (MRDneg) complete response (CR), assessed up 15 months after the last subject is randomized or percentage of subjects with sustained MRDneg CR for 12 months, assessed up approximately 27 months after the last subject is randomized.

In some embodiments, safety secondary endpoints can include number of subjects experiencing adverse events (AEs) and number of subjects experiencing adverse events of special interest (AESI), both assessed up to approximately 60 months after the last subject is randomized. In some embodiments, pharmacokinetic endpoints include maximum observed plasma concentration (Cmax), time of maximum observed plasma concentration (Tmax), area under the curve (AUC) from time zero to 28 days post infusion and time of last measurable observed plasma concentration (Tlast), all assessed up to approximately 60 months after the last subject is randomized.

In some embodiments, secondary endpoints can include time-to-definitive deterioration and health-related quality of life (HRQOL) metrics, assessed up to approximately 49 months after subject randomization. In particular embodiments, HRQOL include mean change from baseline in European Organization for Research and Treatment of Cancer core quality of life questionnaire (EORTC QLQ-C30) and mean change from baseline in European Organization for Research and Treatment of Cancer core quality of life questionnaire for multiple myeloma (EORTC-QLQ-MY20).

In some embodiments, the subject can be screened after administration of one of the steps of the T cell therapy, to determine and identify subjects to receive the remaining steps of the T cell therapy and/or to monitor efficacy of the therapy. In some embodiments, the number, level or amount of administered T cells and/or proliferation and/or activity of the administered T cells is assessed after administration of the engineered T cells.

In some embodiments, the subject can be screened after administration of the T cell therapy, to determine and identify subjects to receive the immunomodulatory agent maintenance therapy and/or to monitor efficacy of the therapy. In some embodiments, the MRD status of the subject is assessed after administration of the T cell therapy. In some embodiments, the presence of HR tumor features is assessed after administration of the T cell therapy.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

Assessment and determination of parameters associated with T cell health, function, activity, and/or outcomes, such as response, efficacy and/or toxicity outcomes, can be assessed at various time points. In some aspects, the assessment can be performed multiple times, e.g., prior to, during, and/or after manufacturing of the cells, prior to, during, and/or after the initiation of administration of the T cell therapy.

In some embodiments, functional attributes of the administered cells and/or cell compositions include monitoring pharmacokinetic (PK) and pharmacodynamics parameters, expansion and persistence of the cells, cell functional assays (e.g., any described herein, such as cytotoxicity assay, cytokine secretion assay and in vivo assays), high-dimensional T cell signaling assessment, and assessment of exhaustion phenotypes and/or signatures of the T cells.

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g., CAR-expressing T cells) can reduce or prevent the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some embodiments, the provided methods result in a decreased tumor burden in treated subjects compared to alternative methods in which the T cell therapy is provided without the immunomodulatory agent maintenance therapy.

It is not necessary that the tumor burden actually be reduced in all subjects receiving the T cell therapy, but that tumor burden is reduced on average in subjects treated, such as based on clinical data, in which a majority of subjects treated with such a T cell therapy exhibit a reduced tumor burden, such as at least 50%, 60%, 70%, 80%, 90%, 95% or more of subjects treated with the T cell therapy, exhibit a reduced tumor burden.

It is not necessary that the tumor burden actually be reduced in all subjects receiving the immunomodulatory agent maintenance therapy, but that tumor burden is reduced on average in subjects treated, such as based on clinical data, in which a majority of subjects treated with the immunomodulatory agent maintenance therapy exhibit a reduced tumor burden, such as at least 50%, 60%, 70%, 80%, 90%, 95% or more of subjects treated with the immunomodulatory agent maintenance therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/l); renal insufficiency attributable to myeloma; anemia (hemoglobin <10 g/dl); and/or bone lesions (lytic lesions or osteoporosis with compression fractures)).

Exemplary methods for assessing disease status or disease burden include: measurement of M protein in biological fluids, such as blood and/or urine, by electrophoresis and immunofixation; quantification of sFLC (K and 2) in blood; skeletal survey; and imaging by positron emission tomography (PET)/computed tomography (CT) in subjects with extramedullary disease. In some embodiments, disease status can be evaluated by bone marrow examination. In some examples, efficacy of the T cell therapy following its administration to the subject is determined by the expansion and persistence of the T cells (e.g., BCMA CAR T cells) in the blood and/or bone marrow. In some embodiments, efficacy of the T cell therapy is determined based on the antitumor activity of the administered cells (e.g., BCMA CAR T cells). In some embodiments antitumor activity is determined by the overall response rate (ORR) and/or International Myeloma Working Group (IMWG) Uniform Response Criteria (see Kumar et al. (2016) Lancet Oncol 17 (8): e328-346). In some embodiments, response is evaluated using minimal residual disease (MRD) assessment. In some embodiments, MRD can be assessed by methods such as flow cytometry and high-throughput sequencing, e.g., deep sequencing. In some aspects, subjects that have a MRD-negative disease include those exhibiting absence of aberrant clonal plasma cells on bone marrow aspirate, ruled out by an assay with a minimum sensitivity of 1 in 10⁵ nucleated cells or higher (i.e., 10⁻⁵ sensitivity), such as flow cytometry (next-generation flow cytometry; NGF) or high-throughput sequencing, e.g., deep sequencing or next-generation sequencing (NGS).

In some aspects, sustained MRD-negative includes subjects that exhibit MRD negativity in the marrow (NGF or NGS, or both) and by imaging as defined below, confirmed minimum of 1 year apart. Subsequent evaluations can be used to further specify the duration of negativity (e.g., MRD-negative at 5 years). In some aspects, flow MRD-negative includes subjects that exhibit an absence of phenotypically aberrant clonal plasma cells by NGF on bone marrow aspirates using the EuroFlow standard operation procedure for MRD detection in multiple myeloma (or validated equivalent method) with a minimum sensitivity of 1 in 10⁵ nucleated cells or higher. In some aspects, sequencing MRD-negative includes subjects that exhibit an absence of clonal plasma cells by NGS on bone marrow aspirate in which presence of a clone is defined as less than two identical sequencing reads obtained after DNA sequencing of bone marrow aspirates using the LymphoSIGHT platform (or validated equivalent method) with a minimum sensitivity of 1 in 10⁵ nucleated cells or higher. In some aspects, imaging plus MRD-negative includes subjects that exhibit MRD negativity as assessed by NGF or NGS plus disappearance of every area of increased tracer uptake found at baseline or a preceding PET/CT or decrease to less mediastinal blood pool SUV or decrease to less than that of surrounding normal tissue (see Kumar et al. (2016) Lancet Oncol 17 (8): e328-346).

In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of tumor burden is specified. In some embodiments, exemplary parameters for determination include particular clinical outcomes indicative of amelioration or improvement in the tumor. Such parameters include: duration of disease control, including objective response (OR), complete response (CR), stringent complete response (sCR), very good partial response (VGPR), partial response (PR), minimal response (MR), Stable disease (SD), Progressive disease (PD) or relapse (see, e.g., International Myeloma Working Group (IMWG) Uniform Response Criteria; see Kumar et al. (2016) Lancet Oncol 17 (8): e328-346), objective response rate (ORR), progression-free survival (PFS) and overall survival (OS). In some embodiments, response is evaluated using minimal residual disease (MRD) assessment. In some embodiments, response is evaluated using complete response (CR) or stringent CR (sCR) assessment. In some embodiments, response is evaluated using complete response (CR) assessment. In some embodiments, response is evaluated using stringent CR (sCR) assessment. Specific thresholds for the parameters can be set to determine the efficacy of the methods provided herein. In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, response is evaluated based on the duration of response following administration of the T cell therapy, e.g., BCMA CAR T cells. In some aspects, the response to the therapy, e.g., according to the provided embodiments, can be measured at a designated time point after the initiation of administration of the T cell therapy. In some embodiments, the designated time point is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks months following initiation of the administration, or within a range defined by any of the foregoing. In some embodiments, the designated time point is at or about 1 month following initiation of the administration. In some embodiments, the designated time point is at or about 3 months following initiation of the administration. In some embodiments, the designated time point is at or about 6 months following initiation of the administration. In some embodiments, the designated time point is at or about 9 months following initiation of the administration. In some embodiments, the designated time point is at or about 12 months following initiation of the administration. In some embodiments, the response is a CR or a sCR. In some embodiments, the response is a CR. In some embodiments, the response is a sCR.

In some embodiments, response is evaluated based on the duration of response following administration of the immunomodulatory agent maintenance therapy. In some aspects, the response to the therapy, e.g., according to the provided embodiments, can be measured at a designated time point after the initiation of administration of the immunomodulatory agent maintenance therapy. In some embodiments, the designated time point is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks following initiation of the administration, or within a range defined by any of the foregoing. In some embodiments, the designated time point is at or about 1 month following initiation of the administration. In some embodiments, the designated time point is at or about 3 months following initiation of the administration. In some embodiments, the designated time point is at or about 6 months following initiation of the administration. In some embodiments, the designated time point is at or about 9 months following initiation of the administration. In some embodiments, the designated time point is at or about 12 months following initiation of the administration. In some embodiments, the response is a CR or a sCR. In some embodiments, the response is a CR. In some embodiments, the response is a sCR.

In some embodiments, the response or outcome determined at or about 3, 6, 9 or 12 months after the designated time point is equal to or improved compared to the response or outcome determined at the initial designated time point. For example, in some aspects, if the response or outcome determined at the initial designated time point is stable disease (SD), progressive disease (PD) or relapse, the subject treated according to the provided embodiments can show an equal or improved response or outcome (e.g., exhibiting a better response outcome according to the International Myeloma Working Group (IMWG) Uniform Response Criteria; see Kumar et al. (2016) Lancet Oncol 17 (8): e328-346) at a subsequent time point, after at or about 3, 6, 9 or 12 months after the initial designated time point, that is equal to the response or outcome at the initial designated time point, or a response or outcome that is objective response (OR), complete response (CR), stringent complete response (sCR), very good partial response (VGPR) or partial response (PR). In some embodiments, the response is a CR or a sCR. In some embodiments, the response is a CR. In some embodiments, the response is a sCR. In some aspects, subjects treated according to the provided embodiments can show a response or outcome that is improved between two time point of determination. In some aspects, the subject can exhibit a PR or VGPR in the initial designated time point for assessment, e.g., at 4 weeks after the initiation of administration, then exhibit an improved response, such as a CR or an sCR, at a later time point, e.g., at 12 weeks after the initiation of administration. In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response or outcome is durable for greater than at or about 3, 6, 9 or 12 months.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sørensen et al., (1993) Br J Cancer 67 (4) 773-775. In some embodiments, the subject that is to be administered according to the methods or treatment regimen provided herein include those with an ECOG performance status of 0 or 1.

In some embodiments, the methods and/or administration of a T cell therapy (e.g., BCMA CAR T cells) decrease(s) disease burden as compared with disease burden at a time immediately prior to the administration of the T cell therapy. In some embodiments, the methods and/or administration of a T cell therapy (e.g., BCMA CAR T cells) changes the HR tumor feature(s) exhibited by the cancer as compared with a time immediately prior to the administration of the T cell therapy. In some embodiments, the methods and/or administration of a T cell therapy (e.g., BCMA CAR T cells) changes the HR CRBN tumor feature(s) exhibited by the cancer as compared with a time immediately prior to the administration of the T cell therapy. In some embodiments, the methods and/or administration of a T cell therapy (e.g., BCMA CAR T cells) results in the subject achieving MRD negative status, as compared with a time immediately prior to the administration of the T cell therapy.

In some aspects, administration of the T cell therapy may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives a T cell therapy in the absence of the subject receiving the immunomodulatory agent maintenance therapy. In some embodiments, disease burden is reduced to a greater extent or for a greater duration following the administration of the immunomodulatory agent maintenance therapy, compared to the reduction that would be effected without provision of the immunomodulatory agent maintenance therapy.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). In some embodiments, the parameter is CR or sCR. In some embodiments, the parameter is CR. In some embodiments, the parameter is sCR. Specific thresholds for the parameters can be set to determine the efficacy of the method of T cell therapy provided herein.

In some embodiments, the subjects treated according to the method achieve a more durable response. In some cases, a measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months, greater than 6 months, or great than 12 months. In some particular embodiments, the subjects treated according to the method achieve a more durable response after the subject previously relapsed following remission in response to the administration of the genetically engineered cells.

In some aspects, disease burden is measured or detected prior to administration of the prior to administration of the T cell therapy, and/or prior to administration of the immunomodulatory agent maintenance therapy. In the context of multiple administration of one or more steps of the T cell therapy, disease burden in some embodiments may be measured prior to or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100% by the provided methods compared to immediately prior to the administration of the T cell therapy. In some embodiments, disease burden, tumor size, tumor volume, tumor mass, and/or tumor load or bulk is reduced following administration of the T cell therapy, by at least at or about 10, 20, 30, 40, 50, 60, 70, 80, 90% or more compared to that immediately prior to the administration of the T cell therapy. In some embodiments, disease burden, tumor size, tumor volume, tumor mass, and/or tumor load or bulk is reduced following administration of the immunomodulatory agent maintenance therapy, by at least at or about 10, 20, 30, 40, 50, 60, 70, 80, 90% or more compared to that immediately prior to the administration of the immunomodulatory agent maintenance therapy.

In some embodiments, reduction of disease burden by the method comprises an induction in morphologic complete remission, for example, as assessed at 1 month, 2 months, 3 months, or more than 3 months, after administration of, e.g., initiation of, the T cell therapy. In some embodiments, reduction of disease burden by the method comprises an induction in morphologic complete remission, for example, as assessed at 1 month, 2 months, 3 months, or more than 3 months, after administration of, e.g., initiation of, the immunomodulatory agent maintenance therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of immunomodulatory agent maintenance therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the provided method is less than about 80%, less than about 70%, less than about 60%, less than about 50%, less than about 40%, less than about 30%, less than about 20%, or less than about 10%.

In some embodiments, the administration can treat the subject despite the subject having become resistant to another therapy. In some embodiments, when administered to subjects according to the embodiments described herein, the dose or the composition is capable of achieving complete response (CR) or stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving complete response (CR), in at least 50%, at least 60%, at least 70%, at least 80%, at least 90%, or at least 95% of subjects that were administered. In some embodiments, when administered to subjects according to the embodiments described herein, the dose or the composition is capable of achieving stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving objective response (OR), in at least 50%, at least 60%, at least 70%, at least 80%, at least 90%, or at least 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, when administered to subjects according to the embodiments described herein, the dose or the composition is capable of achieving stringent complete response (sCR) at least 20%, 30%, 40% 50%, 60% or 70% of subjects that were administered. In some embodiments, when administered to subjects according to the embodiments described herein, the dose or the composition is capable of achieving complete response (CR) at least 20%, 30%, 40% 50%, 60% or 70% of subjects that were administered. In some embodiments, exemplary doses include about 1.0×10⁷, 1.5×10⁷, 2.0×10⁷, 2.5×10⁷, 5.0×10⁷, 1.5×10⁸, 3.0×10⁸, 4.5×10⁸, 4.6×10⁸, 5.4×10⁸, 6.0×10⁸ or 8.0×10⁸ CAR-expressing (CAR+) T cells. In some embodiments, exemplary doses include about 1.5×10⁸, 3.0×10⁸, 4.5×10⁸, 4.6×10⁸, or 5.4×10⁸ CAR-expressing (CAR+) T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17 (8): e328-346).

In some embodiments, toxicity and/or side-effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment.

In some embodiments, Grade 1 adverse events are mild. In some embodiments, Grade 2 adverse events are moderate. In some embodiments, Grade 3 adverse events are severe. In some embodiments, Grade 4 adverse events are life-threatening. In some embodiments, Grade 5 adverse events result in death.

Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities, such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

### II. BCMA-Targeting Therapy and Engineered Cells

Also provided herein are BCMA-targeting therapies. In some embodiments, the BCMA-targeting therapy is provided to a subject having a multiple myeloma. In some embodiments, the BCMA-targeting therapy is an antibody, an antibody-drug conjugate (ADC), or a T cell engager. In some embodiments, the BCMA-targeting therapy is an antibody. In some embodiments, the BCMA-targeting therapy is an antibody-drug conjugate (ADC). In some embodiments, the BCMA-targeting therapy is a T cell engager (TCE). In some embodiments, the BCMA-targeting therapy is a T cell engaging therapy capable of stimulating activity of T cells. In some embodiments, the BCM-targeting therapy is a bispecific T cell engager (BiTE) therapy. In some embodiments, the BCMA-targeting is a cell therapy selected from among the group consisting of a tumor infiltrating lymphocytic (TIL) therapy, an endogenous T cell therapy, a natural kill (NK) cell therapy, a transgenic TCR therapy, and a recombinant-receptor expressing cell therapy, which optionally is a chimeric antigen receptor (CAR)-expressing cell therapy. In some embodiments, the cell therapy is a recombinant receptor-expressing cell therapy. In some embodiments, the cell therapy is a chimeric antigen receptor (CAR)-expressing cell therapy. In any of the embodiments provided herein, the BCMA-targeting therapy is administered to a subject having a multiple myeloma.

Also provided are cells such as engineered cells that contain a recombinant receptor (e.g., a chimeric antigen receptor) such as one that contains an extracellular domain including an anti-BCMA binding moiety, such as an antibody or fragment as described herein. Also provided are populations of such cells, compositions containing such cells and/or enriched for such cells, such as in which cells expressing the BCMA-binding molecule make up at least 50, 60, 70, 80, 90, 91, 92, 93, 94, 95, 96, 97, 98, 99, or more percent of the total cells in the composition or cells of a certain type, such as PBMCs, T cells or CD3+, CD8+ or CD4+ cells.

Among the compositions are pharmaceutical compositions and formulations for administration, such as for adoptive cell therapy. Also provided are therapeutic methods for administering the cells and compositions to subjects, e.g., patients.

Thus, also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

In some embodiments, the T cell therapy for use in accord with the provided methods includes administering engineered T cells expressing recombinant receptors designed to recognize and/or specifically bind to molecules associated with multiple myeloma, for example relapsed and refractory (R/R) multiple myeloma (MM) (e.g., BCMA). In some embodiments, binding to the antigen results in a response, such as an immune response against such molecules upon binding to such molecules. In some embodiments, the cells contain or are engineered to contain an engineered receptor, e.g., an engineered antigen receptor, such as a chimeric antigen receptor (CAR), or a T cell receptor (TCR). The recombinant receptor, such as a CAR, generally includes an extracellular antigen (or ligand) binding domain that is directed against BCMA, linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors, e.g. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In some embodiments of the provided methods and uses, the engineered cells, such as T cells, express a chimeric receptor, such as a chimeric antigen receptor (CAR), that contains one or more domains that combine a ligand-binding domain (e.g., antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (VH) regions capable of specifically binding the antigen, single chain antibody fragments, including single chain variable fragments (scFv), and single domain antibodies (e.g., sdAb, sdFv, nanobody, VHH) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., J Mol Biol, 1997; 273 (4): 927-48 (“Chothia” numbering scheme); MacCallum et al., J. Mol. Biol, 1996; 262:732-745.” (“Contact” numbering scheme); Lefranc M P et al., Dev Comp Immunol, 2003; 27 (1): 55-77 (“IMGT” numbering scheme); Honegger A and Plückthun A, J Mol Biol, 2001; 309 (3): 657-70, (“Aho” numbering scheme); Martin et al., PNAS, 1989; 86 (23): 9268-9272, (“AbM” numbering scheme); and Ye et al., Nucleic Acids Res. 2013; 41 (Web Server issue): W34-40, (“IgBLAST numbering scheme). Details regarding various numbering schemes are also described in, for example, Jarasch et al., Proteins, 2017; 85 (1): 65-71; Martin et al., Bioinformatics tools for antibody engineering. In: Dübel, S. (editor) Handbook of Therapeutic Antibodies, Vol. 1. Wiley-VCH, Weinheim, Germany; Martin, A.C.R. (2010). Protein Sequence and Structure Analysis of Antibody Variable Domains. In: Kontermann, R., Dübel, S. (eds) Antibody Engineering. Springer Protocols Handbooks. Springer, Berlin, Heidelberg; and Martin, A C R, Antibody Information: How to identify the CDRs by looking at a sequence [online] bioinf.org.uk/abs/info.html, all of which are incorporated by reference in their entireties. Various prediction algorithm tools are available and known for numbering antibody residues and CDRs (e.g., AbYsis, Abnum, AbYmod, AbRSA, IgBLAST, IMGT, or ANARCI).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, in some cases with insertions. Insertions in the sequence relative to the standard numbering scheme are indicated using insertion letter codes. For example, residues that are inserted between residues L30 and L31 are indicated as L31A, L31B, etc. Deletions in the sequence relative to the standard scheme are accommodated by skipping numbers. The two schemes place certain insertions and deletions (“indels”) at different positions, resulting in differential numbering. For instance, the Chothia numbering scheme is nearly identical to the Kabat numbering scheme, except that insertions are placed at structural positions and topologically equivalents residues do get assigned the same numbers. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

In some embodiments, Kabat numbering can be determined by known sequence rules as described in, for example, Kabat et al. (1991), “Sequences of Proteins of Immunological Interest,” 5th Ed. Public Health Service, National Institutes of Health, Bethesda, MD. In some embodiments, the Kabat numbering scheme in some aspects can include any of the following rules to designate CDRs: CDR-L1 starts at approximately residue 24 of the light chain, always has a preceding C residue, and always has a following W residue; the end of CDR-L1 is defined by a stretch of 3 residues, where the W residue can be followed by Y, L, or F, followed by Q or L; CDR-1 has a length of 10 to 17 residues; CDR-L2 always starts 16 residues after the end of CDR-L1; the two residues before CDR-L2 are I and Y but can also be V and Y, I and K, or I and F; CDR-L2 is always 7 residues long; CDR-L3 always starts 33 residues after the end of CDR-L2, always has a preceding C residue, and is strictly followed by a F-G-X-G sequence motif, where X is any amino acid; CDR-L3 has a length of 7 to 11 residues; CDR-H1 starts at approximately position 26 of the heavy chain; the first amino acid in CDR-H1 is always 9 residues after a conserved C residue; CDR-H1 is followed by an invariant W residue followed by typically V, but also can be I or A; CDR-H1 has a length of 5 to 7 residues; CDR-H2 always starts at 15 residues after the end of CDR-H1; the first residue in CDR-H2 is usually preceded by the sequence motif L-E-W-I-G but a number of variations exist; the end of CDR-H2 is defined by a motif of 3 residues—the first residue of the motif of 3 residues can be either K or R, the second residue of the motif of 3 residues can be L, I, V, F, T, or A, the third residue of the motif of 3 residues can be T, S, I, or A; CDR-H2 has a length of 16 to 19 residues; CDR-H3 always starts 33 residues after the end of CDR-H2 and is always 3 residues after a C residue. The first residue of CDR-H3 is preceded by the conserved C residue followed by two residues, which are usually A-R; the residues following CDR-H3 is strictly followed by a W-G-X-G sequence motif, where the X is any amino acid; CDR-H3 typically has a length of 3 to 25 residues; CDR-H3 can be much longer than 25 residues.

In some cases, according to the Chothia numbering scheme, exact boundary positions of certain CDRs can differ based on different definitions for the CDRs (See e.g., Martin, A C R, Antibody Information: How to identify the CDRs by looking at a sequence [online] bioinf.org.uk/abs/info.html). For example, in some instances, the boundary positions for CDR-L1 according to Chothia numbering can be L26-L32 (Chothia et al., Science, 1986; 233 (4765): 755-8 and Chothia C. and Lesk A. M. J Mol Biol, 1987; 196 (4): 901-17). In some instances, the boundary positions for CDR-L1 can be L25-L32 (Al-Lazikani et al., J Mol Biol, 1997; 273 (4): 927-48). In some instances, the boundary positions for CDR-L2 can be L50-L52 and for CDR-L3 can be L91-L96 (Chothia et al., Science, 1986; 233 (4765): 755-8; Chothia C. and Lesk A. M. J Mol Biol, 1987; 196 (4): 901-17; and Al-Lazikani et al., J Mol Biol, 1997; 273 (4): 927-48). In some instances, the boundary positions for CDR-H1 according to Chothia numbering can be H26-H32 (Chothia et al., Science, 1986; 233 (4765): 755-8; Chothia C. and Lesk A. M. J Mol Biol, 1987; 196 (4): 901-17; and Al-Lazikani et al., J Mol Biol, 1997; 273 (4): 927-48). In some instances, the boundary positions for CDR-H2 can be H53-H55 (Chothia et al., Science, 1986; 233 (4765): 755-8 and Chothia C. and Lesk A. M. J Mol Biol, 1987, 196 (4): 901-17); H52a-H55 (Tramontano et al., J Mol Biol, 1990, 215 (1): 175-82), or H52-H56 (Al-Lazikani et al., J Mol Biol., 1997; 273 (4): 927-48). In some instances, the boundary positions for CDR-H3 can be H96-H101 (Chothia et al., Science, 1986; 233 (4765): 755-8 and Chothia C. and Lesk A. M. J Mol Biol., 1987; 196 (4): 901-17). In some instances, the boundary positions for CDR-H3 can be H92-H104 (Morea et al., Biophys Chem, 1997; 68 (1-3): 9-16 and Morea et al., J Mol Biol., 1998; 275 (2): 269-94).

Table 2, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., J Mol Biol., 1997; 273(4): 927-48).

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given VH or VL region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given VH region amino acid sequence and a CDR-L1, a CDR-L2, and a CDR-L3 as contained within a given VL region amino acid sequence, the CDRs can be defined by any of the aforementioned schemes, such as Kabat, Chothia, AbM, IgBLAST, IMGT, or Contact method, or other known scheme. In some embodiments, specific CDR sequences are specified. Exemplary CDR sequences of provided antibodies are described using various numbering schemes, although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given VH region amino acid sequence and a FR-L1, a FR-L2, a FR-L3, and a FR-L4 as contained within a given VL region amino acid sequence, the FRs can be defined by any of the aforementioned schemes, such as Kabat, Chothia, AbM, IgBLAST, IMGT, or Contact method, or other known scheme.

The term “variable region” or “variable domain” refers to the domain of an antibody heavy or light chain that is involved in binding the antibody to antigen. The variable regions of the heavy chain and light chain (VH and VL, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; heavy chain variable (VH) regions, single-chain antibody molecules such as scFvs and single-domain antibodies comprising only the VH region; and multispecific antibodies formed from antibody fragments. In some embodiments, the antibody is or comprises an antibody fragment comprising a variable heavy chain (VH) and a variable light chain (VL) region. In particular embodiments, the antibodies are single-chain antibody fragments comprising a heavy chain variable (VH) region and/or a light chain variable (VL) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

In some embodiments, the CAR includes an antigen-binding portion or portions of an antibody molecule, such as a single-chain antibody fragment (scFv) derived from the variable heavy (VH) and variable light (VL) chains of a monoclonal antibody (mAb), or a single domain antibody (sdAb), such as sdFv, nanobody, VHH and VNAR. In some embodiments, an antigen-binding fragment comprises antibody variable regions joined by a flexible linker.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the anti-BCMA antibodies included in the provided CARs are murine antibodies. A “murine antibody” is an antibody with an amino acid sequence corresponding to that of an antibody produced by a murine or a murine cell, or non-murine source that utilizes murine antibody repertoires or other murine antibody-encoding sequences, including murine antibody libraries.

Also among the anti-BCMA antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

Thus, in some embodiments, the chimeric antigen receptor, including TCR-like CARs, includes an extracellular portion containing an antibody or antibody fragment. In some embodiments, the antibody or fragment includes an scFv. In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region.

In some embodiments, the antibody is an antigen-binding fragment, such as a scFv, that includes one or more linkers joining two antibody domains or regions, such as a heavy chain variable (VH) region and a light chain variable (VL) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:26) or GGGS (3GS; SEQ ID NO:27), such as between 2, 3, 4, and 5 repeats of such a sequence. Other exemplary linkers include those having or consisting of a sequence set forth in SEQ ID NOs: 16 or 17. Exemplary linkers include those having or consisting of a sequence set forth in SEQ ID NO:28 (GGGGSGGGGSGGGGS), SEQ ID NO:29 (GSTSGSGKPGSGEGSTKG), SEQ ID NO: 30 (SRGGGGSGGGGSGGGGSLEMA), or SEQ ID NO: 38 (ASGGGGSGGRASGGGGS). In some embodiments, the linker is or comprises the sequence set forth in SEQ ID NO:29.

In some embodiments, the CAR includes a BCMA-binding portion or portions of the antibody molecule, such as a heavy chain variable (VH) region and/or light chain variable (VL) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin. Cancer Res., 2013, 19 (8): 2048-2060; Feng et al., Scand. J. Immunol. (2020) 92: e12910; U.S. Pat. Nos. 9,034,324, 9,765,342; U.S. Patent publication Nos. US2016/0046724, US2017/0183418; and International published PCT App. Nos. WO 2016/090320, WO 2016/090327, WO 2016/094304, WO 2016/014565, WO 2016/014789, WO 2010/104949, WO 2017/025038, WO 2017/173256, WO 2018/085690, or WO 2021/091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO 2017/025038 or WO 2018/028647. In some embodiments, the anti-BCMA CAR contains two single-domain anti-BCMA antibodies. In some embodiments, the two single-domain anti-BCMA antibodies are derived from one or more antibodies described in WO 2017/025038 or WO 2018/028647. In some embodiments, the BCMA binding domain comprises or consists of A37353-G4S-A37917 (G4S being a linker between the two binding domains), described in WO 2017/025038 or WO 2018/028647, and provided, e.g., in SEQ ID NOs: 300, 301 and 302 of WO 2017/025038 or WO 2018/028647. In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO 2016/090320 or WO 2016/090327. In some embodiments, the scFv containing a variable heavy (V_{H}) and/or a variable light (V_{L}) region is derived from an antibody described in WO 2019/090003. In some embodiments, the scFv containing a variable heavy (V_{H}) and/or a variable light (V_{L}) region is derived from an antibody described in WO 2016/094304 or WO 2021/091978. In some embodiments, the scFv containing a variable heavy (V_{H}) and/or a variable light (V_{L}) region is derived from an antibody described in WO 2018/133877. In some embodiments, the scFv containing a variable heavy (V_{H}) and/or a variable light (V_{L}) region is derived from an antibody described in WO 2019/149269. In some embodiments, the anti-BCMA CAR is any as described in WO 2019/173636 or WO 2020/051374. In some embodiments, the anti-BCMA CAR is any as described in WO 2018/102752. In some embodiments, the anti-BCMA CAR is any as described in WO 2020/112796 or WO 2021/173630.

In some embodiments, the antibody, e.g., the anti-BCMA antibody or antigen-binding fragment, contains a heavy and/or light chain variable (VH or VL) region sequence as described, or a sufficient antigen-binding portion thereof. In some embodiments, the anti-BCMA antibody, e.g., antigen-binding fragment, contains a VH region sequence or sufficient antigen-binding portion thereof that contains a CDR-H1, CDR-H2 and/or CDR-H3 as described. In some embodiments, the anti-BCMA antibody, e.g., antigen-binding fragment, contains a VL region sequence or sufficient antigen-binding portion that contains a CDR-L1, CDR-L2 and/or CDR-L3 as described. In some embodiments, the anti-BCMA antibody, e.g., antigen-binding fragment, contains a VH region sequence that contains a CDR-H1, CDR-H2 and/or CDR-H3 as described and contains a VL region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a V_{H} region sequence or a sufficient antigen-binding portion thereof, such as any of the above described V_{H} sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g., human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19 (8): 2048-2060, U.S. Pat. No. 9,765,342, WO 2016/090320, WO 2016/090327, WO 2010/104949, WO 2016/014789, WO 2016/094304, WO 2017/025038, and WO 2017/173256.

In some embodiments, the anti-BCMA CAR contains an antigen-binding domain, such as an scFv, containing a variable heavy (V_{H}) and/or a variable light (V_{L}) region derived from an antibody described in WO 2016/094304 or WO 2021/091978. In some embodiments, the antigen-binding domain is an antibody fragment containing a variable heavy chain (V_{H}) and a variable light chain (V_{L}) region. In some embodiments, the anti-BCMA CAR contains an antigen-binding domain, such as an scFv, containing a variable heavy (V_{H}) and/or a variable light (V_{L}) region derived from an antibody described in WO 2016/090320 or WO 2016/090327.

In some embodiments, the antigen-binding domain is an antibody fragment containing a variable heavy chain (V_{H}) and a variable light chain (V_{L}) region. In some aspects, the V_{H} region is or includes an amino acid sequence having at least 90%, 91%, 92%, 93%, 94%, 95%, 96%, 97%, 98% or 99% sequence identity to the V_{H} region amino acid sequence set forth in any of SEQ ID NOs: 18, 20, 22, 24, 32, 34, 36, 41, 43, 45, 47, 49, 51, 53, 55, 57, 59, 61, 63, 65, 67, 69, 71, 73, 75, 145, 147, 149 and 151; and/or the V_{L} region is or includes an amino acid sequence having at least 90%, 91%, 92%, 93%, 94%, 95%, 96%, 97%, 98%, or 99% sequence identity to the V_{L} region amino acid sequence set forth in any of SEQ ID NOs: 19, 21, 23, 25, 33, 35, 37, 42, 44, 46, 48, 50, 52, 54, 56, 58, 60, 62, 64, 66, 68, 70, 72, 74, 76, 146, 148, 150 and 152.

In some embodiments, the antigen-binding domain, such as an scFv, contains a V_{H} set forth in SEQ ID NO: 18 and a V_{L} set forth in SEQ ID NO:19. In some embodiments, the antigen-binding domain, such as an scFv, contains a V_{H} set forth in SEQ ID NO: 20 and a V_{L} set forth in SEQ ID NO:21. In some embodiments, the antigen-binding domain, such as an scFv, contains a V_{H} set forth in SEQ ID NO: 22 and a V_{L} set forth in SEQ ID NO:23. In some embodiments, the antigen-binding domain, such as an scFv, contains a V_{H} set forth in SEQ ID NO: 24 and a V_{L} set forth in SEQ ID NO:25. In some embodiment the antigen-binding domain, such as an scFv, contains a V_{H} set forth in SEQ ID NO: 32 and a V_{L} set forth in SEQ ID NO:33. In some embodiments, the antigen-binding domain, such as an scFv, contains a V_{H} set forth in SEQ ID NO:34 and a V_{L} set forth in SEQ ID NO:35. In some embodiments, the antigen-binding domain, such as an scFv, contains a V_{H} set forth in SEQ ID NO: 36 and a V_{L} set forth in SEQ ID NO:37. In some embodiments, the antigen-binding domain, such as an scFv, contains a V_{H} set forth in SEQ ID NO: 41 and a V_{L} set forth in SEQ ID NO: 42. In some embodiments, the antigen-binding domain, such as an scFv, contains a V_{H} set forth in SEQ ID NO: 43 and a V_{L} set forth in SEQ ID NO: 44. In some embodiments, the antigen-binding domain, such as an scFv, contains a V_{H} set forth in SEQ ID NO: 45 and a V_{L} set forth in SEQ ID NO: 46. In some embodiments, the antigen-binding domain, such as an scFv, contains a V_{H} set forth in SEQ ID NO: 47 and a V_{L} set forth in SEQ ID NO: 48. In some embodiments, the antigen-binding domain, such as an scFv, contains a V_{H} set forth in SEQ ID NO: 49 and a V_{L} set forth in SEQ ID NO: 50. In some embodiments, the antigen-binding domain, such as an scFv, contains a V_{H} set forth in SEQ ID NO: 51 and a V_{L} set forth in SEQ ID NO: 52. In some embodiments, the antigen-binding domain, such as an scFv, contains a V_{H} set forth in SEQ ID NO: 53 and a V_{L} set forth in SEQ ID NO: 54. In some embodiments, the antigen-binding domain, such as an scFv, contains a V_{H} set forth in SEQ ID NO: 55 and a V_{L} set forth in SEQ ID NO: 56. In some embodiments, the antigen-binding domain, such as an scFv, contains a V_{H} set forth in SEQ ID NO: 57 and a V_{L} set forth in SEQ ID NO: 58. In some embodiments, the antigen-binding domain, such as an scFv, contains a V_{H} set forth in SEQ ID NO: 59 and a V_{L} set forth in SEQ ID NO: 60. In some embodiments, the antigen-binding domain, such as an scFv, contains a V_{H} set forth in SEQ ID NO: 61 and a V_{L} set forth in SEQ ID NO: 62. In some embodiments, the antigen-binding domain, such as an scFv, contains a V_{H} set forth in SEQ ID NO: 63 and a V_{L} set forth in SEQ ID NO: 64. In some embodiments, the antigen-binding domain, such as an scFv, contains a V_{H} set forth in SEQ ID NO: 65 and a V_{L} set forth in SEQ ID NO: 66. In some embodiments, the antigen-binding domain, such as an scFv, contains a V_{H} set forth in SEQ ID NO: 67 and a V_{L} set forth in SEQ ID NO: 68. In some embodiments, the antigen-binding domain, such as an scFv, contains a V_{H} set forth in SEQ ID NO: 69 and a V_{L} set forth in SEQ ID NO: 70. In some embodiments, the antigen-binding domain, such as an scFv, contains a V_{H} set forth in SEQ ID NO: 71 and a V_{L} set forth in SEQ ID NO: 72. In some embodiments, the antigen-binding domain, such as an scFv, contains a V_{H} set forth in SEQ ID NO: 73 and a V_{L} set forth in SEQ ID NO: 74. In some embodiments, the antigen-binding domain, such as an scFv, contains a V_{H} set forth in SEQ ID NO: 75 and a V_{L} set forth in SEQ ID NO: 76. In some embodiments, the antigen-binding domain, such as an scFv, contains a V_{H} set forth in SEQ ID NO: 145 and a V_{L} set forth in SEQ ID NO: 146. In some embodiments, the antigen-binding domain, such as an scFv, contains a V_{H} set forth in SEQ ID NO: 147 and a V_{L} set forth in SEQ ID NO: 148. In some embodiments, the antigen-binding domain, such as an scFv, contains a V_{H} set forth in SEQ ID NO: 149 and a V_{L} set forth in SEQ ID NO: 150. In some embodiments, the antigen-binding domain, such as an scFv, contains a V_{H} set forth in SEQ ID NO: 151 and a V_{L} set forth in SEQ ID NO: 152. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region. In some embodiments, the variable heavy and variable light chains are connected by a linker. In some embodiments, the linker is set forth in SEQ ID NO: 28, 29, 30, or 38.

Among a provided anti-BCMA CAR is a CAR in which the antibody or antigen-binding fragment contains a V_{H} region comprising the sequence set forth in SEQ ID NO: 18 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:18; and contains a V_{L} region comprising the sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:19. In some embodiments, the antibody or antigen-binding fragment of the provided CAR contains a V_{H} region that has a CDRH1, a CDRH2 and a CDRH3 comprising the amino acid sequence of SEQ ID NOS: 189, 190, and 191, respectively and a V_{L} region that has a CDRL1, a CDRL2 and a CDRL3 comprising the amino acid sequence of SEQ ID NOS: 192, 193, and 194, respectively. In some embodiments, the antibody or antigen-binding fragment of the provided CAR contains a V_{H} region that has a CDRH1, a CDRH2 and a CDRH3 comprising the amino acid sequence of SEQ ID NOS: 195, 196, and 197, respectively and a V_{L} region that has a CDRL1, a CDRL2 and a CDRL3 comprising the amino acid sequence of SEQ ID NOS: 198, 199, and 200, respectively. In some embodiments, the antibody or antigen-binding fragment of the provided CAR contains a V_{H} region that has a CDRH1, a CDRH2 and a CDRH3 comprising the amino acid sequence of SEQ ID NOS: 201, 202, and 203, respectively and a V_{L} region that has a CDRL1, a CDRL2 and a CDRL3 comprising the amino acid sequence of SEQ ID NOS: 204, 205, and 206, respectively. In some embodiments, the antibody or antigen-binding fragment of the provided CAR contains a V_{H} region that has a CDRH1, a CDRH2 and a CDRH3 comprising the amino acid sequence of SEQ ID NOS: 207, 208, and 209, respectively and a V_{L} region that has a CDRL1, a CDRL2 and a CDRL3 comprising the amino acid sequence of SEQ ID NOS: 210, 211, and 212, respectively. In some embodiments, the V_{H} region comprises the sequence set forth in SEQ ID NO: 18 and the V_{L} region comprises the sequence set forth in SEQ ID NO:19. In some embodiments, the antibody or antigen-binding fragment is a single-chain antibody fragment, such as an scFv. In some embodiments, the scFv comprises the sequence of amino acids set forth in SEQ ID NO: 213 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:213. In some embodiments, the anti-BCMA CAR has the sequence of amino acids set forth in SEQ NO: 116 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:116. In some embodiments, the anti-BCMA CAR is encoded by the polynucleotide sequence set forth in SEQ NO: 214 or a polynucleotide sequence of at least at or about 90%, at or about 91%, at or about 92%, at or about 93%, at or about 94%, at or about 95%, at or about 96%, at or about 97%, at or about 98%, or at or about 99% identity to SEQ ID NO:214.

Among a provided anti-BCMA CAR is a CAR in which the antibody or antigen-binding fragment contains a V_{H} region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24; and contains a V_{L} region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25. In some embodiments, the antibody or antigen-binding fragment of the provided CAR contains a V_{H} region that has a CDRH1, a CDRH2 and a CDRH3 comprising the amino acid sequence of SEQ ID NOS: 173, 174 and 175, respectively and a V_{L} region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 176, 177 and 175, respectively and a V_{L} region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 178, 179 and 175, respectively and a V_{L} region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 180, 181 and 182, respectively and a V_{L} region that has a CDRL1, a CDRL2 and a CDRL3 comprising the amino acid sequence of SEQ ID NOS: 186, 187 and 185, respectively. In some embodiments, the V_{H} region comprises the sequence set forth in SEQ ID NO:24 and the V_{L} region comprises the sequence set forth in SEQ ID NO:25. In some embodiments, the antibody or antigen-binding fragment is a single-chain antibody fragment, such as an scFv. In some embodiments, the scFv comprises the sequence of amino acids set forth in SEQ ID NO: 188 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:188. In some embodiments, the anti-BCMA CAR has the sequence of amino acids set forth in SEQ NO: 124 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:124. In some embodiments, the anti-BCMA CAR has the sequence of amino acids set forth in SEQ NO: 125 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:125.

In some embodiments, the scFv comprises the amino acid sequence set forth in any one of SEQ ID NOS: 216-247, or an amino acid sequence having at least 90, 95, 96, 97, 98, 99, or 100% sequence identity to a sequence set forth in any one of SEQ ID NOS: 216-247.

In some embodiments, the antigen-binding domain comprises an sdAb. In some embodiments, the antigen-binding domain contains the sequence set forth by SEQ ID NO:77. In some embodiments, the antigen-binding domain comprises a sequence at least or about 50, 60, 70, 80, 85, 90, 95, 96, 97, 98, 99, or 100% identical to the sequence set forth by SEQ ID NO:77.

In some embodiments, the CAR comprises the amino acid sequence set forth in any one of SEQ ID NOS: 90-141, or an amino acid sequence having at least 90, 95, 96, 97, 98, 99, or 100% sequence identity to a sequence set forth in any one of SEQ ID NOS: 90-141.

In some embodiments, among such antibodies or antigen-binding domains in the provided CARs are antibodies capable of binding BCMA protein, such as human BCMA protein, with at least a certain affinity, as measured by any of a number of known methods. In some embodiments, the affinity is represented by an equilibrium dissociation constant (K_{D}); in some embodiments, the affinity is represented by EC50.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for an antigen, e.g., BCMA. For example, in some embodiments, a BIAcore® instrument can be used to determine the binding kinetics and constants of a complex between two proteins (e.g., an antibody or fragment thereof, and an antigen, such as a BCMA cell surface protein, soluble BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to an antigen, e.g., a cell surface BCMA protein or soluble BCMA protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 105 M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding affinity (EC50) and/or the dissociation constant of the antibody (e.g. antigen-binding fragment) or antigen-binding domain of a CAR to a BCMA protein, such as human BCMA protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC50) and/or the equilibrium dissociation constant, K_{D}, of the antibody to a BCMA protein, such as human BCMA protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a BCMA protein, such as human BCMA protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g., antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g., antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC50) and/or the equilibrium dissociation constant, K_{D}, of the binding molecule, e.g., anti-BCMA antibody or fragment thereof or antigen-binding domain of a CAR, to a BCMA protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC50) and/or the dissociation constant of the equilibrium dissociation constant, K_{D}, of the binding molecule, e.g., anti-BCMA antibody or fragment thereof or antigen-binding domain of a CAR, to a BCMA protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody (e.g., anti-BCMA reference antibody).

In some embodiments, the binding affinity of the anti-BCMA antibody or antigen-binding domain of a CAR, for different form or topological type of antigens, e.g., soluble or shed BCMA protein compared to the binding affinity to a membrane-bound BCMA, to determine the preferential binding or relative affinity for a particular form or topological type. For example, in some aspects, an anti-BCMA antibodies or antigen-binding domain of a CAR can exhibit preferential binding to membrane-bound BCMA as compared to soluble or shed BCMA and/or exhibit greater binding affinity for, membrane-bound BCMA compared to soluble or shed BCMA. In some embodiments, the equilibrium dissociation constant, K_{D}, for different form or topological type of BCMA proteins, can be compared to determine preferential binding or relative binding affinity. In some embodiments, the preferential binding or relative affinity to a membrane-bound BCMA compared to soluble or shed BCMA can be high. For example, in some cases, the ratio of K_{D} for soluble or shed BCMA and the K_{D} for membrane-bound BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the ratio of K_{A} for membrane-bound BCMA and the K_{A} for soluble or shed BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the antibody or antigen-binding domain of CAR binds soluble or shed BCMA and membrane-bound BCMA to a similar degree, e.g., the ratio of K_{D} for soluble BCMA and KD for membrane-bound BCMA is or is about 1. In some cases, the antibody or antigen-binding domain of CAR binds soluble or shed BCMA and membrane-bound BCMA to a similar degree, e.g., the ratio of K_{A} for soluble BCMA and K_{A} for membrane-bound BCMA is or is about 1. The degree of preferential binding or relative affinity for membrane-bound BCMA or soluble or shed BCMA can be compared with that of a known antibody, such as a reference antibody (e.g., reference anti-BCMA CAR). In some embodiments, the reference antibody (e.g., reference anti-BCMA CAR) binds to membrane-bound and soluble or shed BCMA protein.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. In some embodiments, an exemplary BCMA-Fc fusion polypeptide is set forth in SEQ ID NO:172.

The spacer can be of a length that provides for increased responsiveness of the cell following antigen binding, as compared to in the absence of the spacer. Exemplary spacers, e.g., hinge regions, include those described in International Patent Application No. PCT/US2013/055862 (International Publication No. WO 2014/031687). In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al., Clin. Cancer Res., 19:3153 (2013), Hudecek et al. (2015) Cancer Immunol Res. 3 (2): 125-135, International Patent Application No. PCT/US2013/055862 (International Publication No. WO 2014/031687), U.S. Pat. No. 8,822,647 or Published App. No. US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine(S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence or an N176Q at position 176, in the C_{H}2 region, of the full-length IgG4 Fc sequence.

In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 31. In some embodiments, the constant region or portion is of IgD. In some embodiments, the spacer has the sequence set forth in SEQ ID NO: 5. In some embodiments, the spacer has the sequence set forth in SEQ ID NO: 89.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 144), is present and forms a linkage between the scFv and the spacer region of the CAR. In some embodiments, the spacer has the sequence set forth in SEQ ID NO: 78. In some embodiments, the spacer has the sequence set forth in SEQ ID NO: 80. In some embodiments, the spacer has the sequence set forth in any of SEQ ID NOs: 81-83, In some embodiments, the spacer has the sequence set forth in SEQ ID NO: 84. In some embodiments, the spacer has the sequence set forth in SEQ ID NO: 86. In some embodiments, the spacer has the sequence set forth in SEQ ID NO: 88.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5, 31, 78, 80, 81, 82, 83, 84, 86, 88, or 89.

In some embodiments, the spacer has the sequence set forth in SEQ ID NOS: 157-165. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 157-165.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the chimeric antigen receptor includes a transmembrane domain linking the extracellular domain and the intracellular signaling domain. In some embodiments, the transmembrane domain is fused to the extracellular domain, such as linked or fused between the extracellular domain (e.g., scFv) and intracellular signaling domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD134, CD137 (4-1BB), CD154, CTLA-4 or PD-1. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28. Exemplary sequences of transmembrane domains are or comprise the sequences set forth in SEQ ID NOs: 8, 79, 85, 87, 142, or 143. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR CD3 chain that mediates T-cell stimulation and/or activation and cytotoxicity, e.g., CD3 zeta chain, CD3 gamma, CD3 delta, CD3 epsilon, FcR gamma, FcR beta, CDS, CD22, CD79a, CD79b and CD66d. In some examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO 2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5 (215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptors to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen that is expressed on the normal cells but not cells of the disease or condition.

In some aspects, the chimeric receptor is or includes an inhibitory CAR (e.g., iCAR) and includes intracellular components that dampen or suppress an immune response, such as an ITAM- and/or co-stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, and EP2/4 adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof, and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the transmembrane domain of the recombinant receptor, e.g., the CAR, is or includes a transmembrane domain of human CD28 (e.g., Accession No. P10747.1), or CD8a (Accession No. P01732.1), or variant thereof, such as a transmembrane domain that comprises the sequence of amino acids set forth in SEQ ID NO: 8, 79, 142, or 143 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 79, 142, or 143. In some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the transmembrane domain is a transmembrane domain from CD8a. In some embodiments, the transmembrane domain is any as described in Milone et al., Mol. Ther. (2009) 12 (9): 1453-64. In some embodiments, the transmembrane domain is or comprises the sequence set forth in SEQ ID NO:143.

In some embodiments, the intracellular signaling component(s) of the recombinant receptor, e.g., the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g., Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular domain comprises an intracellular costimulatory signaling domain of 4-1BB. In some embodiments, the 4-1BB co-stimulatory molecule is any as described in Milone et al., Mol. Ther. (2009) 12 (9): 1453-64. In some embodiments, the co-stimulatory molecular has the sequence set forth in SEQ ID NO: 12.

In some embodiments, the intracellular signaling domain of the recombinant receptor, e.g., the CAR, comprises a human CD3 zeta stimulatory signaling domain or functional variant thereof, such as a 112 AA cytoplasmic domain of isoform 3 of human CD32 (Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15. In some embodiments, the CD3-zeta domain is any as described in Milone et al., Mol. Ther. (2009) 12 (9): 1453-64. In some embodiments, the CD3-zeta is or comprises the sequence set forth in SEQ ID NO: 13.

In some aspects, the spacer contains only a hinge region of an IgG, such as only a hinge of IgG4 or IgG1, such as the hinge only spacer set forth in SEQ ID NO: 1 or SEQ ID NO: 89. In other embodiments, the spacer is or contains an Ig hinge, e.g., an IgG4-derived hinge, optionally linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 4. In some embodiments, the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 81-83, an FcγRIIIa hinge, such as set forth in SEQ ID NO: 88, a CTLA4 hinge, such as set forth in SEQ ID NO: 84, or a PD-1 hinge, such as set forth in SEQ ID NO: 86. In some embodiments the spacer is derived from CD8. In some embodiments, the spacer is a CD8a hinge sequence. In some embodiments, the hinge sequence is any as described in Milone et al., Mol. Ther. (2009) 12 (9): 1453-64. In some embodiments, the hinge is or comprises the sequence set forth in SEQ ID NO:82.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. In some embodiments, the CAR includes an antibody or fragment, such as scFv, a spacer such as any of the Ig-hinge containing spacers, a CD8-derived transmembrane domain, a 4-1BB-derived intracellular signaling domain, and a CD3 zeta-derived signaling domain.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO 2014/031687. For example, the marker can be a truncated EGFR (tEGFR) that is, optionally, linked to a linker sequence, such as a T2A cleavable linker sequence. In some embodiments, such CAR constructs further includes a T2A ribosomal skip element and/or a tEGFR sequence, e.g., downstream of the CAR.

An exemplary polypeptide for a truncated EGFR (e.g., tEGFR) comprises the sequence of amino acids set forth in SEQ ID NO: 7 or 166 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166. An exemplary T2A linker sequence comprises the sequence of amino acids set forth in SEQ ID NO: 6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g., CAR) can also be generated to express a truncated EGFR (EGFRt) as a non-immunogenic selection epitope (e.g., by introduction of a construct encoding the CAR and EGFRt separated by a T2A ribosome switch to express two proteins from the same construct), which then can be used as a marker to detect such cells (see e.g., U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 39, 40 and 153. In some examples the signal peptide is derived from CD8a. In some embodiments, the signal peptide is the sequence set forth in Accession No. NM_001768. In some embodiments, the signal peptide includes the sequences set forth in SEQ ID NO: 39.

In some embodiments, the CAR includes an anti-BCMA antibody or fragment, such as any of the anti-human BCMA antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-BCMA antibody or fragment, such as any of the anti-human BCMA antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. In some embodiments, the CAR specifically binds to BCMA, such as human BCMA, and includes an anti-human BCMA antibody or fragment as described. Non-limiting exemplary CAR sequences, including anti-BCMA CAR sequences, are set forth in SEQ ID NOs: 90-141. In some embodiments, an anti-BCMA CAR includes the amino acid sequence set forth in any of SEQ ID NOS: 90-141 or an amino acid sequence that exhibits at least at or about 90%, at or about 91%, at or about 92%, at or about 93%, at or about 94%, at or about 95%, at or about 96%, at or about 97%, at or about 96%, at or about 97%, at or about 98%, at or about 99% sequence identity to any one of SEQ ID NOS: 90-141, and wherein the CAR specifically binds BCMA, e.g. human BCMA.

In some embodiments, the dose of genetically engineered T cells comprises idecabtagene vicleucel cells (e.g., such as ABECMA® cells); bb21217 cells; orvacabtagene autoleucel cells; CT103A cells; ciltacabtagene autoleucel cells (such as CARVYKTI™ cells); KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells, or GC012F cells. In some embodiments, the dose of genetically engineered T cells comprises idecabtagene vicleucel cells (e.g., such as ABECMA© cells).

### B. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, and lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35 (9): 689-701 (2012). In some embodiments, combining TCM-enriched CD8⁺ T cells and CD4⁺ T cells further enhances efficacy.

In embodiments, memory T cells are present in both CD62L+ and CD62L-subsets of CD8⁺ peripheral blood lymphocytes. PBMC can be enriched for or depleted of CD62L-CD8⁺ and/or CD62L+CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO−, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L− and CD45RO−.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Pub. Number WO 2009/072003, or US20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35 (9): 651-660 (2012), Terakura et al., Blood. 1:72-82 (2012), and Wang et al., J Immunother. 35 (9): 689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1 (5): 355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g., anti-CD3. In some embodiments, the stimulating conditions include one or more agent, e.g., ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35 (9): 651-660 (2012), Terakura et al., Blood. 1:72-82 (2012), and/or Wang et al., J Immunother. 35 (9): 689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### C. Nucleic Acids, Vectors and Methods for Genetic Engineering

In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO:153, CD8a signal peptide set forth in SEQ ID NO:154, GMCSFR alpha chain signal sequence set forth in SEQ ID NO:156 (corresponding polynucleotide sequence set forth in SEQ ID NO:155) or the signal peptide set forth in SEQ ID NO:39 or modified variant thereof. In some embodiments, the signal peptide is the CD8a signal peptide set forth in Accession No. NM_001768.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69 (2): 748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g., promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g., CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 166) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34 (4): 430-434). In some aspects, the marker, e.g., surrogate marker, includes all or part (e.g., truncated form) of CD34, a NGFR, a CD19 or a truncated CD19, e.g., a truncated non-human CD19, or epidermal growth factor receptor (e.g., (EGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g., surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO 2014/031687. For example, the marker can be a truncated EGFR (tEGFR) that is, optionally, linked to a linker sequence, such as a T2A cleavable linker sequence. An exemplary polypeptide for a truncated EGFR (e.g., tEGFR) comprises the sequence of amino acids set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166. An exemplary T2A linker sequence comprises the sequence of amino acids set forth in SEQ ID NO:6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g., CAR) can also be generated to express a truncated EGFR (EGFRt) as a non-immunogenic selection epitope (e.g., by introduction of a construct encoding the CAR and EGFRt separated by a T2A ribosome switch to express two proteins from the same construct), which then can be used as a marker to detect such cells (see e.g., U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166.

In some embodiments, a single promoter may direct expression of an RNA that contains, in a single open reading frame (ORF), two or three genes (e.g., encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A; e.g., SEQ ID NO:171), equine rhinitis A virus (E2A; e.g., SEQ ID NO:170), Thosea asigna virus (T2A, e.g., SEQ ID NO: 6 or 167), and porcine teschovirus-1 (P2A; e.g., SEQ ID NO:168 or 169) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses and adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3; Carlens et al. (2000) Exp Hematol 28 (10): 1137-46; Alonso-Camino et al. (2013) Mol Ther Nucl Acids 2, e93; Park et al., Trends Biotechnol. 2011 Nov. 29 (11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35 (9): 689-701; Cooper et al. (2003) Blood. 101:1637-1644; Verhoeyen et al. (2009) Methods Mol Biol. 506:97-114; and Cavalieri et al. (2003) Blood. 102 (2): 497-505.

In some embodiments, recombinant nucleic acids are transferred into T cells via electroporation (see, e.g., Chicaybam et al, (2013) PLoS ONE 8 (3): e60298 and Van Tedeloo et al. (2000) Gene Therapy 7 (16): 1431-1437). In some embodiments, recombinant nucleic acids are transferred into T cells via transposition (see, e.g., Manuri et al. (2010) Hum Gene Ther 21 (4): 427-437; Sharma et al. (2013) Molec Ther Nucl Acids 2, e74; and Huang et al. (2009) Methods Mol Biol 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle. bombardment (Johnston, Nature, 346:776-777 (1990)); and strontium phosphate DNA co-precipitation (Brash et al., Mol. Cell Biol., 7:2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion, e.g., with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus, e.g., via a de novo introduced receptor). This second type of stimulus may include an antigenic stimulus in form of a peptide/MHC molecule, the cognate (cross-linking) ligand of the genetically introduced receptor (e.g., natural ligand of a CAR) or any ligand (such as an antibody) that directly binds within the framework of the new receptor (e.g., by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65:333-347 (2014).

In some cases, a vector may be used that does not require that the cells, e.g., T cells, are activated. In some such instances, the cells may be selected and/or transduced prior to activation. Thus, the cells may be engineered prior to, or subsequent to, culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### III. Compositions and Formulations

Also provided are compositions for a BCMA-targeted therapy, such as BCMA-targeted CAR T cells, including pharmaceutical compositions and formulations. In some aspects, also provided are compositions, e.g., cell compositions for use in the provided methods and uses, e.g., therapeutic methods and uses. In some embodiments, the provided compositions are capable of achieving certain therapeutic outcomes, e.g., response or safety outcomes, when administered to subjects that have a disease or disorder, e.g., multiple myeloma.

Provided are pharmaceutical formulations comprising BCMA-targeted CAR T cells, a plurality of BCMA-targeted CAR T cells and/or additional agents for combination treatment or therapy. The pharmaceutical compositions and formulations generally include one or more optional pharmaceutically acceptable carrier(s) or excipient(s). In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9:467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, e.g., CAR, a subject is administered the range of at or about one million to at or about 100 billion cells, such as, e.g., 1 million to at or about 50 billion cells (e.g., at or about 5 million cells, at or about 25 million cells, at or about 50 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 100 million cells, at or about 120 million cells, at or about 150 million cells, at or about 250 million cells, at or about 300 million cells, at or about 350 million cells, at or about 450 million cells, at or about 650 million cells, at or about 800 million cells, at or about 900 million cells, at or about 1.2 billion cells, at or about 3 billion cells, at or about 30 billion cells, or at or about 45 billion cells) or any value in between these ranges, and/or such a number of cells per kilogram of body weight of the subject. In some aspects, in the context of genetically engineered cells expressing the binding molecules, e.g., CAR, a composition can contain at least the number of cells for administration for a dose of cell therapy, such as about or at least a number of cells described herein for administration, e.g., in Section B.1.

The cells may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility may be readily accomplished, e.g., by filtration through sterile filtration membranes.

### IV. Definitions

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

“Human BCMA” refers to BCMA found in a human subject, and having, e.g., SEQ ID NO: 215.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-BCMA antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, T cell therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### V. Exemplary Embodiments

Among the provided embodiments are:

1. A method of treating multiple myeloma in a subject who had an early relapse, an inadequate response, or a suboptimal response to one or more anti-myeloma treatment, comprising administering to the subject a BCMA targeted CAR T cell therapy followed by an immunomodulatory agent maintenance therapy.

2. A method of treating multiple myeloma in a subject, the method comprising:
(a) selecting a subject who had an early relapse, an inadequate response, or a suboptimal response to one or more anti-myeloma treatment;
(b) administering to the selected subject a BCMA targeted CAR T cell therapy; and
(c) after administering the BCMA targeted CAR T cell therapy, administering to the subject an immunomodulatory agent maintenance therapy.

3. A method of treating newly diagnosed multiple myeloma (NDMM) in a subject who had an early relapse, inadequate response, or a suboptimal response to one or more anti-myeloma treatment, comprising administering to the subject a BCMA targeted CAR T cell therapy followed by an immunomodulatory agent maintenance therapy.

4. A method of treating newly diagnosed multiple myeloma (NDMM) in a subject, the method comprising:
(a) selecting a subject who had an early relapse, an inadequate response, or a suboptimal response to one or more anti-myeloma treatment;
(b) administering to the selected subject a BCMA targeted CAR T cell therapy; and
(c) after administering the BCMA targeted CAR T cell therapy, administering to the subject an immunomodulatory agent maintenance therapy.

5. A method of maintenance therapy for treating multiple myeloma, the method comprising administering an immunomodulatory agent maintenance therapy to a subject after having received a BCMA targeted CAR T cell therapy for treating multiple myeloma, wherein prior to the BCMA target CAR T cell therapy the subject had an early relapse, an inadequate response, or a suboptimal response to one or more other anti-myeloma treatment.

6. The method of any of embodiments 1-5, wherein the immunomodulatory agent maintenance therapy is a maintenance therapy with lenalidomide.

7. The method of any of embodiments 1-6, wherein the one or more anti-myeloma treatment comprises stem cell transplant therapy.

8. The method of embodiment 7, wherein the stem cell transplant therapy comprises autologous stem cell therapy (ASCT).

9 The method of embodiment 7 or 8, wherein the stem cell transplant therapy comprises an induction therapy followed by a stem cell transplant.

10. The method of any of embodiments 7-9, wherein the stem cell transplant therapy comprises an induction therapy followed by high-dose chemotherapy (HDT) and a stem cell transplant.

11. The method of any of embodiments 8-10, wherein the stem cell transplant therapy consists of an induction therapy followed by a stem cell transplant.

12. The method of embodiment 1-11, wherein the multiple myeloma is a high-risk multiple myeloma.

13. The method of any of embodiments 1-12, wherein the subject is a subject that had an inadequate response to one or more anti-myeloma treatment. 14. The method of any of embodiments 1-13, wherein the inadequate response to the one or more anti-myeloma treatment is characterized by less than very good partial response (VGPR), e.g., at 70-110 days, after last treatment of the one or more anti-myeloma treatment without use of consolidation or maintenance therapy.

15. The method of any of embodiments 1-13, wherein the inadequate response to the one or more anti-myeloma treatment is characterized by less than very good partial response (VGPR), e.g., at 80-120 days, after last treatment of the one or more anti-myeloma treatment without use of consolidation or maintenance therapy.

16. The method of any of embodiments 1-14, wherein the one or more anti-myeloma treatment is an ASCT with prior induction therapy and the inadequate response is characterized by less than very good partial response (VGPR), e.g., at 70-110 days, after last ASCT without use of consolidation or maintenance therapy.

17. The method of any of embodiments 1-13 or 15, wherein the one or more anti-myeloma treatment is an ASCT with prior induction therapy and the inadequate response is characterized by less than very good partial response (VGPR), e.g., at 80-120 days, after last ASCT without use of consolidation or maintenance therapy.

18. A method of treating high-risk multiple myeloma in a subject, comprising administering to the subject a BCMA targeted CAR T cell therapy followed by an immunomodulatory agent maintenance therapy, wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma.

19. A method of treating high-risk multiple myeloma in a subject, the method comprising:
(a) selecting a subject with a multiple myeloma characterized by an inadequate response to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

20. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had an inadequate response to an autologous stem cell transplant (ASCT) after an induction therapy for treating the multiple myeloma.

21. A method of treating high-risk multiple myeloma in a subject, comprising administering to the subject a BCMA targeted CAR T cell therapy,
wherein the subject, at the time of administering the BCMA targeted CAR T cell therapy, has a high-risk multiple myeloma characterized by an inadequate response to autologous stem cell transplant (ASCT) after an induction therapy for treating the multiple myeloma.

22. The method of any of embodiments 17-21, wherein the inadequate response to the autologous stem cell transplant (ASCT) after an induction therapy is characterized by less than very good partial response (VGPR), e.g., at 70-110 days, after last ASCT.

23. The method of any of embodiments 17-21, wherein the inadequate response to the autologous stem cell transplant (ASCT) after an induction therapy is characterized by less than very good partial response (VGPR), e.g., at 80-120 days, after last ASCT.

24. The method of any of embodiments 17-23, wherein the ASCT after the induction therapy is a frontline therapy for treating a newly diagnosed multiple myeloma (NDMM).

25. The method of any of embodiments 1-11, wherein the subject is a subject that had an early relapse to the one or more anti-myeloma treatment.

26. The method of any of embodiments 1-11 and 25, wherein the early relapse is characterized by development of progressive disease (PD) less than 18 months from starting the one or more anti-myeloma treatment.

27. A method of treating high-risk multiple myeloma in a subject, comprising administering to the subject a BCMA targeted CAR T cell therapy followed by an immunomodulatory agent maintenance therapy,
wherein the subject, at the time of administering the BCMA targeted CAR T cell therapy, has a high-risk multiple myeloma characterized by early relapse to autologous stem cell transplant (ASCT) after an induction therapy for treating the multiple myeloma.

28. A method of treating high-risk multiple myeloma in a subject, the method comprising:
(a) selecting a subject with a multiple myeloma characterized by an early relapse to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

29. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had an early relapse to an autologous stem cell transplant (ASCT) after an induction therapy for treating the multiple myeloma.

30. A method of treating multiple myeloma in a subject, comprising administering to the subject a BCMA targeted CAR T cell therapy,
wherein the subject, at the time of administering the BCMA targeted CAR T cell therapy, has a multiple myeloma characterized by an early relapse to an autologous stem cell transplant (ASCT) after an induction therapy for treating the multiple myeloma.

31. The method of any of any of embodiments 27-30, wherein the early relapse is characterized by development of progressive disease (PD) less than 18 months from starting the autologous stem cell transplant (ASCT) after an induction therapy.

32. The method of any of embodiments 1-11, wherein the subject is a subject that had a suboptimal response to one or more anti-myeloma treatment.

33. The method of any of embodiments 1-11 or 32, wherein the suboptimal response to the one or more anti-myeloma treatment is characterized by partial response (PR) or very good partial response (VGPR), e.g., at 80-120 days, after last treatment of the one or more anti-myeloma treatment without use of consolidation or maintenance therapy.

34. The method of any of embodiments 1-11 or 32-33, wherein the suboptimal response to the one or more anti-myeloma treatment is characterized by partial response (PR) or very good partial response (VGPR) at about 100 days after last treatment of the one or more anti-myeloma treatment without use of consolidation or maintenance therapy.

35. The method of any of embodiments 1-11 or 32-33, wherein the one or more anti-myeloma treatment is an ASCT with prior induction therapy and the suboptimal response is characterized by partial response or very good partial response (VGPR), e.g., at 80-120 days, after last ASCT without use of consolidation or maintenance therapy.

36. The method of any of embodiments 1-11 or 32-35, wherein the one or more anti-myeloma treatment is an ASCT with prior induction therapy and the suboptimal response is characterized by partial response or very good partial response (VGPR) at about or at 100 days after last ASCT without use of consolidation or maintenance therapy.

37. The method of embodiment 35 or embodiment 36, wherein the ASCT with prior induction therapy further comprises high-dose chemotherapy (HDT).

38. A method of treating multiple myeloma in a subject, comprising administering to the subject a BCMA targeted CAR T cell therapy followed by an immunomodulatory agent maintenance therapy,
wherein the subject, at the time of administering the BCMA targeted CAR T cell therapy, has a multiple myeloma characterized by a suboptimal response to autologous stem cell transplant (ASCT) after an induction therapy for treating the multiple myeloma.

39. A method of treating multiple myeloma in a subject, the method comprising:
(a) selecting a subject with a multiple myeloma characterized by a suboptimal response to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

40. A method of treating multiple myeloma in a subject, the method comprising:
(a) selecting a subject with a multiple myeloma characterized by a suboptimal response to an autologous stem cell transplant (ASCT) after an induction therapy for treating the multiple myeloma;
(b) administering to the selected subject an immunomodulatory agent maintenance therapy prior to a leukapheresis to obtain T cells from the subject for engineering with a BCMA targeted CAR to produce a BCMA targeted CAR T cell therapy;
(c) administering to the selected subject the BCMA targeted CAR T cell therapy; and
(d) after administering the BCMA targeted CAR T cell therapy, administering to the subject an immunomodulatory agent maintenance therapy.

41. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had a suboptimal response to an autologous stem cell transplant (ASCT) after an induction therapy for treating the multiple myeloma.

42. The method of any of embodiments 38-39 or 41, wherein the subject has been administered an immunomodulatory agent maintenance therapy prior to a leukapheresis to obtain T cells from the subject for engineering with a BCMA targeted CAR to produce a BCMA targeted CAR T cell therapy.

43. A method of treating multiple myeloma in a subject, comprising administering to the subject a BCMA targeted CAR T cell therapy,
wherein the subject, at the time of administering the BCMA targeted CAR T cell therapy, has a multiple myeloma characterized by a suboptimal response an autologous stem cell transplant (ASCT) after an induction therapy for treating the multiple myeloma.

44. The method of any of embodiments 38-43, wherein the suboptimal response to the autologous stem cell transplant (ASCT) after an induction therapy is characterized by partial response (PR) or very good partial response (VGPR), e.g., at 80-120 days, after last ASCT.

45. The method of any of embodiments 38-44, wherein the suboptimal response to the autologous stem cell transplant (ASCT) after an induction therapy is characterized by partial response (PR) or very good partial response (VGPR) at about or at 100 days after last ASCT.

46. The method of any of embodiments 38-45, wherein the ASCT after induction therapy further comprises high-dose chemotherapy (HDT).

47, The method of any of embodiments 38-46, wherein the induction therapy comprises one or more of a proteasome inhibitor and an immunomodulatory agent.

48. The method of any of embodiments 9-47, wherein the induction therapy is one or more of a proteasome inhibitor, an immunomodulatory agent, and dexamethasone.

49. The method of any of embodiments 9-46, wherein the induction therapy is:
(i) bortezomib, lenalidomide and dexamethasone (VRD);
(ii) thalidomide and dexamethasone (TD);
(iii) lenalidomide and low-dose dexamethasone (RD);
(iv) bortezomib and dexamethasone (VD);
(v) bortezomib, thalidomide and dexamethasone (VTD);
(vi) carfilzomib, lenalidomide and dexamethasone (KRd); or
(vii) ixazomib, lenalidomide and dexamethasone (Ixa-Rd).

50. The method of any of embodiments 8-49, wherein the induction therapy is administered in ≥3 cycles.

51. The method of any of embodiments 9-50, wherein the induction therapy is administered in 3-12 cycles.

52. The method of any of embodiments 9-50, wherein the induction therapy is administered in 3-6 cycles.

53. The method of any of embodiments 38-50, wherein the induction therapy is administered in 4-6 cycles.

54. The method of any of embodiments 48-53, wherein each cycle is a 28-day cycle.

55. The method of any of embodiments 48-54, wherein in each cycle of the induction therapy:
bortezomib is administered at or about 1.3 mg/m² intravenously or subcutaneously on days 1, 8 and 15 of the cycle;
lenalidomide is administered at or about 25 mg orally on days 1 to 21 of the cycle or days 1 to 14 of the cycle; and/or
dexamethasone is administered at or about 40 mg on days 1, 8 and 15 of the cycle.

56. The method of any of embodiments 1-55, wherein prior to receiving the BCMA targeted CAR T cell therapy, the subject had not received a prior immunomodulatory agent maintenance therapy.

57. The method of any of embodiments 1-56, wherein prior to receiving the BCMA targeted CAR T cell therapy, the subject had not received a consolidation therapy.

58. The method of any of embodiments 9-55, wherein prior to receiving the BCMA targeted CAR T cell therapy, the subject received a prior immunomodulatory agent maintenance therapy after the ASCT with the induction therapy.

59. The method of any of embodiments 1-58, wherein the subject was diagnosed with multiple myeloma about three years or less before administering the BCMA targeted CAR T cell therapy.

60. The method of any of embodiments 1-58, wherein the subject was diagnosed with multiple myeloma about two years or less before administering the BCMA targeted CAR T cell therapy.

61. The method of any of embodiments 1-58, wherein the subject was diagnosed with multiple myeloma about 1.6 years or less before administering the BCMA targeted CAR T cell therapy.

62. The method of any of embodiments 1-61, wherein at the time of administering the BCMA targeted CAR T cell therapy the subject has R-ISS stage I disease.

63. The method of any of embodiments 1-61, wherein at the time of administering the BCMA targeted CAR T cell therapy the subject has R-ISS stage II disease.

64. The method of any of embodiments 1-61, wherein at the time of administering the BCMA targeted CAR T cell therapy the subject has R-ISS stage III disease.

65. The method of any of embodiments 1-64, wherein at the time of administering the BCMA targeted CAR T cell therapy the subject has high-risk cytogenetics.

66. The method of any of embodiments 1-64, wherein at the time of administering the BCMA targeted CAR T cell therapy, the subject has ultra high-risk cytogenetics.

67. The method of any of embodiments 1-66, wherein at the time of administering the BCMA targeted CAR T cell therapy the subject has bone marrow biopsy-determined high tumor burden, optionally wherein the high tumor burden is ≥50% bone marrow CD138+ plasma cells.

68. The method of any of embodiments 1-67, wherein at the time of administering the BCMA targeted CAR T cell therapy the subject has extramedullary disease.

69. The method of any of embodiments 1-68, wherein at the time of administering the BCMA targeted CAR T cell therapy the multiple myeloma was refractory to treating with one or both of an immunomodulatory agent and a proteasome inhibitor (PI).

70. The method of any of embodiments 1-69, wherein at the time of administering the BCMA targeted CAR T cell therapy the subject had an ECOG performance status (PS)≤1.

71. The method of any of embodiments 1-70, wherein at the time of administering the BCMA targeted CAR T cell therapy the subject had an ECOG PS of 0.

72. The method of any of embodiments 42-71, wherein the immunomodulatory agent maintenance therapy prior to leukapheresis is initiated between or from 10 to 12 weeks prior to administering the BCMA targeted CAR-T cell therapy.

73. The method of embodiment 72, wherein the immunomodulatory agent maintenance therapy prior to leukapheresis is initiated at about 10 weeks prior to administering the BCMA targeted CAR-T cell therapy.

74. The method of embodiment 72, wherein the immunomodulatory agent maintenance therapy prior to leukapheresis is initiated at about 11 weeks prior to administering the BCMA targeted CAR-T cell therapy.

75. The method of embodiment 72, wherein the immunomodulatory agent maintenance therapy prior to leukapheresis is initiated at about 12 weeks prior to administering the BCMA targeted CAR-T cell therapy.

76. The method of any of embodiments 42-75, wherein the immunomodulatory agent maintenance therapy after administering the BCMA targeted CAR T cell therapy is initiated at no later than 6 months after administering the BCMA targeted CAR T cell therapy.

77. The method of any of embodiments 42-76, wherein the immunomodulatory agent maintenance therapy after administering the BCMA targeted CAR T cell therapy is initiated at no later than about 5 months after administering the BCMA targeted CAR T cell therapy.

78. The method of any of embodiments 42-77, wherein the immunomodulatory agent maintenance therapy after administering the BCMA targeted CAR T cell therapy is initiated at no later than about 4 months after administering the BCMA targeted CAR T cell therapy.

79. The method of any of embodiments 42-78, wherein the immunomodulatory agent maintenance therapy after administering the BCMA targeted CAR T cell therapy is initiated at no later than about 3 months after administering the BCMA targeted CAR T cell therapy.

80. The method of any of embodiments 42-79, wherein the immunomodulatory agent maintenance therapy after administering the BCMA targeted CAR T cell therapy is initiated at no later than about 2 months after administering the BCMA targeted CAR T cell therapy.

81. The method of any of embodiments 42-80, wherein the immunomodulatory agent maintenance therapy after administering the BCMA targeted CAR T cell therapy is initiated at no later than about 1 month after administering the BCMA targeted CAR T cell therapy.

82. The method of any of embodiments 42-81, wherein the immunomodulatory agent maintenance therapy after administering the BCMA targeted CAR T cell therapy is initiated at about 1 month after administering the BCMA targeted CAR T cell therapy.

83. The method of any of embodiments 1-71, wherein the immunomodulatory agent maintenance therapy is initiated at Day 15 or later after administering the BCMA targeted CAR T cell therapy.

84. The method of any of embodiments 1-71 or 83, wherein the immunomodulatory agent maintenance therapy is initiated at 1 month or later after administering the BCMA targeted CAR T cell therapy.

85. The method of any of embodiments 1-71 or 83-84, wherein the immunomodulatory agent maintenance therapy is initiated at at least 1 month after administering the BCMA targeted CAR T cell therapy.

86. The method of any of embodiments 1-71 or 83-85, wherein the immunomodulatory agent maintenance therapy is initiated at at least 2 months after administering the BCMA targeted CAR T cell therapy.

87. The method of any of embodiments 1-71 or 83-86, wherein the immunomodulatory agent maintenance therapy is initiated at at least 3 months after administering the BCMA targeted CAR T cell therapy.

88. The method of any of embodiments 1-71 or 83-87, wherein the immunomodulatory agent maintenance therapy is initiated at at least 4 months after administering the BCMA targeted CAR T cell therapy.

89. The method of any of embodiments 1-71 or 83-88, wherein the immunomodulatory agent maintenance therapy is initiated at no later than 24 months after administering the BCMA targeted CAR T cell therapy.

90. The method of any of embodiments 1-71 or 83-89, wherein the immunomodulatory agent is initiated at or between Day 15 and Day 730 after administering the BCMA targeted CAR T cell therapy.

91. The method of any of embodiments 1-71 or 83-90, wherein the immunomodulatory agent maintenance therapy is initiated at or between 1 month and 24 months, 1 month and 12 months, 1 month and 10 months, 1 month and 6 months, or 1 month and 4 months after administering the BCMA targeted CAR T cell therapy.

92. The method of any of embodiments 1-71 or 83-91, wherein the immunomodulatory agent maintenance therapy is initiated at or between 1 month and 6 months after administering the BCMA targeted CAR T cell therapy.

93. The method of any of embodiments 1-71 or 83-91, wherein the immunomodulatory agent maintenance therapy is initiated at or between 4 months and 24 months, 4 months and 12 months, or 4 months and 10 months after administering the BCMA targeted CAR T cell therapy.

94. The method of any of embodiments 1-71, 83-91 or 93, wherein the immunomodulatory agent maintenance therapy is initiated at or between 4 month and 10 months, after administering the BCMA targeted CAR T cell therapy.

95. The method of any of embodiments 1-71 or 83-91, wherein the immunomodulatory agent maintenance therapy is initiated at or between Day 30 and Day 180 after administering the BCMA targeted CAR T cell therapy.

96. The method of any of embodiments 1-85, 90 or 95, wherein the immunomodulatory agent maintenance therapy is initiated at 1 month after administering the BCMA targeted CAR T cell therapy.

97. The method of any of embodiments 1-96, wherein the immunomodulatory agent maintenance therapy continues until disease progression.

98. The method of any of embodiments 1-96, wherein the immunomodulatory agent maintenance therapy continues until the subject achieves complete response (CR).

99. The method of any of embodiments 1-98, wherein the immunomodulatory agent of the immunomodulatory agent maintenance therapy is a compound that interacts with and/or binds to cereblon (CRBN) or one or more members of the CRBN E3 ubiquitin-ligase complex; an inhibitor of Ikaros (IKZF1); an inhibitor of Aiolos (IKZF3); or a compound that enhances or promote ubiquitination and/or degradation of Ikaros (IKZF1) and Aiolos (IKZF3).

100. The method of any of embodiments 1-99, wherein the immunomodulatory agent of the immunomodulatory agent maintenance therapy is thalidomide, a thalidomide analog or a thalidomide derivative.

101. The method of any of embodiments 1-100, wherein the immunomodulatory agent of the immunomodulatory agent maintenance therapy is lenalidomide, pomalidomide, avadomide, iberdomide, CC-92480 or CC-885.

102. The method of any of embodiments 1-101, wherein the immunomodulatory agent of the immunomodulatory agent maintenance therapy is lenalidomide.

103. The method of any of embodiments 1-102, wherein the immunomodulatory agent of the immunomodulatory agent maintenance therapy is administered in an amount of 0.5 mg per day to 50 mg per day.

104. The method of any of embodiments 1-103, wherein the immunomodulatory agent of the immunomodulatory agent maintenance therapy is administered in an amount of 0.5 mg per day to 25 mg per day, 0.5 mg per day to 10 mg per day, 0.5 mg per day to 5 mg per day, 0.5 mg per day to 2.5 mg per day, 0.5 mg per day to 1 mg per day, 1 mg per day to 50 mg per day, 1 mg per day to 25 mg per day, 1 mg per day to 10 mg per day, 1 mg per day to 5 mg per day, 1 mg per day to 2.5 mg per day, 2.5 mg per day to 50 mg per day, 2.5 mg per day to 25 mg per day, 2.5 mg per day to 10 mg per day, 2.5 mg per day to 5 mg per day, 5 mg per day to 50 mg per day, 5 mg per day to 25 mg per day, 5 mg per day to 10 mg per day, 10 mg per day to 50 mg per day, 10 mg per day to 25 mg per day or 25 mg per day to 50 mg per day.

105. The method of any of embodiments 1-104, wherein the immunomodulatory agent of the immunomodulatory agent maintenance therapy is administered in an amount of 2.5 mg per day to 25 mg per day.

106 The method of any of embodiments 1-104, wherein the immunomodulatory agent of the immunomodulatory agent maintenance therapy is administered in an amount of 2.5 mg per day to 10 mg per day.

107. The method of any of embodiments 1-104, wherein the immunomodulatory agent of the immunomodulatory agent maintenance therapy is administered in an amount of at or about 1 mg per day.

108. The method of any of embodiments 1-106, wherein the immunomodulatory agent of the immunomodulatory agent maintenance therapy is administered in an amount of at or about 2.5 mg per day.

109. The method of any of embodiments 1-106, wherein the immunomodulatory agent of the immunomodulatory agent maintenance therapy is administered in an amount of at or about 5 mg per day.

110. The method of any of embodiments 1-106, wherein the immunomodulatory agent of the immunomodulatory agent maintenance therapy is administered in an amount of at or about 10 mg per day.

111. The method of any of embodiments 1-105, wherein the immunomodulatory agent of the immunomodulatory agent maintenance therapy is administered in an amount of at or about 15 mg per day.

112. The method of any of embodiments 103-111, wherein the amount of the immunomodulatory agent of the immunomodulatory agent maintenance therapy is administered daily for 14 days in a 28-day cycle.

113. The method of any of embodiments 103-111, wherein the amount of the immunomodulatory agent of the immunomodulatory agent maintenance therapy is administered daily for 21 days in a 28-day cycle.

114. The method of any of embodiments 103-111, wherein the amount of the immunomodulatory agent of the immunomodulatory agent maintenance therapy is administered daily for 28 days in a 28-day cycle.

115. The method of any one of embodiments 1-114, wherein prior to the administration of the BCMA targeted CAR T cell therapy to the subject, the subject has received a lymphodepleting therapy comprising the administration of fludarabine at or about 20-40 mg/m2 body surface area of the subject (optionally at or about 30 mg/m2) daily for 2-4 days, and/or cyclophosphamide at or about 200-400 mg/m2 body surface area of the subject (optionally at or about 300 mg/m2) daily for 2-4 days.

116. The method of any one of embodiments 1-115, wherein prior to the administration of the BCMA targeted CAR T cell therapy to the subject, the subject has received a lymphodepleting therapy comprising the administration of fludarabine at or about 30 mg/m2 body surface area of the subject, daily, and cyclophosphamide at or about 300 mg/m2 body surface area of the subject, daily, for 3 days.

117. The method of any one of embodiments 1-116, wherein the BCMA targeted CAR T cell therapy comprises a chimeric antigen receptor (CAR) comprising an extracellular antigen-binding domain that binds to BCMA, a transmembrane domain, and an intracellular signaling region.

118. The method of embodiment 117, wherein the extracellular antigen-binding domain comprises a variable heavy chain (VH) region and a variable light chain (VL) region.

119. The method of embodiment 118, wherein:
the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or
the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.

120. The method of embodiment 118 or embodiment 119, wherein:
the VH region comprises an amino acid sequence set forth in SEQ ID NO: 18 and the VL region comprises the amino acid sequence set forth in SEQ ID NO: 19;
or the VH region comprises an amino acid sequence set forth in SEQ ID NO: 24, and the VL region comprises the amino acid sequence set forth in SEQ ID NO: 25.

121. The method of any one of embodiments 117-120, wherein the extracellular antigen-binding domain is a single chain variable fragment (scFv).

122. The method of embodiment 121, wherein the scFv comprises the amino acid sequence set forth in SEQ ID NO: 213 or SEQ ID NO: 188.

123. The method of any one of embodiments 117-120, wherein the intracellular signaling region further comprises a costimulatory signaling domain.

124. The method of embodiment 123, wherein the costimulatory signaling domain comprises an intracellular signaling domain of CD28, 4-1BB, or ICOS, or a signaling portion thereof.

125. The method of embodiment 123 or embodiment 124, wherein the costimulatory signaling domain is between the transmembrane domain and the cytoplasmic signaling domain of a CD3-zeta (CD32) chain.

126. The method of any one of embodiments 117-125, wherein the transmembrane domain is or comprises a transmembrane domain from CD28 or CD8, optionally human CD28 or CD8.

127. The method of any one of embodiments 117-126, wherein the CAR further comprises an extracellular spacer between the antigen binding domain and the transmembrane domain.

128. The method of embodiment 127, wherein the spacer is from CD8, optionally wherein the spacer is a CD8a hinge.

129. The method of embodiment 127 or embodiment 128, wherein the transmembrane domain and the spacer are from CD8.

130. The method of any one of embodiments 117-129, wherein the CAR comprises the amino acid sequence set forth in SEQ ID NO: 116 or SEQ ID NO: 124.

131. The method of any one of embodiments 117-120, wherein the CAR is encoded by the polynucleotide sequence set forth in SEQ ID NO: 214.

132. The method of any of embodiments 1-131, wherein the BCMA targeted CAR T cell therapy comprises CD3+ CAR-expressing T cells.

133. The method of any of embodiments 1-132, wherein the BCMA targeted CAR T cell therapy comprises a combination of CD4+ T cells and CD8+ T cells and/or a combination of CD4+ CAR-expressing T cells and CD8+ CAR-expressing T cells.

134. The method of embodiment 133, wherein the ratio of CD4+ CAR-expressing T cells to CD8+ CAR-expressing T cells and/or of CD4+ T cells to CD8+ T cells, is or is approximately 1:1 or is between at or approximately 1:3 and at or approximately 3:1.

135. The method of any of embodiments 1-134, wherein:
the percentage of naïve-like T cells and/or central memory T cells is greater than or greater than about 60% of the total genetically engineered T cells in the dose, optionally greater than or greater than about 65%, 70%, 80%, 90% or 95%;
the percentage of naïve-like T cells and/or central memory T cells is greater than or greater than about 40% of the total CD4+ genetically engineered T cells in the dose, optionally greater than or greater than about 50%, 60%, 70%, 80%, 90% or 95%; or
the percentage of naïve-like T cells and/or central memory T cells is greater than or greater than about 40% of the total CD8+ genetically engineered T cells in the dose, optionally greater than or greater than about 50%, 60%, 70%, 80%, 90% or 95%.

136. The method of embodiment 135, wherein the naïve-like T cells are CCR7+CD45RA+, CD27+CCR7+, or CD62L-CCR7+.

137. The method of any one of embodiments 1-136, wherein the BCMA targeted CAR T cell therapy is: idecabtagene vicleucel; bb21217 cells; orvacabtagene autoleucel; CT103A; ciltacabtagene autoleucel; KITE585; CT053; BCMA-CS1 cCAR (BC1cCAR); P-BCMA-101; P-BCMA-ALLO1; C-CAR088; Descartes-08; PBCAR269A; ALLO-715; PHE885; AUTO8; CTX120; CB-011; ALLO-605 (TuboCAR/MM); pCDCAR1 (TriCAR-Z136), or GC012F.

138. The method of any one of embodiments 1-137, wherein the BCMA targeted CAR T cell therapy is idecabtagene vicleucel.

139. The method of any one of embodiments 1-138, wherein the dose of the BCMA targeted CAR T cell therapy is between about 100×10⁶ CAR-positive T cells and about 600×106 CAR-positive T cells.

140. The method of any one of embodiments 1-139, wherein the dose of the BCMA targeted CAR T cell therapy is between about 150×10⁶ and about 540×10⁶ CAR-positive T cells.

141. The method of any one of embodiments 1-140, wherein the dose of the BCMA targeted CAR T cell therapy is between about 150×10⁶ and about 450×10⁶ CAR-positive T cells.

142. The method of any one of embodiments 1-139, wherein the dose of the BCMA targeted CAR T cell therapy is between about 300×10⁶ and about 540×10⁶ CAR-positive T cells.

143. The method of any one of embodiments 1-139 or 142, wherein the dose of the BCMA targeted CAR T cell therapy is between about 300×10⁶ and about 460×10⁶ CAR-positive T cells.

144. The method of any one of embodiments 1-139 or 142, wherein the dose of the BCMA targeted CAR T cell therapy is between about 300×10⁶ and about 450×10⁶ CAR-positive T cells.

145. The method of any one of embodiments 1-140, wherein the dose of the BCMA targeted CAR T cell therapy is about 150×10⁶, about 300×10⁶, about 450×10⁶, about 460×10⁶, or about 540×10⁶ CAR-positive T cells.

146. The method of any one of embodiments 1-140 or 142, wherein the dose of the BCMA targeted CAR T cell therapy is about 300×10⁶, about 450×10⁶, about 460×10⁶, or about 540×10⁶ CAR-positive T cells.

147. The method of any one of embodiments 1-146, wherein the T cells of the BCMA targeted CAR T cell therapy were obtained from the subject, optionally by apheresis or leukapheresis, and the T cells were engineered ex vivo with the BCMA targeted CAR.

148. The method of any one of embodiments 1-147, wherein the dose of the BCMA targeted CAR T cell therapy is autologous to the subject.

149. The method of any one of embodiments 1-147, wherein the dose of the BCMA targeted CAR T cell therapy is allogeneic to the subject.

150. The method of any of embodiments 1-149, wherein the subject received a bridging therapy prior to receiving the BCMA targeted CAR T cell therapy, optionally wherein the bridging therapy was administered to the subject in the period between obtaining the T cells from the subject and administering the BCMA targeted CAR T cell therapy to the subject.

151. The method of any of embodiments 1-149, further comprising administering to the subject a bridging therapy prior to administering the BCMA targeted CAR T cell therapy to the subject, optionally wherein the bridging therapy is administered to the subject in the period between obtaining the T cells from the subject and administering the BCMA targeted CAR T cell therapy to the subject.

152. The method of embodiment 150 or embodiment 151, wherein the bridging therapy comprises one or more of corticosteroids, alkylating agents, immunomodulatory agents, proteasome inhibitors (PI), or anti-CD38 antibodies.

153. The method of any of embodiments 1-152, wherein the subject is human.

154. The method of any one of embodiments 1-153, wherein the subject is 18 year of age or older.

### VI Examples

The following examples are included for illustrative purposes only and are not intended to limit the scope of the invention.

### Example 1: Treatment with Anti-BCMA CAR-T Cells in Patients with Clinical High-Risk Multiple Myeloma Due to Inadequate Response to Frontline Autologous Stem Cell Transplantation

Chimeric antigen-receptor (CAR)-expressing T cell compositions containing autologous T cells expressing a CAR specific for B-cell maturation antigen (BCMA) were administered to human subjects with multiple myeloma (MM) who had an inadequate response after frontline therapy with autologous stem cell transplant (ASCT). Induction therapy followed by ASCT is the standard of care (SoC) in transplant-eligible patients with newly diagnosed multiple myeloma (NDMM).

The administered T cell compositions were generated by obtaining peripheral-blood mononuclear cells (PBMCs) from leukapheresis samples from individual subjects to be treated, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8a, and a CD137 (4-1BB) co-stimulatory domain followed by the intracellular signaling domain of a CD35 chain. The polynucleotide sequence encoding the exemplary BCMA CAR is set forth in SEQ ID NO: 214, and the polypeptide sequence of the exemplary BCMA CAR is set forth in SEQ ID NO: 116.

### A. Eligible Subjects and Treatment

Eligible subjects were adult (≥18 years of age) human subjects with MM who had received ≥3 cycles of induction therapy (must include a proteasome inhibitor, an immunomodulatory agent, and dexamethasone), and ASCT (single or tandem) but who exhibited an inadequate response to the ASCT, defined as less than very good partial response (VGPR) at 70-110 days after last ASCT, without use of consolidation or maintenance therapy. Subjects had an Eastern Cooperative Oncology Group (ECOG) performance status (PS)≤1. Additionally, subjects have measurable disease. Measurable disease was determined by M-protein (serum protein electrophoresis ≥0.5 g/dL or urine protein electrophoresis ≥200 mg/24 hours) and/or light chain MM without measurable disease in serum or urine (serum immunoglobulin free light chain ≥10 mg/dL and abnormal serum immunoglobulin kappa lambda free light chain ratio).

Baseline demographics and clinical characteristics of subjects are depicted in Table E1. High tumor burden was determined by bone marrow biopsy CD138+ plasma cell (low <50%, high ≥50% bone marrow CD138+ plasma cells). High-risk cytogenetics were defined as del (17p), t(4; 14), t(14; 16). R-ISS is derived using baseline ISS stage, cytogenetic abnormality, and serum lactate dehydrogenase; R-ISS represents stage at study entry. At study entry, 41.9% of subjects had R-ISS stage I and 16.1% had stage II disease. Induction regimens were used in >15% of subjects; 1 subject (3.2%) had daratumumab as induction therapy. Best overall response (BOR) to ASCT was partial response (PR; 87.1%).

TABLE E1
Baseline Demographics and Clinical Characteristics

| Characteristics | All (N = 31) | |
|---|---|---|
| Median age (range), years | 64.0 | (46.0-72.0) |
| Male sex (%) | 67.7 | |
| Race, n (%) | | |
| Asian | 2 | (6.5) |
| Black or African American | 4 | (12.9) |
| White | 24 | (77.4) |
| Unknown | 1 | (3.2) |
| Median time from initial diagnosis | 1.0 | (0.7-1.9) |
| to screening (range), years | | |
| Extramedullary disease, n (%) | 2 | (6.5) |
| High tumor burden, n (%) | 1 | (3.2) |
| ECOG PS (0/1), % | 67.7/32.3 | |
| Unknown Cytogenetics, n (%) | | |
| High-risk | 3 | (9.7) |
| Standard risk | 14 | (45.2) |
| Non-evaluable/missing data | 14 | (45.2) |
| R-ISS Stage, n (%) | | |
| I | 13 | (41.9) |
| II | 5 | (16.1) |
| III | 0 | (0) |
| Missing | 13 | (41.9) |
| BOR to ASCT, n (%) | | |
| PR | 27 | (87.1%) |
| MR | 2 | (6.5%) |
| SD | 2 | (6.5%) |
| Induction Therapies, n (%) | | |
| Lenalidomide | 31 | (100.0) |
| Bortezomib | 25 | (80.6) |
| Cyclophosphamide | 11 | (35.5) |
| Carfilzomib | 12 | (38.7) |
| Dexamethasone | 31 | (100.0) |

Eligible subjects received a lymphodepleting chemotherapy (LDC) with fludarabine (flu, 30 mg/m²/day) and cyclophosphamide (cy, 300 mg/m²/day) for 3 consecutive days (at days −5, −4 and −3). Subjects received a single infusion of anti-BCMA CAR-T cells at a dose range of 150-450×10⁶ CAR+ T cells, up to an additional 20%. The median dose of infused CAR+ T cells was 440×10⁶ CAR+ T cells (range 244.9 to 514.5×10⁶ CAR+ T cells).

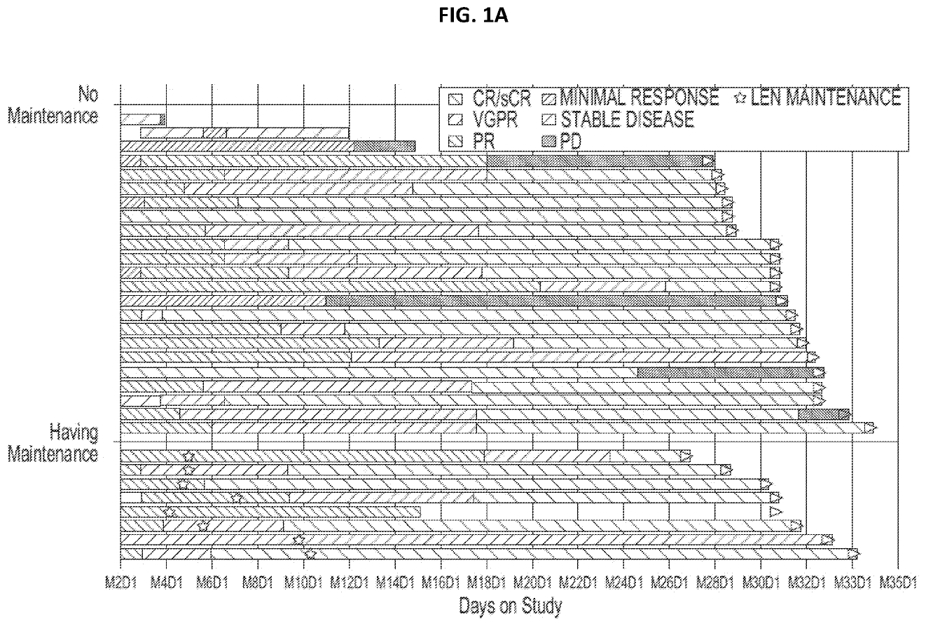

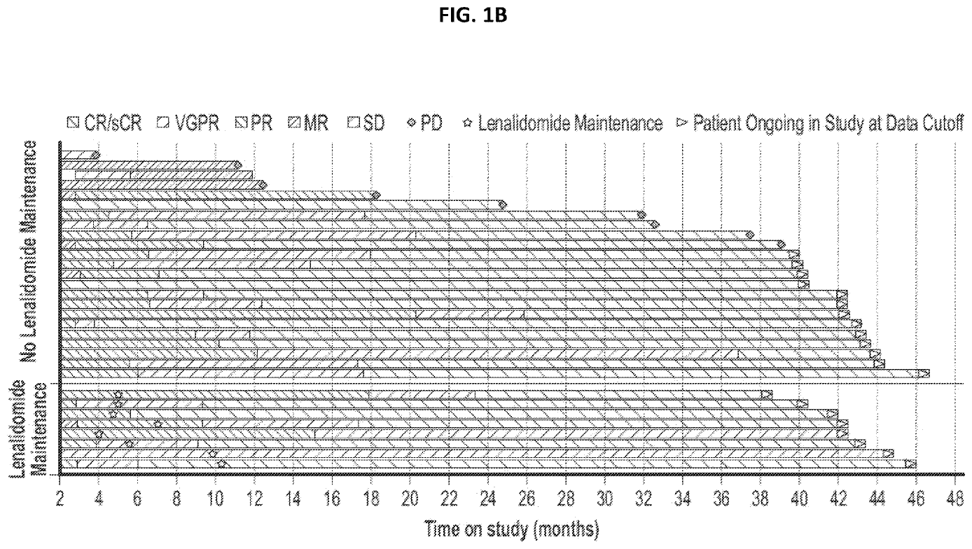

The post-treatment period included (1) following the subjects for a minimum of 24 months post-infusion or until PD, whichever was longer, and (2) continuing post-treatment follow-up discontinuation (PTFD) visit. Visits occurred at Days 2-8, 10, 12, 15, 18, 22, and 25 in month 1, once per month at months 2-7, 10, 13, 16, 19, 22, 25, 28, 31, and 34, up to Year 3; then every 3 months. After PD, survival follow-up occurred every 3 months, up to 5 years, after the last subject received the first anti-BCMA CAR-T cell infusion. At investigator discretion, certain subjects further received maintenance therapy with lenalidomide post anti-BCMA CAR-T cell infusion. For subjects receiving lenalidomide maintenance therapy, lenalidomide maintenance therapy was initiated between approximately 4 and 10 months from anti-BCMA CAR T cell infusion as shown by FIG. 1A. Eight subjects received lenalidomide maintenance. FIG. 1B depicts the same subjects over additional months, up to 48 months.

The primary efficacy endpoint was complete response (CR) rate (CRR; CR and stringent CR (sCR)) per International Myeloma Working Group (IMWG) Uniform Response Criteria. Secondary endpoints included overall response rate (ORR; ≥partial response), very good partial response (VGPR) rate (VGPRR), time to response (TTR), duration of response (DOR), progression-free survival (PFS), time to progression (TTP), overall survival (OS), safety, pharmacokinetics (PK), immunogenicity (anti-CAR antibody response), and health-related quality of life (HRQOL). PK was determined by time course of vector transgene copies per microgram of genomic DNA as measured by qPCR in evaluable subjects prior to anti-BCMA CAR T cell infusion. HRQOL was assessed using European Organization for Research and Treatment of Cancer (EORTC) Quality of Life Core 30 Questionnaire (QLQ-C30) and Quality of Life Multiple Myeloma 20 Questionnaire (QLQ-MY20).

Exploratory endpoints, which included assessment of biomarkers (serum level of soluble BCMA (sBCMA) and inflammatory cytokines) by Luminex assay and minimal residual disease negativity (MRD−) by using aggregation of next-generation sequencing and 8-color flow cytometry (EuroFlow; sensitivity <10⁻⁵ nucleated cells) data with interpolation to reduce missing/indeterminate data points. sBCMA levels were evaluated at leukapheresis; baseline; days 1, 4, and 8; and until disease progression. Plasma levels of immune-related inflammatory cytokines (interferon-γ (IFNγ), interleukin-6 (IL-6), interleukin-8 (IL-8) and interleukin-10 (IL-10)) were evaluated at leukapheresis; baseline; days 1-8, 10, 12, 15 18 and 22; and months 2 and 4. Efficacy and safety were analyzed in all subjects who received anti-BCMA CAR-T cells. PK, sBCMA, and MRD-were analyzed in evaluable subjects.

Anti-BCMA CAR-T cells were manufactured and infused in 31/32 subjects. One subject was not treated due to manufacturing failure. Two subjects received a bridging therapy following leukapheresis for a median duration of 38.0 days (range: 22.0-54.0). The median age of the subjects was 64 years old. 67.7% of the subjects had an ECOG PS of 0. Of subjects who were eligible at study entry, 41.9% of subjects had revised International Staging System (R-ISS) stage I disease, 16.1% of subjects had R-ISS stage II disease, and no subject had R-ISS stage III disease; 9.7% of subjects had high-risk cytogenetics; 3.2% had bone marrow biopsy-determined high tumor burden (≥50% bone marrow CD138+ plasma cells); and 6.5% had extramedullary disease.

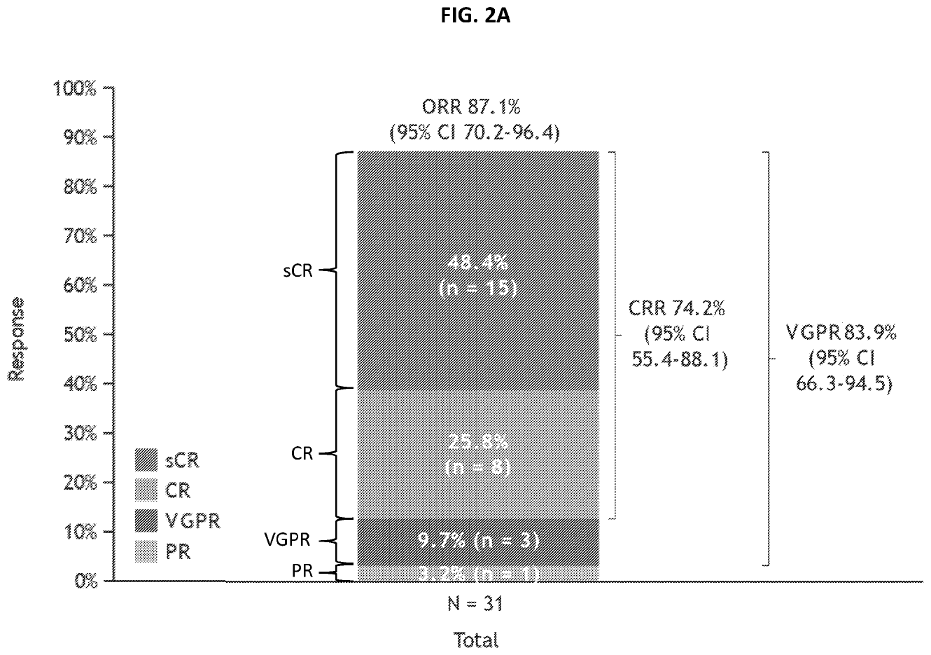

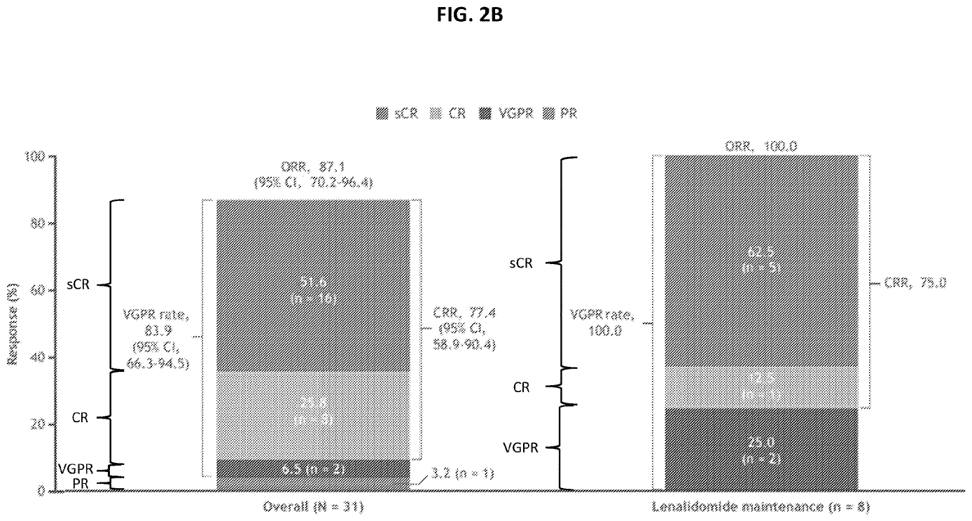

At an earlier time of analysis, the median follow-up was 27.9 months (range 24-32). At a median follow-up of 27.9 months, there were no deaths. CRR was 74.2% (95% CI 55.4-88.1), and ORR was 87.1% (95% CI 70.2-96.4), as shown by FIG. 2A and Table E2. Median TTR was 1 month (range 0.9-2.8) for subjects with PR or better. Most subjects responded to the anti-BCMA CAR-T cells. Two subjects achieved minimal response, two had stable disease and none had PD. Primary endpoint was met, with 74.2% of subjects achieving at least CR (P<0.0001). As shown by FIG. 2A, 84% of subjects had a VGPR or better.

At a subsequent time of analysis (after an additional 11.5 months of median follow-up), the median follow-up was 39.4 months (range 31.6-44.7). CRR was 77.4% and ORR was 87.1%, as shown by FIG. 2B. Since the earlier time of analysis, best of response (BOR) in one subject improved from VGPR to sCR. Median TTR was 1 month (range 0.9-2.8) for subjects with PR or better. Median time to CR was 8.3 months (range 1.0-35.9).

For subjects who received lenalidomide therapy, 6 out of 8 subjects (75%) achieved a complete response or better. The other two subjects achieved a very good partial response. The median (range) time from anti-BCMA CAR T cell infusion to the start of lenalidomide maintenance was 4 months (3.1-9.3). For these same 8 subjects, at the subsequent time of analysis, all eight subjects were still receiving lenalidomide therapy and all responses were ongoing. At the subsequent time of analysis, the median (range) duration of lenalidomide maintenance was 35.6 months (7.9-38.5).

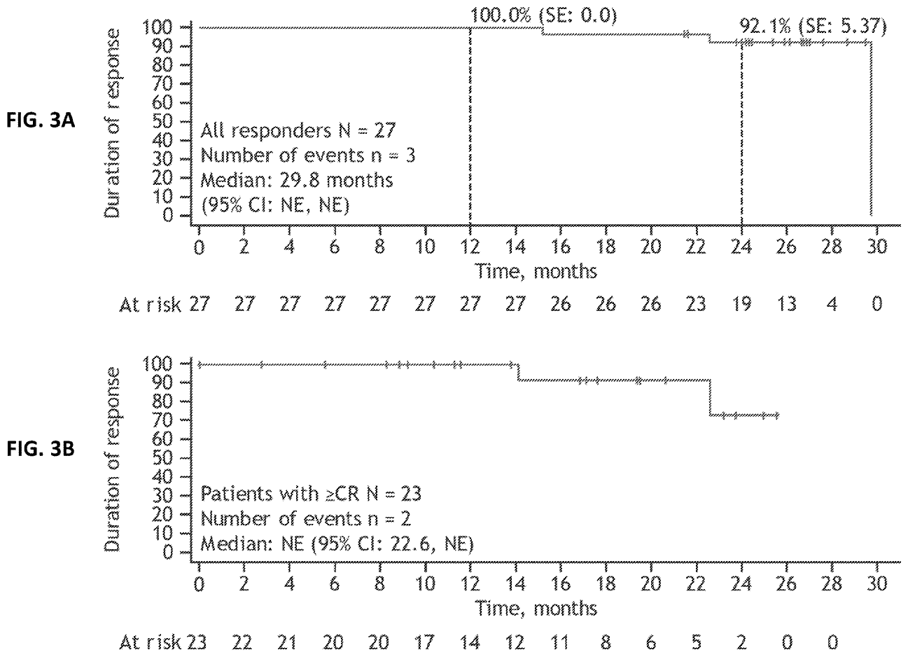

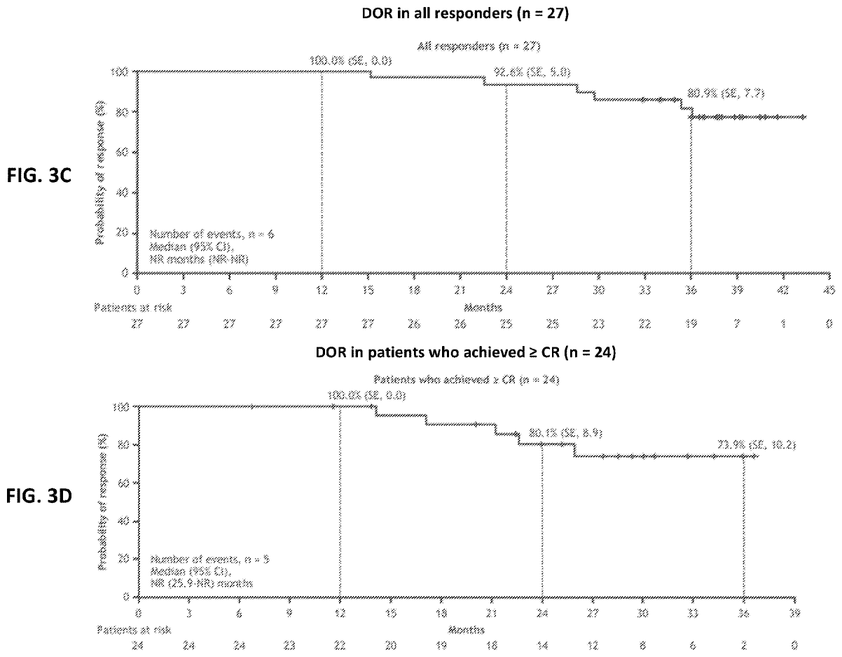

Time to event endpoint results are also depicted in Table E2. Durable responses were observed in all responders (100%) at month 12, and in 92.1% of subjects evaluated at month 24 (FIG. 3A). FIG. 3B shows responses in subjects who achieved ≥CR. Of the 6 subjects who progressed as of the date of results presented in this example, 4 did not achieve a response and 2 achieved ≥CR and progressed at months 24 and 30. None of the subjects who progressed received a lenalidomide maintenance.

At the subsequent time of analysis, median duration of response (DOR) was not reached. The 36 months event-free rate was 80.9% (FIG. 3C). FIG. 3D shows responses in subjects who achieved ≥CR. In the 8 subjects who received lenalidomide after anti-BCMA CAR T cell therapy, the median DOR was 37.7 months (95% CL, 32.9-40.6).

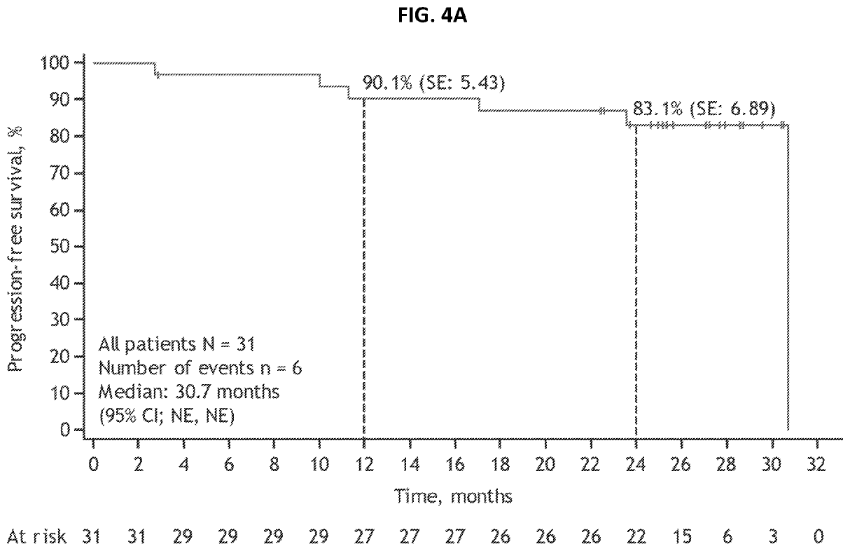

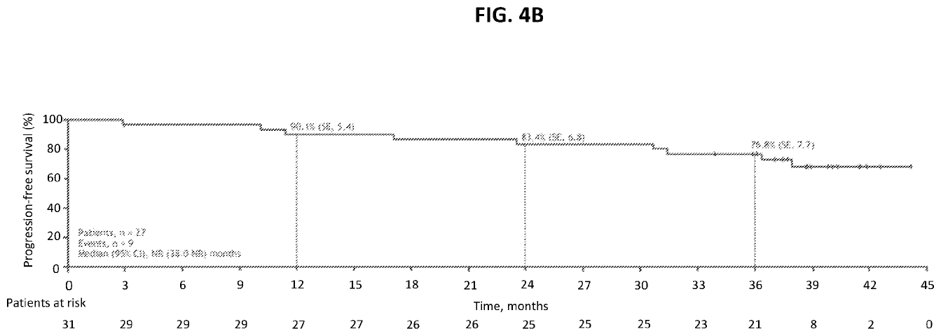

Progression-free survival (PFS) rate at months 12 and 24 was 90.1 and 83.1%, respectively, as shown in FIG. 4A. At the subsequent time of analysis, the PFS rate was 76.8% at 36 months, as shown in FIG. 4B. Median PFS was not reached.

At the earlier time of analysis, the median follow-up duration was 27.9 months. All subjects remained alive at time of analysis. None of the subjects who progressed received lenalidomide maintenance (e.g., no progressive disease events occurred in subjects who received maintenance). At the subsequent time of analysis, the median follow up was 39.4 months. Specifically, of the 32 originally enrolled subjects (or the 31 subjects who were treated with the anti-BCMA CAR=T cells), 28 subjects (90.3%) remained in follow-up while 3 subjects (9.7%) discontinued due to PD. All 31 subjects who were treated with the anti-BCMA CAR-T cells remained alive at the subsequent time of analysis. Median overall survival (OS) was not reached.

TABLE E2
Efficacy and Safety outcomes following
anti-BCMA CAR-T cell infusions

| | |
|---|---|
| Efficacy outcome | N = 31 |

Best overall response

| ORR, n | 27 | |
|---|---|---|
| ORR, % (95% CI¹) | 87.1 | (70.2-96.4) |
| VGPRR, n | 26 | |
| VGPRR, % (95% CI¹) | 83.9 | (66.3-94.5) |
| CRR, n | 23 | |
| CRR, % (95% CI¹) | 74.2 | (55.4-88.1) |
| Stringent CR, n (%) | 15 | (48.4) |
| CR, n (%) | 8 | (25.8) |
| VGPR, n (%) | 3 | (9.7) |
| PR, n (%) | 1 | (3.2) |
| Minimal response, n (%) | 2 | (6.5) |
| Stable disease, n (%) | 2 | (6.5) |

| | |
|---|---|
| Progressive disease, n | 0 |

Time to event rates

| | | |
|---|---|---|
| 12 mo DOR rate, % (SE) | 100.0 | (0.00) |
| 24 mo DOR rate, % (SE) | 92.1 | (5.37) |
| 12 mo PFS rate, % (SE) | 90.1 | (5.43) |
| 24 mo PFS rate, % (SE) | 83.1 | (6.89) |
| 12 mo OS rate, % (SE) | 100.0 | (0.00) |
| 24 mo OS rate, % (SE) | 100.0 | (0.00) |

| | N = 31 | | | |
|---|---|---|---|---|
| AEs of special interest | CRS | | NT | |
| Number of subjects with at least one | 18 | (58.1) | 2 | (6.5) |
| event of any grade, n (%) | | | | |
| Number of subjects with at least one | 0 | | 1 | (3.2) |
| grade 3/4 event, n (%) | | | | |
| Time to first onset, median, days (range) | 2.0 | (1-4) | 10.0 | (2-18) |
| Duration of event, median, days (range) | 3.0 | (1-7) | 3.5 | (2-5) |

CI, confidence interval; CR, complete response; CRR, complete response rate; CRS, cytokine release syndrome; DOR, duration of response; ORR, overall response rate; OS, overall survival; PFS, progression-free survival; PR, partial response; SE, standard error; VGPR, very good partial response; VGPRR, very good partial response rate.
¹Clopper-Pearson confidence interval.

Cytokine release syndrome (graded by Lee Criteria 2014) was low grade. Grade 1 CRS occurred in 14 subjects (45.2%), Grade 2 in 4 subjects (12.9%), and ≥Grade 3 in 0 subject. Median duration of cytokine release syndrome was 3 (range 1-7) days. Moreover, investigator identified neurotoxicity (iiNT) occurred in 2 subjects (6.5%). One subject had a Grade 1 iiNT (3.2%) while the other had a Grade 3 iiNT (3.2%). Grade 3 iiNT signs and symptoms were aphasia, bradykinesia, abnormal reflexes, and tremor. Median duration of neurotoxicity was 3.5 (range 2-5) days.

Additionally, Grade 3 parkinsonism (signs and symptoms included dysphasia, parkinsonian-like reflexes, bradykinesia and tremor) was reported in 1 subject (3.2%) at day 22 post-anti-BCMA CAR-T cell infusion. For this subject, AE of neurotoxicity (Grade 3) was reported Day 18-22, and was resolved with sequela. Parkinsonism (Grade 3), bradykinesia (Grade 3), and tremor (Grade 2) were reported on day 22. Parkinsonism recovered to grade 1 and bradykinesia resolved on day 120, and tremor was still ongoing at time of analysis. The subject did not have a high tumor burden. Moreover, the subject had achieved sCR as reported at 750 days post-infusion.

Table E3 details the incidence, kinetics, and management of CRS and investigator-identified neurotoxicity (iiNT). CRS and neurotoxicity were managed with tocilizumab and glucocorticoid.

TABLE E3
Incidence, Kinetics, and Management of CRS and iiNT

| (N = 31) | CRS | | iiNT | |
|---|---|---|---|---|
| Patients with ≥1 event of any grade, n | 18 | (58.1) | 2 | (6.5) |
| (%) | | | | |
| Patients with ≥1 grade 3/4 event, n (%) | 0 | | 1 | (3.2) |
| Time to first onset, median, days (range) | 2.0 | (1-4) | 10.0 | (2-18) |
| Duration of event, median, days (range) | 3.0 | (1-7) | 3.5 | (2-5) |
| Tocilizumab use, n (%) | 12 | (38.7) | 0 | |
| 1 dose | 9 | (29.0) | 0 | |
| >1 dose | 3 | (9.7) | 0 | |
| Glucocorticoid use, n (%) | 4 | (12.9) | 1 | (3.2) |

Grade 3-4 adverse events (AEs) on or after infusion of CAR-expressing T cell compositions containing autologous T cells expressing a CAR specific for BCMA were reported in 29 subjects (93.5%), most commonly, neutropenia in 25 (80.6%) subjects, leukopenia in 9 (29.0%), and anemia in 7 (22.6%). No Grade 5 AEs were reported.

For Grade ≥3 thrombocytopenia and neutropenia occurring within 1 month of anti-BCMA CAR-T cell infusion, and not resolved at month 1, recovery was defined as attaining laboratory results ≤grade 2. Median time to recovery for Grade ≥3 neutropenia subjects was 1.64 months (95% CI 1.38-NE), n=6 (19.4%). Median time to recovery for Grade ≥3 thrombocytopenia subjects was 3.71 months (95% CI NE-NE), n=1 (3.2%)

Common adverse events on or after anti-BCMA CAR-T cell infusion at the earlier time of analysis are shown in Table E4A. Grade 1 AEs are mild; Grade 2 AEs are moderate; Grade 3 AEs are severe; Grade 4 AEs are life-threatening; and Grade 5 AEs result in death. No grade 5 adverse events (AEs) were reported. 94% of patients experienced a Grade 3 or 4 adverse event. Grade 3/4 infections included pneumonia (n=1, 3.2%) human ehrlichiosis (n=1, 3.2%), viral pneumonia (n=1, 3.2%) and upper respiratory infection (n=1, 3.2%). Notably, despite a high incidence of Grade 3 or 4 neutropenia, there were few grade 3 or 4 infections. For grade ≥3 neutropenia and thrombocytopenia occurring within 1 month of anti-BCMA CAR T cell infusion and not resolved at month 1, recovery was defined as attaining laboratory results ≤grade 2 (time to recovery was calculated by recovery/censoring date−infusion date+1). Specifically, for these subjects, there were 6 subjects (19.4%) whose median time to recovery for grade ≥3 neutropenia was 1.6 months (95% CI, 1.4-NE) and 1 subject (3.2%) whose median time to recovery for grade ≥3 thrombocytopenia was 3.7 months (95% CI, NE-NE).

TABLE E4A
Common Adverse Events On or After
Anti-BCMA CAR-T Cell Infusion

| | Any Grade, | | Grade 3/4, | |
|---|---|---|---|---|
| Adverse Event (N = 31) | n (%) | | n (%) | |
| Any AE | 31 | (100) | 29 | (93.5) |
| Hematologic AEs | 28 | (90.3) | 28 | (90.3) |
| Neutropenia | 25 | (80.6) | 25 | (80.6) |
| Anemia | 10 | (32.3) | 7 | (22.6) |
| Leukopenia | 9 | (29.0) | 9 | (29.0) |
| Thrombocytopenia | 8 | (25.8) | 4 | (12.9) |

Nonhematologic AEs

| Metabolic disorders | 23 | (74.2) | 9 | (29.0) |
|---|---|---|---|---|
| Hypophosphatemia | 14 | (45.2) | 6 | (19.4) |
| Hypokalemia | 5 | (16.1) | 0 | |
| Gastrointestinal disorders | 23 | (74.2) | 1 | (3.2) |
| Diarrhea | 11 | (35.5) | 1 | (3.2) |
| Nausea | 10 | (32.3) | 0 | |
| Constipation | 8 | (25.8) | 0 | |
| General disorders and administration | 22 | (71.0) | 0 | |
| site conditions | | | | |
| Fatigue | 10 | (32.3) | 0 | |
| Pyrexia | 8 | (25.8) | 0 | |
| Chills | 7 | (22.6) | 0 | |
| Infections and infestation | 18 | (58.1) | 4 | (12.9) |
| Upper respiratory tract infections | 9 | (29.0) | 1 | (3.2) |
| Immune system disorders | 19 | (61.3) | 2 | (6.5) |
| Cytokine release syndrome | 18 | (58.1) | 0 | |
| Respiratory, thoracic, and mediastinal | 15 | (48.8) | 0 | |
| disorders | | | | |
| Cough | 11 | (35.5) | 0 | |
| Nervous system disorders | 13 | (41.9) | 1 | (3.2) |
| Headache | 8 | (25.8) | 0 | |
| Dizziness | 7 | (22.6) | 0 | |
| Vascular disorders | 11 | (35.5) | 2 | (6.5) |
| Hypertension | 7 | (22.6) | 1 | (3.2) |

Common adverse events on or after anti-BCMA CAR-T cell infusion at the subsequent time of analysis are shown in Table E4B. Safety results were generally consistent with those observed at the earlier time of analysis. Specifically, any-grade CRS and iiNT were unchanged from the earlier time of analysis. All subjects experienced ≥1 any-grade adverse event (AE). The most common any-grade AEs were hematologic, including neutropenia (80.6%), anemia (32.3%), leukopenia (29.0%), and thrombocytopenia (29.0%). Any-grade infection and infestation AEs occurred in 64.5% of subjects. A total of 29 (93.5%) patients experienced grade 3/4 AEs. The most common grade 3/4 AEs were hematologic, including neutropenia (80.6%), leukopenia (29.0%), and anemia (22.6%). Grade 3/4 infection and infestation AEs occurred 12.9% of subjects. No grade 5 AEs were reported. There were no new reports of cytokine release syndrome (CRS) and investigator-identified neurotoxicity (iiNT) since the earlier time of analysis (58.1% and 6.5%, respectively). Additionally, there were no new cases of parkinsonism reported. There was, however, one subsequent malignancy event (Bowen's disease) reported in a subject who did not receive lenalidomide after anti-BCMA CAR-T cell infusion.

TABLE E4B
Common Adverse Events On or After
Anti-BCMA CAR-T Cell Infusion

| | Any Grade, | | Grade 3/4, | |
|---|---|---|---|---|
| Adverse Event (N = 31)^{1, 2} | n (%) | | n (%) | |
| Any AE | 31 | (100) | 29 | (93.5) |
| Hematologic AEs | 28 | (90.3) | 28 | (90.3) |
| Neutropenia | 25 | (80.6) | 25 | (80.6) |
| Anemia | 10 | (32.3) | 7 | (22.6) |
| Leukopenia | 9 | (29.0) | 9 | (29.0) |
| Thrombocytopenia | 9 | (29.0) | 4 | (12.9) |

Nonhematologic AEs

| Metabolic disorders | 23 | (74.2) | 9 | (29.0) |
|---|---|---|---|---|
| Hypophosphatemia | 14 | (45.2) | 6 | (19.4) |
| Gastrointestinal disorders | 23 | (74.2) | 1 | (3.2) |
| Diarrhea | 11 | (35.5) | 1 | (3.2) |
| Nausea | 10 | (32.3) | 0 | |
| Constipation | 9 | (29.0) | 0 | |
| General disorders and administration | 24 | (77.4) | 0 | |
| site conditions | | | | |
| Fatigue | 10 | (32.3) | 0 | |
| Pyrexia | 10 | (32.3) | 0 | |
| Chills | 8 | (25.8) | 0 | |
| Infections and infestation | 20 | (64.5) | 4 | (12.9) |
| Upper respiratory tract infections | 8 | (25.8) | 0 | |
| Immune system disorders | 19 | (61.3) | 0 | |
| Cytokine release syndrome | 18 | (58.1) | 0 | |
| Respiratory, thoracic, and mediastinal | 17 | (54.8) | | |
| disorders | | | | |
| Cough | 11 | (35.5) | 0 | |
| Nervous system disorders | 16 | (51.6) | 1 | (3.2) |
| Headache | 10 | (32.3) | 0 | |
| Dizziness | 7 | (22.6) | 0 | |
| Vascular disorders | 11 | (35.5) | 2 | (6.5) |

| | | | |
|---|---|---|---|
| Hypertension | 7 | (22.6) | 0 |

¹AEs occurring in <20% of patients.
²Coded using Medical Dictionary for Regulatory Activities (MedDRA) version 26.0. A patient is counted only once for multiple events within preferred term/system organ class.

Specifically, in the 8 subjects who received lenalidomide maintenance, AEs of special interest are presented in Table E4C. Grade 3/4 AEs occurred in 5/8 (62.5%) subjects after initiation of lenalidomide. Eight (100.0%) subjects experienced grade 3/4 neutropenia after anti-BCMA CAR-T cell infusion and prior to initiation of lenalidomide; 4 (50.0%) experienced grade 3/4 neutropenia after initiation of lenalidomide. No grade 3/4 thrombocytopenia was reported post-anti-BCMA CAR-T cell infusion or during lenalidomide maintenance.

TABLE E4C
Adverse Events of Special Interest in Subjects who Received Lenalidomide Maintenance

| | | | After anti-BCMA CAR-T | | After | |
|---|---|---|---|---|---|---|
| | No lenalidomide | | cell infusion and before | | lenalidomide | |
| | maintenance | | lenalidomide maintenance | | maintenance | |
| AE,¹ | (n = 23) | | (n = 8) | | (n = 8) | |
| n (%) | Any grade | Grade 3/4 | Any grade | Grade 3/4 | Any grade | Grade 3/4 |
| Any AE | 23 (100.0) | 21 (91.3) | 8 (100.0) | 8 (100.0) | 6 (75.0) | 5 (62.5) |
| Hematologic AEs | 20 (87.0) | 20 (87.0) | 8 (100.0) | 8 (100.0) | 4 (50.0) | 4 (50.0) |
| Neutropenia | 17 (73.9) | 17 (73.9) | 8 (100.0) | 8 (100.0) | 4 (50.0) | 4 (50.0) |
| Thrombocytopenia | 6 (26.1) | 4 (17.4) | 2 (25.0) | 0 | 1 (12.5) | 0 |
| Leukopenia | 5 (21.7) | 5 (21.7) | 4 (50.0) | 4 (50.0) | 0 | 0 |
| Anemia | 9 (39.1) | 7 (30.4) | 1 (12.5) | 0 | 0 | 0 |
| Other AEs² | | | | | | |
| Infections | 14 (60.9) | 3 (13.0) | 5 (62.5) | 0 | 3 (37.5) | 1 (12.5)³ |
| CRS | 13 (56.5) | 0 | 5 (62.5) | 0 | 0 | 0 |

¹Coded using MedDRA version 26.0. A patient is counted only once for multiple events within preferred term/system organ class;
²CRS is both the system organ class and preferred term; Infections is the system organ class;
³Pneumonia haemophilus, Metapneumovirus pneumonia, and pneumonia viral more than 1 year after initiation of lenalidomide.

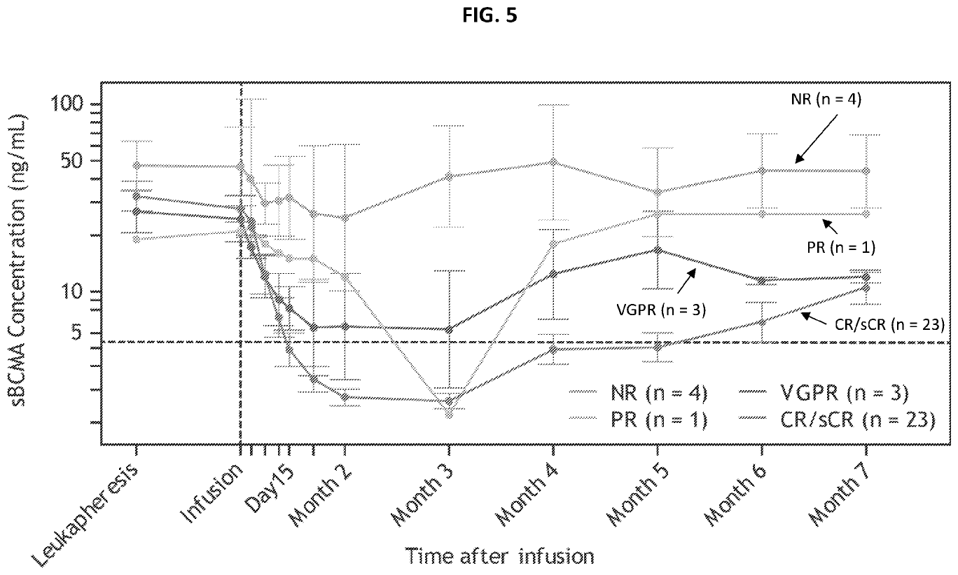

Soluble BCMA was assessed in evaluable subjects (e.g., by a Luminex assay). Serum level of sBCMA was analyzed at day 1 as a measure of tumor burden. sBCMA was cleared (defined as below the limit of detection of the assay, which is 4.4 mg/mL) within 2 months post anti-BCMA CAR-T cell infusion in 24/31 (77.4%) subjects, including in 22 who had ≥CR. (FIG. 5). For subjects who had BOR≥CR, sBCMA was cleared within 2 months in 22 of 23 (96%).

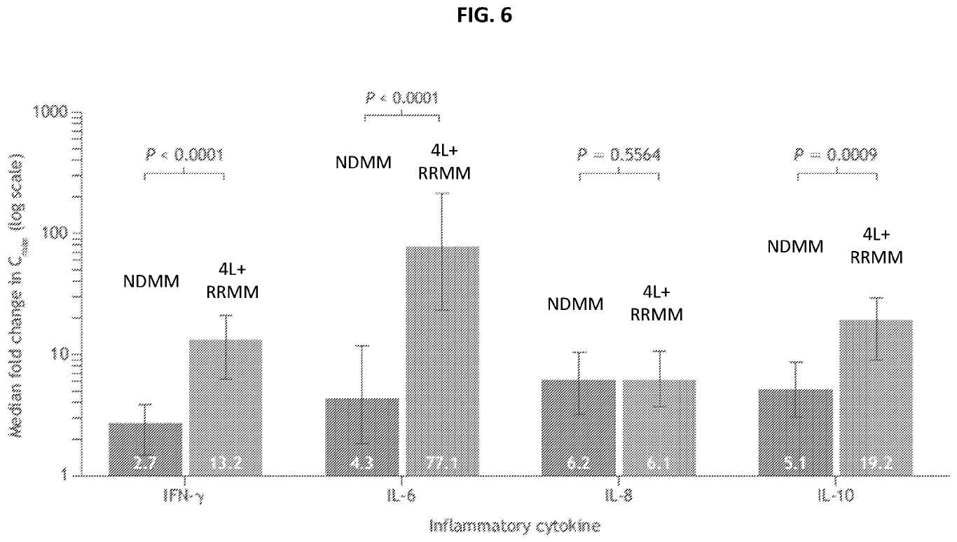

Lower levels of inflammatory cytokines were seen in the NDMM subjects in this study when compared to relapsed and refractory multiple myeloma (R/R MM) subjects from another study who received at least three previous lines of prior therapy and received the anti-BCMA CAR T cell infusion as a fourth line or beyond (4L+) therapy (FIG. 6). Specifically, for the NDMM subjects in this study, induction of inflammatory cytokines was observed with a median fold change from baseline to peak serum concentration (Cₘₐₓ) of 2.7 for IFN-γ, 4.3 for IL-6, 6.2 for IL-8 and 5.1 for IL-10. These median fold changes from baseline to Cₘₐₓ, with the exception of IL-8, were all lower than those from R/R MM subjects who received the anti-BCMA CAR T cell infusion as a 4L+ therapy.

At the earlier time of analysis, robust cell expansion was observed in 31 evaluable subjects (depicted in Table E5A) despite low tumor burden, as assessed by sBCMA levels (range 6.5-154.0 ng/mL at infusion, n=30). Anti-BCMA CAR-T cell expansion was higher in subjects who achieved ≥CR vs those who did not (Table E5A). Anti-BCMA CAR-T cell showed durable persistence in blood, with a median Tₗₐₛₜ (time of last measurable transgene level) of 169 days (range: 27 to 841 days) in all evaluable subjects.

Although high intersubject variability was observed at all doses, upper quartiles of exposure (area under the curve of the transgene level from time of dose to 28 days [AUC₀₋₂₈ days]) were observed more frequently at higher doses as shown in Table E5A.

TABLE E5A
Summary of pharmacokinetic parameters.¹

| Pharmacokinetic | Evaluable subjects | | Evaluable subjects | | Evaluable subjects | |
|---|---|---|---|---|---|---|
| parameter² | (n = 31) | | with ≥CR (n = 23) | | with <CR (n = 8) | |
| Cₘₐₓ, copies/μg (% | 233,417 | (115) | 328,671 | (80) | 87,262 | (88) |
| geometric CV)³ | | | | | | |
| AUC_{0-28 days}, | 2,910,792 | (127) | 4,217,970 | (84) | 1,049,561 | (104) |
| days*copies/μg (% | | | | | | |
| geometric CV)³ | | | | | | |
| Tₘₐₓ, median, days | 11 | (7-27) | 11 | (7-21) | 9 | (7-27) |

(range)

AUC_{0-28 days}, area under the curve of the transgene level from time of dose to 28 days post-infusion; Cₘₐₓ, peak transgene level; CV, coefficient of variation; Tₘₐₓ, time to peak transgene level.
¹At earlier data cutoff date.
²The lower limit of quantification for a qPCR assay is 75 copies/μg; derived using the transgene levels quantified by the qPCR assay in CD3+ sorted T cells.
³Data are presented as geometric mean.

At the subsequent time of analysis, robust cell expansion was again observed in 31 evaluable subjects (depicted in Table E5B). Anti-BCMA CAR-T cell expansion was higher in subjects who achieved ≥CR vs those who did not (Table E5A). Anti-BCMA CAR-T cell showed durable persistence in blood, with a median Tₗₐₛₜ (time of last measurable transgene level) of 254 days (range: 28 to 1345 days) in all evaluable subjects.

TABLE E5B
Summary of pharmacokinetic parameters.¹

| Pharmacokinetic | Evaluable subjects | | Evaluable subjects | | Evaluable subjects | |
|---|---|---|---|---|---|---|
| parameter¹ | (n = 31) | | with ≥CR (n = 24) | | with <CR (n = 7) | |
| Cₘₐₓ, copies/μg (% | 93,686 | (229) | 148,992 | (158) | 19,092 | (84) |
| geometric CV)² | | | | | | |
| AUC_{0-28 days}, | 721,017 | (197) | 1,127,157 | (137) | 155,836 | (65) |
| days*copies/μg (% | | | | | | |
| geometric CV)² | | | | | | |
| Tₘₐₓ, median, days | 9 | (7-21) | 9 | (7-21) | 11 | (7-14) |

(range)

AUC_{0-28 days}, area under the curve of the transgene level from time of dose to 28 days post-infusion; Cₘₐₓ, peak transgene level; CV, coefficient of variation; Tₘₐₓ, time to peak transgene level.
¹At later data cutoff date.
²Data are presented as geometric mean. Cₘₐₓ, peak serum concentration; C_{V}, coefficient of variation.

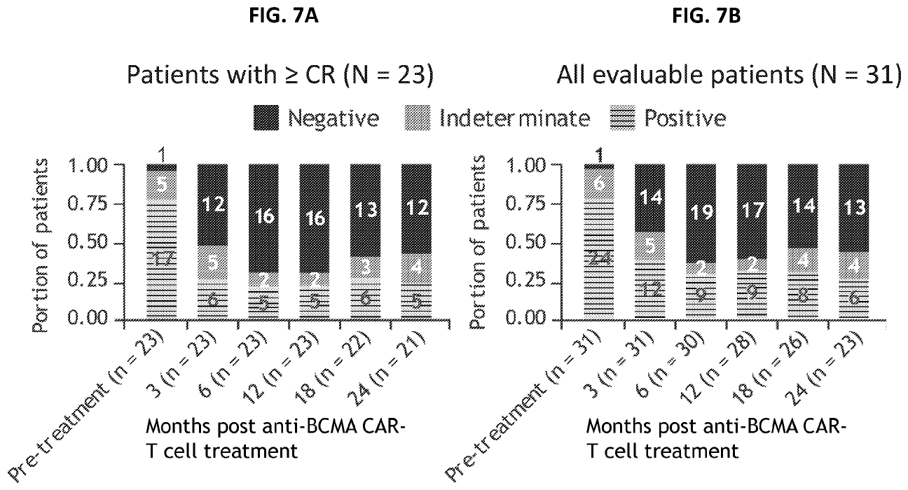

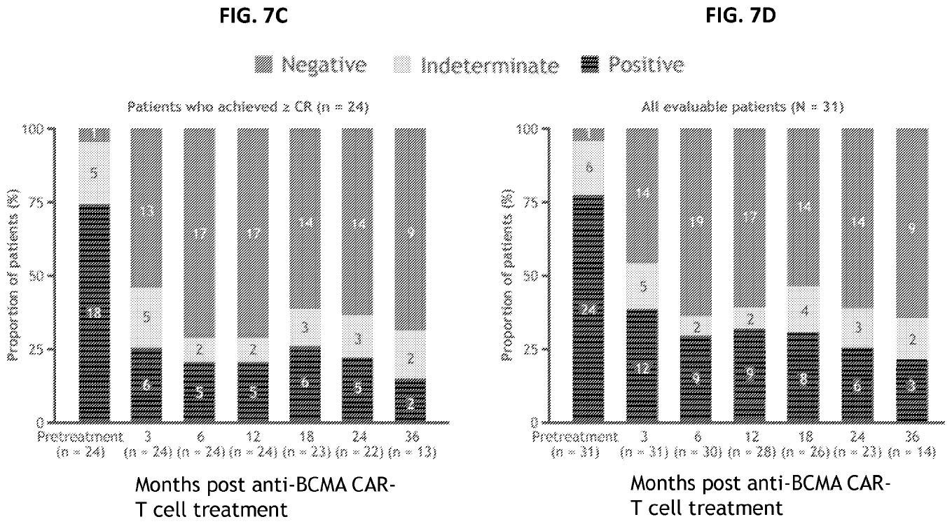

At the earlier time of analysis, among 23 subjects with ≥CR, 16 (69.6%; 95% CI, 49.1-84.4) subjects had MRD− at 6 months post-anti-BCMA CAR-T cell infusion (FIG. 7A). At 12 months, MRD− was observed in 17/28 (60.7%; 95% CI 42.4-76.4) evaluable subjects (FIG. 7B), regardless of CR status and in 16/23 (69.6%; 95% CI 49.1-84.4) evaluable subjects who had ≥CR. Specifically, at 12 months, MRD− was observed in 16/23 subjects with a complete response or better. Of note, of the 16 subjects who had MRD− at 6 months post-anti-BCMA CAR-T cell infusion, 11 sustained MRD− at 24 months; 2 subjects did not have 24 months data available, and data for 3 subjects were indeterminate.

At the subsequent time of analysis, among the 14 subjects evaluable at 36 months, 9 had MRD-negative status (FIG. 7C). Specifically, 9 subjects achieved ≥CR (FIG. 7D). At the subsequent time of analysis, taking into account 1 subject had improved from BVPR to sCR since the earlier time of analysis, of the 24 subjects (77.4%) who achieved ≥CR, MRD-negativity was maintained at 12 months in 17/24 (70.8%; 95% CI, 50.8-85.1), at 24 months in 14/22 (63.6%; 95% CI, 43.0-80.3), and at 36 months in 9/13 (69.2%; 95% CI, 42.4-87.3) evaluable subjects.

Additionally, at the subsequent time of analysis, for the 8 subjects who received lenalidomide maintenance therapy, MRD-negativity was maintained at 12 months in 4/6 (66.7%; 95% CI, 30.0-90.3), at 24 months in 4/6 (66.7%; 95% CI, 30.0-90.3) and at 36 months in 2/3 (66.7%; 95% CI, 20.8-93.9) evaluable subjects. Four subjects remained MRD-positive but progression events have not been observed.

The treatment with the anti-BCMA CAR-T cells demonstrated frequent, deep, and durable responses in subjects who experienced an inadequate response to frontline therapy with ASCT. Early deep clearance of tumor was observed in subjects with ≥CR after anti-BCMA CAR-T cell treatment and was sustained at the two year timepoint.

Lower incidence of CRS, thrombocytopenia and NT were seen in these subjects versus those subjects who were treated with anti-BCMA CAR-T cell treatment in later lines of therapy. No high-grade CRS was reported. Finally, despite experiencing a high incidence of grade 3/4 neutropenia, subjects had a low incidence of grade 3/4 infections.

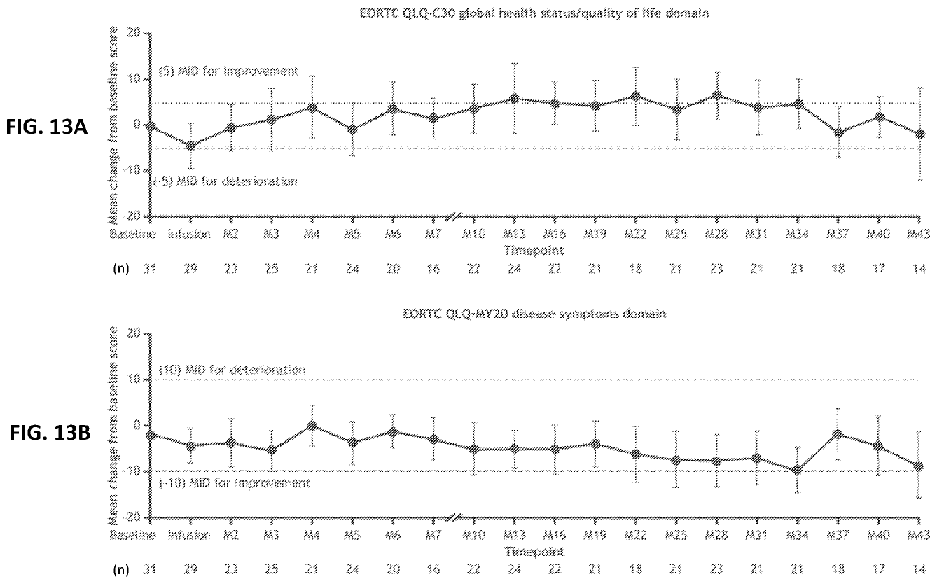

Subject-reported outcomes on health-related quality of life (HRQOL) are shown in FIGS. 13A-13B. Based on EORTC QLQ-C30 global health status/quality of life (QoL) domain, as shown in FIG. 13A, HRQOL improved following anti-BCMA CAR-T cell treatment. Specifically, by month 3, >75% of subjects had improved or stable global health status/QoL. Based on EORTC QLQ-MY20, as shown in FIG. 13B, it was demonstrated that MM disease symptoms were stable over time. In particular, by month 3, >75% of subjects had improved or stable MM disease symptoms (including pain in different locations of the body). Most subjects had stable or improved HRQOL and disease symptoms after anti-BCMA CAR-T cell treatment.

Taken together, the results support a favorable clinical risk benefit profile of anti-BCMA CAR-T cell treatment post-ASCT in NDMM subjects or subjects with a suboptimal response to ASCT. At the subsequent time of analysis, subjects with NDMM who had an inadequate response to ASCT continued to demonstrate a favorable risk profile following anti-BCMA CAR-T cell treatment. Anti-BCMA CAR-T cell treatment deepened responses in subjects with an inadequate response to frontline ASCT. All treated subjects remained alive and no new safety signals were observed at the time of subsequent analysis (e.g., during extended follow-up).

### Example 2: Treatment with Anti-BCMA CAR-T Cells in Patients with Clinical High-Risk Multiple Myeloma Due to Early Relapse to Frontline Autologous Stem Cell Transplantation

Chimeric antigen-receptor (CAR)-expressing T cell compositions containing autologous T cells expressing a CAR specific for B-cell maturation antigen (BCMA) were administered to human subjects with multiple myeloma (MM) who had an early relapse after frontline therapy with autologous stem cell transplant (ASCT). Subjects with early relapse had early relapse within 18 months of frontline treatment initiation, including induction, ASCT, and lenalidomide maintenance.

The administered T cell compositions were generated by obtaining peripheral-blood mononuclear cells (PBMCs) from leukapheresis samples from individual subjects to be treated, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8α, and a CD137 (4-1BB) co-stimulatory domain followed by the intracellular signaling domain of a CD3ζ chain. The polynucleotide sequence encoding the exemplary BCMA CAR is set forth in SEQ ID NO: 214, and the polypeptide sequence of the exemplary BCMA CAR is set forth in SEQ ID NO: 116.

### A. Eligible Subjects and Treatment

Eligible subjects were adult (≥18 years of age) human subjects who had received a frontline treatment containing induction (≥3 cycles of induction therapy, including a proteasome inhibitor, immunomodulatory agent, and dexamethasone), ASCT (single or tandem) and lenalidomide-containing maintenance, but who exhibited early relapse defined as progressive disease (PD)<18 months from starting the frontline therapy. Subjects had Eastern Cooperative Oncology Group (ECOG) performance status (PS)≤1. Additionally, subjects had measurable disease. Subjects had one prior anti-myeloma treatment regimen. Induction with or without hematopoietic stem cell transplant (HSCT) and with or without maintenance therapy is considered a single regimen.

Only 24.3% of subjects experienced a deep response (≥CR) to first-line therapy, including ASCT. A relatively high proportion of subjects had high-risk or ultra-high-risk cytogenetics.

Eligible subjects received a lymphodepleting chemotherapy (LDC) with fludarabine (flu, 30 mg/m²/day) and cyclophosphamide (cy, 300 mg/m²/day) for 3 consecutive days. Subjects received a single infusion of anti-BCMA CAR-T cells at a dose range of 150-450×10⁶ CAR+ T cells, up to an additional 20%. The median dose of infused CAR+ T cells was 425.0×10⁶ CAR+ T cells (range 300.2 to 525.6×10⁶ CAR+ T cells). Some subjects also received a bridging therapy (corticosteroids, alkylating agents, immunomodulatory agents, proteasome inhibitors (PI), and/or anti-CD38 antibodies) before CAR-T cell infusion.

The post-treatment period included (1) following the subjects for a minimum of 24 months post-infusion or until PD, whichever was longer, and (2) continuing post-treatment follow-up discontinuation (PTFD) visit. Survival follow-up occurred every 3 months, up to 5 years, after the last subject received the first anti-BCMA CAR-T cell infusion.

Baseline demographics and clinical characteristics of this group of subjects are depicted in Table E6. High tumor burden was determined by bone marrow biopsy CD138+ plasma cell. R-ISS was calculated using baseline values of albumin and beta-2-microglobulin; R-ISS was derived using baseline ISS stage, cytogenetic abnormality, and serum lactate dehydrogenase. Ultra-high-risk cytogenetics was defined as two or more high-risk cytogenetic features: del (17p), t(4; 14), t(14; 16), t(14; 20), 1q amp. Double refractory is defined as refractory to at least one immunomodulatory agent and one proteasome inhibitor; triple refractory is defined as refractory to at least one immunomodulatory agent, one proteasome inhibitor, and one CD38.

TABLE E6
Baseline Demographics and Clinical Characteristics

| Characteristics | All (N = 37) | |
|---|---|---|
| Median age (range), years | 57.0 | (36.0-77.0) |
| Male sex (%) | 56.8 | |
| Race, n (%) | | |
| Asian | 29 | (78.4) |
| Black or African American | 4 | (10.8) |
| White | 2 | (5.4) |
| Unknown | 2 | (5.4) |
| Median time from initial diagnosis to | 1.6 | (1.3-1.8) |
| screening (range), years | | |
| Extramedullary disease, n (%) | 3 | (8.1) |
| High tumor burden, n (%) | 7 | (18.9) |
| ECOG PS (0/1), % | 62.2/37.8 | |
| R-ISS Stage, n (%)¹ | | |
| I | 5 | (13.5) |
| II | 19 | (51.4) |
| III | 2 | (5.4) |
| High Risk Cytogenetics, n (%) | | |
| Missing | 15 | (40.5) |
| Any | 12 | (32.4) |
| del (17p) | 7 | (18.9) |
| t (4; 14) | 6 | (16.2) |
| t (14; 16) | 1 | (2.7) |
| Ultra high-risk cytogenetics, n (%) | 5 | (13.5) |
| LEN post-ASCT maintenance therapy, n (%)² | 30 | (81.1) |
| BOR to first-line therapy (including | | |
| ASCT), n (%) | | |
| CR/sCR | 9 | (24.3) |
| VGPR | 19 | (51.4) |
| PR | 8 | (21.6) |
| PD | 1 | (2.7) |
| PD <12 months of initiation of first-line | 11 | (29.7) |
| therapy, n (%) | | |
| PD <12 months of ASCT, n (%) | 33 | (89.2) |
| Refractory status, n (%) | | |
| Immunomodulatory agent | 32 | (86.5) |
| Proteasome inhibitor | 33 | (89.2) |

| | |
|---|---|
| Anti-CD38 mAb | 0 |
| Double/triple refractory | 32 (86.5)/0 |

¹Represents stage at study entry.
²Two subjects received lenalidomide consolidation, two subjects received bortezomib maintenance only, and three subjects had PD shortly after ASCT.

The primary efficacy endpoint was complete response (CR) rate (CRR; CR and stringent CR) per International Myeloma Working Group (IMWG) Uniform Response Criteria. Secondary endpoints included overall response rate (ORR; ≥partial response), very good partial response (VGPR) rate (VGPRR), time to response (TTR), duration of response (DOR), progression-free survival (PFS), overall survival (OS), time to progression (TTP), safety, pharmacokinetics (PK) immunogenicity (anti-CAR antibody response), and health-related quality of life (HRQOL). Exploratory endpoints included assessment of soluble BCMA (sBCMA) level and minimal residual disease negativity (MRD−) by next-generation sequencing and EuroFlow (<10⁻⁵ nucleated cells) data with interpolation to reduce missing/indeterminate data points. Efficacy and safety were analyzed in all subjects who received CAR-T cells. PK, sBCMA, and MRD were analyzed in evaluable subjects.

Anti-BCMA CAR-T cells were manufactured and infused in 37/39 subjects. One subject discontinued due to physician decision and another subject discontinued due to withdrawal of consent. The median age of the subjects was 57 years and median time since diagnosis was 1.6 years. A total of 62.2% of subjects had Eastern Cooperative Oncology Group (ECOG) PS 0, and 70.3% of subjects (26 subjects) received bridging therapy for MM. Bridging therapy was allowed during manufacturing but stopped at least 14 days before lymphodepletion and restricted to certain drug classes or drugs previously received. Median duration of bridging therapy was 15.0 days (range 11.0-20.0 days).

Of subjects who were eligible at study entry, 13.5% of subjects had revised International Staging System (R-ISS) stage 1, 51.4% of subjects had R-ISS stage II and 5.4% of subjects had R-ISS stage III disease; 32.4% had high-risk cytogenetics; 18.9% had bone marrow biopsy-determined high tumor burden (≥50% bone marrow CD138+ plasma cells); and 8.1% had extramedullary disease. Most subjects had disease refractory to an immunomodulatory agent (86.5%) or proteasome inhibitor (PI; 89.2%), with 86.5% exhibiting double refractory disease.

15 infused subjects discontinued the study. Of the 15 subjects, the reason for discontinuation included death (4 subjects; 10.8%), withdrawal by subject (9 subjects; 24.3%); or physician decision (2 subjects, 5.4%). For the 22 evaluable subjects, 12 had high-risk cytogenetics at baseline; 5 subjects also had ultra-high risk cytogenetics.

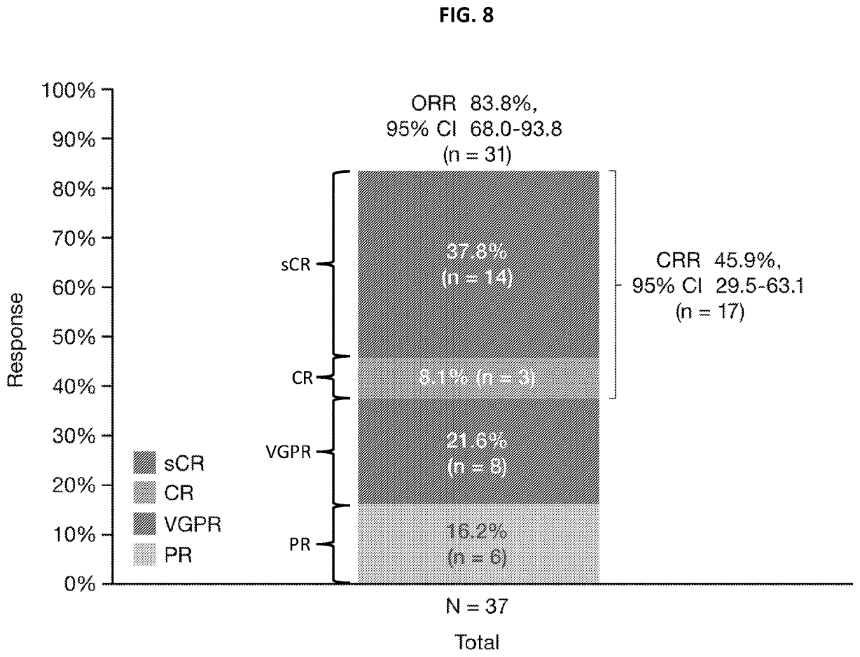

At time of data analysis, the median follow-up was 21.5 months (range 2-31 months). 73% of subjects had a follow-up >12 months. Only 24.3% of subjects experienced a deep response (≥CR). CRR was 45.9% (95% CI 29.5-63.1; P<0.0001) for subjects with sCR or CR, and ORR was 83.8% (95% CI 68.0-93.8) for subjects with PR or better (two subjects had minimal response, four had stable disease and none had PD), as shown by FIG. 8 and Table E7.

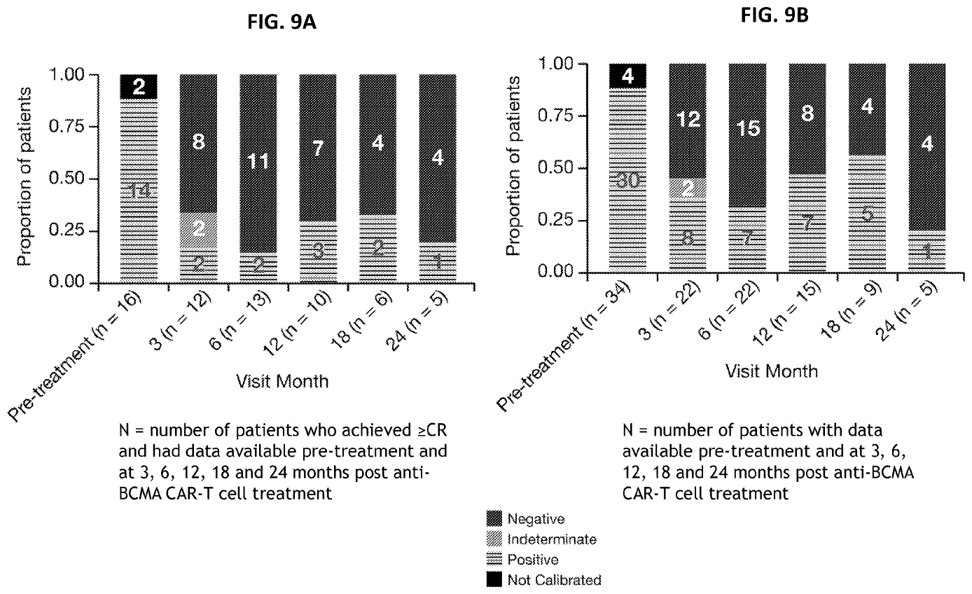

At 6 months post-anti-BCMA CAR-T cell infusion, MRD− was observed in 11/13 (85%; 95% CI 57.8-95.7) subjects (subjects who achieved ≥CR; FIG. 9A). 4 of 5 (80.0%) subjects who achieved VGPR were MRD− at 6 months. At 6 months post-anti-BCMA CAR-T cell infusion, MRD− was observed in 68% of all subjects (95% CI 45.1-85.3; FIG. 9B).

At 12 months post-anti-BCMA CAR-T cell infusion, MRD− was observed in 7/10 (70%; 95% CI 39.7-89.2) subjects (these subjects achieved ≥CR; FIG. 9A). Of the 7 subjects, 1 had PD at 20.8 months, 5 sustained MRD− at ≥18 months, and 1 subject had unavailable >12 month data. Median TTR was 1 month (range 0.9-2.9) for subjects with PR or better based on IMWG criteria by investigator assessment, measured from infusion. At 12 months post-anti-BCMA CAR-T cell infusion, MRD− was observed in 53% of all subjects (95% CI 27.4-77.7) subjects (subjects who achieved ≥CR; FIG. 9B).

One year after anti-BCMA CAR-T cell treatment, an estimated 88% of patients were alive and 48% had no disease or death. Two years after anti-BCMA CAR-T cell treatment, an estimated 85% of patients were alive and 26% had no disease progression or death.

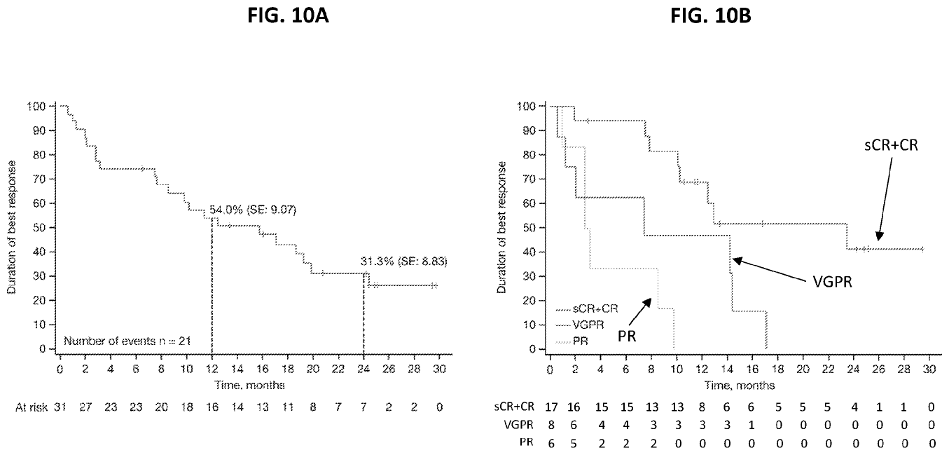

Median duration of best response (for responder subjects only, based on IMWG criteria by investigator assessment) was 15.7 months (95% CI 7.62-19.81; FIG. 10A). Median duration of best response in subjects who achieved ≥CR was 23.5 months (95% CI: 10.2-Not Evaluable; FIG. 10B). Median DOR in subjects who achieved VGPR was 7.5 months (95% CI: 0.6-14.4; FIG. 10B). Median DOR in subjects who had PR was 3 months (95% CI: 1.0-Not Evaluable; FIG. 10B).

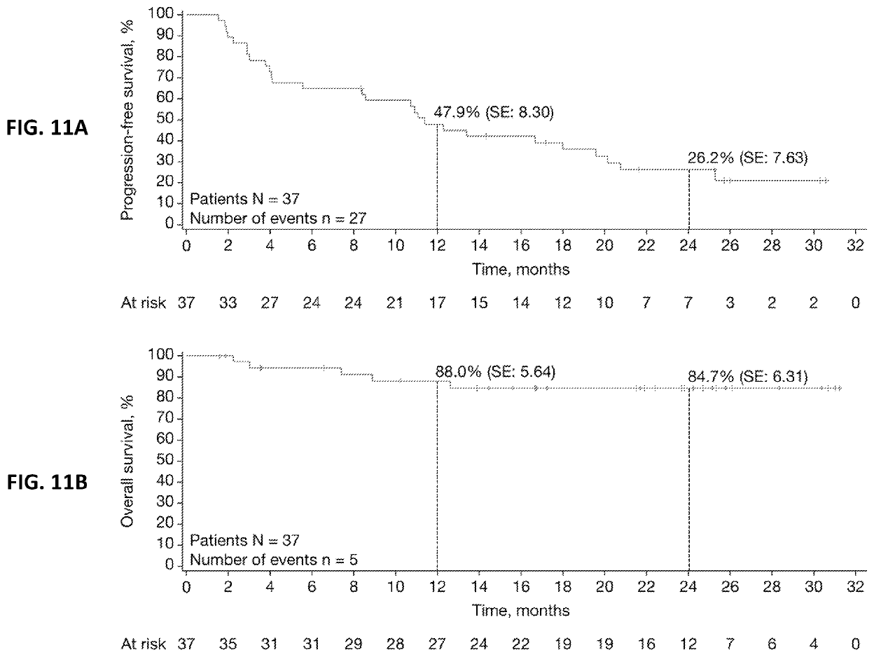

Median PFS was 11.4 months (95% CI 5.55-19.58; FIG. 11A). Progression-free survival rate at 1 year was 47.9% while progression-free survival rate at 2 years was 26.2%. Median OS was not reached (FIG. 11B). Duration of disease control was longer with anti-BCMA CAR-T cells than on first-line treatment as indicated by 12-month PFS rate. An OS event-free rate of 84.7% (SE: 6.31) was observed at 24 months (FIG. 11B). Time to event endpoint results for DOR, PFS, and OS are shown in Table E7.

Grade 1/2 cytokine release syndrome (CRS) occurred in 30 subjects (81.1%); 1 subject (2.7%) had a Grade 3 event (Table E7). Median duration of cytokine release syndrome was 3 (range 1-11) days. CRS events were primarily Grade 1/2, with only one Grade ≥3 event. CRS was primarily managed with tocilizumab and glucocorticoids; one subject received anakinra.

Grades 1 and 2 investigator-identified neurotoxicity (NT) occurred in 8 subjects (21.6%). No subject had a ≥Gr 3 NT and no late iiNTs were reported. Glucocorticoids were used in 3 subjects with iiNT; no other agents were administered. Median duration of neurotoxicity was 3.5 (range 2-7) days.

TABLE E7
Efficacy and safety outcomes following anti-BCMA CAR-T cell infusions

| | |
|---|---|
| Efficacy outcome | N = 37 |

Best overall response

| ORR, n | 31 | |
|---|---|---|
| ORR, % (95% CI) | 83.8 | (68.0-93.8) |
| CRR, n | 17 | |
| CRR, % (95% CI) | 45.9 | (29.5-63.1) |
| Stringent CR, n (%) | 14 | (37.8) |
| CR, n (%) | 3 | (8.1) |
| Very good PR, n (%) | 8 | (21.6) |
| PR, n (%) | 6 | (16.2) |
| Minimal response | 2 | (5.4) |
| Stable disease | 4 | (10.8) |

| | |
|---|---|
| Progressive disease | 0 |

Time to event rates

| | | |
|---|---|---|
| 12 mo DOR rate, % (SE) | 54.0 | (9.07) |
| 24 mo DOR rate, % (SE) | 31.3 | (8.83) |
| 12 mo PFS rate, % (SE) | 47.9 | (8.30) |
| 24 mo PFS rate, % (SE) | 26.2 | (7.63) |
| 12 mo OS rate, % (SE) | 88.0 | (5.64) |
| 24 mo OS rate, % (SE) | 84.7 | (6.31) |

| | N = 37 | | | |
|---|---|---|---|---|
| AEs of special interest | CRS | | NT | |
| Number of subjects with at least one event of any grade, n (%) | 31 | (83.8) | 8 | (21.6) |
| Number of subjects with at least one grade 3/4 event, n (%) | 1 | (2.7) | 0 | |
| Time to first onset, median, days (range) | 2.0 | (1-15) | 3.0 | (1-12) |
| Duration of event, median, days (range) | 3.0 | (1-11) | 3.5 | (2-7) |

CI, confidence interval; CR, complete response; CRR, complete response rate; CRS, cytokine release syndrome; DOR, duration of response; ORR, overall response rate; OS, overall survival; PFS, progression-free survival; PR, partial response; SE, standard error; VGPR, very good partial response; VGPRR, very good partial response rate.
¹Clopper-Pearson confidence interval.

Median time to recovery for grade ≥3 neutropenia subjects was 1.45 months (95% CI 1.45-1.87), n=6 (19.4%). Recovery was defined as attaining laboratory results ≤grade 2 neutropenia. Median time to recovery for grade ≥3 thrombocytopenia subjects was 1.87 months (95% CI 1.45-Not Evaluable). Grade ≥3 thrombocytopenia was defined as occurring within 1 month anti-BCMA CAR-T cell infusion and not resolved at month 1.

Common adverse events on or after anti-BCMA CAR-T cell infusion are shown in Table E8. Grade 1 AEs are mild; Grade 2 AEs are moderate; Grade 3 AEs are severe; Grade 4 AEs are life-threatening; and Grade 5 AEs result in death. 94% of patients experienced a Grade 3 or 4 adverse event. Grade (Gr) 3-4 adverse events (AEs) on or after anti-BCMA CAR-T cell infusion occurred in all subjects, most commonly neutropenia in 35 subjects (94.6%), anemia in 17 subjects (45.9%), and thrombocytopenia in 14 subjects (37.8%). Two subjects died due to AEs, one due to pneumonia (67 days post anti-BCMA CAR-T cell infusion) and one due to pseudomonal sepsis (91 days post-anti-BCMA CAR-T cell infusion).

TABLE E8
Common Adverse Events On or After
Anti-BCMA CAR-T Cell Infusion

| | Any Grade, | | Grade 3/4, | | Grade 5, | |
|---|---|---|---|---|---|---|
| Adverse Event (N = 31) | n (%) | | n (%) | | n (%) | |
| Any AE | 37 | (100) | 37 | (100) | 2 | (5.4) |
| Hematologic AEs | | | | | | |
| Neutropenia | 35 | (94.6) | 35 | (94.6) | 0 | |
| Anemia | 21 | (56.8) | 17 | (45.9) | 0 | |
| Thrombocytopenia | 19 | (51.4) | 14 | (37.8) | 0 | |
| Leukopenia | 12 | (32.4) | 12 | (32.4) | 0 | |
| Nonhematologic AEs | | | | | | |
| Infections and infestation | 22 | (59.5) | 8 | (21.6) | 2 | (5.4) |

| Cytokine release syndrome | 31 | (83.8) | 1 | (2.7) | 0 |
|---|---|---|---|---|---|
| Metabolism and nutrition | 26 | (70.3) | 13 | (35.1) | |
| disorders | | | | | |
| Hypophosphatemia | 14 | (37.8) | 9 | (24.3) | |
| Hypokalemia | 9 | (24.3) | 0 | | |
| Hypocalcemia | 8 | (21.6) | 2 | (5.4) | |
| Nervous system disorders | 26 | (70.3) | 3 | (8.1) | |
| Headache | 14 | (37.8) | 1 | (2.7) | |
| Gastrointestinal disorders | 26 | (70.3) | 0 | | |
| Diarrhea | 11 | (29.7) | 0 | | |
| Nausea | 9 | (24.3) | 0 | | |
| Constipation | 8 | (21.6) | 0 | | |
| General disorders and | 23 | (62.2) | 1 | (2.7) | |
| administration site conditions | | | | | |
| Pyrexia | 8 | (21.6) | 0 | | |
| Respiratory, thoracic, and | 19 | (51.4) | 3 | (8.1) | |
| mediastinal disorders | | | | | |
| Cough | 8 | (21.6) | 0 | | |
| Psychiatric disorders | 16 | (43.2) | 2 | (5.4) | |

| | | | |
|---|---|---|---|
| Insomnia | 8 | (21.6) | 0 |

Table E9 details the incidence, kinetics, and management of CRS and investigator-identified neurotoxicity (iiNT).

TABLE E9
Incidence, Kinetics, and Management of CRS and iiNT

| (N = 37) | CRS | | iiNT | |
|---|---|---|---|---|
| ≥1 grade 3/4 event | 1 | (2.7) | 0 | |
| Time to first onset, median (range), days | 2.0 | (1-15) | 3.0 | (1-12) |
| Duration of event, median (range), days | 3.0 | (1-11) | 3.5 | (2-7) |
| Tocilizumab use, n (%) | 21 | (56.8) | 0 | |
| 1 dose | 11 | (29.7) | 0 | |
| >1 dose | 10 | (27.0) | 0 | |
| Glucocorticoid use, n (%) | 6 | (16.2) | 3 | (8.1) |

Robust cell expansion was seen in 36 evaluable subjects (Table E10). Anti-BCMA CAR-T cell cellular expansion levels were higher in subjects who had achieved ≥CR versus those who had <CR with anti-BCMA CAR-T infusion (Table E10). Anti-BCMA CAR-T cells showed durable persistence in blood, with a median Tₗₐₛₜ (time of last measurable transgene level) of 168 days (range: 28 to 658 days) in all evaluable subjects.

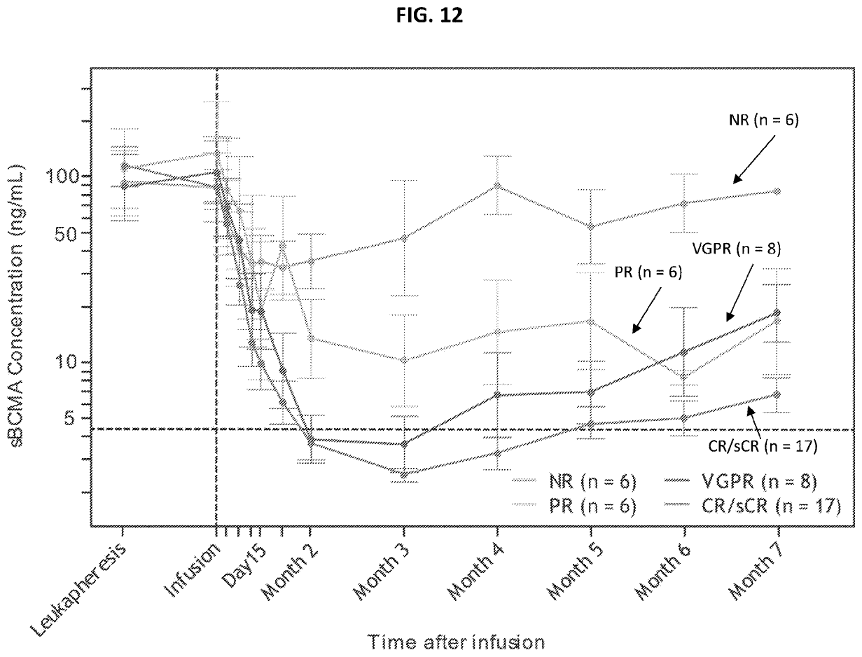

sBCMA (range 15.0-737.0 ng/mL at infusion, n=36) was cleared within 2 months post-anti-BCMA CAR-T cell infusion in 25/37 (67.6%) subjects, including all subjects who had ≥CR (FIG. 12). sBCMA was cleared, defined as below the limit of detection of the assay (4.4 mg/mL), within 2 months post-anti-BCMA CAR-T cell infusion in all subjects who achieved ≥CR (FIG. 12).

TABLE E10
Summary of pharmacokinetic parameters.¹

| Pharmacokinetic | Evaluable subjects | | Evaluable subjects | | Evaluable subjects | |
|---|---|---|---|---|---|---|
| parameter | (n = 36) | | with ≥CR (n = 16) | | with <CR (n = 20) | |
| Cₘₐₓ, copies/μg (% | 286,462 | (136) | 404,171 | (70) | 217,505 | (178) |
| geometric CV)² | | | | | | |
| AUC_{0-28 days}, | 3,360,086 | (170) | 4,745,793 | (102) | 2,549,081 | (219) |
| days*copies/μg (% | | | | | | |
| geometric CV)² | | | | | | |
| Tₘₐₓ, median, days | 11 | (7-23) | 11 | (9-17) | 11 | (7-23) |

(range)

AUC_{0-28 days}, area under the curve of the transgene level from time of dose to 28 days post-infusion; Cₘₐₓ, peak transgene level; CV, coefficient of variation; Tₘₐₓ, time to peak transgene level.
¹At earlier data cutoff date; derived using the transgene levels quantified by the qPCR assay in CD3+ sorted T cells (lower limit of quantification, 75 copies/μg.
²Data are presented as geometric mean.

The treatment with the anti-BCMA CAR-T cells demonstrated frequent, deep responses in subjects with clinical high-risk MM who experienced an early relapse within 18 months of initiating frontline therapy, including ASCT.

MRD− was sustained in a subset of subjects >18 months. Without wishing to be bound by theory, the maintenance therapy with lenalidomide after ASCT may potentiate the durability in response of the anti-BCMA CAR-T cells. The incidence of CRS and NT were numerically lower (e.g., less common) in these subjects versus those in subjects treated with anti-BCMA CAR-T cells in later lines of therapy. T cell expansion and persistence in these subjects were similar to those of subjects treated with anti-BCMA CAR-T cells in later lines of therapy.

Taken together, these results support a favorable clinical benefit-risk profile of the anti-BCMA CAR-T cell therapy in a clinically risk patient population and its potential use in earlier lines of treatment.

### Example 3: Treatment with Anti-BCMA CAR-T Cells in Patients with Newly Diagnosed Multiple Myeloma (NDMM) with Suboptimal Response to Autologous Stem Cell Transplantation

Chimeric antigen-receptor (CAR)-expressing T cell compositions containing autologous T cells expressing a CAR specific for B-cell maturation antigen (BCMA) are administered to human subjects with newly diagnosed multiple myeloma (NDMM) who have a suboptimal after frontline therapy with autologous stem cell transplant (ASCT). Subjects, prior to CAR T cell administration, receive induction therapy followed by high-dose chemotherapy and autologous stem cell transplantation (ASCT), without subsequent consolidation or maintenance.

The subjects are enrolled in a study to compare the efficacy, safety, and tolerability of the CAR T cell administration with lenalidomide (LEN) maintenance to that of LEN maintenance alone in subjects with NDMM who have achieved a suboptimal response post ASCT.

### A. Eligible Subjects and Treatment

Eligible subjects are adult (≥18 years of age) human subjects with NDMM who have received induction therapy followed by high-dose chemotherapy (HDT) and autologous stem cell transplantation (ASCT), without subsequent consolidation or maintenance. Subjects must have received 4 to 6 cycles of induction therapy, which must contain, at minimum, an immunomodulatory drugs (IMiD) and a proteasome inhibitor (with or without anti-CD38 monoclonal antibody). If a subject receives ≤7 days of lenalidomide maintenance therapy, the subject can be eligible if an investigator documents that there is no impact to the overall benefit/risk assessment due to the lenalidomide.

Eligible subjects must have a single ASCT within 80 to 120 days prior to being screened (or prior to consent) for the study. The screening for a subject typically occurs 9 to 10 weeks prior to apheresis. In particular, eligible subjects exhibit a suboptimal response to the ASCT, defined as having achieved documented partial response (PR) or very good partial response (VGPR) to the ASCT at approximately 100 days after ASCT. This response must be maintained at screening.

Subjects have an Eastern Cooperative Oncology Group (ECOG) performance status (PS)≤1. Subject with ECOG 2 (due to pain of underlying myeloma-associated bone lesions) may be eligible per investigator's discretion. Additionally, subjects must have recovered to ≤Grade 1 for any nonhematologic toxicities due to prior treatments, excluding alopecia and Grade 2 neuropathy.

The primary efficacy endpoint is progression free survival (PFS), up to approximately 49 months after the first subject is randomized. PFS is as assessed by Independent Review Committee (IRC). Secondary endpoints include overall survival (OS), event-free survival (EFS), duration of response (DOR), percentage of subjects with complete response (CR), complete response rate (CRR) as assessed by IRC, time to progression (TTP), progression post-next line of treatment (PFS2) and time to next treatment (TTNT), all assessed up to approximately 60 months after the last subject is randomized. Secondary endpoints also include percentage of subjects with minimal residual disease negative (MRDneg) complete response (CR), assessed up 15 months after the last subject is randomized or percentage of subjects with sustained MRDneg CR for 12 months, assessed up approximately 27 months after the last subject is randomized.

Safety secondary endpoints include number of subjects experiencing adverse events (AEs) and number of subjects experiencing adverse events of special interest (AESI), both assessed up to approximately 60 months after the last subject is randomized. Pharmacokinetic endpoints include maximum observed plasma concentration (Cmax), time of maximum observed plasma concentration (Tmax), area under the curve (AUC) from time zero to 28 days post infusion and time of last measurable observed plasma concentration (Tlast), all assessed up to approximately 60 months after the last subject is randomized.

Secondary endpoints also include time-to-definitive deterioration and health-related quality of life (HRQoL) metrics, assessed up to approximately 49 months after subject randomization. In particular, HRQOL include mean change from baseline in European Organization for Research and Treatment of Cancer core quality of life questionnaire (EORTC QLQ-C30) and mean change from baseline in European Organization for Research and Treatment of Cancer core quality of life questionnaire for multiple myeloma (EORTC-QLQ-MY20).

The administered T cell compositions are generated by obtaining peripheral-blood mononuclear cells (PBMCs) from leukapheresis samples from individual subjects to be treated, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contain an anti-BCMA scFv, a hinge and transmembrane domain from a CD8α, and a CD137 (4-1BB) co-stimulatory domain followed by the intracellular signaling domain of a CD3ζ chain. The polynucleotide sequence encoding the exemplary BCMA CAR is set forth in SEQ ID NO: 214, and the polypeptide sequence of the exemplary BCMA CAR is set forth in SEQ ID NO: 116.

For subjects receiving CAR T cell administration with LEN maintenance, a LEN preleukapheresis (1 cycle) is administered orally at a dose of 10 mg once daily on Days 1 to 28 of a 28-day cycle starting within 7 days after randomization. In other words, the subject is administered a lenalidomide maintenance prior to a leukapheresis to obtain T cells from the subject for engineering with a BCMA targeted CAR to produce a BCMA targeted CAR T cell therapy. Leukapheresis is performed at least 14 days but not more than 42 days after completion of the preleukapheresis LEN administration. Subjects receive a 3-day cycle of lymphodepletion starting at 5 days prior to CAR T cell administration (with fludarabine (flu, 30 mg/m²/day) and cyclophosphamide (cy, 300 mg/m²/day) for 3 consecutive days (at days −5, −4 and −3)). Subjects received a single infusion of anti-BCMA CAR-T cells at a dose range of 300-460×10⁶ CAR+ T cells.

LEN maintenance will be resumed at 1-month post CAR T cell infusion, contingent on blood cell count recovery. LEN maintenance must be started no later than 6 months after CAR T cell infusion. LEN will be administered continuously on Days 1 to 28 of repeated 28-day cycles starting at 5 mg once daily for the first cycle and then increasing to 10 mg once daily with the second cycle, if well tolerated. After 3 cycles of LEN maintenance therapy at 10 mg daily, the dose can be increased to 15 mg once daily, if tolerated and at the investigator's discretion and as described in the current label.

For subjects receiving only LEN maintenance, LEN maintenance is initiated within 7 days of randomization. LEN is administered at a dose of 10 mg once daily on Days 1 to 28 of a 28-day cycle. After 3 cycles of LEN maintenance therapy at 10 mg daily, the dose can be increased to 15 mg once daily, if tolerated and at the investigator's discretion and as described in the current label.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO. | SEQUENCE | DESCRIPTION |
| 1 | ESKYGPPCPPCP | spacer |
| | | (IgG4hinge) (aa) |
| | | Homo sapiens |
| 2 | GAATCTAAGTACGGACCGCCCTGCCCCCCTTGCCCT | spacer |
| | | (IgG4hinge) (nt) |
| | | Homo sapiens |
| 3 | ESKYGPPCPPCPGQPREPQVYTLPPSQEEMTKNQVSLTCLVKGFYP | Hinge-CH3 |
| | SDIAVEWESNGQPENNYKTTPPVLDSDGSFFLYSRLTVDKSRWQE | spacer |
| | GNVFSCSVMHEALHNHYTQKSLSLSLGK | Homo sapiens |
| 4 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVL | spacer |
| | HQDWLNGKEYKCKVSNKGLPSSIEKTISKAKGQPREPQVYTLPPSQ | Homo sapiens |
| | EEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSL | |
| | GK | |
| 5 | RWPESPKAQASSVPTAQPQAEGSLAKATTAPATTRNTGRGGEEKK | IgD-hinge-Fc |
| | KEKEKEEQEERETKTPECPSHTQPLGVYLLTPAVQDLWLRDKATF | spacer |
| | TCFVVGSDLKDAHLTWEVAGKVPTGGVEEGLLERHSNGSQSQHS | Homo sapiens |
| | RLTLPRSLWNAGTSVTCTLNHPSLPPQRLMALREPAAQAPVKLSL | |
| | NLLASSDPPEAASWLLCEVSGFSPPNILLMWLEDQREVNTSGFAPA | |
| | RPPPQPGSTTFWAWSVLRVPAPPSPQPATYTCVVSHEDSRTLLNAS | |
| | RSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | artificial |
| | TKQHGQFSLAVVSLNITSLGLRSLKEISDGDVIISGNKNLCYANTIN | |
| | WKKLFGTSGQKTKIISNRGENSCKATGQVCHALCSPEGCWGPEPR | |
| | DCVSCRNVSRGRECVDKCNLLEGEPREFVENSECIQCHPECLPQA | |
| | MNITCTGRGPDNCIQCAHYIDGPHCVKTCPAGVMGENNTLVWKY | |
| | ADAGHVCHLCHPNCTYGCTGPGLEGCPTNGPKIPSIATGMVGALL | |
| | LLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVLVVV | CD28 (amino |
| | GGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to |
| | | GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 14 | RVKFSRSAEPPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 15 | RVKFSRSADAPAYKQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 16 | PGGG-(SGGGG)5-P-wherein P is proline, | Linker |
| | G is glycine and S is serine | |
| 17 | GSADDAKKDAAKKDGKS | Linker |
| 18 | QIQLVQSGPELKKPGETVKISCKASGYTFTDYSINWVKRAPGKGLK | Variable heavy |
| | WMGWINTETREPAYAYDFRGRFAFSLETSASTAYLQINNLKYEDT | (VH) Anti- |
| | ATYFCALDYSYAMDYWGQGTSVTVSS | BCMA |
| 19 | DIVLTQSPPSLAMSLGKRATISCRASESVTILGSHLIHWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 20 | QIQLVQSGPDLKKPGETVKLSCKASGYTFTNFGMNWVKQAPGKG | Variable heavy |
| | FKWMAWINTYTGESYFADDFKGRFAFSVETSATTAYLQINNLKTE | (VH) Anti- |
| | DTATYFCARGEIYYGYDGGFAYWGQGTLVTVSA | BCMA |
| 21 | DVVMTQSHRFMSTSVGDRVSITCRASQDVNTAVSWYQQKPGQSP | Variable light |
| | KLLIFSASYRYTGVPDRFTGSGSGADFTLTISSVQAEDLAVYYCQQ | (VL) Anti- |
| | HYSTPWTFGGGTKLDIK | BCMA |
| 22 | EVQLVQSGAEVKKPGESLKISCKGSGYSFTSYWIGWVRQMPGKGL | Variable heavy |
| | EWMGIIYPGDSDTRYSPSFQGHVTISADKSISTAYLQWSSLKASDT | (VH) Anti- |
| | AMYYCARYSGSFDNWGQGTLVTVSS | BCMA |
| 23 | SYELTQPPSASGTPGQRVTMSCSGTSSNIGSHSVNWYQQLPGTAPK | Variable light |
| | LLIYTNNQRPSGVPDRFSGSKSGTSASLAISGLQSEDEADYYCAAW | (VL) Anti- |
| | DGSLNGLVFGGGTKLTVLG | BCMA |
| 24 | EVQLVQSGAEMKKPGASLKLSCKASGYTFIDYYVYWMRQAPGQG | Variable heavy |
| | LESMGWINPNSGGTNYAQKFQGRVTMTRDTSISTAYMELSRLRSD | (VH) Anti- |
| | DTAMYYCARSQRDGYMDYWGQGTLVTVSS | BCMA |
| 25 | QSALTQPASVSASPGQSIAISCTGTSSDVGWYQQHPGKAPKLMIYE | Variable light |
| | DSKRPSGVSNRFSGSKSGNTASLTISGLQAEDEADYYCSSNTRSSTL | (VL) Anti- |
| | VFGGGTKLTVLG | BCMA |
| 26 | GGGGS | Linker |
| 27 | GGGS | Linker |
| 28 | GGGGSGGGGSGGGGS | Linker |
| 29 | GSTSGSGKPGSGEGSTKG | Linker |
| 30 | SRGGGGSGGGGSGGGGSLEMA | Linker |
| 31 | ESKYGPPCPPCPAPPVAGPSVFLFPPKPKDTLMISRTPEVTCVVVDV | Hinge-CH2-CH3 |
| | SQEDPEVQFNWYVDGVEVHNAKTKPREEQFQSTYRVVSVLTVLH | spacer |
| | QDWLNGKEYKCKVSNKGLPSSIEKTISKAKGQPREPQVYTLPPSQE | Homo sapiens |
| | EMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLG | |
| | K | |
| 32 | EVQLVQSGAEVKKPGSSVKVSCKASGGTFSSYAISWVRQAPGQGL | Variable heavy |
| | EWMGRIIPILGIANYAQKFQGRVTMTEDTSTDTAYMELSSLRSEDT | (VH) Anti- |
| | AVYYCARSGYSKSIVSYMDYWGQGTLVTVSS | BCMA |
| 33 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 34 | QVQLVQSGAEVKKPGSSVKVSCKASGGTFSSYAISWVRQAPGQGL | Variable heavy |
| | EWMGRIIPILGTANYAQKFQGRVTITADESTSTAYMELSSLRSEDT | (VH) Anti- |
| | AVYYCARSGYGSYRWEDSWGQGTLVTVSS | BCMA |
| 35 | QAVLTQPPSASGTPGQRVTISCSGSSSNIGSNYVFWYQQLPGTAPK | Variable light |
| | LLIYSNNQRPSGVPDRFSGSKSGTSASLAISGLRSEDEADYYCAAW | (VL) Anti- |
| | DDSLSASYVFGTGTKVTVLG | BCMA |
| 36 | QVQLVQSGAEVKKPGASVKVSCKASGYTFTDYYMHWVRQAPGQ | Variable heavy |
| | RLEWMGWINPNSGGTNYAQKFQDRITVTRDTSSNTGYMELTRLRS | (VH) Anti- |
| | DDTAVYYCARSPYSGVLDKWGQGTLVTVSS | BCMA |
| 37 | QSVLTQPPSVSGAPGQRVTISCTGSSSNIGAGFDVHWYQQLPGTAP | Variable light |
| | KLLIYGNSNRPSGVPDRFSGSKSGTSASLAITGLQAEDEADYYCQS | (VL) Anti- |
| | YDSSLSGYVFGTGTKVTVLG | BCMA |
| 38 | ASGGGGSGGRASGGGGS | Linker |
| 39 | MALPVTALLLPLALLLHAARP | CD8a signal |
| | | peptide |
| 40 | METDTLLLWVLLLWVPGSTG | signal peptide |
| 41 | EVQLLESGGGLVQPGGSLRLSCAASGFTFSSYAMSWVRQAPGKGL | Variable heavy |
| | EWVSAISGSGGSTYYADSVKGRFTISRDNSKNTLYLQMNSLRAED | (VH) Anti- |
| | TAVYYCARAEMGAVFDIWGQGTMVTVSS | BCMA |
| 42 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRIS | (VL) Anti- |
| | WPFTFGGGTKVEIK | BCMA |
| 43 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCARDGTYLGGLWYFDLWGRGTLVTVSS | BCMA |
| 44 | DIVMTQSPLSLPVTPGEPASISCRSSQSLLHSNGYNYLDWYLQKPG | Variable light |
| | QSPQLLIYLGSNRASGVPDRFSGSGSGTDFTLKISRVEAEDVGVYY | (VL) Anti- |
| | CMQGLGLPLTFGGGTKVEIK | BCMA |
| 45 | QVQLVQSGAEVKKPGASVKVSCKASGYTFTSYYMHWVRQAPGQ | Variable heavy |
| | GLEWMGIINPGGGSTSYAQKFQGRVTMTRDTSTSTVYMELSSLRS | (VH) Anti- |
| | EDTAVYYCARESWPMDVWGQGTTVTVSS | BCMA |
| 46 | EIVMTQSPATLSVSPGERATLSCRASQSVSSNLAWYQQKPGQAPRL | Variable light |
| | LIYGASTRATGIPARFSGSGSGTEFTLTISSLQSEDFAVYYCQQYAA | (VL) Anti- |
| | YPTFGGGTKVEIK | BCMA |
| 47 | QLQLQESGPGLVKPSETLSLTCTVSGGSISSSSYYWGWIRQPPGKG | Variable heavy |
| | LEWIGSISYSGSTYYNPSLKSRVTISVDTSKNQFSLKLSSVTAADTA | (VH) Anti- |
| | VYYCARGRGYATSLAFDIWGQGTMVTVSS | BCMA |
| 48 | EIVLTQSPATLSLSPGERATLSCRASQSVSSYLAWYQQKPGQAPRL | Variable light |
| | LIYDASNRATGIPARFSGSGSGTDFTLTISSLEPEDFAVYYCQQRHV | (VL) Anti- |
| | WPPTFGGGTKVEIK | BCMA |
| 49 | EVQLVESGGGLVQPGGSLRLSCAASGFTFSSYSMNWVRQAPGKGL | Variable heavy |
| | EWVSTISSSSSTIYYADSVKGRFTISRDNAKNSLYLQMNSLRAEDT | (VH) Anti- |
| | AVYYCARGSQEHLIFDYWGQGTLVTVSS | BCMA |
| 50 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRFY | (VL) Anti- |
| | YPWTFGGGTKVEIK | BCMA |
| 51 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCARTDFWSGSPPGLDYWGQGTLVTVSS | BCMA |
| 52 | DIQLTQSPSSVSASVGDRVTITCRASQGISSWLAWYQQKPGKAPKL | Variable light |
| | LIYGASSLQSGVPSRFSGSGSGTDFTLTISSLQPEDFATYYCQQIYTF | (VL) Anti- |
| | PFTFGGGTKVEIK | BCMA |
| 53 | QVQLVQSGAEVKKPGSSVKVSCKASGGTFSSYAISWVRQAPGQGL | Variable heavy |
| | EWMGGIIPIFGTANYAQKFQGRVTITADESTSTAYMELSSLRSEDT | (VH) Anti- |
| | AVYYCARTPEYSSSIWHYYYGMDVWGQGTTVTVSS | BCMA |
| 54 | DIVMTQSPDSLAVSLGERATINCKSSQSVLYSSNNKNYLAWYQQK | Variable light |
| | PGQPPKLLIYWASTRESGVPDRFSGSGSGTDFTLTISSLQAEDVAVY | (VL) Anti- |
| | YCQQFAHTPFTFGGGTKVEIK | BCMA |
| 55 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCVKGPLQEPPYDYGMDVWGQGTTVTVSS | BCMA |
| 56 | EIVMTQSPATLSVSPGERATLSCRASQSVSSNLAWYQQKPGQAPRL | Variable light |
| | LIYSASTRATGIPARFSGSGSGTEFTLTISSLQSEDFAVYYCQQHHV | (VL) Anti- |
| | WPLTFGGGTKVEIK | BCMA |
| 57 | QVQLVQSGAEVKKPGSSVKVSCKASGGTFSSYAISWVRQAPGQGL | Variable heavy |
| | EWMGRIIPILGIANYAQKFQGRVTITADKSTSTAYMELSSLRSEDTA | (VH) Anti- |
| | VYYCARGGYYSHDMWSEDWGQGTLVTVSS | BCMA |
| 58 | LPVLTQPPSASGTPGQRVTISCSGRSSNIGSNSVNWYRQLPGAAPKL | Variable light |
| | LIYSNNQRPPGVPVRFSGSKSGTSASLAISGLQSEDEATYYCATWD | (VL) Anti- |
| | DNLNVHYVFGTGTKVTVLG | BCMA |
| 59 | QVQLVQSGSELKKPGASVKVSCKASGYTFTDYSINWVRQAPGQGL | Variable heavy |
| | EWMGWINTETREPAYAYDFRGRFVFSLDTSVSTAYLQISSLKAEDT | (VH) Anti- |
| | AVYYCARDYSYAMDYWGQGTLVTVSS | BCMA |
| 60 | DIVLTQSPASLAVSLGERATINCRASESVSVIGAHLIHWYQQKPGQP | Variable light |
| | PKLLIYLASNLETGVPARFSGSGSGTDFTLTISSLQAEDAAIYYCLQ | (VL) Anti- |
| | SRIFPRTFGQGTKLEIK | BCMA |
| 61 | EVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 62 | DIQLTQSPSSLSASVGDRVTITCRASQSISSYLNWYQQKPGKAPKLL | Variable light |
| | IYAASSLQSGVPSRFSGSGSGTDFTLTISSLQPEDFATYYCQQSYSTP | (VL) Anti- |
| | YTFGQGTKVEIK | BCMA |
| 63 | QVQLVESGGGLVQPGRSLRLSCAASGFTFSNYAMSWVRQAPGKG | Variable heavy |
| | LGWVSGISRSGENTYYADSVKGRFTISRDNSKNTLYLQMNSLRDE | (VH) Anti- |
| | DTAVYYCARSPAHYYGGMDVWGQGTTVTVSS | BCMA |
| 64 | DIVLTQSPGTLSLSPGERATLSCRASQSISSSFLAWYQQKPGQAPRL | Variable light |
| | LIYGASRRATGIPDRFSGSGSGTDFTLTISRLEPEDSAVYYCQQYHS | (VL) Anti- |
| | SPSWTFGQGTKLEIK | BCMA |
| 65 | QVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 66 | DIRLTQSPSPLSASVGDRVTITCQASEDINKFLNWYHQTPGKAPKLL | Variable light |
| | IYDASTLQTGVPSRFSGSGSGTDFTLTINSLQPEDIGTYYCQQYESLP | (VL) Anti- |
| | LTFGGGTKVEIK | BCMA |
| 67 | EVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 68 | EIVLTQSPGTLSLSPGERATLSCRASQSIGSSSLAWYQQKPGQAPRL | Variable light |
| | LMYGASSRASGIPDRFSGSGSGTDFTLTISRLEPEDFAVYYCQQYA | (VL) Anti- |
| | GSPPFTFGQGTKVEIK | BCMA |
| 69 | QIQLVQSGPELKKPGETVKISCKASGYTFRHYSMNWVKQAPGKGL | Variable heavy |
| | KWMGRINTESGVPIYADDFKGRFAFSVETSASTAYLVINNLKDEDT | (VH) Anti- |
| | ASYFCSNDYLYSLDFWGQGTALTVSS | BCMA |
| 70 | DIVLTQSPPSLAMSLGKRATISCRASESVTILGSHLIYWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 71 | QIQLVQSGPELKKPGETVKISCKASGYTFTHYSMNWVKQAPGKGL | Variable heavy |
| | KWMGRINTETGEPLYADDFKGRFAFSLETSASTAYLVINNLKNED | (VH) Anti- |
| | TATFFCSNDYLYSCDYWGQGTTLTVSS | BCMA |
| 72 | DIVLTQSPASLAMSLGKRATISCRASESVSVIGAHLIHWYQQKPGQ | Variable light |
| | PPKLLIYLASNLETGVPARFSGSGSGTDFTLTIDPVEEDDVAIYSCL | (VL) Anti- |
| | QSRIFPRTFGGGTKLEIK | BCMA |
| 73 | QVQLVQSGAEVKKPGASVKVSCKASGYSFPDYYINWVRQAPGQG | Variable heavy |
| | LEWMGWIYFASGNSEYNQKFTGRVTMTRDTSINTAYMELSSLTSE | (VH) Anti- |
| | DTAVYFCASLYDYDWYFDVWGQGTMVTVSS | BCMA |
| 74 | DIVMTQTPLSLSVTPGQPASISCKSSQSLVHSNGNTYLHWYLQKPG | Variable light |
| | QSPQLLIYKVSNRFSGVPDRFSGSGSGTDFTLKISRVEAEDVGIYYC | (VL) Anti- |
| | SQSSIYPWTFGQGTKLEIK | BCMA |
| 75 | QVQLVQSGAEVKKPGASVKVSCKASGYSFPDYYINWVRQAPGQG | Variable heavy |
| | LEWMGWIYFASGNSEYNQKFTGRVTMTRDTSSSTAYMELSSLRSE | (VH) Anti- |
| | DTAVYFCASLYDYDWYFDVWGQGTMVTVSS | BCMA |
| 76 | DIVMTQTPLSLSVTPGEPASISCKSSQSLVHSNGNTYLHWYLQKPG | Variable light |
| | QSPQLLIYKVSNRFSGVPDRFSGSGSGADFTLKISRVEAEDVGVYY | (VL) Anti- |
| | CAETSHVPWTFGQGTKLEIK | BCMA |
| 77 | QVQLVESGGGLVQPGGSLRLSCEASGFTLDYYAIGWFRQAPGKER | Anti-BCMA |
| | EGVICISRSDGSTYYADSVKGRFTISRDNAKKTVYLQMISLKPEDT | sdAb |
| | AAYYCAAGADCSGYLRDYEFRGQGTQVTVSS | |
| 78 | IEVMYPPPYLDNEKSNGTIIHVKGKHLCPSPLFPGPSKP | CD28 spacer |
| 79 | IYIWAPLAGTCGVLLLSLVITLYCN | CD8a TM |
| 80 | LDNEKSNGTIIHVKGKHLCPSPLFPGPSKP | CD28 spacer |
| | | (truncated) |
| 81 | PTTTPAPRPPTPAPTIASQPLSLRPEACRPAAGGAVHTRGLDFACD | CD8a hinge |
| 82 | TTTPAPRPPTPAPTIASQPLSLRPEACRPAAGGAVHTRGLDFACD | CD8a hinge |
| 83 | FVPVFLPAKPTTTPAPRPPTPAPTIASQPLSLRPEACRPAAGGAVHT | CD8a hinge |
| | RGLDFACD | |
| 84 | DTGLYICKVELMYPPPYYLGIGNGTQIYVIDPEPCPDSD | CTLA4 hinge |
| 85 | FLLWILAAVSSGLFFYSFLLTAVS | CTLA4 TM |
| 86 | QIKESLRAELRVTERRAEVPTAHPSPSPRPAGQFQTLV | PD-1 hinge |
| 87 | VGVVGGLLGSLVLLVWVLAVI | PD-1 TM |
| 88 | GLAVSTISSFFPPGYQ | Fc(gamma)RIIIa |
| | | hinge |
| 89 | EPKSPDKTHTCPPCPAPPVAGPSVFLFPPKPKDTLMIARTPEVTCVV | IgG1 hinge |
| | VDVSHEDPEVKFNWYVDGVEVHNAKTKPREEQYNSTYRVVSVLT | |
| | VLHQDWLNGKEYKCKVSNKALPAPIEKTISKAKGQPREPQVYTLP | |
| | PSRDELTKNQVSLTCLVKGFYPSDIAVEWESNGQPENNYKTTPPVL | |
| | DSDGSFFLYSKLTVDKSRWQQGNVFSCSVMHEALHNHYTQKSLSL | |
| | SPGK | |
| 90 | EVQLLESGGGLVQPGGSLRLSCAASGFTFSSYAMSWVRQAPGKGL | anti-BCMA CAR |
| | EWVSAISGSGGSTYYADSVKGRFTISRDNSKNTLYLQMNSLRAED | |
| | TAVYYCARAEMGAVFDIWGQGTMVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIKRAAALDNEKSNGTIIHVKGKHLCPSPLFPGPS | |
| | KPFWVLVVVGGVLACYSLLVTVAFIIFWVRSKRSRLLHSDYMNMT | |
| | PRRPGPTRKHYQPYAPPRDFAAYRSRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| 91 | EIVLTQSPATLSLSPGERATLSCRASQSVSRYLAWYQQKPGQAPRL | anti-BCMA CAR |
| | LIYDASNRATGIPARFSGSGSGTDFTLTISSLEPEDFAVYYCQQRIS | |
| | WPFTFGGGTKVEIKRGSTSGSGKPGSGEGSTKGEVQLLESGGGLV | |
| | QPGGSLRLSCAASGFTFSSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSSAAALDNEKSNGTIIHVKGKHLCPSPLFP | |
| | GPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 92 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIKRAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 93 | DIVMTQSPLSLPVTPGEPASISCRSSQSLLHSNGYNYLDWYLQKPG | anti-BCMA CAR |
| | QSPQLLIYLGSNRASGVPDRFSGSGSGTDFTLKISRVEAEDVGVYY | |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSSAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 94 | QVQLVQSGAEVKKPGASVKVSCKASGYTFTSYYMHWVRQAPGQ | anti-BCMA CAR |
| | GLEWMGIINPGGGSTSYAQKFQGRVTMTRDTSTSTVYMELSSLRS | |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQY | |
| | AAYPTFGGGTKVEIKRAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 95 | EIVMTQSPATLSVSPGERATLSCRASQSVSSNLAWYQQKPGQAPRL | anti-BCMA CAR |
| | LIYGASTRATGIPARFSGSGSGTEFTLTISSLQSEDFAVYYCQQYAA | |
| | YPTFGGGTKVEIKRGSTSGSGKPGSGEGSTKGQVQLVQSGAEVKK | |
| | PGASVKVSCKASGYTFTSYYMHWVRQAPGQGLEWMGIINPGGGS | |
| | TSYAQKFQGRVTMTRDTSTSTVYMELSSLRSEDTAVYYCARESWP | |
| | MDVWGQGTTVTVSSAAALDNEKSNGTIIHVKGKHLCPSPLFPGPS | |
| | KPFWVLVVVGGVLACYSLLVTVAFIIFWVRSKRSRLLHSDYMNMT | |
| | PRRPGPTRKHYQPYAPPRDFAAYRSRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| 96 | QLQLQESGPGLVKPSETLSLTCTVSGGSISSSSYYWGWIRQPPGKG | anti-BCMA CAR |
| | LEWIGSISYSGSTYYNPSLKSRVTISVDTSKNQFSLKLSSVTAADTA | |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIKRAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 97 | EIVLTQSPATLSLSPGERATLSCRASQSVSSYLAWYQQKPGQAPRL | anti-BCMA CAR |
| | LIYDASNRATGIPARFSGSGSGTDFTLTISSLEPEDFAVYYCQQRHV | |
| | WPPTFGGGTKVEIKRGSTSGSGKPGSGEGSTKGQLQLQESGPGLVK | |
| | PSETLSLTCTVSGGSISSSSYYWGWIRQPPGKGLEWIGSISYSGSTY | |
| | YNPSLKSRVTISVDTSKNQFSLKLSSVTAADTAVYYCARGRGYATS | |
| | LAFDIWGQGTMVTVSSAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 98 | EVQLVESGGGLVQPGGSLRLSCAASGFTFSSYSMNWVRQAPGKGL | anti-BCMA CAR |
| | EWVSTISSSSSTIYYADSVKGRFTISRDNAKNSLYLQMNSLRAEDT | |
| | AVYYCARGSQEHLIFDYWGQGTLVTVSSGSTSGSGKPGSGEGSTK | |
| | GEIVLTQSPATLSLSPGERATLSCRASQSVSRYLAWYQQKPGQAPR | |
| | LLIYDASNRATGIPARFSGSGSGTDFTLTISSLEPEDFAVYYCQQRF | |
| | YYPWTFGGGTKVEIKRAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 99 | EIVLTQSPATLSLSPGERATLSCRASQSVSRYLAWYQQKPGQAPRL | anti-BCMA CAR |
| | LIYDASNRATGIPARFSGSGSGTDFTLTISSLEPEDFAVYYCQQRFY | |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSSAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 100 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCARTDFWSGSPPGLDYWGQGTLVTVSSGSTSGSGKPGSG | |
| | EGSTKGDIQLTQSPSSVSASVGDRVTITCRASQGISSWLAWYQQKP | |
| | GKAPKLLIYGASSLQSGVPSRFSGSGSGTDFTLTISSLQPEDFATYY | |
| | CQQIYTFPFTFGGGTKVEIKRAAALDNEKSNGTIIHVKGKHLCPSPL | |
| | FPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 101 | DIQLTQSPSSVSASVGDRVTITCRASQGISSWLAWYQQKPGKAPKL | anti-BCMA CAR |
| | LIYGASSLQSGVPSRFSGSGSGTDFTLTISSLQPEDFATYYCQQIYTF | |
| | PFTFGGGTKVEIKRGSTSGSGKPGSGEGSTKGQVQLVESGGGVVQP | |
| | GRSLRLSCAASGFTFSSYGMHWVRQAPGKGLEWVAVISYDGSNK | |
| | YYADSVKGRFTISRDNSKNTLYLQMNSLRAEDTAVYYCARTDFW | |
| | SGSPPGLDYWGQGTLVTVSSAAALDNEKSNGTIIHVKGKHLCPSPL | |
| | FPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 102 | QVQLVQSGAEVKKPGSSVKVSCKASGGTFSSYAISWVRQAPGQGL | anti-BCMA CAR |
| | EWMGGIIPIFGTANYAQKFQGRVTITADESTSTAYMELSSLRSEDT | |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIKRAAALDNEKSNGTII | |
| | HVKGKHLCPSPLFPGPSKPFWVLVVVGGVLACYSLLVTVAFIIFWV | |
| | RSKRSRLLHSDYMNMTPRRPGPTRKHYQPYAPPRDFAAYRSRVKF | |
| | SRSADAPAYQQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 103 | DIVMTQSPDSLAVSLGERATINCKSSQSVLYSSNNKNYLAWYQQK | anti-BCMA CAR |
| | PGQPPKLLIYWASTRESGVPDRFSGSGSGTDFTLTISSLQAEDVAVY | |
| | YCQQFAHTPFTFGGGTKVEIKRGSTSGSGKPGSGEGSTKGQVQLV | |
| | QSGAEVKKPGSSVKVSCKASGGTFSSYAISWVRQAPGQGLEWMG | |
| | GIIPIFGTANYAQKFQGRVTITADESTSTAYMELSSLRSEDTAVYYC | |
| | ARTPEYSSSIWHYYYGMDVWGQGTTVTVSSAAALDNEKSNGTIIH | |
| | VKGKHLCPSPLFPGPSKPFWVLVVVGGVLACYSLLVTVAFIIFWVR | |
| | SKRSRLLHSDYMNMTPRRPGPTRKHYQPYAPPRDFAAYRSRVKFS | |
| | RSADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 104 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIKRAAALDNEKSNGTIIHVKGKHLCP | |
| | SPLFPGPSKPFWVLVVVGGVLACYSLLVTVAFIIFWVRSKRSRLLH | |
| | SDYMNMTPRRPGPTRKHYQPYAPPRDFAAYRSRVKFSRSADAPAY | |
| | QQGQNQLYNELNLGRREEYDVLDKRRGRDPEMGGKPRRKNPQEG | |
| | LYNELQKDKMAEAYSEIGMKGERRRGKGHDGLYQGLSTATKDTY | |
| | DALHMQALPPR | |
| 105 | EIVMTQSPATLSVSPGERATLSCRASQSVSSNLAWYQQKPGQAPRL | anti-BCMA CAR |
| | LIYSASTRATGIPARFSGSGSGTEFTLTISSLQSEDFAVYYCQQHHV | |
| | WPLTFGGGTKVEIKRGSTSGSGKPGSGEGSTKGQVQLVESGGGVV | |
| | QPGRSLRLSCAASGFTFSSYGMHWVRQAPGKGLEWVAVISYDGS | |
| | NKYYADSVKGRFTISRDNSKNTLYLQMNSLRAEDTAVYYCVKGP | |
| | LQEPPYDYGMDVWGQGTTVTVSSAAALDNEKSNGTIIHVKGKHL | |
| | CPSPLFPGPSKPFWVLVVVGGVLACYSLLVTVAFIIFWVRSKRSRL | |
| | LHSDYMNMTPRRPGPTRKHYQPYAPPRDFAAYRSRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTATK | |
| | DTYDALHMQALPPR | |
| 106 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSAAAIEVMYPPPYLDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 107 | QSVLTQPPSVSGAPGQRVTISCTGSSSNIGAGFDVHWYQQLPGTAP | anti-BCMA CAR |
| | KLLIYGNSNRPSGVPDRFSGSKSGTSASLAITGLQAEDEADYYCQS | |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAIEVMYPPPYLDNE | |
| | KSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 108 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSAAAIEVMYPPPYLDNEKSNGTIIHV | |
| | KGKHLCPSPLFPGPSKPFWVLVVVGGVLACYSLLVTVAFIIFWVRS | |
| | KRSRLLHSDYMNMTPRRPGPTRKHYQPYAPPRDFAAYRSRVKFSR | |
| | SADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 109 | LPVLTQPPSASGTPGQRVTISCSGRSSNIGSNSVNWYRQLPGAAPKL | anti-BCMA CAR |
| | LIYSNNQRPPGVPVRFSGSKSGTSASLAISGLQSEDEATYYCATWD | |
| | DNLNVHYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGIANYAQKFQGRVTITADKSTSTAYMELSSLRSEDTAVY | |
| | YCARGGYYSHDMWSEDWGQGTLVTVSSAAAIEVMYPPPYLDNE | |
| | KSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 110 | QAVLTQPPSASGTPGQRVTISCSGSSSNIGSNYVFWYQQLPGTAPK | anti-BCMA CAR |
| | LLIYSNNQRPSGVPDRFSGSKSGTSASLAISGLRSEDEADYYCAAW | |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAVY | |
| | YCARSGYGSYRWEDSWGQGTLVTVSSAAAIEVMYPPPYLDNEKS | |
| | NGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYR | |
| | SRVKFSRSADAPAYQQGQNQLYNELNLGRREEYDVLDKRRGRDP | |
| | EMGGKPRRKNPQEGLYNELQKDKMAEAYSEIGMKGERRRGKGH | |
| | DGLYQGLSTATKDTYDALHMQALPPR | |
| 111 | LPVLTQPPSASGTPGQRVTISCSGRSSNIGSNSVNWYRQLPGAAPKL | anti-BCMA CAR |
| | LIYSNNQRPPGVPVRFSGSKSGTSASLAISGLQSEDEATYYCATWD | |
| | DNLNVHYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGIANYAQKFQGRVTITADKSTSTAYMELSSLRSEDTAVY | |
| | YCARGGYYSHDMWSEDWGQGTLVTVSSAAAPTTTPAPRPPTPAP | |
| | TIASQPLSLRPEACRPAAGGAVHTRGLDFACDIYIWAPLAGTCGVL | |
| | LLSLVITLYCNKRGRKKLLYIFKQPFMRPVQTTQEEDGCSCRFPEEE | |
| | EGGCELRVKFSRSAEPPAYQQGQNQLYNELNLGRREEYDVLDKRR | |
| | GRDPEMGGKPRRKNPQEGLYNELQKDKMAEAYSEIGMKGERRRG | |
| | KGHDGLYQGLSTATKDTYDALHMQALPPR | |
| 112 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSAAAPTTTPAPRPPTPAPTIASQPLSL | |
| | RPEACRPAAGGAVHTRGLDFACDIYIWAPLAGTCGVLLLSLVITLY | |
| | CNKRGRKKLLYIFKQPFMRPVQTTQEEDGCSCRFPEEEEGGCELRV | |
| | KFSRSAEPPAYQQGQNQLYNELNLGRREEYDVLDKRRGRDPEMG | |
| | GKPRRKNPQEGLYNELQKDKMAEAYSEIGMKGERRRGKGHDGLY | |
| | QGLSTATKDTYDALHMQALPPR | |
| 113 | QAVLTQPPSASGTPGQRVTISCSGSSSNIGSNYVFWYQQLPGTAPK | anti-BCMA CAR |
| | LLIYSNNQRPSGVPDRFSGSKSGTSASLAISGLRSEDEADYYCAAW | |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAVY | |
| | YCARSGYGSYRWEDSWGQGTLVTVSSAAAPTTTPAPRPPTPAPTIA | |
| | SQPLSLRPEACRPAAGGAVHTRGLDFACDIYIWAPLAGTCGVLLLS | |
| | LVITLYCNKRGRKKLLYIFKQPFMRPVQTTQEEDGCSCRFPEEEEG | |
| | GCELRVKFSRSAEPPAYQQGQNQLYNELNLGRREEYDVLDKRRG | |
| | RDPEMGGKPRRKNPQEGLYNELQKDKMAEAYSEIGMKGERRRGK | |
| | GHDGLYQGLSTATKDTYDALHMQALPPR | |
| 114 | QSVLTQPPSVSGAPGQRVTISCTGSSSNIGAGFDVHWYQQLPGTAP | anti-BCMA CAR |
| | KLLIYGNSNRPSGVPDRFSGSKSGTSASLAITGLQAEDEADYYCQS | |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAPTTTPAPRPPTPAP | |
| | TIASQPLSLRPEACRPAAGGAVHTRGLDFACDIYIWAPLAGTCGVL | |
| | LLSLVITLYCNKRGRKKLLYIFKQPFMRPVQTTQEEDGCSCRFPEEE | |
| | EGGCELRVKFSRSAEPPAYQQGQNQLYNELNLGRREEYDVLDKRR | |
| | GRDPEMGGKPRRKNPQEGLYNELQKDKMAEAYSEIGMKGERRRG | |
| | KGHDGLYQGLSTATKDTYDALHMQALPPR | |
| 115 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSAAAPTTTPAPRPPTPAPTIASQPLSLR | |
| | PEACRPAAGGAVHTRGLDFACDIYIWAPLAGTCGVLLLSLVITLYC | |
| | NKRGRKKLLYIFKQPFMRPVQTTQEEDGCSCRFPEEEEGGCELRVK | |
| | FSRSAEPPAYQQGQNQLYNELNLGRREEYDVLDKRRGRDPEMGG | |
| | KPRRKNPQEGLYNELQKDKMAEAYSEIGMKGERRRGKGHDGLYQ | |
| | GLSTATKDTYDALHMQALPPR | |
| 116 | DIVLTQSPPSLAMSLGKRATISCRASESVTILGSHLIHWYQQKPGQP | anti-BCMA CAR |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSSAAATTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 117 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAATTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELRVKFSRSA | |
| | DAPAYQQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 118 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAADTGLYICKVELMYPPPYYLGIGNGTQI | |
| | YVIDPEPCPDSDFLLWILAAVSSGLFFYSFLLTAVSKRGRKKLLYIF | |
| | KQPFMRPVQTTQEEDGCSCRFPEEEEGGCELRVKFSRSADAPAYQ | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 119 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAAQIKESLRAELRVTERRAEVPTAHPSPS | |
| | PRPAGQFQTLVVGVVGGLLGSLVLLVWVLAVICSKRGRKKLLYIF | |
| | KQPFMRPVQTTQEEDGCSCRFPEEEEGGCELRVKFSRSADAPAYQ | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 120 | EVQLVESGGGLVKPGGSLRLSCAASGFTFSDYYMSWIRQAPGKGL | anti-BCMA CAR |
| | EWVSYISSSGSTIYYADSVKGRFTISRDNAKNSLYLQMNSLRAEDT | |
| | AVYYCAKVDGDYTEDYWGQGTLVTVSSGGGGSGGGGSGGGGSQ | |
| | SALTQPASVSGSPGQSITISCTGSSSDVGKYNLVSWYQQPPGKAPK | |
| | LIIYDVNKRPSGVSNRFSGSKSGNTATLTISGLQGDDEADYYCSSY | |
| | GGSRSYVFGTGTKVTVLESKYGPPCPPCPAPPVAGPSVFLFPPKPK | |
| | DTLMISRTPEVTCVVVDVSQEDPEVQFNWYVDGVEVHNAKTKPR | |
| | EEQFQSTYRVVSVLTVLHQDWLNGKEYKCKVSNKGLPSSIEKTISK | |
| | AKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVM | |
| | HEALHNHYTQKSLSLSLGKMFWVLVVVGGVLACYSLLVTVAFIIF | |
| | WVKRGRKKLLYIFKQPFMRPVQTTQEEDGCSCRFPEEEEGGCELR | |
| | VKFSRSADAPAYQQGQNQLYNELNLGRREEYDVLDKRRGRDPEM | |
| | GGKPRRKNPQEGLYNELQKDKMAEAYSEIGMKGERRRGKGHDGL | |
| | YQGLSTATKDTYDALHMQALPPR | |
| 121 | EVQLVQSGGGLVQPGRSLRLSCTASGFTFGDYAMSWFKQAPGKG | anti-BCMA CAR |
| | LEWVGFIRSKAYGGTTEYAASVKGRFTISRDDSKSIAYLQMNSLKT | |
| | EDTAVYYCAAWSAPTDYWGQGTLVTVSSGGGGSGGGGSGGGGS | |
| | DIQMTQSPAFLSASVGDRVTVTCRASQGISNYLAWYQQKPGNAPR | |
| | LLIYSASTLQSGVPSRFRGTGYGTEFSLTIDSLQPEDFATYYCQQSY | |
| | TSRQTFGPGTRLDIKESKYGPPCPPCPAPPVAGPSVFLFPPKPKDTL | |
| | MISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAK | |
| | GQPREPQVYTLPPSQEEMTKNQVSLTCLVKGFYPSDIAVEWESNG | |
| | QPENNYKTTPPVLDSDGSFFLYSRLTVDKSRWQEGNVFSCSVMHE | |
| | ALHNHYTQKSLSLSLGKMFWVLVVVGGVLACYSLLVTVAFIIFWV | |
| | KRGRKKLLYIFKQPFMRPVQTTQEEDGCSCRFPEEEEGGCELRVKF | |
| | SRSADAPAYQQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 122 | EVQLVESGGGLVKPGGSLRLSCAASGFTFSDYYMSWIRQAPGKGL | anti-BCMA CAR |
| | EWVSYISSSGSTIYYADSVKGRFTISRDNAKNSLYLQMNSLRAEDT | |
| | AVYYCAKVDGPPSFDIWGQGTMVTVSSGGGGSGGGGSGGGGSSY | |
| | VLTQPPSVSVAPGQTARITCGANNIGSKSVHWYQQKPGQAPMLVV | |
| | YDDDDRPSGIPERFSGSNSGNTATLTISGVEAGDEADYFCHLWDRS | |
| | RDHYVFGTGTKLTVLESKYGPPCPPCPAPPVAGPSVFLFPPKPKDT | |
| | LMISRTPEVTCVVVDVSQEDPEVQFNWYVDGVEVHNAKTKPREE | |
| | QFQSTYRVVSVLTVLHQDWLNGKEYKCKVSNKGLPSSIEKTISKA | |
| | KGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMH | |
| | EALHNHYTQKSLSLSLGKMFWVLVVVGGVLACYSLLVTVAFIIFW | |
| | VKRGRKKLLYIFKQPFMRPVQTTQEEDGCSCRFPEEEEGGCELRVK | |
| | FSRSADAPAYQQGQNQLYNELNLGRREEYDVLDKRRGRDPEMGG | |
| | KPRRKNPQEGLYNELQKDKMAEAYSEIGMKGERRRGKGHDGLYQ | |
| | GLSTATKDTYDALHMQALPPR | |
| 123 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSESKYGPPCPPCPAPPVAGPSVFLFP | |
| | PKPKDTLMISRTPEVTCVVVDVSQEDPEVQFNWYVDGVEVHNAK | |
| | TKPREEQFQSTYRVVSVLTVLHQDWLNGKEYKCKVSNKGLPSSIE | |
| | KTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFS | |
| | CSVMHEALHNHYTQKSLSLSLGKMFWVLVVVGGVLACYSLLVTV | |
| | AFIIFWVKRGRKKLLYIFKQPFMRPVQTTQEEDGCSCRFPEEEEGG | |
| | CELRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 124 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSESKYGPPCPPCPAPPVAGPSVFLFPP | |
| | KPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEK | |
| | TISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSC | |
| | SVMHEALHNHYTQKSLSLSLGKMFWVLVVVGGVLACYSLLVTVA | |
| | FIIFWVKRGRKKLLYIFKQPFMRPVQTTQEEDGCSCRFPEEEEGGCE | |
| | LRVKFSRSADAPAYQQGQNQLYNELNLGRREEYDVLDKRRGRDP | |
| | EMGGKPRRKNPQEGLYNELQKDKMAEAYSEIGMKGERRRGKGH | |
| | DGLYQGLSTATKDTYDALHMQALPPR | |
| 125 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSESKYGPPCPPCPAPPVAGPSVFLFPP | |
| | KPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEK | |
| | TISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSC | |
| | SVMHEALHNHYTQKSLSLSLGKMFWVLVVVGGVLACYSLLVTVA | |
| | FIIFWVRSKRSRLLHSDYMNMTPRRPGPTRKHYQPYAPPRDFAAY | |
| | RSRVKFSRSADAPAYQQGQNQLYNELNLGRREEYDVLDKRRGRD | |
| | PEMGGKPRRKNPQEGLYNELQKDKMAEAYSEIGMKGERRRGKGH | |
| | DGLYQGLSTATKDTYDALHMQALPPR | |
| 126 | EVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIQ | |
| | LTQSPSSLSASVGDRVTITCRASQSISSYLNWYQQKPGKAPKLLIYA | |
| | ASSLQSGVPSRFSGSGSGTDFTLTISSLQPEDFATYYCQQSYSTPYTF | |
| | GQGTKVEIKTTTPAPRPPTPAPTIASQPLSLRPEACRPAAGGAVHTR | |
| | GLDFACDIYIWAPLAGTCGVLLLSLVITLYCKRGRKKLLYIFKQPF | |
| | MRPVQTTQEEDGCSCRFPEEEEGGCELRVKFSRSADAPAYKQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 127 | QVQLVESGGGLVQPGRSLRLSCAASGFTFSNYAMSWVRQAPGKG | anti-BCMA CAR |
| | LGWVSGISRSGENTYYADSVKGRFTISRDNSKNTLYLQMNSLRDE | |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIKTTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELRVKFSRSA | |
| | DAPAYKQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 128 | QVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIR | |
| | LTQSPSPLSASVGDRVTITCQASEDINKFLNWYHQTPGKAPKLLIY | |
| | DASTLQTGVPSRFSGSGSGTDFTLTINSLQPEDIGTYYCQQYESLPL | |
| | TFGGGTKVEIKTTTPAPRPPTPAPTIASQPLSLRPEACRPAAGGAVH | |
| | TRGLDFACDIYIWAPLAGTCGVLLLSLVITLYCKRGRKKLLYIFKQ | |
| | PFMRPVQTTQEEDGCSCRFPEEEEGGCELRVKFSRSADAPAYKQG | |
| | QNQLYNELNLGRREEYDVLDKRRGRDPEMGGKPRRKNPQEGLYN | |
| | ELQKDKMAEAYSEIGMKGERRRGKGHDGLYQGLSTATKDTYDAL | |
| | HMQALPPR | |
| 129 | EVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSEIV | |
| | LTQSPGTLSLSPGERATLSCRASQSIGSSSLAWYQQKPGQAPRLLM | |
| | YGASSRASGIPDRFSGSGSGTDFTLTISRLEPEDFAVYYCQQYAGSP | |
| | PFTFGQGTKVEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 130 | QIQLVQSGPDLKKPGETVKLSCKASGYTFTNFGMNWVKQAPGKG | anti-BCMA CAR |
| | FKWMAWINTYTGESYFADDFKGRFAFSVETSATTAYLQINNLKTE | |
| | DTATYFCARGEIYYGYDGGFAYWGQGTLVTVSAGGGGSGGGGSG | |
| | GGGSDVVMTQSHRFMSTSVGDRVSITCRASQDVNTAVSWYQQKP | |
| | GQSPKLLIFSASYRYTGVPDRFTGSGSGADFTLTISSVQAEDLAVYY | |
| | CQQHYSTPWTFGGGTKLDIKTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYKQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 131 | QIQLVQSGPELKKPGETVKISCKASGYTFTDYSINWVKRAPGKGLK | anti-BCMA CAR |
| | WMGWINTETREPAYAYDFRGRFAFSLETSASTAYLQINNLKYEDT | |
| | ATYFCALDYSYAMDYWGQGTSVTVSSGGGGSGGGGSGGGGSQIQ | |
| | LVQSGPELKKPGETVKISCKASGYTFTDYSINWVKRAPGKGLKWM | |
| | GWINTETREPAYAYDFRGRFAFSLETSASTAYLQINNLKYEDTATY | |
| | FCALDYSYAMDYWGQGTSVTVSSTTTPAPRPPTPAPTIASQPLSLR | |
| | PEACRPAAGGAVHTRGLDFACDIYIWAPLAGTCGVLLLSLVITLYC | |
| | KRGRKKLLYIFKQPFMRPVQTTQEEDGCSCRFPEEEEGGCELRVKF | |
| | SRSADAPAYKQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 132 | QIQLVQSGPELKKPGETVKISCKASGYTFTDYSINWVKRAPGKGLK | anti-BCMA CAR |
| | WMGWINTETREPAYAYDFRGRFAFSLETSASTAYLQINNLKYEDT | |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 133 | QIQLVQSGPELKKPGETVKISCKASGYTFRHYSMNWVKQAPGKGL | anti-BCMA CAR |
| | KWMGRINTESGVPIYADDFKGRFAFSVETSASTAYLVINNLKDEDT | |
| | ASYFCSNDYLYSLDFWGQGTALTVSSGGGGSGGGGSGGGGSDIVL | |
| | TQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTI | |
| | PRTFGGGTKLEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 134 | QIQLVQSGPELKKPGETVKISCKASGYTFTHYSMNWVKQAPGKGL | anti-BCMA CAR |
| | KWMGRINTETGEPLYADDFKGRFAFSLETSASTAYLVINNLKNED | |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIKTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 135 | DIVLTQSPPSLAMSLGKRATISCRASESVTILGSHLIHWYQQKPGQP | anti-BCMA CAR |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSSFVPVFLPAKPTTTPAPRPPTPAPTIASQPLS | |
| | LRPEACRPAAGGAVHTRGLDFACDIYIWAPLAGTCGVLLLSLVITL | |
| | YCNHRNRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 136 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKGLAVSTISSFFPPGYQIYIWAPLAGTC | |
| | GVLLLSLVITLYCKRGRKKLLYIFKQPFMRPVQTTQEEDGCSCRFP | |
| | EEEEGGCELRVKFSRSADAPAYQQGQNQLYNELNLGRREEYDVL | |
| | DKRRGRDPEMGGKPRRKNPQEGLYNELQKDKMAEAYSEIGMKGE | |
| | RRRGKGHDGLYQGLSTATKDTYDALHMQALPPR | |
| 137 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 138 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKEPKSPDKTHTCPPCPAPPVAGPSVFL | |
| | FPPKPKDTLMIARTPEVTCVVVDVSHEDPEVKFNWYVDGVEVHN | |
| | AKTKPREEQYNSTYRVVSVLTVLHQDWLNGKEYKCKVSNKALPA | |
| | PIEKTISKAKGQPREPQVYTLPPSRDELTKNQVSLTCLVKGFYPSDI | |
| | AVEWESNGQPENNYKTTPPVLDSDGSFFLYSKLTVDKSRWQQGN | |
| | VFSCSVMHEALHNHYTQKSLSLSPGKIYIWAPLAGTCGVLLLSLVI | |
| | TLYCKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 139 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKGLAVSTISSFFPPGYQIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMKG | |
| | ERRRGKGHDGLYQGLSTATKDTYDALHMQALPPR | |
| 140 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKTTTPAPRPPTPAPTIASQPLSLRPE | |
| | ACRPAAGGAVHTRGLDFACDIYIWAPLAGTCGVLLLSLVITLYCK | |
| | RGRKKLLYIFKQPFMRPVQTTQEEDGCSCRFPEEEEGGCELRVKFS | |
| | RSADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 141 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKEPKSPDKTHTCPPCPAPPVAGPSV | |
| | FLFPPKPKDTLMIARTPEVTCVVVDVSHEDPEVKFNWYVDGVEVH | |
| | NAKTKPREEQYNSTYRVVSVLTVLHQDWLNGKEYKCKVSNKALP | |
| | APIEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKIYIWAPLAGTCGVLLLSLVI | |
| | TLYCKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 142 | IYIWAPLAGTCGVLLLSLVITLYCNHRN | CD8a TM |
| 143 | IYIWAPLAGTCGVLLLSLVIT | CD8a TM |
| 144 | RAAA | linking peptide |
| 145 | EVQLVESGGGLVKPGGSLRLSCAASGFTFSDYYMSWIRQAPGKGL | Variable heavy |
| | EWVSYISSSGSTIYYADSVKGRFTISRDNAKNSLYLQMNSLRAEDT | (VH) Anti- |
| | AVYYCAKVDGDYTEDYWGQGTLVTVSS | BCMA |
| 146 | QSALTQPASVSGSPGQSITISCTGSSSDVGKYNLVSWYQQPPGKAP | Variable light |
| | KLIIYDVNKRPSGVSNRFSGSKSGNTATLTISGLQGDDEADYYCSS | (VL) Anti- |
| | YGGSRSYVFGTGTKVTVL | BCMA |
| 147 | EVQLVQSGGGLVQPGRSLRLSCTASGFTFGDYAMSWFRQAPGKG | Variable heavy |
| | LEWVGFIRSKAYGGTTEYAASVKGRFTISRDDSKSIAYLQMNSLKT | (VH) Anti- |
| | EDTAVYYCAAWSAPTDYWGQGTLVTVSS | BCMA |
| 148 | DIQMTQSPAFLSASVGDRVTVTCRASQGISNYLAWYQQKPGNAPR | Variable light |
| | LLIYSASTLQSGVPSRFRGTGYGTEFSLTIDSLQPEDFATYYCQQSY | (VL) Anti- |
| | TSRQTFGPGTRLDIK | BCMA |
| 149 | EVQLVESGGGLVKPGGSLRLSCAASGFTFSDYYMSWIRQAPGKGL | Variable heavy |
| | EWVSYISSSGSTIYYADSVKGRFTISRDNAKNSLYLQMNSLRAEDT | (VH) Anti- |
| | AVYYCAKVDGPPSFDIWGQGTMVTVSS | BCMA |
| 150 | SYVLTQPPSVSVAPGQTARITCGANNIGSKSVHWYQQKPGQAPML | Variable light |
| | VVYDDDDRPSGIPERFSGSNSGNTATLTISGVEAGDEADYFCHLW | (VL) Anti- |
| | DRSRDHYVFGTGTKLTVL | BCMA |
| 151 | EVQLVQSGAEVKKPGSSVKVSCKASGGTFSSYAISWVRQAPGQGL | Variable heavy |
| | EWMGRIIPILGIANYAQKFQGRVTMTEDTSTDTAYMELSSLRSEDT | (VH) Anti- |
| | AVYYCARSGYSKSIVSYMDYWGQGTLVTVSS | BCMA |
| 152 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 153 | MPLLLLLPLLWAGALA | CD33 Signal |
| | | peptide |
| 154 | MALPVTALLLPLALLLHA | CD8 alpha signal |
| | | peptide |
| 155 | atgcttctcctggtgacaagccttctgctctgtgagttaccacacc | GMCSFR alpha |
| | cagcattcctcctgatccca | chain signal |
| | | sequence |
| 156 | MLLLVTSLLLCELPHPAFLLIP | GMCSFR alpha |
| | | chain signal |
| | | sequence |
| 157 | Glu Val Val Val Lys Tyr Gly Pro Pro Cys Pro Pro Cys Pro | Exemplary IgG |
| | | Hinge |
| 158 | X1PPX2P | Exemplary IgG |
| | X1 is glycine, cysteine or arginine | Hinge |
| | X2 is cysteine or threonine | |
| 159 | Glu Pro Lys Ser Cys Asp Lys Thr His Thr Cys Pro Pro Cys Pro | Exemplary IgG |
| | | Hinge |
| 160 | Glu Arg Lys Cys Cys Val Glu Cys Pro Pro Cys Pro | Exemplary IgG |
| | | Hinge |
| 161 | ELKTPLGDTHTCPRCPEPKSCDTPPPCPRCPEPKSCDTPPPCPRCPEP | Exemplary IgG |
| | KSCDTPPPCPRCP | Hinge |
| 162 | Glu Ser Lys Tyr Gly Pro Pro Cys Pro Ser Cys Pro | Exemplary IgG |
| | | Hinge |
| 163 | Glu Ser Lys Tyr Gly Pro Pro Cys Pro Pro Cys Pro | Exemplary IgG |
| | | Hinge |
| 164 | Tyr Gly Pro Pro Cys Pro Pro Cys Pro | Exemplary IgG |
| | | Hinge |
| 165 | Lys Tyr Gly Pro Pro Cys Pro Pro Cys Pro | Exemplary IgG |
| | | Hinge |
| 166 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | artificial |
| | QAWPENRTDLHAFENLEIIRGRTKQHGQFSLAVVSLNITSLGLRSL | |
| | KEISDGDVIISGNKNLCYANTINWKKLFGTSGQKTKIISNRGENSCK | |
| | ATGQVCHALCSPEGCWGPEPRDCVSCRNVSRGRECVDKCNLLEG | |
| | EPREFVENSECIQCHPECLPQAMNITCTGRGPDNCIQCAHYIDGPHC | |
| | VKTCPAGVMGENNTLVWKYADAGHVCHLCHPNCTYGCTGPGLE | |
| | GCPTNGPKIPSIATGMVGALLLLLVVALGIGLFM | |
| 167 | EGRGSLLTCGDVEENPGP | T2A artificial |
| 168 | GSGATNFSLLKQAGDVEENPGP | P2A |
| 169 | ATNFSLLKQAGDVEENPGP | P2A |
| 170 | QCTNYALLKLAGDVESNPGP | E2A |
| 171 | VKQTLNFDLLKLAGDVESNPGP | F2A |
| 172 | MLQMAGQCSQNEYFDSLLHACIPCQLRCSSNTPPLTCQRYCNASV | BCMA-Fc fusion |
| | TNSVKGTNAGGGGSPKSSDKTHTCPPCPAPEAEGAPSVFLFPPKPK | polypeptide |
| | DTLMISRTPEVTCVVVDVSHEDPEVKFNWYVDGVEVHNAKTKPR | |
| | EEQYNSTYRVVSVLTVLHQDWLNGKEYKCKVSNKALPSSIEKTIS | |
| | KAKGQPREPQVYTLPPSRDELTKNQVSLTCLVKGFYPSDIAVEWES | |
| | NGQPENNYKTTPPVLDSDGSFFLYSKLTVDKSRWQQGNVFSCSVM | |
| | HEALHNHYTQKSLSLSPGK | |
| 173 | DYYVY | CDR-H1 |
| 174 | WINPNSGGTNYAQKFQG | CDR-H2 |
| 175 | SQRDGYMDY | CDR-H3 |
| 176 | GYTFIDY | CDR-H1 |
| 177 | NPNSGG | CDR-H2 |
| 178 | GYTFIDYYVY | CDR-H1 |
| 179 | WINPNSGGTN | CDR-H2 |
| 180 | GYTFIDYY | CDR-H1 |
| 181 | INPNSGGT | CDR-H2 |
| 182 | ARSQRDGYMDY | CDR-H3 |
| 183 | TGTSSDVG | CDR-L1 |
| 184 | EDSKRPS | CDR-L2 |
| 185 | SSNTRSSTLV | CDR-L3 |
| 186 | ISCTGTSSD | CDR-L1 |
| 187 | EDS | CDR-L2 |
| 188 | QSALTQPASVSASPGQSIAISCTGTSSDVGWYQQHPGKAPKLMIYE | anti-BCMA scFv |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSS | |
| 189 | DYSIN | CDR-H1 |
| 190 | WINTETREPAYAYDFRG | CDR-H2 |
| 191 | DYSYAMDY | CDR-H3 |
| 192 | RASESVTILGSHLIH | CDR-L1 |
| 193 | LASNVQT | CDR-L2 |
| 194 | LQSRTIPRT | CDR-L3 |
| 195 | GYTFTDY | CDR-H1 |
| 196 | NTETRE | CDR-H2 |
| 197 | DYSYAMDY | CDR-H3 |
| 198 | RASESVTILGSHLIH | CDR-L1 |
| 199 | LASNVQT | CDR-L2 |
| 200 | LQSRTIPRT | CDR-L3 |
| 201 | GYTFTDYSIN | CDR-H1 |
| 202 | WINTETREPA | CDR-H2 |
| 203 | DYSYAMDY | CDR-H3 |
| 204 | RASESVTILGSHLIH | CDR-L1 |
| 205 | LASNVQT | CDR-L2 |
| 206 | LQSRTIPRT | CDR-L3 |
| 207 | GYTFTDYS | CDR-H1 |
| 208 | INTETREP | CDR-H2 |
| 209 | ALDYSYAMDY | CDR-H3 |
| 210 | ESVTILGSHL | CDR-L1 |
| 211 | LA | CDR-L2 |
| 212 | LQSRTIPRT | CDR-L3 |
| 213 | DIVLTQSPPSLAMSLGKRATISCRASESVTILGSHLIHWYQQKPGQP | scFv |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDERGRFAFSLETSASTAYLQINNLKYEDTATYFCALDYS | |
| | YAMDYWGQGTSVTVSS | |
| 214 | atggcactcc ccgtcaccgc ccttctcttg cccctcgccc tgctgctgca tgctgccagg | BCMA CAR (nt) |
| | cccgacattg tgctcactca gtcacctccc agcctggcca tgagcctggg aaaaagggcc | |
| | accatctcct gtagagccag tgagtccgtc acaatcttgg ggagccatct tattcactgg | |
| | tatcagcaga agcccgggca gcctccaacc cttcttattc agctcgcgtc aaacgtccag | |
| | acgggtgtac ctgccagatt ttctggtagc gggtcccgca ctgattttac actgaccata | |
| | gatccagtgg aagaagacga tgtggccgtg tattattgtc tgcagagcag aacgattcct | |
| | cgcacatttg gtgggggtac taagctggag attaagggaa gcacgtccgg ctcagggaag | |
| | ccgggctccg gcgagggaag cacgaagggg caaattcagc tggtccagag cggacctgag | |
| | ctgaaaaaac ccggcgagac tgttaagatc agttgtaaag catctggcta taccttcacc | |
| | gactacagca taaattgggt gaaacgggcc cctggaaagg gcctcaaatg gatgggttgg | |
| | atcaataccg aaactaggga gcctgcttat gcatatgact tccgcgggag attcgccttt | |
| | tcactcgaga catctgcctc tactgcttac ctccaaataa acaacctcaa gtatgaagat | |
| | acagccactt acttttgcgc cctcgactat agttacgcca tggactactg gggacaggga | |
| | acctccgtta ccgtcagttc cgcggccgca accacaacac ctgctccaag gccccccaca | |
| | cccgctccaa ctatagccag ccaaccattg agcctcagac ctgaagcttg caggcccgca | |
| | gcaggaggcg ccgtccatac gcgaggcctg gacttcgcgt gtgatattta tatttgggcc | |
| | cctttggccg gaacatgtgg ggtgttgctt ctctcccttg tgatcactct gtattgtaag | |
| | cgcgggagaa | |
| | agaagctcct gtacatcttc aagcagcctt ttatgcgacc tgtgcaaacc actcaggaag | |
| | aagatgggtg ttcatgccgc ttccccgagg aggaagaagg agggtgtgaa ctgagggtga | |
| | aattttctag aagcgccgat gctcccgcat atcagcaggg tcagaatcag | |
| | ctctacaatg aattgaatct cggcaggcga gaagagtacg atgttctgga caagagacgg | |
| | gccagggatc ccgagatggg gggaaagccc cggagaaaaa atcctcagga ggggttgtac | |
| | aatgagctgc agaaggacaa gatggctgaa gcctatagcg agatcggaat gaaaggcgaa | |
| | agacgcagag gcaaggggca tgacggtctg taccagggtc tctctacagc caccaaggac | |
| | acttatgatg cgttgcatat gcaagccttg ccaccccgct aatga | |
| 215 | MLQMAGQCSQNEYFDSLLHACIPCQLRCSSNTPPLTCQRYCNASV | Human BCMA |
| | TNSVKGTNAILWTCLGLSLIISLAVFVLMFLLRKINSEPLKDEFKNT | |
| | GSGLLGMANIDLEKSRTGDEIILPRGLEYTVEECTCEDCIKSKPKVD | |
| | SDHCFPLPAMEEGATILVTTKTNDYCKSLPAALSATEIEKSISAR | |
| 216 | EVQLLESGGGLVQPGGSLRLSCAASGFTFSSYAMSWVRQAPGKGL | Anti-BCMA scFv |
| | EWVSAISGSGGSTYYADSVKGRFTISRDNSKNTLYLQMNSLRAED | |
| | TAVYYCARAEMGAVFDIWGQGTMVTVSSGSTSGSGKPGSGEGST | |
| | KGBIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIK | |
| 217 | EIVLTQSPATLSLSPGERATLSCRASQSVSRYLAWYQQKPGQAPRL | Anti-BCMA scFv |
| | LIYDASNRATGIPARFSGSGSGTDFTLTISSLEPEDFAVYYCQQRIS | |
| | WPFTFGGGTKVEIKRGSTSGSGKPGSGEGSTKGEVQLLESGGGLV | |
| | QPGGSLRLSCAASGFTESSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSS | |
| 218 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIK | |
| 219 | DIVMTQSPLSLPVTPGEPASISCRSSQSLLHSNGYNYLDWYLQKPG | Anti-BCMA scFv |
| | QSPQLLIYLGSNRASGVPDRFSGSGSGTDFTLKISRVEAEDVGVYY | |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSS | |
| 220 | QVQLVQSGAEVKKPGASVKVSCKASGYTFTSYYMHWVRQAPGQ | Anti-BCMA scFv |
| | GLEWMGIINPGGGSTSYAQKFQGRVTMTRDTSTSTVYMELSSLRS | |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQY | |
| | AAYPTFGGGTKVEIK | |
| 221 | EIVMTQSPATLSVSPGERATLSCRASQSVSSNLAWYQQKPGQAPRL | Anti-BCMA scFv |
| | LIYGASTRATGIPARFSGSGSGTEFTLTISSLQSEDFAVYYCQQYAA | |
| | YPTFGGGTKVEIKRGSTSGSGKPGSGEGSTKGQVQLVQSGAEVKK | |
| | PGASVKVSCKASGYTFTSYYMHWVRQAPGQGLEWMGIINPGGGS | |
| | TSYAQKFQGRVTMTRDTSTSTVYMELSSLRSEDTAVYYCARESWP | |
| | MDVWGQGTTVTVSS | |
| 222 | QLQLQESGPGLVKPSETLSLTCTVSGGSISSSSYYWGWIRQPPGKG | Anti-BCMA scFv |
| | LEWIGSISYSGSTYYNPSLKSRVTISVDTSKNQFSLKLSSVTAADTA | |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIK | |
| 223 | EIVLTQSPATLSLSPGERATLSCRASQSVSSYLAWYQQKPGQAPRL | Anti-BCMA scFv |
| | LIYDASNRATGIPARFSGSGSGTDFTLTISSLEPEDFAVYYCQQRHV | |
| | WPPTFGGGTKVEIKRGSTSGSGKPGSGEGSTKGQLQLQESGPGLVK | |
| | PSETLSLTCTVSGGSISSSSYYWGWIRQPPGKGLEWIGSISYSGSTY | |
| | YNPSLKSRVTISVDTSKNQFSLKLSSVTAADTAVYYCARGRGYATS | |
| | LAFDIWGQGTMVTVSS | |
| 224 | EVQLVESGGGLVQPGGSLRLSCAASGFTFSSYSMNWVRQAPGKGL | Anti-BCMA scFv |
| | EWVSTISSSSSTIYYADSVKGRFTISRDNAKNSLYLQMNSLRAEDT | |
| | AVYYCARGSQEHLIFDYWGQGTLVTVSSGSTSGSGKPGSGEGSTK | |
| | GEIVLTQSPATLSLSPGERATLSCRASQSVSRYLAWYQQKPGQAPR | |
| | LLIYDASNRATGIPARFSGSGSGTDFTLTISSLEPEDFAVYYCQQRF | |
| | YYPWTFGGGTKVEIK | |
| 225 | EIVLTQSPATLSLSPGERATLSCRASQSVSRYLAWYQQKPGQAPRL | Anti-BCMA scFv |
| | LIYDASNRATGIPARFSGSGSGTDFTLTISSLEPEDFAVYYCQQRFY | |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSS | |
| 226 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCARTDFWSGSPPGLDYWGQGTLVTVSSGSTSGSGKPGSG | |
| | EGSTKGDIQLTQSPSSVSASVGDRVTITCRASQGISSWLAWYQQKP | |
| | GKAPKLLIYGASSLQSGVPSRFSGSGSGTDFTLTISSLQPEDFATYY | |
| | CQQIYTFPFTFGGGTKVEIK | |
| 227 | DIQLTQSPSSVSASVGDRVTITCRASQGISSWLAWYQQKPGKAPKL | Anti-BCMA scFv |
| | LIYGASSLQSGVPSRFSGSGSGTDFTLTISSLQPEDFATYYCQQIYTF | |
| | PFTFGGGTKVEIKRGSTSGSGKPGSGEGSTKGQVQLVESGGGVVQP | |
| | GRSLRLSCAASGFTFSSYGMHWVRQAPGKGLEWVAVISYDGSNK | |
| | YYADSVKGRFTISRDNSKNTLYLQMNSLRAEDTAVYYCARTDFW | |
| | SGSPPGLDYWGQGTLVTVSS | |
| 228 | QVQLVQSGAEVKKPGSSVKVSCKASGGTFSSYAISWVRQAPGQGL | Anti-BCMA scFv |
| | EWMGGIIPIFGTANYAQKFQGRVTITADESTSTAYMELSSLRSEDT | |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIK | |
| 229 | DIVMTQSPDSLAVSLGERATINCKSSQSVLYSSNNKNYLAWYQQK | Anti-BCMA scFv |
| | PGQPPKLLIYWASTRESGVPDRFSGSGSGTDFTLTISSLQAEDVAVY | |
| | YCQQFAHTPFTFGGGTKVEIKRGSTSGSGKPGSGEGSTKGQVQLV | |
| | QSGAEVKKPGSSVKVSCKASGGTFSSYAISWVRQAPGQGLEWMG | |
| | GIIPIFGTANYAQKFQGRVTITADESTSTAYMELSSLRSEDTAVYYC | |
| | ARTPEYSSSIWHYYYGMDVWGQGTTVTVSS | |
| 230 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIK | |
| 231 | EIVMTQSPATLSVSPGERATLSCRASQSVSSNLAWYQQKPGQAPRL | Anti-BCMA scFv |
| | LIYSASTRATGIPARFSGSGSGTEFTLTISSLQSEDFAVYYCQQHHV | |
| | WPLTFGGGTKVEIKRGSTSGSGKPGSGEGSTKGQVQLVESGGGVV | |
| | QPGRSLRLSCAASGFTFSSYGMHWVRQAPGKGLEWVAVISYDGS | |
| | NKYYADSVKGRFTISRDNSKNTLYLQMNSLRAEDTAVYYCVKGP | |
| | LQEPPYDYGMDVWGQGTTVTVSS | |
| 232 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 233 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 234 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 235 | EVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIQ | |
| | LTQSPSSLSASVGDRVTITCRASQSISSYLNWYQQKPGKAPKLLIYA | |
| | ASSLQSGVPSRFSGSGSGTDFTLTISSLQPEDFATYYCQQSYSTPYTF | |
| | GQGTKVEIK | |
| 236 | QVQLVESGGGLVQPGRSLRLSCAASGFTFSNYAMSWVRQAPGKG | Anti-BCMA scFv |
| | LGWVSGISRSGENTYYADSVKGRFTISRDNSKNTLYLQMNSLRDE | |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIK | |
| 237 | QVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIR | |
| | LTQSPSPLSASVGDRVTITCQASEDINKFLNWYHQTPGKAPKLLIY | |
| | DASTLQTGVPSRFSGSGSGTDFTLTINSLQPEDIGTYYCQQYESLPL | |
| | TFGGGTKVEIK | |
| 238 | EVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSEIV | |
| | LTQSPGTLSLSPGERATLSCRASQSIGSSSLAWYQQKPGQAPRLLM | |
| | YGASSRASGIPDRFSGSGSGTDFTLTISRLEPEDFAVYYCQQYAGSP | |
| | PFTFGQGTKVEIK | |
| 239 | QIQLVQSGPELKKPGETVKISCKASGYTFTDYSINWVKRAPGKGLK | Anti-BCMA scFv |
| | WMGWINTETREPAYAYDFRGRFAFSLETSASTAYLQINNLKYEDT | |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIK | |
| 240 | QIQLVQSGPELKKPGETVKISCKASGYTFRHYSMNWVKQAPGKGL | Anti-BCMA scFv |
| | KWMGRINTESGVPIYADDFKGRFAFSVETSASTAYLVINNLKDEDT | |
| | ASYFCSNDYLYSLDFWGQGTALTVSSGGGGSGGGGSGGGGSDIVL | |
| | TQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTI | |
| | PRTFGGGTKLEIK | |
| 241 | QIQLVQSGPELKKPGETVKISCKASGYTFTHYSMNWVKQAPGKGL | Anti-BCMA scFv |
| | KWMGRINTETGEPLYADDFKGRFAFSLETSASTAYLVINNLKNED | |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIK | |
| 242 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 243 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 244 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 245 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |
| 246 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |
| 247 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |

1. A method of treating multiple myeloma in a subject who had an early relapse, an inadequate response, or a suboptimal response to one or more anti-myeloma treatment, comprising administering to the subject a BCMA targeted CAR T cell therapy followed by an immunomodulatory agent maintenance therapy.

2. A method of treating multiple myeloma in a subject, the method comprising:
(a) selecting a subject who had an early relapse, an inadequate response, or a suboptimal response to one or more anti-myeloma treatment;
(b) administering to the selected subject a BCMA targeted CAR T cell therapy; and
(c) after administering the BCMA targeted CAR T cell therapy, administering to the subject an immunomodulatory agent maintenance therapy.

3. A method of treating newly diagnosed multiple myeloma (NDMM) in a subject who had an early relapse, inadequate response, or a suboptimal response to one or more anti-myeloma treatment, comprising administering to the subject a BCMA targeted CAR T cell therapy followed by an immunomodulatory agent maintenance therapy.

4. A method of treating newly diagnosed multiple myeloma (NDMM) in a subject, the method comprising:
(a) selecting a subject who had an early relapse, an inadequate response, or a suboptimal response to one or more anti-myeloma treatment;
(b) administering to the selected subject a BCMA targeted CAR T cell therapy; and
(c) after administering the BCMA targeted CAR T cell therapy, administering to the subject an immunomodulatory agent maintenance therapy.

5. A method of maintenance therapy for treating multiple myeloma, the method comprising administering an immunomodulatory agent maintenance therapy to a subject after having received a BCMA targeted CAR T cell therapy for treating multiple myeloma, wherein prior to the BCMA target CAR T cell therapy the subject had an early relapse, an inadequate response, or a suboptimal response to one or more other anti-myeloma treatment.

6. The method of any of claims 1-5, wherein the immunomodulatory agent maintenance therapy is a maintenance therapy with lenalidomide or a pharmaceutically acceptable salt thereof.

7. The method of any of claims 1-6, wherein the one or more anti-myeloma treatment comprises stem cell transplant therapy.

8. The method of claim 7, wherein the stem cell transplant therapy comprises autologous stem cell therapy (ASCT).

9. The method of claim 7 or 8, wherein the stem cell transplant therapy comprises an induction therapy followed by a stem cell transplant.

10. The method of any of claims 7-9, wherein the stem cell transplant therapy comprises an induction therapy followed by high-dose chemotherapy (HDT) and a stem cell transplant.

11. The method of any of claims 8-10, wherein the stem cell transplant therapy consists of an induction therapy followed by a stem cell transplant.

12. The method of claim 1-11, wherein the multiple myeloma is a high-risk multiple myeloma.

13. The method of any of claims 1-12, wherein the subject is a subject that had an inadequate response to one or more anti-myeloma treatment.

14. The method of any of claims 1-13, wherein the inadequate response to the one or more anti-myeloma treatment is characterized by less than very good partial response (VGPR), e.g., at 70-110 days, after last treatment of the one or more anti-myeloma treatment without use of consolidation or maintenance therapy.

15. The method of any of claims 1-13, wherein the inadequate response to the one or more anti-myeloma treatment is characterized by less than very good partial response (VGPR), e.g., at 80-120 days, after last treatment of the one or more anti-myeloma treatment without use of consolidation or maintenance therapy.

16. The method of any of claims 1-14, wherein the one or more anti-myeloma treatment is an ASCT with prior induction therapy and the inadequate response is characterized by less than very good partial response (VGPR), e.g., at 70-110 days, after last ASCT without use of consolidation or maintenance therapy.

17. The method of any of claim 1-13 or 15, wherein the one or more anti-myeloma treatment is an ASCT with prior induction therapy and the inadequate response is characterized by less than very good partial response (VGPR), e.g., at 80-120 days, after last ASCT without use of consolidation or maintenance therapy.

18. A method of treating high-risk multiple myeloma in a subject, comprising administering to the subject a BCMA targeted CAR T cell therapy followed by an immunomodulatory agent maintenance therapy,
wherein the subject, at the time of administering the BCMA targeted CAR T cell therapy, has a high-risk multiple myeloma characterized by an inadequate response to an autologous stem cell transplant (ASCT) after an induction therapy for treating the multiple myeloma.

19. A method of treating high-risk multiple myeloma in a subject, the method comprising:
(a) selecting a subject with a multiple myeloma characterized by an inadequate response to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

20. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had an inadequate response to an autologous stem cell transplant (ASCT) after an induction therapy for treating the multiple myeloma.

21. A method of treating high-risk multiple myeloma in a subject, comprising administering to the subject a BCMA targeted CAR T cell therapy,
wherein the subject, at the time of administering the BCMA targeted CAR T cell therapy, has a high-risk multiple myeloma characterized by an inadequate response to autologous stem cell transplant (ASCT) after an induction therapy for treating the multiple myeloma.

22. The method of any of claims 17-21, wherein the inadequate response to the autologous stem cell transplant (ASCT) after an induction therapy is characterized by less than very good partial response (VGPR), e.g., at 70-110 days, after last ASCT.

23. The method of any of claims 17-21, wherein the inadequate response to the autologous stem cell transplant (ASCT) after an induction therapy is characterized by less than very good partial response (VGPR), e.g., at 80-120 days, after last ASCT.

24. The method of any of claims 17-23, wherein the ASCT after the induction therapy is a frontline therapy for treating a newly diagnosed multiple myeloma (NDMM).

25. The method of any of claims 1-11, wherein the subject is a subject that had an early relapse to the one or more anti-myeloma treatment.

26. The method of any of claims 1-11 and 25, wherein the early relapse is characterized by development of progressive disease (PD) less than 18 months from starting the one or more anti-myeloma treatment.

27. A method of treating high-risk multiple myeloma in a subject, comprising administering to the subject a BCMA targeted CAR T cell therapy followed by an immunomodulatory agent maintenance therapy,
wherein the subject, at the time of administering the BCMA targeted CAR T cell therapy, has a high-risk multiple myeloma characterized by early relapse to autologous stem cell transplant (ASCT) after an induction therapy for treating the multiple myeloma.

28. A method of treating high-risk multiple myeloma in a subject, the method comprising:
(a) selecting a subject with a multiple myeloma characterized by an early relapse to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

29. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had an early relapse to an autologous stem cell transplant (ASCT) after an induction therapy for treating the multiple myeloma.

30. A method of treating multiple myeloma in a subject, comprising administering to the subject a BCMA targeted CAR T cell therapy,
wherein the subject, at the time of administering the BCMA targeted CAR T cell therapy, has a multiple myeloma characterized by an early relapse to an autologous stem cell transplant (ASCT) after an induction therapy for treating the multiple myeloma.

31. The method of any of any of claims 27-30, wherein the early relapse is characterized by development of progressive disease (PD) less than 18 months from starting the autologous stem cell transplant (ASCT) after an induction therapy.

32. The method of any of claims 1-11, wherein the subject is a subject that had a suboptimal response to one or more anti-myeloma treatment.

33. The method of any of claim 1-11 or 32, wherein the suboptimal response to the one or more anti-myeloma treatment is characterized by partial response (PR) or very good partial response (VGPR), e.g., at 80-120 days, after last treatment of the one or more anti-myeloma treatment without use of consolidation or maintenance therapy.

34. The method of any of claim 1-11 or 32-33, wherein the suboptimal response to the one or more anti-myeloma treatment is characterized by partial response (PR) or very good partial response (VGPR) at about 100 days after last treatment of the one or more anti-myeloma treatment without use of consolidation or maintenance therapy.

35. The method of any of claim 1-11 or 32-33, wherein the one or more anti-myeloma treatment is an ASCT with prior induction therapy and the suboptimal response is characterized by partial response or very good partial response (VGPR), e.g., at 80-120 days, after last ASCT without use of consolidation or maintenance therapy.

36. The method of any of claim 1-11 or 32-35, wherein the one or more anti-myeloma treatment is an ASCT with prior induction therapy and the suboptimal response is characterized by partial response or very good partial response (VGPR) at about or at 100 days after last ASCT without use of consolidation or maintenance therapy.

37. The method of claim 35 or claim 36, wherein the ASCT with prior induction therapy further comprises high-dose chemotherapy (HDT).

38. A method of treating multiple myeloma in a subject, comprising administering to the subject a BCMA targeted CAR T cell therapy followed by an immunomodulatory agent maintenance therapy,
wherein the subject, at the time of administering the BCMA targeted CAR T cell therapy, has a multiple myeloma characterized by a suboptimal response to autologous stem cell transplant (ASCT) after an induction therapy for treating the multiple myeloma.

39. A method of treating multiple myeloma in a subject, the method comprising:
(a) selecting a subject with a multiple myeloma characterized by a suboptimal response to an autologous stem cell transplant (ASCT) after an induction therapy for treating the multiple myeloma;
(b) administering to the selected subject a BCMA targeted CAR T cell therapy; and
(c) after administering the BCMA targeted CAR T cell therapy, administering to the subject an immunomodulatory agent maintenance therapy.

40. A method of treating multiple myeloma in a subject, the method comprising:
(a) selecting a subject with a multiple myeloma characterized by a suboptimal response to an autologous stem cell transplant (ASCT) after an induction therapy for treating the multiple myeloma;
(b) administering to the selected subject an immunomodulatory agent maintenance therapy prior to a leukapheresis to obtain T cells from the subject for engineering with a BCMA targeted CAR to produce a BCMA targeted CAR T cell therapy;
(c) administering to the selected subject the BCMA targeted CAR T cell therapy; and
(d) after administering the BCMA targeted CAR T cell therapy, administering to the subject an immunomodulatory agent maintenance therapy.

41. A method of maintenance therapy for treating multiple myeloma in a subject, the method comprising administering an immunomodulatory agent maintenance therapy to a subject after having received a BCMA targeted CAR T cell therapy for treating multiple myeloma, wherein prior to the BCMA target CAR T cell therapy the subject had a suboptimal response to an autologous stem cell transplant (ASCT) after an induction therapy for treating the multiple myeloma.

42. The method of any of claim 38-39 or 41, wherein the subject has been administered an immunomodulatory agent maintenance therapy prior to a leukapheresis to obtain T cells from the subject for engineering with a BCMA targeted CAR to produce a BCMA targeted CAR T cell therapy.

43. A method of treating multiple myeloma in a subject, comprising administering to the subject a BCMA targeted CAR T cell therapy,
wherein the subject, at the time of administering the BCMA targeted CAR T cell therapy, has a multiple myeloma characterized by a suboptimal response an autologous stem cell transplant (ASCT) after an induction therapy for treating the multiple myeloma.

44. The method of any of claims 38-43, wherein the suboptimal response to the autologous stem cell transplant (ASCT) after an induction therapy is characterized by partial response (PR) or very good partial response (VGPR), e.g., at 80-120 days, after last ASCT.

45. The method of any of claims 38-44, wherein the suboptimal response to the autologous stem cell transplant (ASCT) after an induction therapy is characterized by partial response (PR) or very good partial response (VGPR) at about or at 100 days after last ASCT.

46. The method of any of claims 38-45, wherein the ASCT after induction therapy further comprises high-dose chemotherapy (HDT).

47. The method of any of claims 38-46, wherein the induction therapy comprises one or more of a proteasome inhibitor and an immunomodulatory agent.

48. The method of any of claims 9-47, wherein the induction therapy is one or more of a proteasome inhibitor, an immunomodulatory agent, and dexamethasone.

49. The method of any of claims 9-46, wherein the induction therapy is:
(i) bortezomib, lenalidomide or a pharmaceutically acceptable salt thereof, and dexamethasone (VRD);
(ii) thalidomide and dexamethasone (TD);
(iii) lenalidomide or a pharmaceutically acceptable salt thereof and low-dose dexamethasone (RD);
(iv) bortezomib and dexamethasone (VD);
(v) bortezomib, thalidomide and dexamethasone (VTD);
(vi) carfilzomib, lenalidomide or a pharmaceutically acceptable salt thereof, and dexamethasone (KRd); or
(vii) ixazomib, lenalidomide or a pharmaceutically acceptable salt thereof, and dexamethasone (Ixa-Rd).

50. The method of any of claims 8-49, wherein the induction therapy is administered in ≥3 cycles.

51. The method of any of claims 9-50, wherein the induction therapy is administered in 3-12 cycles.

52. The method of any of claims 9-50, wherein the induction therapy is administered in 3-6 cycles.

53. The method of any of claims 38-50, wherein the induction therapy is administered in 4-6 cycles.

54. The method of any of claims 48-53, wherein each cycle is a 28-day cycle.

55. The method of any of claims 48-54, wherein in each cycle of the induction therapy:
bortezomib is administered at or about 1.3 mg/m² intravenously or subcutaneously on days 1, 8 and 15 of the cycle;
lenalidomide or a pharmaceutically acceptable salt thereof is administered at or about 25 mg orally on days 1 to 21 of the cycle or days 1 to 14 of the cycle; and/or
dexamethasone is administered at or about 40 mg on days 1, 8 and 15 of the cycle.

56. The method of any of claims 1-55, wherein prior to receiving the BCMA targeted CAR T cell therapy, the subject had not received a prior immunomodulatory agent maintenance therapy.

57. The method of any of claims 1-56, wherein prior to receiving the BCMA targeted CAR T cell therapy, the subject had not received a consolidation therapy.

58. The method of any of claims 9-55, wherein prior to receiving the BCMA targeted CAR T cell therapy, the subject received a prior immunomodulatory agent maintenance therapy after the ASCT with the induction therapy.

59. The method of any of claims 1-58, wherein the subject was diagnosed with multiple myeloma about three years or less before administering the BCMA targeted CAR T cell therapy.

60. The method of any of claims 1-58, wherein the subject was diagnosed with multiple myeloma about two years or less before administering the BCMA targeted CAR T cell therapy.

61. The method of any of claims 1-58, wherein the subject was diagnosed with multiple myeloma about 1.6 years or less before administering the BCMA targeted CAR T cell therapy.

62. The method of any of claims 1-61, wherein at the time of administering the BCMA targeted CAR T cell therapy the subject has R-ISS stage I disease.

63. The method of any of claims 1-61, wherein at the time of administering the BCMA targeted CAR T cell therapy the subject has R-ISS stage II disease.

64. The method of any of claims 1-61, wherein at the time of administering the BCMA targeted CAR T cell therapy the subject has R-ISS stage III disease.

65. The method of any of claims 1-64, wherein at the time of administering the BCMA targeted CAR T cell therapy the subject has high-risk cytogenetics.

66. The method of any of claims 1-64, wherein at the time of administering the BCMA targeted CAR T cell therapy, the subject has ultra high-risk cytogenetics.

67. The method of any of claims 1-66, wherein at the time of administering the BCMA targeted CAR T cell therapy the subject has bone marrow biopsy-determined high tumor burden, optionally wherein the high tumor burden is ≥50% bone marrow CD138+ plasma cells.

68. The method of any of claims 1-67, wherein at the time of administering the BCMA targeted CAR T cell therapy the subject has extramedullary disease.

69. The method of any of claims 1-68, wherein at the time of administering the BCMA targeted CAR T cell therapy the multiple myeloma was refractory to treating with one or both of an immunomodulatory agent and a proteasome inhibitor (PI).

70. The method of any of claims 1-69, wherein at the time of administering the BCMA targeted CAR T cell therapy the subject had an ECOG performance status (PS)≤1.

71. The method of any of claims 1-70, wherein at the time of administering the BCMA targeted CAR T cell therapy the subject had an ECOG PS of 0.

72. The method of any of claims 42-71, wherein the immunomodulatory agent maintenance therapy prior to leukapheresis is initiated between or from 10 to 12 weeks prior to administering the BCMA targeted CAR-T cell therapy.

73. The method of claim 72, wherein the immunomodulatory agent maintenance therapy prior to leukapheresis is initiated at about 10 weeks prior to administering the BCMA targeted CAR-T cell therapy.

74. The method of claim 72, wherein the immunomodulatory agent maintenance therapy prior to leukapheresis is initiated at about 11 weeks prior to administering the BCMA targeted CAR-T cell therapy.

75. The method of claim 72, wherein the immunomodulatory agent maintenance therapy prior to leukapheresis is initiated at about 12 weeks prior to administering the BCMA targeted CAR-T cell therapy.

76. The method of any of claims 42-75, wherein the immunomodulatory agent maintenance therapy after administering the BCMA targeted CAR T cell therapy is initiated at no later than 6 months after administering the BCMA targeted CAR T cell therapy.

77. The method of any of claims 42-76, wherein the immunomodulatory agent maintenance therapy after administering the BCMA targeted CAR T cell therapy is initiated at no later than about 5 months after administering the BCMA targeted CAR T cell therapy.

78. The method of any of claims 42-77, wherein the immunomodulatory agent maintenance therapy after administering the BCMA targeted CAR T cell therapy is initiated at no later than about 4 months after administering the BCMA targeted CAR T cell therapy.

79. The method of any of claims 42-78, wherein the immunomodulatory agent maintenance therapy after administering the BCMA targeted CAR T cell therapy is initiated at no later than about 3 months after administering the BCMA targeted CAR T cell therapy.

80. The method of any of claims 42-79, wherein the immunomodulatory agent maintenance therapy after administering the BCMA targeted CAR T cell therapy is initiated at no later than about 2 months after administering the BCMA targeted CAR T cell therapy.

81. The method of any of claims 42-80, wherein the immunomodulatory agent maintenance therapy after administering the BCMA targeted CAR T cell therapy is initiated at no later than about 1 month after administering the BCMA targeted CAR T cell therapy.

82. The method of any of claims 42-81, wherein the immunomodulatory agent maintenance therapy after administering the BCMA targeted CAR T cell therapy is initiated at about 1 month after administering the BCMA targeted CAR T cell therapy.

83. The method of any of claims 1-71, wherein the immunomodulatory agent maintenance therapy is initiated at Day 15 or later after administering the BCMA targeted CAR T cell therapy.

84. The method of any of claim 1-71 or 83, wherein the immunomodulatory agent maintenance therapy is initiated at 1 month or later after administering the BCMA targeted CAR T cell therapy.

85. The method of any of claim 1-71 or 83-84, wherein the immunomodulatory agent maintenance therapy is initiated at at least 1 month after administering the BCMA targeted CAR T cell therapy.

86. The method of any of claim 1-71 or 83-85, wherein the immunomodulatory agent maintenance therapy is initiated at at least 2 months after administering the BCMA targeted CAR T cell therapy.

87. The method of any of claim 1-71 or 83-86, wherein the immunomodulatory agent maintenance therapy is initiated at at least 3 months after administering the BCMA targeted CAR T cell therapy.

88. The method of any of claim 1-71 or 83-87, wherein the immunomodulatory agent maintenance therapy is initiated at at least 4 months after administering the BCMA targeted CAR T cell therapy.

89. The method of any of claim 1-71 or 83-88, wherein the immunomodulatory agent maintenance therapy is initiated at no later than 24 months after administering the BCMA targeted CAR T cell therapy.

90. The method of any of claim 1-71 or 83-89, wherein the immunomodulatory agent is initiated at or between Day 15 and Day 730 after administering the BCMA targeted CAR T cell therapy.

91. The method of any of claim 1-71 or 83-90, wherein the immunomodulatory agent maintenance therapy is initiated at or between 1 month and 24 months, 1 month and 12 months, 1 month and 10 months, 1 month and 6 months, or 1 month and 4 months after administering the BCMA targeted CAR T cell therapy.

92. The method of any of claim 1-71 or 83-91, wherein the immunomodulatory agent maintenance therapy is initiated at or between 1 month and 6 months after administering the BCMA targeted CAR T cell therapy.

93. The method of any of claim 1-71 or 83-91, wherein the immunomodulatory agent maintenance therapy is initiated at or between 4 months and 24 months, 4 months and 12 months, or 4 months and 10 months after administering the BCMA targeted CAR T cell therapy.

94. The method of any of claim 1-71, 83-91 or 93, wherein the immunomodulatory agent maintenance therapy is initiated at or between 4 month and 10 months, after administering the BCMA targeted CAR T cell therapy.

95. The method of any of claim 1-71 or 83-91, wherein the immunomodulatory agent maintenance therapy is initiated at or between Day 30 and Day 180 after administering the BCMA targeted CAR T cell therapy.

96. The method of any of claim 1-85, 90 or 95, wherein the immunomodulatory agent maintenance therapy is initiated at 1 month after administering the BCMA targeted CAR T cell therapy.

97. The method of any of claims 1-96, wherein the immunomodulatory agent maintenance therapy continues until disease progression.

98. The method of any of claims 1-96, wherein the immunomodulatory agent maintenance therapy continues until the subject achieves complete response (CR).

99. The method of any of claims 1-98, wherein the immunomodulatory agent of the immunomodulatory agent maintenance therapy is a compound that interacts with and/or binds to cereblon (CRBN) or one or more members of the CRBN E3 ubiquitin-ligase complex; an inhibitor of Ikaros (IKZF1); an inhibitor of Aiolos (IKZF3); or a compound that enhances or promote ubiquitination and/or degradation of Ikaros (IKZF1) and Aiolos (IKZF3).

100. The method of any of claims 1-99, wherein the immunomodulatory agent of the immunomodulatory agent maintenance therapy is thalidomide, a thalidomide analog or a thalidomide derivative.

101. The method of any of claims 1-100, wherein the immunomodulatory agent of the immunomodulatory agent maintenance therapy is lenalidomide or a pharmaceutically acceptable salt, pomalidomide, avadomide, iberdomide, CC-92480 or CC-885.

102. The method of any of claims 1-101, wherein the immunomodulatory agent of the immunomodulatory agent maintenance therapy is lenalidomide or a pharmaceutically acceptable salt.

103. The method of any of claims 1-102, wherein the immunomodulatory agent of the immunomodulatory agent maintenance therapy is administered in an amount of 0.5 mg per day to 50 mg per day.

104. The method of any of claims 1-103, wherein the immunomodulatory agent of the immunomodulatory agent maintenance therapy is administered in an amount of 0.5 mg per day to 25 mg per day, 0.5 mg per day to 10 mg per day, 0.5 mg per day to 5 mg per day, 0.5 mg per day to 2.5 mg per day, 0.5 mg per day to 1 mg per day, 1 mg per day to 50 mg per day, 1 mg per day to 25 mg per day, 1 mg per day to 10 mg per day, 1 mg per day to 5 mg per day, 1 mg per day to 2.5 mg per day, 2.5 mg per day to 50 mg per day, 2.5 mg per day to 25 mg per day, 2.5 mg per day to 10 mg per day, 2.5 mg per day to 5 mg per day, 5 mg per day to 50 mg per day, 5 mg per day to 25 mg per day, 5 mg per day to 10 mg per day, 10 mg per day to 50 mg per day, 10 mg per day to 25 mg per day or 25 mg per day to 50 mg per day.

105. The method of any of claims 1-104, wherein the immunomodulatory agent of the immunomodulatory agent maintenance therapy is administered in an amount of 2.5 mg per day to 25 mg per day.

106. The method of any of claims 1-104, wherein the immunomodulatory agent of the immunomodulatory agent maintenance therapy is administered in an amount of 2.5 mg per day to 10 mg per day.

107. The method of any of claims 1-104, wherein the immunomodulatory agent of the immunomodulatory agent maintenance therapy is administered in an amount of at or about 1 mg per day.

108. The method of any of claims 1-106, wherein the immunomodulatory agent of the immunomodulatory agent maintenance therapy is administered in an amount of at or about 2.5 mg per day.

109. The method of any of claims 1-106, wherein the immunomodulatory agent of the immunomodulatory agent maintenance therapy is administered in an amount of at or about 5 mg per day.

110. The method of any of claims 1-106, wherein the immunomodulatory agent of the immunomodulatory agent maintenance therapy is administered in an amount of at or about 10 mg per day.

111. The method of any of claims 1-105, wherein the immunomodulatory agent of the immunomodulatory agent maintenance therapy is administered in an amount of at or about 15 mg per day.

112. The method of any of claims 103-111, wherein the amount of the immunomodulatory agent of the immunomodulatory agent maintenance therapy is administered daily for 14 days in a 28-day cycle.

113. The method of any of claims 103-111, wherein the amount of the immunomodulatory agent of the immunomodulatory agent maintenance therapy is administered daily for 21 days in a 28-day cycle.

114. The method of any of claims 103-111, wherein the amount of the immunomodulatory agent of the immunomodulatory agent maintenance therapy is administered daily for 28 days in a 28-day cycle.

115. The method of any one of claims 1-114, wherein prior to the administration of the BCMA targeted CAR T cell therapy to the subject, the subject has received a lymphodepleting therapy comprising the administration of fludarabine at or about 20-40 mg/m² body surface area of the subject (optionally at or about 30 mg/m²) daily for 2-4 days, and/or cyclophosphamide at or about 200-400 mg/m² body surface area of the subject (optionally at or about 300 mg/m²) daily for 2-4 days.

116. The method of any one of claims 1-115, wherein prior to the administration of the BCMA targeted CAR T cell therapy to the subject, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

117. The method of any one of claims 1-116, wherein the BCMA targeted CAR T cell therapy comprises a chimeric antigen receptor (CAR) comprising an extracellular antigen-binding domain that binds to BCMA, a transmembrane domain, and an intracellular signaling region.

118. The method of claim 117, wherein the extracellular antigen-binding domain comprises a variable heavy chain (VH) region and a variable light chain (VL) region.

119. The method of claim 118, wherein:
the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or
the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.

120. The method of claim 118 or claim 119, wherein:
the VH region comprises an amino acid sequence set forth in SEQ ID NO: 18 and the VL region comprises the amino acid sequence set forth in SEQ ID NO: 19;
or the VH region comprises an amino acid sequence set forth in SEQ ID NO: 24, and the VL region comprises the amino acid sequence set forth in SEQ ID NO: 25.

121. The method of any one of claims 117-120, wherein the extracellular antigen-binding domain is a single chain variable fragment (scFv).

122. The method of claim 121, wherein the scFv comprises the amino acid sequence set forth in SEQ ID NO: 213 or SEQ ID NO: 188.

123. The method of any one of claims 117-120, wherein the intracellular signaling region further comprises a costimulatory signaling domain.

124. The method of claim 123, wherein the costimulatory signaling domain comprises an intracellular signaling domain of CD28, 4-1BB, or ICOS, or a signaling portion thereof.

125. The method of claim 123 or claim 124, wherein the costimulatory signaling domain is between the transmembrane domain and the cytoplasmic signaling domain of a CD3-zeta (CD3ζ) chain.

126. The method of any one of claims 117-125, wherein the transmembrane domain is or comprises a transmembrane domain from CD28 or CD8, optionally human CD28 or CD8.

127. The method of any one of claims 117-126, wherein the CAR further comprises an extracellular spacer between the antigen binding domain and the transmembrane domain.

128. The method of claim 127, wherein the spacer is from CD8, optionally wherein the spacer is a CD8a hinge.

129. The method of claim 127 or claim 128, wherein the transmembrane domain and the spacer are from CD8.

130. The method of any one of claims 117-129, wherein the CAR comprises the amino acid sequence set forth in SEQ ID NO: 116 or SEQ ID NO: 124.

131. The method of any one of claims 117-120, wherein the CAR is encoded by the polynucleotide sequence set forth in SEQ ID NO: 214.

132. The method of any of claims 1-131, wherein the BCMA targeted CAR T cell therapy comprises CD3+ CAR-expressing T cells.

133. The method of any of claims 1-132, wherein the BCMA targeted CAR T cell therapy comprises a combination of CD4+ T cells and CD8+ T cells and/or a combination of CD4+ CAR-expressing T cells and CD8+ CAR-expressing T cells.

134. The method of claim 133, wherein the ratio of CD4+ CAR-expressing T cells to CD8+ CAR-expressing T cells and/or of CD4+ T cells to CD8+ T cells, is or is approximately 1:1 or is between at or approximately 1:3 and at or approximately 3:1.

135. The method of any of claims 1-134, wherein:
the percentage of naïve-like T cells and/or central memory T cells is greater than or greater than about 60% of the total genetically engineered T cells in the dose, optionally greater than or greater than about 65%, 70%, 80%, 90% or 95%;
the percentage of naïve-like T cells and/or central memory T cells is greater than or greater than about 40% of the total CD4+ genetically engineered T cells in the dose, optionally greater than or greater than about 50%, 60%, 70%, 80%, 90% or 95%; or
the percentage of naïve-like T cells and/or central memory T cells is greater than or greater than about 40% of the total CD8+ genetically engineered T cells in the dose, optionally greater than or greater than about 50%, 60%, 70%, 80%, 90% or 95%.

136. The method of claim 135, wherein the naïve-like T cells are CCR7+CD45RA+, CD27+CCR7+, or CD62L-CCR7+.

137. The method of any one of claims 1-136, wherein the BCMA targeted CAR T cell therapy is: idecabtagene vicleucel; bb21217 cells; orvacabtagene autoleucel; CT103A; ciltacabtagene autoleucel; KITE585; CT053; BCMA-CS1 cCAR (BC1cCAR); P-BCMA-101; P-BCMA-ALLO1; C-CAR088; Descartes-08; PBCAR269A; ALLO-715; PHE885; AUTO8; CTX120; CB-011; ALLO-605 (TuboCAR/MM); pCDCAR1 (TriCAR-Z136), or GC012F.

138. The method of any one of claims 1-137, wherein the BCMA targeted CAR T cell therapy is idecabtagene vicleucel.

139. The method of any one of claims 1-138, wherein the dose of the BCMA targeted CAR T cell therapy is between about 100×10⁶ CAR-positive T cells and about 600×10⁶ CAR-positive T cells.

140. The method of any one of claims 1-139, wherein the dose of the BCMA targeted CAR T cell therapy is between about 150×10⁶ and about 540×10⁶ CAR-positive T cells.

141. The method of any one of claims 1-140, wherein the dose of the BCMA targeted CAR T cell therapy is between about 150×10⁶ and about 450×10⁶ CAR-positive T cells.

142. The method of any one of claims 1-139, wherein the dose of the BCMA targeted CAR T cell therapy is between about 300×10⁶ and about 540×10⁶ CAR-positive T cells.

143. The method of any one of claim 1-139 or 142, wherein the dose of the BCMA targeted CAR T cell therapy is between about 300×10⁶ and about 460×10⁶ CAR-positive T cells.

144. The method of any one of claim 1-139 or 142, wherein the dose of the BCMA targeted CAR T cell therapy is between about 300×10⁶ and about 450×10⁶ CAR-positive T cells.

145. The method of any one of claims 1-140, wherein the dose of the BCMA targeted CAR T cell therapy is about 150×10⁶, about 300×10⁶, about 450×10⁶, about 460×10⁶, or about 540×10⁶ CAR-positive T cells.

146. The method of any one of claim 1-140 or 142, wherein the dose of the BCMA targeted CAR T cell therapy is about 300×10⁶, about 450×10⁶, about 460×10⁶, or about 540×10⁶ CAR-positive T cells.

147. The method of any one of claims 1-146, wherein the T cells of the BCMA targeted CAR T cell therapy were obtained from the subject, optionally by apheresis or leukapheresis, and the T cells were engineered ex vivo with the BCMA targeted CAR.

148. The method of any one of claims 1-147, wherein the dose of the BCMA targeted CAR T cell therapy is autologous to the subject.

149. The method of any one of claims 1-147, wherein the dose of the BCMA targeted CAR T cell therapy is allogeneic to the subject.

150. The method of any of claims 1-149, wherein the subject received a bridging therapy prior to receiving the BCMA targeted CAR T cell therapy, optionally wherein the bridging therapy was administered to the subject in the period between obtaining the T cells from the subject and administering the BCMA targeted CAR T cell therapy to the subject.

151. The method of any of claims 1-149, further comprising administering to the subject a bridging therapy prior to administering the BCMA targeted CAR T cell therapy to the subject, optionally wherein the bridging therapy is administered to the subject in the period between obtaining the T cells from the subject and administering the BCMA targeted CAR T cell therapy to the subject.

152. The method of claim 150 or claim 151, wherein the bridging therapy comprises one or more of corticosteroids, alkylating agents, immunomodulatory agents, proteasome inhibitors (PI), or anti-CD38 antibodies.

153. The method of any of claims 1-152, wherein the subject is human.

154. The method of any one of claims 1-153, wherein the subject is 18 year of age or older.
